(19) United States
(12) Patent Application Publication
(10) Pub. No.: US 2023/0037538 A1
(43) Pub. Date: Feb. 9, 2023

(54) ANTI-ALS COMPOUNDS

(71) Applicant: Takeda Pharmaceutical Company Limited, Osaka (JP)

(72) Inventors: Dennis Solas, San Francisco, CA (US); Anatoliy Kitaygorodskyy, San Francisco, CA (US); Kumar Paulvannan, San Jose, CA (US); Vishwanath R. Lingappa, San Francisco, CA (US); Masato Yoshikawa, Fujisawa-shi (JP); Masahiro Ito, Fujisawa-shi (JP); Yuta Tanaka, Fujisawa-shi (JP); Keiko Kakegawa, Fujisawa-shi (JP); Tomohiro Ohashi, Fujisawa-shi (JP); Takuto Kojima, Fujisawa-shi (JP); Akinori Toita, Fujisawa-shi (JP); Osamu Kubo, Fujisawa-shi (JP); Fumiaki Kikuchi, Fujisawa-shi (JP); Florian Pünner, Fujisawa-shi (JP); Junsi Wang, Fujisawa-shi (JP)

(21) Appl. No.: 17/847,456

(22) Filed: Jun. 23, 2022

Related U.S. Application Data
(60) Provisional application No. 63/214,395, filed on Jun. 24, 2021.

(51) Int. Cl. C07D 217/20; C07D 405/06; C07D 471/04; C07D 401/10; C07D 405/10; C07D 413/10; C07D 405/12; C07D 401/12; A61P 25/28

(52) U.S. Cl. CPC C07D 217/20; C07D 405/06; C07D 471/04; C07D 401/10; C07D 405/10; C07D 413/10; C07D 405/12; C07D 401/12; A61P 25/28

(57) ABSTRACT

The present invention relates to compounds expected to be useful in the prevention and/or treatment of diseases such as amyotrophic lateral sclerosis, frontotemporal dementia, chronic traumatic encephalopathy, Alzheimer's disease, frontotemporal lobar degeneration, multisystem proteinopathy and the like, a method for preventing or treating such diseases, and the like.

### TECHNICAL FIELD

The present invention relates to compounds expected to be useful in the prevention and/or treatment of diseases such as amyotrophic lateral sclerosis, frontotemporal dementia, chronic traumatic encephalopathy, Alzheimer's disease, frontotemporal lobar degeneration, multisystem proteinopathy and the like, a method for preventing or treating such diseases, and the like.

### BACKGROUND OF THE INVENTION

There is a need in the art for compounds useful in the prevention and/or treatment of diseases such as amyotrophic lateral sclerosis, frontotemporal dementia, chronic traumatic encephalopathy, Alzheimer's disease, frontotemporal lobar degeneration, multisystem proteinopathy and the like.

Patent Document 1 and Patent Document 2 describe tetrahydroisoquinoline compounds having anti-HIV action, represented by the following general formula:

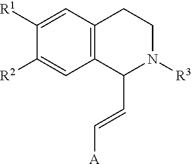

wherein each symbol is as described in the documents.

### Document List

### Patent Document

Patent Document 1: U.S. Pat. No. 9,518,022 B2
Patent Document 2: U.S. Ser. No. 10/195,189 B2

### SUMMARY OF THE INVENTION

### Problems to be Solved by the Invention

The present invention aims to provide compounds expected to be useful in the prevention and/or treatment of diseases such as amyotrophic lateral sclerosis, frontotemporal dementia, chronic traumatic encephalopathy, Alzheimer's disease, frontotemporal lobar degeneration, multisystem proteinopathy and the like.

### Means of Solving the Problems

The present inventors have conducted intensive studies to solve the above-mentioned problems, and have found that a compound represented by the following formula (I) (including a compound represented by the following formula (Ia) and a compound represented by the following formula (Ib)) is surprisingly effective against diseases such as amyotrophic lateral sclerosis, frontotemporal dementia, chronic traumatic encephalopathy, Alzheimer's disease, frontotemporal lobar degeneration, multisystem proteinopathy and the like, and completed the present invention based on these findings.

Accordingly, the present invention provides the following.

[1] A method for preventing and/or treating amyotrophic lateral sclerosis, frontotemporal dementia, chronic traumatic encephalopathy, Alzheimer's disease, frontotemporal lobar degeneration, or multisystem proteinopathy in a mammal, which comprises administering an effective amount of a compound represented by the formula (I):

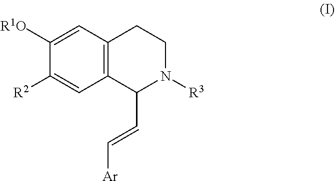

wherein
R¹ is unsubstituted C₁₋₆ alkyl, substituted or unsubstituted phenyl substituted C₁₋₆ alkyl, substituted or unsubstituted C₃₋₁₀ cycloalkyl substituted C₁₋₆ alkyl, or substituted or unsubstituted pyridyl substituted C₁₋₆ alkyl;
R² is H, CF₃, unsubstituted C₁₋₆ alkoxy, or phenyl substituted C₁₋₆ alkoxy;
R³ is H, —C(O)OR⁴, or —C(O)R⁴ or —C(O)NR⁴R⁵ wherein R⁴ and R⁵ are independently selected from unsubstituted C₁₋₆ alkyl, unsubstituted phenyl, and unsubstituted pyridyl; and Ar is substituted or unsubstituted phenyl, substituted or unsubstituted benzofuryl, substituted or unsubstituted pyrrolopyridyl, substituted or unsubstituted imidazopyrimidinyl, substituted or unsubstituted imidazopyrazinyl, substituted or unsubstituted imidazopyridazinyl, or substituted or unsubstituted imidazopyridyl;
or a hydrate, solvate, or salt thereof (hereinafter, also to be referred to as compound (I)), to the mammal.
[1a] The method of the above [1], wherein the compound, or a hydrate, solvate, or salt thereof is part of a pharmaceutical composition, and said pharmaceutical composition further comprises a pharmaceutically acceptable carrier.
[1b] The method of the above [1], wherein the disease is amyotrophic lateral sclerosis.
[1c] The method of the above [1], wherein the disease is frontotemporal dementia.
[1d] The method of the above [1], wherein the mammal is a human.
[2] A compound represented by the formula (IA):

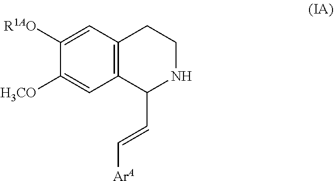

wherein
R^{1A} is a C₁₋₆ alkyl group substituted by phenyl group(s) optionally substituted by 1 to 3 halogen atoms; and Ar^{A} is
(1) a phenyl group substituted by 2 or 3 substituents selected from
(a) a C₁₋₆ alkyl group,
(b) a C₁₋₆ alkoxy group optionally substituted by 1 to 3 of 5- or 6-membered monocyclic aromatic heterocyclic groups optionally substituted by 1 to 3 halogen atoms, and
(c) a 5- or 6-membered monocyclic aromatic heterocyclic group optionally substituted by 1 to 3 C₁₋₆ alkyl groups, or
   (2) a pyrrolopyridyl group substituted by 2 substituents selected from
(a) a C₁₋₆ alkyl group, and
(b) a C₁₋₆ alkoxy group,
   or a hydrate, solvate, or salt thereof (hereinafter, also to be referred to as compound (IA)).
   [3] A compound represented by the formula (IB):

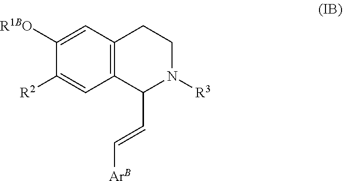

wherein
R^{1B} is unsubstituted C₁₋₆ alkyl, or substituted or unsubstituted phenyl substituted C₁₋₆ alkyl;
R² is H, CF₃, unsubstituted C₁₋₆ alkoxy, or phenyl substituted C₁₋₆ alkoxy;
R³ is H, —C(O)OR⁴, or —C(O)R⁴ or —C(O)NR⁴R⁵ wherein R⁴ and R⁵ are independently selected from unsubstituted C₁₋₆ alkyl, unsubstituted phenyl, and unsubstituted pyridyl; and
Ar^{B} is substituted or unsubstituted imidazo[1,2-a]pyrimidinyl, substituted or unsubstituted imidazo[1,2-a]pyrazinyl, substituted or unsubstituted imidazo[1,2-b]pyridazinyl, or substituted or unsubstituted imidazo[1,2-a]pyridyl;
or a hydrate, solvate, or salt thereof (hereinafter, also to be referred to as compound (IB)).
[3a] The compound of the above [3], wherein Ar^{B} is substituted or unsubstituted imidazo[1,2-a]pyrazinyl, or a hydrate, solvate, or salt thereof.
[3b] The compound of the above [3], wherein Ar^{B} is substituted or unsubstituted imidazo[1,2-b]pyridazinyl, or a hydrate, solvate, or salt thereof.
[3c] The compound of the above [3b], which is

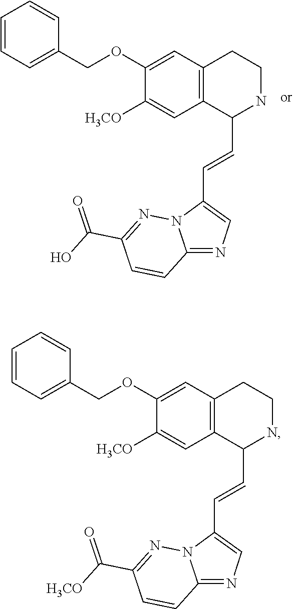

or a hydrate, solvate, or salt thereof.
[3d] The compound of the above [3], wherein Ar^{B} is substituted or unsubstituted imidazo[1,2-a]pyridyl, or a hydrate, solvate, or salt thereof.
[4] A method for preventing and/or treating amyotrophic lateral sclerosis, frontotemporal dementia, chronic traumatic encephalopathy, Alzheimer's disease, frontotemporal lobar degeneration, or multisystem proteinopathy in a mammal, which comprises administering an effective amount of the compound of the above [2], or a hydrate, solvate, or salt thereof, or the compound of the above [3], or a hydrate, solvate, or salt thereof, to the mammal.
[4a] The method of the above [4], which comprises administering an effective amount of the compound of the above [2], or a hydrate, solvate, or salt thereof, to the mammal.
[4b] The method of the above [4], which comprises administering an effective amount of the compound of the above [3], or a hydrate, solvate, or salt thereof, to the mammal.
[5] A pharmaceutical composition comprising the compound of the above [2], or a hydrate, solvate, or salt thereof, and a pharmaceutically acceptable carrier.
[5a] The composition of the above [5], for preventing and/or treating amyotrophic lateral sclerosis, frontotemporal dementia, chronic traumatic encephalopathy, Alzheimer's disease, frontotemporal lobar degeneration, or multisystem proteinopathy in a mammal.
[6] A pharmaceutical composition comprising the compound of the above [3], or a hydrate, solvate, or salt thereof, and a pharmaceutically acceptable carrier.
[6a] The composition of the above [6], for preventing and/or treating amyotrophic lateral sclerosis, frontotemporal dementia, chronic traumatic encephalopathy, Alzheimer's disease, frontotemporal lobar degeneration, or multisystem proteinopathy in a mammal.
[7] A compound represented by the formula (I):

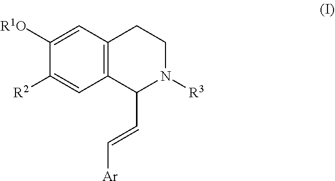

wherein
R¹ is unsubstituted C₁₋₆ alkyl, substituted or unsubstituted phenyl substituted C₁₋₆ alkyl, substituted or unsubstituted C₃₋₁₀ cycloalkyl substituted C₁₋₆ alkyl, or substituted or unsubstituted pyridyl substituted C₁₋₆ alkyl;
R² is H, CF₃, unsubstituted C₁₋₆ alkoxy, or phenyl substituted C₁₋₆ alkoxy;
R³ is H, —C(O)OR⁴, or —C(O)R⁴ or —C(O)NR⁴R⁵ wherein R⁴ and R⁵ are independently selected from unsubstituted C₁₋₆ alkyl, unsubstituted phenyl, and unsubstituted pyridyl; and
Ar is substituted or unsubstituted phenyl, substituted or unsubstituted benzofuryl, substituted or unsubstituted pyrrolopyridyl, substituted or unsubstituted imidazopyrimidinyl, substituted or unsubstituted imidazopyrazinyl, substituted or unsubstituted imidazopyridazinyl, or substituted or unsubstituted imidazopyridyl;
or a hydrate, solvate, or salt thereof, for use in the prevention and/or treatment of amyotrophic lateral sclerosis, frontotemporal dementia, chronic traumatic encephalopathy, Alzheimer's disease, frontotemporal lobar degeneration, or multisystem proteinopathy in a mammal.
[8] The compound of the above [2], or a hydrate, solvate, or salt thereof, for use in the prevention and/or treatment of amyotrophic lateral sclerosis, frontotemporal dementia, chronic traumatic encephalopathy, Alzheimer's disease, frontotemporal lobar degeneration, or multisystem proteinopathy in a mammal.
[9] A method for reducing stress granules and/or preventing the formation of stress granules in a neuron of a mammal, which comprises contacting the neuron of the mammal with a compound represented by the formula (I):

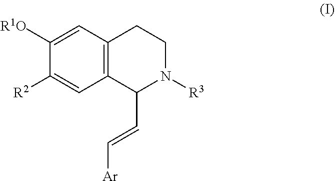

wherein
R¹ is unsubstituted C₁₋₆ alkyl, substituted or unsubstituted phenyl substituted C₁₋₆ alkyl, substituted or unsubstituted C₃₋₁₀ cycloalkyl substituted C₁₋₆ alkyl, or substituted or unsubstituted pyridyl substituted C₁₋₆ alkyl;
R² is H, CF₃, unsubstituted C₁₋₆ alkoxy, or phenyl substituted C₁₋₆ alkoxy;
R³ is H, —C(O)OR⁴, or —C(O)R⁴ or —C(O)NR⁴R⁵ wherein R⁴ and R⁵ are independently selected from unsubstituted C₁₋₆ alkyl, unsubstituted phenyl, and unsubstituted pyridyl; and
Ar is substituted or unsubstituted phenyl, substituted or unsubstituted benzofuryl, substituted or unsubstituted pyrrolopyridyl, substituted or unsubstituted imidazopyrimidinyl, substituted or unsubstituted imidazopyrazinyl, substituted or unsubstituted imidazopyridazinyl, or substituted or unsubstituted imidazopyridyl;
or a hydrate, solvate, or salt thereof.
[10] A pharmaceutical composition for use in the prevention and/or treatment of amyotrophic lateral sclerosis, frontotemporal dementia, chronic traumatic encephalopathy, Alzheimer's disease, frontotemporal lobar degeneration, or multisystem proteinopathy in a mammal, which comprises an effective amount of a compound represented by the formula (I):

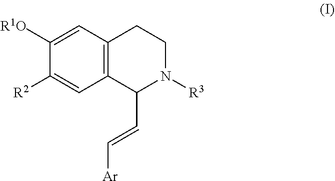

wherein
R¹ is unsubstituted C₁₋₆ alkyl, substituted or unsubstituted phenyl substituted C₁₋₆ alkyl, substituted or unsubstituted C₃₋₁₀ cycloalkyl substituted C₁₋₆ alkyl, or substituted or unsubstituted pyridyl substituted C₁₋₆ alkyl;
R² is H, CF₃, unsubstituted C₁₋₆ alkoxy, or phenyl substituted C₁₋₆ alkoxy;
R³ is H, —C(O)OR⁴, or —C(O)R⁴ or —C(O)NR⁴R⁵ wherein R⁴ and R⁵ are independently selected from unsubstituted C₁₋₆ alkyl, unsubstituted phenyl, and unsubstituted pyridyl; and
Ar is substituted or unsubstituted phenyl, substituted or unsubstituted benzofuryl, substituted or unsubstituted pyrrolopyridyl, substituted or unsubstituted imidazopyrimidinyl, substituted or unsubstituted imidazopyrazinyl, substituted or unsubstituted imidazopyridazinyl, or substituted or unsubstituted imidazopyridyl;
or a hydrate, solvate, or salt thereof, and pharmaceutically acceptable carrier.
[11] A compound selected from a group of;
6-(benzyloxy)-7-methoxy-1-[(E)-2-(5-methoxy-1-methyl-1H-pyrrolo[2,3-b]pyridin-3-yl)ethenyl]-1,2,3,4-tetrahydroisoquinoline;
6-(benzyloxy)-7-methoxy-1-{(E)-2-[2-methyl-5-(pyrimidin-5-yl)phenyl]ethenyl}-1,2,3,4-tetrahydroisoquinoline;
6-[(3-chlorophenyl)methoxy]-7-methoxy-1-[(E)-2-(5-methoxy-1-methyl-1H-pyrrolo[2,3-b]pyridin-3-yl)ethenyl]-1,2,3,4-tetrahydroisoquinoline;
6-(benzyloxy)-7-methoxy-1-[(E)-2-{4-methoxy-2-methyl-5-[(pyridin-4-yl)methoxy]phenyl}ethenyl]-1,2,3,4-tetrahydroisoquinoline;
6-(benzyloxy)-7-methoxy-1-{(E)-2-[2-methyl-4-(1-methyl-1H-pyrazol-4-yl)phenyl]ethenyl}-1,2,3,4-tetrahydroisoquinoline; and
6-(benzyloxy)-7-methoxy-1-[(E)-2-{4-methoxy-5-[(6-methoxypyridin-2-yl)methoxy]-2-methylphenyl}ethenyl]-1,2,3,4-tetrahydroisoquinoline,
   or a hydrate, solvate, or salt thereof.
   [11a] A method for preventing and/or treating amyotrophic lateral sclerosis, frontotemporal dementia, chronic traumatic encephalopathy, Alzheimer's disease, frontotemporal lobar degeneration, or multisystem proteinopathy in a mammal, which comprises administering an effective amount of the compound of above [11], or a hydrate, solvate, or salt thereof, to the mammal.

### Effect of the Invention

According to the present invention, compounds expected to be useful in the prevention and/or treatment of diseases such as amyotrophic lateral sclerosis, frontotemporal dementia, chronic traumatic encephalopathy, Alzheimer's disease, frontotemporal lobar degeneration, multisystem proteinopathy and the like, can be provided.

### DETAILED DESCRIPTION OF THE INVENTION

### I. Definitions and Abbreviations

As used herein, the singular forms “a,” “an”, and “the” include plural references unless the context clearly dictates otherwise. For example, reference to “an active agent” includes a single active agent as well as two or more different active agents in combination. It is to be understood that present teaching is not limited to the specific dosage forms, carriers, or the like, disclosed herein and as such may vary.

The abbreviations used herein generally have their conventional meaning within the chemical and biological arts.

The following abbreviations have been used: Ac is acetyl; AcOH is acetic acid; ACTBr is cetyltrimethylammonium bromide; AIBN is azobisisobutyronitrile or 2,2 azobisisobutyronitrile; aq. is aqueous; Ar is aryl; B₂pin₂ is bis(pinacolato)diboron; Bn is, in general, benzyl [see Cbz for one example of an exception]; (BnS)₂ is benzyl disulfide; BnSH is benzyl thiol or benzyl mercaptan; BnBr is benzyl bromide; Boc is tert-butoxycarbonyl; Boc₂O is di-tert-butyl dicarbonate; Bz is, in general, benzoyl; BzOOH is benzoyl peroxide; Cbz or Z is benzyloxycarbonyl or carboxybenzyl; Cs₂CO₃ is cesium carbonate; CSA is camphor sulfonic acid; CTAB is cetyltrimethylammonium bromide; Cy is cyclohexyl; DABCO is 1,4-diazabicyclo[2.2.2]octane; DCM is dichloromethane or methylene chloride; DHP is dihydropyran; DIAD is diisopropyl azodicarboxylate; DIEA or DIPEA is N,N-diisopropylethylamine; DMAP is 4-(dimethylamino)pyridine; DME is 1,2-dimethoxyethane; DMF is N,N-dimethylformamide; DMSO is dimethyl sulfoxide; equiv or eq. is equivalent; EtOAc is ethyl acetate; EtOH is ethanol; Et₂O is diethyl ether; EDCI is N-(3-dimethylaminopropyl)-N′-ethylcarbodiimide hydrochloride; ELS is evaporative light scattering; equiv or eq is equivalent; h is hours; HATU is O-(7-azabenzotriazol-1-yl)-N,N,N′N′-tetramethyluronium hexafluorophosphate; HOBt is N-hydroxybenzotriazole; HCl is hydrochloric acid; HPLC is high pressure liquid chromatography; ISCO Companion is automated flash chromatography equipment with fraction analysis by UV absorption available from Presearch; KOAc or AcOK is potassium acetate; K₂CO₃ is potassium carbonate; LiAlH₄ or LAH is lithium aluminum hydride; LDA is lithium diisopropylamide; LHMDS is lithium bis(trimethylsilyl) amide; KHMDS is potassium bis(trimethylsilyl) amide; LiOH is lithium hydroxide; m-CPBA is 3-chloroperoxybenzoic acid; MeCN or ACN is methyl cyanide or cyanomethane or ethanenitrile or acetonitrile which are all names for the same compound; MeOH is methanol; MgSO₄ is magnesium sulfate; mins or min is minutes; Mp or MP is melting point; NaCNBH₃ is sodium cyanoborohydride; NaOH is sodium hydroxide; Na₂SO₄ is sodium sulfate; NBS is N-bromosuccinimide; NH₄Cl is ammonium chloride; NIS is N-iodosuccinimide; N₂ is nitrogen; NMM is N-methylmorpholine; n-BuLi is n-butyllithium; overnight is O/N; PdCl₂(pddf) is 1,1′-Bis(diphenylphosphino) ferrocene]dichloropalladium(II); Pd/C is the catalyst known as palladium on carbon; Pd₂(dba)₃ is an organometallic catalyst known as tris(dibenzylideneacetone) dipalladium(0); Ra Ni or Raney Ni is Raney nickel; Ph is phenyl; PMB is p-methoxybenzyl; PrOH is 1-propanol; iPrOH is 2-propanol; POCl₃ is phosphorus chloride oxide; PTSA is para-toluene sulfonic acid; Pyr. or Pyr or Py as used herein means Pyridine; RT or rt or r.t. is room temperature; sat. is saturated; Si-amine or Si—NH₂ is amino-functionalized silica, available from SiliCycle; Si-pyr is pyridyl-functionalized silica, available from SiliCycle; TEA or Et₃N is triethylamine; TFA is trifluoroacetic acid; Tf₂O is trifluoromethanesulfonic anhydride; THF is tetrahydrofuran; TFAA is trifluoroacetic anhydride; THP is tetrahydropyranyl; TMSI is trimethylsilyl iodide; H₂O is water; diNO₂PhSO₂Cl is dinitrophenyl sulfonyl chloride; 3-F-4-NO₂-PhSO₂Cl is 3-fluoro-4-nitrophenylsulfonyl chloride; 2-MeO-4-NO₂-PhSO₂Cl is 2-methoxy-4-nitrophenylsulfonyl chloride; and (EtO)₂POCH₂COOEt is a triethylester of phosphonoacetic acid known as triethyl phosphonoacetate.

“Compound of the invention” as used herein refers to the compounds discussed herein, salts (e.g., pharmaceutically acceptable salts), prodrugs, solvates and hydrates of these compounds.

Where substituent groups are specified by their conventional chemical formulae, written from left to right, they equally encompass the chemically identical substituents, which would result from writing the structure from right to left, e.g., —CH₂O— is intended to also recite —OCH₂—.

The term “poly” as used herein means at least 2. For example, a polyvalent metal ion is a metal ion having a valency of at least 2.

“Moiety” refers to a radical of a molecule that is attached to the remainder of the molecule.

The symbol [symbol] , whether utilized as a bond or displayed perpendicular to a bond, indicates the point at which the displayed moiety is attached to the remainder of the molecule.

The definition of each substituent used in the present specification is described in detail in the following. Unless otherwise specified, each substituent has the following definition.

In the present specification, examples of the “halogen atom” include fluorine, chlorine, bromine and iodine.

In the present specification, examples of the “C₁₋₆ alkyl group” include methyl, ethyl, propyl, isopropyl, butyl, isobutyl, sec-butyl, tert-butyl, pentyl, isopentyl, neopentyl, 1-ethylpropyl, hexyl, isohexyl, 1,1-dimethylbutyl, 2,2-dimethylbutyl, 3,3-dimethylbutyl and 2-ethylbutyl.

In the present specification, examples of the “optionally halogenated C₁₋₆ alkyl group” include a C₁₋₆ alkyl group optionally having 1 to 7, preferably 1 to 5, halogen atoms. Specific examples thereof include methyl, chloromethyl, difluoromethyl, trichloromethyl, trifluoromethyl, ethyl, 2-bromoethyl, 2,2,2-trifluoroethyl, tetrafluoroethyl, pentafluoroethyl, propyl, 2,2-difluoropropyl, 3,3,3-trifluoropropyl, isopropyl, butyl, 4,4,4-trifluorobutyl, isobutyl, sec-butyl, tert-butyl, pentyl, isopentyl, neopentyl, 5,5,5-trifluoropentyl, hexyl and 6,6,6-trifluorohexyl.

In the present specification, examples of the “C₂₋₆ alkenyl group” include ethenyl, 1-propenyl, 2-propenyl, 2-methyl-1-propenyl, 1-butenyl, 2-butenyl, 3-butenyl, 3-methyl-2-butenyl, 1-pentenyl, 2-pentenyl, 3-pentenyl, 4-pentenyl, 4-methyl-3-pentenyl, 1-hexenyl, 3-hexenyl and 5-hexenyl.

In the present specification, examples of the “C₂₋₆ alkynyl group” include ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, 5-hexynyl and 4-methyl-2-pentynyl.

In the present specification, examples of the “C₃₋₁₀ cycloalkyl group” include cyclopropyl, cyclobutyl, cyclopentyl, cyclohexyl, cycloheptyl, cyclooctyl, bicyclo[2.2.1]heptyl, bicyclo[2.2.2]octyl, bicyclo[3.2.1]octyl and adamantyl.

In the present specification, examples of the “optionally halogenated C₃₋₁₀ cycloalkyl group” include a C₃₋₁₀ cycloalkyl group optionally having 1 to 7, preferably 1 to 5, halogen atoms. Specific examples thereof include cyclopropyl, 2,2-difluorocyclopropyl, 2,3-difluorocyclopropyl, cyclobutyl, difluorocyclobutyl, cyclopentyl, cyclohexyl, cycloheptyl and cyclooctyl.

In the present specification, examples of the “C₃₋₁₀ cycloalkenyl group” include cyclopropenyl, cyclobutenyl, cyclopentenyl, cyclohexenyl, cycloheptenyl and cyclooctenyl.

In the present specification, examples of the “C₆₋₁₄ aryl group” include phenyl, 1-naphthyl, 2-naphthyl, 1-anthryl, 2-anthryl and 9-anthryl.

In the present specification, examples of the “C₇₋₁₆ aralkyl group” include benzyl, phenethyl, naphthylmethyl and phenylpropyl.

In the present specification, examples of the “C₁₋₆ alkoxy group” include methoxy, ethoxy, propoxy, isopropoxy, butoxy, isobutoxy, sec-butoxy, tert-butoxy, pentyloxy and hexyloxy.

In the present specification, examples of the “optionally halogenated C₁₋₆ alkoxy group” include a C₁₋₆ alkoxy group optionally having 1 to 7, preferably 1 to 5, halogen atoms. Specific examples thereof include methoxy, difluoromethoxy, trifluoromethoxy, ethoxy, 2,2,2-trifluoroethoxy, propoxy, isopropoxy, butoxy, 4,4,4-trifluorobutoxy, isobutoxy, sec-butoxy, pentyloxy and hexyloxy.

In the present specification, examples of the “C₃₋₁₀ cycloalkyloxy group” include cyclopropyloxy, cyclobutyloxy, cyclopentyloxy, cyclohexyloxy, cycloheptyloxy and cyclooctyloxy.

In the present specification, examples of the “C₁₋₆ alkylthio group” include methylthio, ethylthio, propylthio, isopropylthio, butylthio, sec-butylthio, tert-butylthio, pentylthio and hexylthio.

In the present specification, examples of the “optionally halogenated C₁₋₆ alkylthio group” include a C₁₋₆ alkylthio group optionally having 1 to 7, preferably 1 to 5, halogen atoms. Specific examples thereof include methylthio, difluoromethylthio, trifluoromethylthio, ethylthio, propylthio, isopropylthio, butylthio, 4,4,4-trifluorobutylthio, pentylthio and hexylthio.

In the present specification, examples of the “C₁₋₆ alkyl-carbonyl group” include acetyl, propanoyl, butanoyl, 2-methylpropanoyl, pentanoyl, 3-methylbutanoyl, 2-methylbutanoyl, 2,2-dimethylpropanoyl, hexanoyl and heptanoyl.

In the present specification, examples of the “optionally halogenated C₁₋₆ alkyl-carbonyl group” include a C₁₋₆ alkyl-carbonyl group optionally having 1 to 7, preferably 1 to 5, halogen atoms. Specific examples thereof include acetyl, chloroacetyl, trifluoroacetyl, trichloroacetyl, propanoyl, butanoyl, pentanoyl and hexanoyl.

In the present specification, examples of the “C₁₋₆ alkoxy-carbonyl group” include methoxycarbonyl, ethoxycarbonyl, propoxycarbonyl, isopropoxycarbonyl, butoxycarbonyl, isobutoxycarbonyl, sec-butoxycarbonyl, tert-butoxycarbonyl, pentyloxycarbonyl and hexyloxycarbonyl.

In the present specification, examples of the “C₆₋₁₄ aryl-carbonyl group” include benzoyl, 1-naphthoyl and 2-naphthoyl.

In the present specification, examples of the “C₇₋₁₆ aralkyl-carbonyl group” include phenylacetyl and phenylpropionyl.

In the present specification, examples of the “5- to 14-membered aromatic heterocyclylcarbonyl group” include nicotinoyl, isonicotinoyl, thenoyl and furoyl.

In the present specification, examples of the “3- to 14-membered non-aromatic heterocyclylcarbonyl group” include morpholinylcarbonyl, piperidinylcarbonyl and pyrrolidinylcarbonyl.

In the present specification, examples of the “mono- or di-C₁₋₆ alkyl-carbamoyl group” include methylcarbamoyl, ethylcarbamoyl, dimethylcarbamoyl, diethylcarbamoyl and N-ethyl-N-methylcarbamoyl.

In the present specification, examples of the “mono- or di-C₇₋₁₆ aralkyl-carbamoyl group” include benzylcarbamoyl and phenethylcarbamoyl.

In the present specification, examples of the “C₁₋₆ alkylsulfonyl group” include methylsulfonyl, ethylsulfonyl, propylsulfonyl, isopropylsulfonyl, butylsulfonyl, sec-butylsulfonyl and tert-butylsulfonyl.

In the present specification, examples of the “optionally halogenated C₁₋₆ alkylsulfonyl group” include a C₁₋₆ alkylsulfonyl group optionally having 1 to 7, preferably 1 to 5, halogen atoms. Specific examples thereof include methylsulfonyl, difluoromethylsulfonyl, trifluoromethylsulfonyl, ethylsulfonyl, propylsulfonyl, isopropylsulfonyl, butylsulfonyl, 4,4,4-trifluorobutylsulfonyl, pentylsulfonyl and hexylsulfonyl.

In the present specification, examples of the “C₆₋₁₄ arylsulfonyl group” include phenylsulfonyl, 1-naphthylsulfonyl and 2-naphthylsulfonyl.

In the present specification, examples of the “substituent” include a halogen atom, a cyano group, a nitro group, an optionally substituted hydrocarbon group, an optionally substituted heterocyclic group, an acyl group, an optionally substituted amino group, an optionally substituted carbamoyl group, an optionally substituted thiocarbamoyl group, an optionally substituted sulfamoyl group, an optionally substituted hydroxy group, an optionally substituted sulfanyl (SH) group and an optionally substituted silyl group.

In the present specification, examples of the “hydrocarbon group” (including “hydrocarbon group” of “optionally substituted hydrocarbon group”) include a C₁₋₆ alkyl group, a C₂₋₆ alkenyl group, a C₂₋₆ alkynyl group, a C₃₋₁₀ cycloalkyl group, a C₃₋₁₀ cycloalkenyl group, a C₆₋₁₄ aryl group and a C₇₋₁₆ aralkyl group.

In the present specification, examples of the “optionally substituted hydrocarbon group” include a hydrocarbon group optionally having substituent(s) selected from the following Substituent group A.

### [Substituent Group A]

(1) a halogen atom,
(2) a nitro group,
(3) a cyano group,
(4) an oxo group,
(5) a hydroxy group,
(6) an optionally halogenated C₁₋₆ alkoxy group,
(7) a C₆₋₁₄ aryloxy group (e.g., phenoxy, naphthoxy),
(8) a C₇₋₁₆ aralkyloxy group (e.g., benzyloxy),
(9) a 5- to 14-membered aromatic heterocyclyloxy group (e.g., pyridyloxy),
(10) a 3- to 14-membered non-aromatic heterocyclyloxy group (e.g., morpholinyloxy, piperidinyloxy),
(11) a C₁₋₆ alkyl-carbonyloxy group (e.g., acetoxy, propanoyloxy),
(12) a C₆₋₁₄ aryl-carbonyloxy group (e.g., benzoyloxy, 1-naphthoyloxy, 2-naphthoyloxy),
(13) a C₁₋₆ alkoxy-carbonyloxy group (e.g., methoxycarbonyloxy, ethoxycarbonyloxy, propoxycarbonyloxy, butoxycarbonyloxy),
(14) a mono- or di-C₁₋₆ alkyl-carbamoyloxy group (e.g., methylcarbamoyloxy, ethylcarbamoyloxy, dimethylcarbamoyloxy, diethylcarbamoyloxy),
(15) a C-14 aryl-carbamoyloxy group (e.g., phenylcarbamoyloxy, naphthylcarbamoyloxy),
(16) a 5- to 14-membered aromatic heterocyclylcarbonyloxy group (e.g., nicotinoyloxy),
(17) a 3- to 14-membered non-aromatic heterocyclylcarbonyloxy group (e.g., morpholinylcarbonyloxy, piperidinylcarbonyloxy),
(18) an optionally halogenated C₁₋₆ alkylsulfonyloxy group (e.g., methylsulfonyloxy, trifluoromethylsulfonyloxy),
(19) a C₆₋₁₄ arylsulfonyloxy group optionally substituted by a C₁₋₆ alkyl group (e.g., phenylsulfonyloxy, toluenesulfonyloxy),
(20) an optionally halogenated C₁₋₆ alkylthio group,
(21) a 5- to 14-membered aromatic heterocyclic group,
(22) a 3- to 14-membered non-aromatic heterocyclic group,
(23) a formyl group,
(24) a carboxy group,
(25) an optionally halogenated C₁₋₆ alkyl-carbonyl group,
(26) a C₆₋₁₄ aryl-carbonyl group,
(27) a 5- to 14-membered aromatic heterocyclylcarbonyl group,
(28) a 3- to 14-membered non-aromatic heterocyclylcarbonyl group,
(29) a C₁₋₆ alkoxy-carbonyl group,
(30) a C₆₋₁₄ aryloxy-carbonyl group (e.g., phenyloxycarbonyl, 1-naphthyloxycarbonyl, 2-naphthyloxycarbonyl),
(31) a C₇₋₁₆ aralkyloxy-carbonyl group (e.g., benzyloxycarbonyl, phenethyloxycarbonyl),
(32) a carbamoyl group,
(33) a thiocarbamoyl group,
(34) a mono- or di-C₁₋₆ alkyl-carbamoyl group,
(35) a C₆₋₁₄ aryl-carbamoyl group (e.g., phenylcarbamoyl),
(36) a 5- to 14-membered aromatic heterocyclylcarbamoyl group (e.g., pyridylcarbamoyl, thienylcarbamoyl),
(37) a 3- to 14-membered non-aromatic heterocyclylcarbamoyl group (e.g., morpholinylcarbamoyl, piperidinylcarbamoyl),
(38) an optionally halogenated C₁₋₆ alkylsulfonyl group,
(39) a C₆₋₁₄ arylsulfonyl group,
(40) a 5- to 14-membered aromatic heterocyclylsulfonyl group (e.g., pyridylsulfonyl, thienylsulfonyl),
(41) an optionally halogenated C₁₋₆ alkylsulfinyl group,
(42) a C₆₋₁₄ arylsulfinyl group (e.g., phenylsulfinyl, 1-naphthylsulfinyl, 2-naphthylsulfinyl),
(43) a 5- to 14-membered aromatic heterocyclylsulfinyl group (e.g., pyridylsulfinyl, thienylsulfinyl),
(44) an amino group,
(45) a mono- or di-C₁₋₆ alkylamino group (e.g., methylamino, ethylamino, propylamino, isopropylamino, butylamino, dimethylamino, diethylamino, dipropylamino, dibutylamino, N-ethyl-N-methylamino),
(46) a mono- or di-C₆₋₁₄ arylamino group (e.g., phenylamino),
(47) a 5- to 14-membered aromatic heterocyclylamino group (e.g., pyridylamino),
(48) a C₇₋₁₆ aralkylamino group (e.g., benzylamino),
(49) a formylamino group,
(50) a C₁₋₆ alkyl-carbonylamino group (e.g., acetylamino, propanoylamino, butanoylamino),
(51) a (C₁₋₆ alkyl) (C₁₋₆ alkyl-carbonyl) amino group (e.g., N-acetyl-N-methylamino),
(52) a C₆₋₁₄ aryl-carbonylamino group (e.g., phenylcarbonylamino, naphthylcarbonylamino),
(53) a C₁₋₆ alkoxy-carbonylamino group (e.g., methoxycarbonylamino, ethoxycarbonylamino, propoxycarbonylamino, butoxycarbonylamino, tert-butoxycarbonylamino),
(54) a C₇₋₁₆ aralkyloxy-carbonylamino group (e.g., benzyloxycarbonylamino),
(55) a C₁₋₆ alkylsulfonylamino group (e.g., methylsulfonylamino, ethylsulfonylamino),
(56) a C₆₋₁₄ arylsulfonylamino group optionally substituted by a C₁₋₆ alkyl group (e.g., phenylsulfonylamino, toluenesulfonylamino),
(57) an optionally halogenated C₁₋₆ alkyl group,
(58) a C₂₋₆ alkenyl group,
(59) a C₂₋₆ alkynyl group,
(60) a C₃₋₁₀ cycloalkyl group,
(61) a C₃₋₁₀ cycloalkenyl group, and
(62) a C₆₋₁₄ aryl group.

The number of the above-mentioned substituents in the “optionally substituted hydrocarbon group” is, for example, 1 to 5, preferably 1 to 3. When the number of the substituents is two or more, the respective substituents may be the same or different.

In the present specification, examples of the “heterocyclic group” (including “heterocyclic group” of “optionally substituted heterocyclic group”) include (i) an aromatic heterocyclic group, (ii) a non-aromatic heterocyclic group and (iii) a 7- to 10-membered bridged heterocyclic group, each containing, as a ring-constituting atom besides carbon atom, 1 to 4 heteroatoms selected from a nitrogen atom, a sulfur atom and an oxygen atom.

In the present specification, examples of the “aromatic heterocyclic group” (including “5- to 14-membered aromatic heterocyclic group”) include a 5- to 14-membered (preferably 5- to 10-membered) aromatic heterocyclic group containing, as a ring-constituting atom besides carbon atom, 1 to 4 heteroatoms selected from a nitrogen atom, a sulfur atom and an oxygen atom.

Preferable examples of the “aromatic heterocyclic group” include 5- or 6-membered monocyclic aromatic heterocyclic groups such as thienyl, furyl, pyrrolyl, imidazolyl, pyrazolyl, thiazolyl, isothiazolyl, oxazolyl, isoxazolyl, pyridyl, pyrazinyl, pyrimidinyl, pyridazinyl, 1,2,4-oxadiazolyl, 1,3,4-oxadiazolyl, 1,2,4-thiadiazolyl, 1,3,4-thiadiazolyl, triazolyl, tetrazolyl, triazinyl and the like; and 8- to 14-membered fused polycyclic (preferably bi- or tri-cyclic) aromatic heterocyclic groups such as benzothiophenyl, benzofuranyl, benzimidazolyl, benzoxazolyl, benzisoxazolyl, benzothiazolyl, benzisothiazolyl, benzotriazolyl, imidazopyridinyl, thienopyridinyl, furopyridinyl, pyrrolopyridinyl, pyrazolopyridinyl, oxazolopyridinyl, thiazolopyridinyl, imidazopyrazinyl, imidazopyrimidinyl, thienopyrimidinyl, furopyrimidinyl, pyrrolopyrimidinyl, pyrazolopyrimidinyl, oxazolopyrimidinyl, thiazolopyrimidinyl, pyrazolotriazinyl, naphtho[2,3-b]thienyl, phenoxathiinyl, indolyl, isoindolyl, 1H-indazolyl, purinyl, isoquinolyl, quinolyl, phthalazinyl, naphthyridinyl, quinoxalinyl, quinazolinyl, cinnolinyl, carbazolyl, β-carbolinyl, phenanthridinyl, acridinyl, phenazinyl, phenothiazinyl, phenoxazinyl and the like.

In the present specification, examples of the “non-aromatic heterocyclic group” (including “3- to 14-membered non-aromatic heterocyclic group”) include a 3- to 14-membered (preferably 4- to 10-membered) non-aromatic heterocyclic group containing, as a ring-constituting atom besides carbon atom, 1 to 4 heteroatoms selected from a nitrogen atom, a sulfur atom and an oxygen atom.

Preferable examples of the “non-aromatic heterocyclic group” include 3- to 8-membered monocyclic non-aromatic heterocyclic groups such as aziridinyl, oxiranyl, thiiranyl, azetidinyl, oxetanyl, thietanyl, tetrahydrothienyl, tetrahydrofuranyl, pyrrolinyl, pyrrolidinyl, imidazolinyl, imidazolidinyl, oxazolinyl, oxazolidinyl, pyrazolinyl, pyrazolidinyl, thiazolinyl, thiazolidinyl, tetrahydroisothiazolyl, tetrahydrooxazolyl, tetrahydroisooxazolyl, piperidinyl, piperazinyl, tetrahydropyridinyl, dihydropyridinyl, dihydrothiopyranyl, tetrahydropyrimidinyl, tetrahydropyridazinyl, dihydropyranyl, tetrahydropyranyl, tetrahydrothiopyranyl, morpholinyl, thiomorpholinyl, azepanyl, diazepanyl, azepinyl, oxepanyl, azocanyl, diazocanyl and the like; and 9- to 14-membered fused polycyclic (preferably bi- or tri-cyclic) non-aromatic heterocyclic groups such as dihydrobenzofuranyl, dihydrobenzimidazolyl, dihydrobenzoxazolyl, dihydrobenzothiazolyl, dihydrobenzisothiazolyl, dihydronaphtho[2,3-b]thienyl, tetrahydroisoquinolyl, tetrahydroquinolyl, 4H-quinolizinyl, indolinyl, isoindolinyl, tetrahydrothieno[2,3-c]pyridinyl, tetrahydrobenzazepinyl, tetrahydroquinoxalinyl, tetrahydrophenanthridinyl, hexahydrophenothiazinyl, hexahydrophenoxazinyl, tetrahydrophthalazinyl, tetrahydronaphthyridinyl, tetrahydroquinazolinyl, tetrahydrocinnolinyl, tetrahydrocarbazolyl, tetrahydro-β-carbolinyl, tetrahydroacrydinyl, tetrahydrophenazinyl, tetrahydrothioxanthenyl, octahydroisoquinolyl and the like.

In the present specification, preferable examples of the “7- to 10-membered bridged heterocyclic group” include quinuclidinyl and 7-azabicyclo[2.2.1]heptanyl.

In the present specification, examples of the “nitrogen-containing heterocyclic group” include a “heterocyclic group” containing at least one nitrogen atom as a ring-constituting atom.

In the present specification, examples of the “optionally substituted heterocyclic group” include a heterocyclic group optionally having substituent(s) selected from the above-mentioned Substituent group A.

The number of the substituents in the “optionally substituted heterocyclic group” is, for example, 1 to 3. When the number of the substituents is two or more, the respective substituents may be the same or different.

In the present specification, examples of the “acyl group” include a formyl group, a carboxy group, a carbamoyl group, a thiocarbamoyl group, a sulfino group, a sulfo group, a sulfamoyl group and a phosphono group, each optionally having “1 or 2 substituents selected from a C₁₋₆ alkyl group, a C₂₋₆ alkenyl group, a C₃₋₁₀ cycloalkyl group, a C₃₋₁₀ cycloalkenyl group, a C₆₋₁₄ aryl group, a C₇₋₁₆ aralkyl group, a 5- to 14-membered aromatic heterocyclic group, a 3- to 14-membered non-aromatic heterocyclic group, an amino group and a mono- or di-C₁₋₆ alkyl-amino group, each of which optionally has 1 to 3 substituents selected from a halogen atom, an optionally halogenated C₁₋₆ alkoxy group, a hydroxy group, a nitro group, a cyano group, an amino group and a carbamoyl group”.

Examples of the “acyl group” also include a hydrocarbon-sulfonyl group, a heterocyclylsulfonyl group, a hydrocarbon-sulfinyl group and a heterocyclylsulfinyl group.

Here, the hydrocarbon-sulfonyl group means a hydrocarbon group-bonded sulfonyl group, the heterocyclylsulfonyl group means a heterocyclic group-bonded sulfonyl group, the hydrocarbon-sulfinyl group means a hydrocarbon group-bonded sulfinyl group and the heterocyclylsulfinyl group means a heterocyclic group-bonded sulfinyl group.

Preferable examples of the “acyl group” include a formyl group, a carboxy group, a C₁₋₆ alkyl-carbonyl group, a C₂₋₆ alkenyl-carbonyl group (e.g., crotonoyl), a C₃₋₁₀ cycloalkyl-carbonyl group (e.g., cyclobutanecarbonyl, cyclopentanecarbonyl, cyclohexanecarbonyl, cycloheptanecarbonyl), a C₃₋₁₀ cycloalkenyl-carbonyl group (e.g., 2-cyclohexenecarbonyl), a C₆₋₁₄ aryl-carbonyl group, a C₇₋₁₆ aralkyl-carbonyl group, a 5- to 14-membered aromatic heterocyclylcarbonyl group, a 3- to 14-membered non-aromatic heterocyclylcarbonyl group, a C₁₋₆ alkoxy-carbonyl group, a C₆₋₁₄ aryloxy-carbonyl group (e.g., phenyloxycarbonyl, naphthyloxycarbonyl), a C₇₋₁₆ aralkyloxy-carbonyl group (e.g., benzyloxycarbonyl, phenethyloxycarbonyl), a carbamoyl group, a mono- or di-C₁₋₆ alkyl-carbamoyl group, a mono- or di-C₂₋₆ alkenyl-carbamoyl group (e.g., diallylcarbamoyl), a mono- or di-C₃₋₁₀ cycloalkyl-carbamoyl group (e.g., cyclopropylcarbamoyl), a mono- or di-C₆₋₁₄ aryl-carbamoyl group (e.g., phenylcarbamoyl), a mono- or di-C₇₋₁₆ aralkyl-carbamoyl group, a 5- to 14-membered aromatic heterocyclylcarbamoyl group (e.g., pyridylcarbamoyl), N—C₁₋₆ alkyl-N′,N′-di-C₁₋₆ alkylhydrazine-carbonyl group, a thiocarbamoyl group, a mono- or di-C₁₋₆ alkyl-thiocarbamoyl group (e.g., methylthiocarbamoyl, N-ethyl-N-methylthiocarbamoyl), a mono- or di-C₂₋₆ alkenyl-thiocarbamoyl group (e.g., diallylthiocarbamoyl), a mono- or di-C₃₋₁₀ cycloalkyl-thiocarbamoyl group (e.g., cyclopropylthiocarbamoyl, cyclohexylthiocarbamoyl), a mono- or di-C₆₋₁₄ aryl-thiocarbamoyl group (e.g., phenylthiocarbamoyl), a mono- or di-C₇₋₁₆ aralkyl-thiocarbamoyl group (e.g., benzylthiocarbamoyl, phenethylthiocarbamoyl), a 5- to 14-membered aromatic heterocyclylthiocarbamoyl group (e.g., pyridylthiocarbamoyl), a sulfino group, a C₁₋₆ alkylsulfinyl group (e.g., methylsulfinyl, ethylsulfinyl), a sulfo group, a C₁₋₆ alkylsulfonyl group, a C₆₋₁₄ arylsulfonyl group, a phosphono group and a mono- or di-C₁₋₆ alkylphosphono group (e.g., dimethylphosphono, diethylphosphono, diisopropylphosphono, dibutylphosphono).

In the present specification, examples of the “optionally substituted amino group” include an amino group optionally having “1 or 2 substituents selected from a C₁₋₆ alkyl group, a C₂₋₆ alkenyl group, a C₃₋₁₀ cycloalkyl group, a C₆₋₁₄ aryl group, a C₇₋₁₆ aralkyl group, a C₁₋₆ alkyl-carbonyl group, a C₆₋₁₄ aryl-carbonyl group, a C₇₋₁₆ aralkyl-carbonyl group, a 5- to 14-membered aromatic heterocyclylcarbonyl group, a 3- to 14-membered non-aromatic heterocyclylcarbonyl group, a C₁₋₆ alkoxy-carbonyl group, a 5- to 14-membered aromatic heterocyclic group, a carbamoyl group, a mono- or di-C₁₋₆ alkyl-carbamoyl group, a mono- or di-C₇₋₁₆ aralkyl-carbamoyl group, a C₁₋₆ alkylsulfonyl group and a C₆₋₁₄ arylsulfonyl group, each of which optionally has 1 to 3 substituents selected from Substituent group A”.

Preferable examples of the optionally substituted amino group include an amino group, a mono- or di-(optionally halogenated C₁₋₆ alkyl) amino group (e.g., methylamino, trifluoromethylamino, dimethylamino, ethylamino, diethylamino, propylamino, dibutylamino), a mono- or di-C₂₋₆ alkenylamino group (e.g., diallylamino), a mono- or di-C₃₋₁₀ cycloalkylamino group (e.g., cyclopropylamino, cyclohexylamino), a mono- or di-C₆₋₁₄ arylamino group (e.g., phenylamino), a mono- or di-C₇₋₁₆ aralkylamino group (e.g., benzylamino, dibenzylamino), a mono- or di-(optionally halogenated C₁₋₆ alkyl)-carbonylamino group (e.g., acetylamino, propionylamino), a mono- or di-C₆₋₁₄ aryl-carbonylamino group (e.g., benzoylamino), a mono- or di-C₇₋₁₆ aralkyl-carbonylamino group (e.g., benzylcarbonylamino), a mono- or di-5- to 14-membered aromatic heterocyclylcarbonylamino group (e.g., nicotinoylamino, isonicotinoylamino), a mono- or di-3- to 14-membered non-aromatic heterocyclylcarbonylamino group (e.g., piperidinylcarbonylamino), a mono- or di-C₁₋₆ alkoxy-carbonylamino group (e.g., tert-butoxycarbonylamino), a 5- to 14-membered aromatic heterocyclylamino group (e.g., pyridylamino), a carbamoylamino group, a (mono- or di-C₁₋₆ alkyl-carbamoyl) amino group (e.g., methylcarbamoylamino), a (mono- or di-C₇₋₁₆ aralkyl-carbamoyl) amino group (e.g., benzylcarbamoylamino), a C₁₋₆ alkylsulfonylamino group (e.g., methylsulfonylamino, ethylsulfonylamino), a C₆₋₁₄ arylsulfonylamino group (e.g., phenylsulfonylamino), a (C₁₋₆ alkyl) (C₁₋₆ alkyl-carbonyl) amino group (e.g., N-acetyl-N-methylamino) and a (C₁₋₆ alkyl) (C₆₋₁₄ aryl-carbonyl) amino group (e.g., N-benzoyl-N-methylamino).

In the present specification, examples of the “optionally substituted carbamoyl group” include a carbamoyl group optionally having “1 or 2 substituents selected from a C₁₋₆ alkyl group, a C₂₋₆ alkenyl group, a C₃₋₁₀ cycloalkyl group, a C₆₋₁₄ aryl group, a C₇₋₁₆ aralkyl group, a C₁₋₆ alkyl-carbonyl group, a C₆₋₁₄ aryl-carbonyl group, a C₇₋₁₆ aralkyl-carbonyl group, a 5- to 14-membered aromatic heterocyclylcarbonyl group, a 3- to 14-membered non-aromatic heterocyclylcarbonyl group, a C₁₋₆ alkoxy-carbonyl group, a 5- to 14-membered aromatic heterocyclic group, a carbamoyl group, a mono- or di-C₁₋₆ alkyl-carbamoyl group and a mono- or di-C₇₋₁₆ aralkyl-carbamoyl group, each of which optionally has 1 to 3 substituents selected from Substituent group A”.

Preferable examples of the optionally substituted carbamoyl group include a carbamoyl group, a mono- or di-C₁₋₆ alkyl-carbamoyl group, a mono- or di-C₂₋₆ alkenyl-carbamoyl group (e.g., diallylcarbamoyl), a mono- or di-C₃₋₁₀ cycloalkyl-carbamoyl group (e.g., cyclopropylcarbamoyl, cyclohexylcarbamoyl), a mono- or di-C₆₋₁₄ aryl-carbamoyl group (e.g., phenylcarbamoyl), a mono- or di-C₇₋₁₆ aralkyl-carbamoyl group, a mono- or di-C₁₋₆ alkyl-carbonyl-carbamoyl group (e.g., acetylcarbamoyl, propionylcarbamoyl), a mono- or di-C₆₋₁₄ aryl-carbonyl-carbamoyl group (e.g., benzoylcarbamoyl) and a 5- to 14-membered aromatic heterocyclylcarbamoyl group (e.g., pyridylcarbamoyl).

In the present specification, examples of the “optionally substituted thiocarbamoyl group” include a thiocarbamoyl group optionally having “1 or 2 substituents selected from a C₁₋₆ alkyl group, a C₂₋₆ alkenyl group, a C₃₋₁₀ cycloalkyl group, a C₆₋₁₄ aryl group, a C₇₋₁₆ aralkyl group, a C₁₋₆ alkyl-carbonyl group, a C₆₋₁₄ aryl-carbonyl group, a C₇₋₁₆ aralkyl-carbonyl group, a 5- to 14-membered aromatic heterocyclylcarbonyl group, a 3- to 14-membered non-aromatic heterocyclylcarbonyl group, a C₁₋₆ alkoxy-carbonyl group, a 5- to 14-membered aromatic heterocyclic group, a carbamoyl group, a mono- or di-C₁₋₆ alkyl-carbamoyl group and a mono- or di-C₇₋₁₆ aralkyl-carbamoyl group, each of so which optionally has 1 to 3 substituents selected from Substituent group A”.

Preferable examples of the optionally substituted thiocarbamoyl group include a thiocarbamoyl group, a mono- or di-C₁₋₆ alkyl-thiocarbamoyl group (e.g., methylthiocarbamoyl, ethylthiocarbamoyl, dimethylthiocarbamoyl, diethylthiocarbamoyl, N-ethyl-N-methylthiocarbamoyl), a mono- or di-C₂₋₆ alkenyl-thiocarbamoyl group (e.g., diallylthiocarbamoyl), a mono- or di-C₃₋₁₀ cycloalkyl-thiocarbamoyl group (e.g., cyclopropylthiocarbamoyl, cyclohexylthiocarbamoyl), a mono- or di-C₆₋₁₄ aryl-thiocarbamoyl group (e.g., phenylthiocarbamoyl), a mono- or di-C₇₋₁₆ aralkyl-thiocarbamoyl group (e.g., benzylthiocarbamoyl, phenethylthiocarbamoyl), a mono- or di-C₁₋₆ alkyl-carbonyl-thiocarbamoyl group (e.g., acetylthiocarbamoyl, propionylthiocarbamoyl), a mono- or di-C₆₋₁₄ aryl-carbonyl-thiocarbamoyl group (e.g., benzoylthiocarbamoyl) and a 5- to 14-membered aromatic heterocyclylthiocarbamoyl group (e.g., pyridylthiocarbamoyl).

In the present specification, examples of the “optionally substituted sulfamoyl group” include a sulfamoyl group optionally having “1 or 2 substituents selected from a C₁₋₆ alkyl group, a C₂₋₆ alkenyl group, a C₃₋₁₀ cycloalkyl group, a C₆₋₁₄ aryl group, a C₇₋₁₆ aralkyl group, a C₁₋₆ alkyl-carbonyl group, a C₆₋₁₄ aryl-carbonyl group, a C₇₋₁₆ aralkyl-carbonyl group, a 5- to 14-membered aromatic heterocyclylcarbonyl group, a 3- to 14-membered non-aromatic heterocyclylcarbonyl group, a C₁₋₆ alkoxy-carbonyl group, a 5- to 14-membered aromatic heterocyclic group, a carbamoyl group, a mono- or di-C₁₋₆ alkyl-carbamoyl group and a mono- or di-C₇₋₁₆ aralkyl-carbamoyl group, each of which optionally has 1 to 3 substituents selected from Substituent group A”.

Preferable examples of the optionally substituted sulfamoyl group include a sulfamoyl group, a mono- or di-C₁₋₆ alkyl-sulfamoyl group (e.g., methylsulfamoyl, ethylsulfamoyl, dimethylsulfamoyl, diethylsulfamoyl, N-ethyl-N-methylsulfamoyl), a mono- or di-C₂₋₆ alkenyl-sulfamoyl group (e.g., diallylsulfamoyl), a mono- or di-C₃₋₁₀ cycloalkyl-sulfamoyl group (e.g., cyclopropylsulfamoyl, cyclohexylsulfamoyl), a mono- or di-C₆₋₁₄ aryl-sulfamoyl group (e.g., phenylsulfamoyl), a mono- or di-C₇₋₁₆ aralkyl-sulfamoyl group (e.g., benzylsulfamoyl, phenethylsulfamoyl), a mono- or di-C₁₋₆ alkyl-carbonyl-sulfamoyl group (e.g., acetylsulfamoyl, propionylsulfamoyl), a mono- or di-C₆₋₁₄ aryl-carbonyl-sulfamoyl group (e.g., benzoylsulfamoyl) and a 5- to 14-membered aromatic heterocyclylsulfamoyl group (e.g., pyridylsulfamoyl).

In the present specification, examples of the “optionally substituted hydroxy group” include a hydroxy group optionally having “a substituent selected from a C₁₋₆ alkyl group, a C₂₋₅ alkenyl group, a C₃₋₁₀ cycloalkyl group, a C₆₋₁₄ aryl group, a C₇₋₁₆ aralkyl group, a C₁₋₆ alkyl-carbonyl group, a C₆₋₁₄ aryl-carbonyl group, a C₇₋₁₆ aralkyl-carbonyl group, a 5- to 14-membered aromatic heterocyclylcarbonyl group, a 3- to 14-membered non-aromatic heterocyclylcarbonyl group, a C₁₋₆ alkoxy-carbonyl group, a 5- to 14-membered aromatic heterocyclic group, a carbamoyl group, a mono- or di-C₁₋₆ alkyl-carbamoyl group, a mono- or di-C₇₋₁₆ aralkyl-carbamoyl group, a C₁₋₆ alkylsulfonyl group and a C₆₋₁₄ arylsulfonyl group, each of which optionally has 1 to 3 substituents selected from Substituent group A”.

Preferable examples of the optionally substituted hydroxy group include a hydroxy group, a C₁₋₆ alkoxy group, a C₂₋₆ alkenyloxy group (e.g., allyloxy, 2-butenyloxy, 2-pentenyloxy, 3-hexenyloxy), a C₃₋₁₀ cycloalkyloxy group (e.g., cyclohexyloxy), a C₆₋₁₄ aryloxy group (e.g., phenoxy, naphthyloxy), a C₇₋₁₆ aralkyloxy group (e.g., benzyloxy, phenethyloxy), a C₁₋₆ alkyl-carbonyloxy group (e.g., acetyloxy, propionyloxy, butyryloxy, isobutyryloxy, pivaloyloxy), a C₆₋₁₄ aryl-carbonyloxy group (e.g., benzoyloxy), a C₇₋₁₆ aralkyl-carbonyloxy group (e.g., benzylcarbonyloxy), a 5- to 14-membered aromatic heterocyclylcarbonyloxy group (e.g., nicotinoyloxy), a 3- to 14-membered non-aromatic heterocyclylcarbonyloxy group (e.g., piperidinylcarbonyloxy), a C₁₋₆ alkoxy-carbonyloxy group (e.g., tert-butoxycarbonyloxy), a 5- to 14-membered aromatic heterocyclyloxy group (e.g., pyridyloxy), a carbamoyloxy group, a C₁₋₆ alkyl-carbamoyloxy group (e.g., methylcarbamoyloxy), a C₇₋₁₆ aralkyl-carbamoyloxy group (e.g., benzylcarbamoyloxy), a C₁₋₆ alkylsulfonyloxy group (e.g., methylsulfonyloxy, ethylsulfonyloxy) and a C₆₋₁₄ arylsulfonyloxy group (e.g., phenylsulfonyloxy).

In the present specification, examples of the “optionally substituted sulfanyl group” include a sulfanyl group optionally having “a substituent selected from a C₁₋₆ alkyl group, a C₂₋₆ alkenyl group, a C₃₋₁₀ cycloalkyl group, a C₆₋₁₄ aryl group, a C₇₋₁₆ aralkyl group, a C₁₋₆ alkyl-carbonyl group, a C₆₋₁₄ aryl-carbonyl group and a 5- to 14-membered aromatic heterocyclic group, each of which optionally has 1 to 3 substituents selected from Substituent group A” and a halogenated sulfanyl group.

Preferable examples of the optionally substituted sulfanyl group include a sulfanyl (—SH) group, a C₁₋₆ alkylthio group, a C₂₋₆ alkenylthio group (e.g., allylthio, 2-butenylthio, 2-pentenylthio, 3-hexenylthio), a C₃₋₁₀ cycloalkylthio group (e.g., cyclohexylthio), a C₆₋₁₄ arylthio group (e.g., phenylthio, naphthylthio), a C₇₋₁₆ aralkylthio group (e.g., benzylthio, phenethylthio), a C₁₋₆ alkyl-carbonylthio group (e.g., acetylthio, propionylthio, butyrylthio, isobutyrylthio, pivaloylthio), a C₆₋₁₄ aryl-carbonylthio group (e.g., benzoylthio), a 5- to 14-membered aromatic heterocyclylthio group (e.g., pyridylthio) and a halogenated thio group (e.g., pentafluorothio).

In the present specification, examples of the “optionally substituted silyl group” include a silyl group optionally having “1 to 3 substituents selected from a C₁₋₆ alkyl group, a C₂₋₆ alkenyl group, a C₃₋₁₀ cycloalkyl group, a C₆₋₁₄ aryl group and a C₇₋₁₆ aralkyl group, each of which optionally has 1 to 3 substituents selected from Substituent group A”.

Preferable examples of the optionally substituted silyl group include a tri-C₁₋₆ alkylsilyl group (e.g., trimethylsilyl, tert-butyl(dimethyl)silyl).

In the present specification, examples of the “hydrocarbon ring” include a C₆₋₁₄ aromatic hydrocarbon ring, C₃₋₁₀ cycloalkane and C₃₋₁₀ cycloalkene.

In the present specification, examples of the “C₆₋₁₄ aromatic hydrocarbon ring” include benzene and naphthalene.

In the present specification, examples of the “C₃₋₁₀ cycloalkane” include cyclopropane, cyclobutane, cyclopentane, cyclohexane, cycloheptane and cyclooctane.

In the present specification, examples of the “C₃₋₁₀ cycloalkene” include cyclopropene, cyclobutene, cyclopentene, cyclohexene, cycloheptene and cyclooctene.

In the present specification, examples of the “heterocycle” include an aromatic heterocycle and a non-aromatic heterocycle, each containing, as a ring-constituting atom besides carbon atom, 1 to 4 hetero atoms selected from a nitrogen atom, a sulfur atom and an oxygen atom.

In the present specification, examples of the “aromatic heterocycle” include a 5- to 14-membered (preferably 5- to 10-membered) aromatic heterocycle containing, as a ring-constituting atom besides carbon atom, 1 to 4 hetero atoms selected from a nitrogen atom, a sulfur atom and an oxygen atom. Preferable examples of the “aromatic heterocycle” include 5- or 6-membered monocyclic aromatic heterocycles such as thiophene, furan, pyrrole, imidazole, pyrazole, thiazole, isothiazole, oxazole, isoxazole, pyridine, pyrazine, pyrimidine, pyridazine, 1,2,4-oxadiazole, 1,3,4-oxadiazole, 1,2,4-thiadiazole, 1,3,4-thiadiazole, triazole, tetrazole, triazine and the like; and

8- to 14-membered fused polycyclic (preferably bi- or tri-cyclic) aromatic heterocycles such as benzothiophene, benzofuran, benzimidazole, benzoxazole, benzisoxazole, benzothiazole, benzisothiazole, benzotriazole, imidazopyridine, thienopyridine, furopyridine, pyrrolopyridine, pyrazolopyridine, oxazolopyridine, thiazolopyridine, imidazopyrazine, imidazopyrimidine, thienopyrimidine, furopyrimidine, pyrrolopyrimidine, pyrazolopyrimidine, oxazolopyrimidine, thiazolopyrimidine, pyrazolopyrimidine, pyrazolotriazine, naphtho[2,3-b]thiophene, phenoxathiin, indole, isoindole, 1H-indazole, purine, isoquinoline, quinoline, phthalazine, naphthyridine, quinoxaline, quinazoline, cinnoline, carbazole, β-carboline, phenanthridine, acridine, phenazine, phenothiazine, phenoxazine and the like.

In the present specification, examples of the “non-aromatic heterocycle” include a 3- to 14-membered (preferably 4- to 10-membered) non-aromatic heterocycle containing, as a ring-constituting atom besides carbon atom, 1 to 4 hetero atoms selected from a nitrogen atom, a sulfur atom and an oxygen atom. Preferable examples of the “non-aromatic heterocycle” include 3- to 8-membered monocyclic non-aromatic heterocycles such as aziridine, oxirane, thiirane, azetidine, oxetane, thietane, tetrahydrothiophene, tetrahydrofuran, pyrroline, pyrrolidine, imidazoline, imidazolidine, oxazoline, oxazolidine, pyrazoline, pyrazolidine, thiazoline, thiazolidine, tetrahydroisothiazole, tetrahydrooxazole, tetrahydroisoxazole, piperidine, piperazine, tetrahydropyridine, dihydropyridine, dihydrothiopyran, tetrahydropyrimidine, tetrahydropyridazine, dihydropyran, tetrahydropyran, tetrahydrothiopyran, morpholine, thiomorpholine, azepane, diazepane, azepine, azocane, diazocane, oxepane and the like; and 9- to 14-membered fused polycyclic (preferably bi- or tri-cyclic) non-aromatic heterocycles such as dihydrobenzofuran, dihydrobenzimidazole, dihydrobenzoxazole, dihydrobenzothiazole, dihydrobenzisothiazole, dihydronaphtho[2,3-b]thiophene, tetrahydroisoquinoline, tetrahydroquinoline, 4H-quinolizine, indoline, isoindoline, tetrahydrothieno[2,3-c]pyridine, tetrahydrobenzazepine, tetrahydroquinoxaline, tetrahydrophenanthridine, hexahydrophenothiazine, hexahydrophenoxazine, tetrahydrophthalazine, tetrahydronaphthyridine, tetrahydroquinazoline, tetrahydrocinnoline, tetrahydrocarbazole, tetrahydro-β-carboline, tetrahydroacridine, tetrahydrophenazine, tetrahydrothioxanthene, octahydroisoquinoline and the like.

In the present specification, examples of the “nitrogen-containing heterocycle” include a heterocycle containing at least one nitrogen atom as a ring-constituting atom, from among the “heterocycle”.

In the present specification, the “8- to 14-membered bi-cyclic aromatic heterocyclic group” means a 8- to 14-membered fused bi-cyclic aromatic heterocyclic groups, from among the “8- to 14-membered fused polycyclic aromatic heterocyclic groups”.

By “effective” amount of a drug, pharmaceutical composition, or permeate is meant a sufficient amount of an active agent to provide the desired local or systemic effect. A “pharmaceutically effective” or “therapeutically effective” amount refers to the amount of drug needed to effect the desired therapeutic result.

The term “pharmaceutically acceptable salt” is meant to include a salt of a compound of the invention which is prepared with relatively nontoxic acids or bases, depending on the particular substituents found on the compounds described herein. When compounds of the invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino (such as choline or diethylamine or amino acids such as d-arginine, l-arginine, d-lysine, l-lysine), or magnesium salt, or a similar salt. When compounds of the invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science 66: 1-19 (1977)). Certain specific compounds of the invention contain both basic and acidic functionalities that allow the compounds to be converted into either base or acid addition salts.

The neutral forms of the compounds are preferably regenerated by contacting the salt with a base or acid and isolating the parent compounds in the conventional manner. The parent form of the compound differs from the various salt forms in certain physical properties, such as solubility in polar solvents.

In addition to salt forms, the invention provides compounds which are in a prodrug form. Prodrugs of the compounds described herein readily undergo chemical changes under physiological conditions to provide the compounds of the invention. Additionally, prodrugs can be converted to the compounds of the invention by chemical or biochemical methods in an ex vivo environment.

Certain compounds of the invention can exist in unsolvated forms as well as solvated forms, including hydrated forms. In general, the solvated forms are equivalent to unsolvated forms and are encompassed within the scope of the invention. Certain compounds of the invention may exist in multiple crystalline or amorphous forms.

Certain compounds of the invention possess asymmetric carbon atoms (optical centers) or double bonds; the racemates, diastereomers, geometric isomers and individual isomers are encompassed within the scope of the invention. The graphic representations of racemic, ambiscalemic and scalemic or enantiomerically pure compounds used herein are taken from Maehr, J. Chem. Ed. 1985, 62: 114-120. Solid and broken wedges are used to denote the absolute configuration of a stereocenter unless otherwise noted. When the compounds described herein contain olefinic double bonds or other centers of geometric asymmetry, and unless specified otherwise, it is intended that the compounds include both E and Z geometric isomers. Likewise, all tautomeric forms are included.

Compounds of the invention can exist in particular geometric or stereoisomeric forms. The invention contemplates all such compounds, including cis- and trans-isomers, (−)- and (+)-enantiomers, (R)- and (S)-enantiomers, diastereomers, (D)-isomers, (L)-isomers, the racemic mixtures thereof, and other mixtures thereof, such as enantiomerically or diastereomerically enriched mixtures, as falling within the scope of the invention. Additional asymmetric carbon atoms can be present in a substituent such as an alkyl group. All such isomers, as well as mixtures thereof, are intended to be included in this invention.

Optically active (R)- and (S)-isomers and d and l isomers can be prepared using chiral synthons or chiral reagents, or resolved using conventional techniques. If, for instance, a particular enantiomer of a compound of the invention is desired, it can be prepared by asymmetric synthesis, or by derivatization with a chiral auxiliary, where the resulting diastereomeric mixture is separated and the auxiliary group cleaved to provide the pure desired enantiomers. Alternatively, where the molecule contains a basic functional group, such as an amino group, or an acidic functional group, such as a carboxyl group, diastereomeric salts can be formed with an appropriate optically active acid or base, followed by resolution of the diastereomers thus formed by fractional crystallization or chromatographic means known in the art, and subsequent recovery of the pure enantiomers. In addition, separation of enantiomers and diastereomers is frequently accomplished using chromatography employing chiral, stationary phases, optionally in combination with chemical derivatization (e.g., formation of carbamates from amines).

The compounds of the invention may also contain unnatural proportions of atomic isotopes at one or more of the atoms that constitute such compounds. For example, the compounds may be radiolabeled with radioactive isotopes, such as for example tritium (³H), iodine-125 (¹²⁵I) or carbon-14 (¹⁴C). The compounds may also be labeled with stable isotopes such as deuterium. All isotopic variations of the compounds of the invention, whether radioactive or not, are intended to be encompassed within the scope of the invention.

The term “pharmaceutically acceptable carrier” or “pharmaceutically acceptable vehicle” refers to any composition or carrier medium that provides the appropriate delivery of an effective amount of an active agent as defined herein, does not interfere with the effectiveness of the biological activity of the active agent, and that is sufficiently non-toxic to the mammal. Representative carriers include water, oils, both vegetable and mineral, cream bases, lotion bases, ointment bases and the like. These bases include suspending agents, thickeners, penetration enhancers, and the like. Their formulation is well known to those in the pharmaceutical arts. Additional information concerning carriers can be found in Remington: The Science and Practice of Pharmacy, 21st Ed., Lippincott, Williams & Wilkins (2005) which is incorporated herein by reference.

The term “pharmaceutically acceptable additive” refers to preservatives, antioxidants, fragrances, emulsifiers, dyes and excipients known or used in the field of drug formulation and that do not unduly interfere with the effectiveness of the biological activity of the active agent, and that is sufficiently non-toxic to the mammal. Additives for topical formulations are well-known in the art, and may be added to the topical formulation, as long as they are pharmaceutically acceptable and not deleterious to the epithelial cells or their function. Further, they should not cause deterioration in the stability of the formulation. For example, inert fillers, anti-irritants, tackifiers, excipients, fragrances, opacifiers, antioxidants, gelling agents, stabilizers, surfactant, emollients, coloring agents, preservatives, buffering agents, other permeation enhancers, and other conventional components of topical or transdermal delivery formulations as are known in the art.

The terms “enhancement,” “penetration enhancement” or “permeation enhancement” relate to an increase in the permeability of the skin, nail, hair, claw or hoof to a drug, so as to increase the rate at which the drug permeates through the skin, nail, hair, claw or hoof. The enhanced permeation effected through the use of such enhancers can be observed, for example, by measuring the rate of diffusion of the drug through animal skin, nail, hair, claw or hoof using a diffusion cell apparatus. A diffusion cell is described by Merritt et al. Diffusion Apparatus for Skin Penetration, J of Controlled Release, 1 (1984) pp. 161-162. The term “permeation enhancer” or “penetration enhancer” intends an agent or a mixture of agents, which, alone or in combination, act to increase the permeability of the skin, nail, hair or hoof to a drug.

The term “excipients” is conventionally known to mean carriers, diluents and/or vehicles used in formulating drug compositions effective for the desired use.

The terms “effective amount” or a “therapeutically effective amount” of a drug or pharmacologically active agent refers to a nontoxic but sufficient amount of the drug or agent to provide the desired effect. In the oral dosage forms of the present disclosure, an “effective amount” of one active of the combination is the amount of that active that is effective to provide the desired effect when used in combination with the other active of the combination. The amount that is “effective” will vary from subject to subject, depending on the age and general condition of the individual, the particular active agent or agents, and the appropriate “effective” amount in any individual case may be determined by one of ordinary skill in the art using routine experimentation.

The phrases “active ingredient”, “therapeutic agent”, “active”, or “active agent” mean a chemical entity which can be effective in treating a targeted disorder, disease or condition.

The phrase “pharmaceutically acceptable” means moieties or compounds that are, within the scope of medical judgment, suitable for use in humans without causing undesirable biological effects such as undue toxicity, irritation, allergic response, and the like, for example.

The phrase “unit”, as used herein, refers to the number of discrete objects to be administered which comprise the dosage form. In some embodiments, the dosage form includes a compound of the invention in one capsule. This is a single unit. In some embodiments, the dosage form includes a compound of the invention as part of a therapeutically effective dosage of a cream or ointment. This is also a single unit. In some embodiments, the dosage form includes a compound of the invention and another active ingredient contained within one capsule, or as part of a therapeutically effective dosage of a cream or ointment. This is a single unit, whether or not the interior of the capsule includes multiple discrete granules of the active ingredient. In some embodiments, the dosage form includes a compound of the invention in one capsule, and the active ingredient in a second capsule. This is a two unit dosage form, such as two capsules or tablets, and so such units are contained in a single package. Thus, the term ‘unit’ refers to the object which is administered to the mammal, not to the interior components of the object.

“Biological medium” as used herein refers to both in vitro and in vivo biological milieus. Exemplary in vitro “biological media” include, but are not limited to, cell culture, tissue culture, homogenates, plasma and blood. In vivo applications are generally performed in mammals, preferably mouse, rat, hamster, rabbit, cat, dog, bovine, sheep, monkey, and humans.

“Salt counterion”, as used herein, refers to positively charged ions that associate with a compound of the invention. Examples of salt counterions include H⁺, H₃O⁺, ammonium, potassium, calcium, magnesium, organic amino (such as choline or diethylamine or amino acids such as d-arginine, l-arginine, d-lysine, l-lysine), and sodium.

### II. Introduction

The invention provides compounds described herein, as well as pharmaceutical compositions containing such compounds, which can be used for, among other things, preventing and/or treating diseases such as amyotrophic lateral sclerosis (hereinafter, also to be referred to as ALS), frontotemporal dementia, chronic traumatic encephalopathy, Alzheimer's disease, frontotemporal lobar degeneration, multisystem proteinopathy and the like, particularly ALS.

### III. The Compounds

### III.a)

In one aspect, the invention provides a compound of the invention. In an exemplary embodiment, the invention is a compound described herein. In an exemplary embodiment, the invention is the following compound (I).

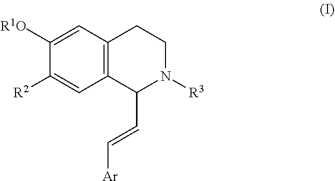

In compound (I), R¹ is unsubstituted C₁₋₆ alkyl, substituted or unsubstituted phenyl substituted C₁₋₆ alkyl, substituted or unsubstituted C₃₋₁₀ cycloalkyl substituted C₁₋₆ alkyl, or substituted or unsubstituted pyridyl substituted C₁₋₆ alkyl.

R¹ is preferably

(1) a C₁₋₆ alkyl group (e.g., methyl, ethyl) substituted by phenyl group(s) optionally substituted by 1 to 3 substituents selected from
(a) a halogen atom (e.g., a chlorine atom), and
(b) an optionally halogenated C₁₋₆ alkyl group (e.g., methyl, trifluoromethyl),
   (2) a C₁₋₆ alkyl group (e.g., methyl) substituted by C₃₋₁₀ cycloalkyl group(s) (e.g., cyclopropyl), or
   (3) a C₁₋₆ alkyl group (e.g., methyl) substituted by pyridyl group(s).

R¹ is more preferably a C₁₋₆ alkyl group (e.g., methyl) substituted by phenyl group(s) optionally substituted by 1 to 3 halogen atoms (e.g., a chlorine atom).

R¹ is particularly preferably a benzyl group optionally substituted by 1 to 3 halogen atoms (e.g., a chlorine atom).

In compound (I), R² is H, CF₃, unsubstituted C₁₋₆ alkoxy, or phenyl substituted C₁₋₆ alkoxy.

R² is preferably a C₁₋₆ alkoxy group (e.g., methoxy).

R² is particularly preferably a methoxy group.

In compound (I), R³ is H, —C(O)OR⁴, or —C(O)R⁴ or —C(O)NR⁴R⁵ wherein R⁴ and R⁵ are independently selected from unsubstituted C₁₋₆ alkyl, unsubstituted phenyl, and unsubstituted pyridyl.

R³ is preferably H.

In compound (I), Ar is substituted or unsubstituted phenyl, substituted or unsubstituted benzofuryl, substituted or unsubstituted pyrrolopyridyl, substituted or unsubstituted imidazopyrimidinyl, substituted or unsubstituted imidazopyrazinyl, substituted or unsubstituted imidazopyridazinyl, or substituted or unsubstituted imidazopyridyl.

Ar is preferably

(1) a phenyl group optionally substituted by 1 to 3 substituents selected from
(a) a halogen atom (e.g., a chlorine atom, a bromine atom),
(b) a C₁₋₆ alkyl group (e.g., methyl),
(c) a C₁₋₆ alkoxy group (e.g., methoxy) optionally substituted by 1 to 3 substituents selected from
   (i) a C₃₋₁₀ cycloalkyl group (e.g., cyclopropyl, cyclobutyl),
   (ii) a 5- or 6-membered monocyclic aromatic heterocyclic group (e.g., pyridyl, pyridazinyl) optionally substituted by 1 to 3 substituents selected from a halogen atom (e.g., a chlorine atom), an amino group, a C₁₋₆ alkyl group (e.g., methyl) and a C₁₋₆ alkoxy group (e.g., methoxy),
   (iii) a 3- to 8-membered monocyclic non-aromatic heterocyclic group (e.g., tetrahydropyranyl, tetrahydrofuryl, piperidyl) optionally substituted by 1 to 3 substituents selected from an oxo group and a C₁₋₆ alkyl group (e.g., methyl), and
   (iv) a 8- to 14-membered bicyclic aromatic heterocyclic group (e.g., imidazopyridyl, benzotriazolyl, indazolyl, quinolyl) optionally substituted by 1 to 3 C₁₋₆ alkyl groups (e.g., methyl),
(d) a C₂₋₆ alkynyloxy group (e.g., 2-propynyloxy),
(e) a C₁₋₃ alkylenedioxy group (e.g., methylenedioxy, ethylenedioxy),
(f) a C₆₋₁₄ aryl group (e.g., phenyl) optionally substituted by 1 to 3 substituents selected from
   (i) a C₁₋₆ alkoxy group (e.g., methoxy), and
   (ii) a 5- or 6-membered monocyclic aromatic heterocyclic group (e.g., pyrazolyl),
(g) a C₇₋₁₆ aralkyloxy group (e.g., benzyloxy) optionally substituted by 1 to 3 substituents selected from
   (i) a cyano group,
   (ii) a C₁₋₆ alkyl group (e.g., methyl) optionally substituted by 1 to 3 amino groups,
   (iii) an optionally halogenated C₁₋₆ alkoxy group (e.g., methoxy, difluoromethoxy),
   (iv) a C₁₋₆ alkyl-carbonyl group (e.g., acetyl),
   (v) a C₁₋₆ alkoxy-carbonyl group (e.g., methoxycarbonyl), and
   (vi) a C₁₋₆ alkylsulfonyl group (e.g., methylsulfonyl),
(h) a 5- or 6-membered monocyclic aromatic heterocyclic group (e.g., imidazolyl, pyrazolyl, isoxazolyl, pyridyl, pyrimidinyl, pyridazinyl, pyrazinyl) optionally substituted by 1 to 3 substituents selected from
   (i) a cyano group,
   (ii) an optionally halogenated C₁₋₆ alkyl group (e.g., methyl, trifluoromethyl),
   (iii) an optionally halogenated C₁₋₆ alkoxy group (e.g., methoxy, 2,2,2-trifluoroethoxy),
   (iv) a mono-C₁₋₆ alkylamino group (e.g., methylamino),
   (v) a C₁₋₆ alkoxy-carbonyl group (e.g., ethoxycarbonyl), and
   (vi) a C₇₋₁₆ aralkyl group (e.g., benzyl),
(i) a 8- to 14-membered bicyclic aromatic heterocyclic group (e.g., quinoxalinyl, pyrazolopyridyl, imidazopyridyl), and
(j) a 3- to 8-membered monocyclic non-aromatic heterocyclic group (e.g., dihydropyranyl),
   (2) a benzofuryl group optionally substituted by 1 to 3 C₁₋₆ alkoxy groups (e.g., methoxy),
   (3) a pyrrolopyridyl group (e.g., pyrrolo[2.3-b]pyridyl) optionally substituted by 1 to 3 substituents selected from
(a) a hydroxy group,
(b) a C₁₋₆ alkyl group (e.g., methyl),
(c) a C₁₋₆ alkoxy group (e.g., methoxy, ethoxy, propoxy) optionally substituted by 1 to 3 substituents selected from
   (i) a halogen atom (e.g., a fluorine atom),
   (ii) a hydroxy group,
   (iii) a 3- to 8-membered monocyclic non-aromatic heterocyclic group (e.g., diazirinyl) optionally substituted by 1 to 3 substituents selected from a C₁₋₆ alkyl group (e.g., methyl) and a C₂₋₆ alkynyl group (e.g., 3-butynyl), and
(d) a C₂₋₆ alkynyl group (e.g., 2-propynyl),
   (4) an imidazopyrimidinyl group (e.g., imidazo[1.2-a]pyrimidinyl) optionally substituted by 1 to 3 substituents selected from
(a) a halogen atom (e.g., a fluorine atom, a chlorine atom),
(b) a carboxy group,
(c) an optionally halogenated C₁₋₆ alkyl group (e.g., methyl, trifluoromethyl),
(d) a C₁₋₆ alkoxy group (e.g., methoxy), and
(e) a C₁₋₆ alkoxy-carbonyl group (e.g., ethoxycarbonyl),
   (5) an imidazopyrazinyl group (e.g., imidazo[1.2-a]pyrazinyl) optionally substituted by 1 to 3 carboxy groups,
   (6) an imidazopyridazinyl group (e.g., imidazo[1.2-b]pyridazinyl) optionally substituted by 1 to 3 substituents selected from
(a) a carboxy group, and
(b) a C₁₋₆ alkoxy-carbonyl group (e.g., methoxycarbonyl), or
   (7) an imidazopyridyl group (e.g., imidazo[1.2-a]pyridyl) optionally substituted by 1 to 3 substituents selected from
(a) a carboxy group, and
(b) a C₁₋₆ alkoxy-carbonyl group (e.g., methoxycarbonyl).

Ar is more preferably

(1) a phenyl group substituted by 2 or 3 substituents selected from
(a) a C₁₋₆ alkyl group (e.g., methyl),
(b) a C₁₋₆ alkoxy group (e.g., methoxy) optionally substituted by 1 to 3 of 5- or 6-membered monocyclic aromatic heterocyclic groups (e.g., pyridyl) optionally substituted by 1 to 3 halogen atoms (e.g., a chlorine atom), and
(c) a 5- or 6-membered monocyclic aromatic heterocyclic group (e.g., pyrazolyl, pyrimidinyl) optionally substituted by 1 to 3 C₁₋₆ alkyl groups (e.g., methyl), or
   (2) a pyrrolopyridyl group (e.g., pyrrolo[2.3-b]pyridyl) substituted by 2 substituents selected from
(a) a C₁₋₆ alkyl group (e.g., methyl), and
(b) a C₁₋₆ alkoxy group (e.g., methoxy).

Ar is particularly preferably

(1) a phenyl group substituted by 2 or 3 substituents selected from
(a) a C₁₋₆ alkyl group (e.g., methyl),
(b) a C₁₋₆ alkoxy group (e.g., methoxy) optionally substituted by one pyridyl group optionally substituted by 1 to 3 halogen atoms (e.g., a chlorine atom), and
(c) a pyrazolyl or pyrimidinyl group optionally substituted by 1 to 3 C₁₋₆ alkyl groups (e.g., methyl), or
   (2) a pyrrolopyridyl group (e.g., pyrrolo[2.3-b]pyridyl) substituted by 2 substituents selected from
(a) a C₁₋₆ alkyl group (e.g., methyl), and
(b) a C₁₋₆ alkoxy group (e.g., methoxy).

In one aspect, the invention also provides the following compound (IA), which is included in compound (I).

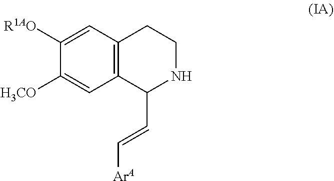

In compound (IA), R^{1A} is a C₁₋₆ alkyl group substituted by phenyl group(s) optionally substituted by 1 to 3 halogen atoms.

R^{1A} is preferably a benzyl group optionally substituted by 1 to 3 halogen atoms (e.g., a chlorine atom).

In compound (IA), Ar^{A} is

(1) a phenyl group substituted by 2 or 3 substituents selected from
(a) a C₁₋₆ alkyl group,
(b) a C₁₋₆ alkoxy group optionally substituted by 1 to 3 of 5- or 6-membered monocyclic aromatic heterocyclic groups optionally substituted by 1 to 3 halogen atoms, and
(c) a 5- or 6-membered monocyclic aromatic heterocyclic group optionally substituted by 1 to 3 C₁₋₆ alkyl groups, or
   (2) a pyrrolopyridyl group substituted by 2 substituents selected from
(a) a C₁₋₆ alkyl group, and
(b) a C₁₋₆ alkoxy group.

Ar^{A} is preferably

(1) a phenyl group substituted by 2 or 3 substituents selected from
(a) a C₁₋₆ alkyl group (e.g., methyl),
(b) a C₁₋₆ alkoxy group (e.g., methoxy) optionally substituted by one pyridyl group optionally substituted by 1 to 3 halogen atoms (e.g., a chlorine atom), and
(c) a pyrazolyl or pyrimidinyl group optionally substituted by 1 to 3 C₁₋₆ alkyl groups (e.g., methyl), or
   (2) a pyrrolopyridyl group (e.g., pyrrolo[2.3-b]pyridyl) substituted by 2 substituents selected from
(a) a C₁₋₆ alkyl group (e.g., methyl), and
(b) a C₁₋₆ alkoxy group (e.g., methoxy).

In one aspect, the invention also provides the following compound (IB), which is included in compound (I).

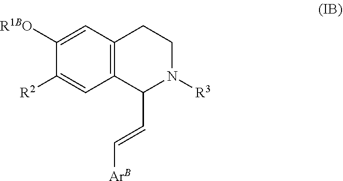

In compound (IB), R^{1B} is unsubstituted C₁₋₆ alkyl, or substituted or unsubstituted phenyl substituted C₁₋₆ alkyl.

R^{1B} is preferably a C₁₋₆ alkyl group (e.g., methyl, ethyl) substituted by phenyl group(s) optionally substituted by 1 to 3 of optionally halogenated C₁₋₆ alkyl group(s) (e.g., methyl, trifluoromethyl).

R^{1B} is particularly preferably a benzyl group.

In compound (IB), R² and R³ are as defined in compound (I).

R² is preferably a C₁₋₆ alkoxy group (e.g., methoxy).

R² is particularly preferably a methoxy group.

R³ is preferably H.

In compound (IB), Ar^{B} is substituted or unsubstituted imidazo[1,2-a]pyrimidinyl, substituted or unsubstituted imidazo[1,2-a]pyrazinyl, substituted or unsubstituted imidazo[1,2-b]pyridazinyl, or substituted or unsubstituted imidazo[1,2-a]pyridyl.

Ar^{B} is preferably

(1) imidazo[1.2-a]pyrimidinyl optionally substituted by 1 to 3 substituents selected from
(a) a halogen atom (e.g., a fluorine atom, a chlorine atom),
(b) a carboxy group,
(c) an optionally halogenated C₁₋₆ alkyl group (e.g., methyl, trifluoromethyl),
(d) a C₁₋₆ alkoxy group (e.g., methoxy), and
(e) a C₁₋₆ alkoxy-carbonyl group (e.g., ethoxycarbonyl),
   (2) imidazo[1.2-a]pyrazinyl optionally substituted by 1 to 3 carboxy groups,
   (3) imidazo[1.2-b]pyridazinyl optionally substituted by 1 to 3 substituents selected from
(a) a carboxy group, and
(b) a C₁₋₆ alkoxy-carbonyl group (e.g., methoxycarbonyl), or
   (4) imidazo[1.2-a]pyridyl optionally substituted by 1 to 3 substituents selected from
(a) a carboxy group, and
(b) a C₁₋₆ alkoxy-carbonyl group (e.g., methoxycarbonyl).

In another embodiment, Ar^{B} is preferably substituted or unsubstituted imidazo[1,2-a]pyrazinyl, substituted or unsubstituted imidazo[1,2-b]pyridazinyl, or substituted or unsubstituted imidazo[1,2-a]pyridyl, more preferably substituted or unsubstituted imidazo[1,2-b]pyridazinyl.

Ar^{B} is further more preferably imidazo[1.2-b]pyridazinyl substituted by one substituent selected from
(a) a carboxy group, and
(b) a C₁₋₆ alkoxy-carbonyl group (e.g., methoxycarbonyl).

In another embodiment, the present invention provides a compound represented by the formula (II):

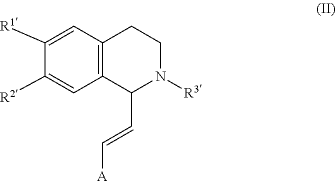

wherein
R^{1′} is H, substituted or unsubstituted C₁₋₆ alkyl, unsubstituted alkoxy, or phenyl substituted alkoxy;
R^{2′} is H, CF₃, substituted or unsubstituted C₁₋₆ alkyl, unsubstituted alkoxy, or phenyl substituted alkoxy;
R^{3′} is H or —C(O)OR⁴ or —C(O)R⁴ or —C(O)NR⁴R⁵, wherein R⁴ and R⁵ are independently selected from unsubstituted alkyl, unsubstituted phenyl, or unsubstituted pyridinyl; and
A is substituted or unsubstituted imidazo[1,2-a]pyrimidin-3-yl. Also provided are hydrates, salts and solvates of these compounds.

In an exemplary embodiment of the formula (II), A, R^{2′} and R^{3′} are as described herein, and R^{1′} is —CH₃. In an exemplary embodiment, A, R^{2′} and R^{3′} are as described herein, and R^{1′} is

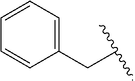

In an exemplary embodiment, A, R^{2′} and R^{3′} are as described herein, and R^{1′} is substituted benzyloxy. In an exemplary embodiment, A, R^{2′} and R^{3′} are as described herein, and R^{1′} is benzyloxy, substituted with one or two or three R^{1a}, wherein each R^{1a} is independently selected from the group consisting of substituted or unsubstituted alkyl and substituted or unsubstituted heteroalkyl. In an exemplary embodiment, A, R^{2′} and R^{3′} are as described herein, and R^{1′} is benzyloxy, substituted with one R^{1a}, wherein said R^{1a} is methyl or trifluoromethyl. In an exemplary embodiment, A, R^{2′} and R^{3′} are as described herein, and R^{1′} is

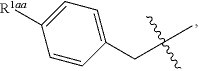

wherein R^{1aa} is substituted or unsubstituted alkyl or substituted or unsubstituted heteroalkyl. In an exemplary embodiment, A, R^{2′} and R^{3′} are as described herein, and R^{1′} is or

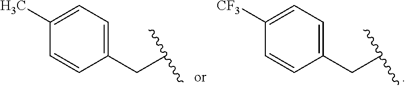

In an exemplary embodiment, A, R^{2′} and R^{3′} are as described herein, and R^{1′} is

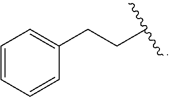 [SMILES: *CCc1ccccc1]

In an exemplary embodiment, A, R^{2′} and R^{3′} are as described herein, and R^{1′} is substituted phenylethoxy. In an exemplary embodiment, A, R^{2′} and R^{3′} are as described herein, and R^{1′} is H.

In an exemplary embodiment of formula (II), A, R^{1′} and R^{3′} are as described herein, and R^{2′} is —CH₃. In an exemplary embodiment, A, R^{1′} and R^{3′} are as described herein, and R^{2′} is

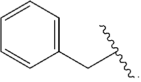 [SMILES: *Cc1ccccc1]

In an exemplary embodiment, A, R^{1′} and R^{3′} are as described herein, and R^{2′} is H.

In an exemplary embodiment of the formula (II), A, R^{1′} and R^{2′} are as described herein, and R^{3′} is H. In an exemplary embodiment, A, R^{1′} and R^{2′} are as described herein, and R^{3′} is —C(O)OCH₃. In an exemplary embodiment, A, R^{1′} and R^{2′} are as described herein, and R^{3′} is —C(O)OC(CH₃)₃. In an exemplary embodiment, A, R^{1′} and R^{2′} are as described herein, and R^{3′} is

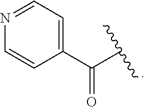 [SMILES: *C(=O)c1ccncc1]

In an exemplary embodiment, A, R^{1′} and R^{2′} are as described herein, and R^{3′} is

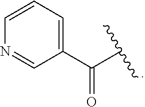 [SMILES: *C(=O)c1cccnc1]

In an exemplary embodiment, A, R^{1′} and R^{2′} are as described herein, and R^{3′} is

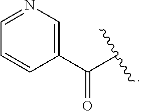 [SMILES: *C(=O)c1cccnc1]

In an exemplary embodiment, A, R^{1′} and R^{2′} are as described herein, and R^{3′} is

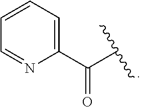 [SMILES: *C(=O)c1ccccn1]

In an exemplary embodiment, A, R^{1′} and R^{2′} are as described herein, and R^{3′} is

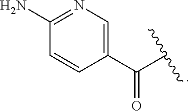 [SMILES: *C(=O)c1ccc(N)nc1]

In an exemplary embodiment, A, R^{1′} and R^{2′} are as described herein, and R^{3′} is

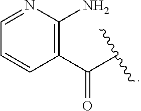 [SMILES: *C(=O)c1cccnc1N]

In an exemplary embodiment, A, R^{1′} and R^{2′} are as described herein, and R^{3′} is

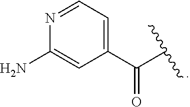 [SMILES: *C(=O)c1ccnc(N)c1]

In an exemplary embodiment, A, R^{1′} and R^{2′} are as described herein, and R^{3′} is

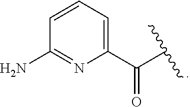 [SMILES: *C(=O)c1cccc(N)n1]

In an exemplary embodiment, A, R^{1′} and R^{2′} are as described herein, and R^{3′} is

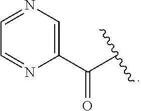 [SMILES: *C(=O)c1cnccn1]

In an exemplary embodiment, A, R^{1′} and R^{2′} are as described herein, and R^{3′} is —C(O)NH₂. In an exemplary embodiment, A, R^{1′} and R^{2′} are as described herein, and R^{3′} is —C(O)NHC(CH₃)₃. In an exemplary embodiment, A, R^{1′} and R^{2′} are as described herein, and R^{3′} is —(CH₂)ₙCH₃, wherein n is an integer selected from 0 or 1 or 2 or 3 or 4 or 5. In an exemplary embodiment, A, R^{1′} and R^{2′} are as described herein, and R^{3′} is —(CH₂)ₙCH₃, wherein n is an integer selected from 6 or 7 or 8 or 9 or 10. In an exemplary embodiment, A, R^{1′} and R^{2′} are as described herein, and R^{3′} is —(CH₂)ₙCH₃, wherein n is an integer selected from 2 or 3 or 4. In an exemplary embodiment, A, R^{1′} and R^{2′} are as described herein, and R^{3′} is —(CH₂)₃CH₃. In an exemplary embodiment, A, R^{1′} and R^{2′} are as described herein, and R^{3′} is —CH₃.

In an exemplary embodiment of the formula (II), R^{1′}, R^{2′} and R^{3′} are as described herein, and A is

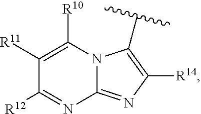

wherein R¹⁰, R¹¹, R¹² and R¹⁴ are each independently selected from H, halogen, cyano, nitro, OR^{10a}, NR^{10a}R^{11a}, SR^{10a}, —C(O)OR^{10a}, —C(O)R^{10a}, —C(O)NR^{10a}R^{11a}, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl, wherein R^{10a} and R^{11a} are independently selected from the group consisting of H, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. In an exemplary embodiment, R^{1′}, R^{2′} and R^{3′} are as described herein, and A is

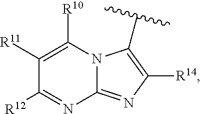

wherein R¹⁰, R¹¹, R¹² and R¹⁴ are each independently as described herein, with the proviso that at least one member selected from R¹⁰, R¹¹, R¹² and R¹⁴ comprises —C(O)OH.

In an exemplary embodiment of the formula (II), R^{1′}, R^{2′} and R^{3′} are as described herein, and A is

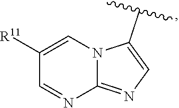

wherein R¹¹ is selected from the group consisting of H, halogen, OR^{10a}, —C(O)OR^{10a}, and substituted or unsubstituted alkyl, wherein R^{10a} is H or substituted or unsubstituted alkyl. In an exemplary embodiment, R^{1′}, R^{2′} and R^{3′} are as described herein, and A is

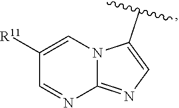

wherein R¹¹ is selected from the group consisting of F, Cl, halogen-substituted alkyl, unsubstituted alkyl, OR^{10a}, and —C(O)OR^{10a}, wherein Rica is unsubstituted alkyl. In an exemplary embodiment, R^{1′}, R^{2′} and R^{3′} are as described herein, and A is

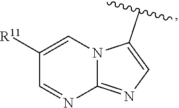

wherein R¹¹ is selected from the group consisting of F, Cl, —CF₃, —CH₃, —OCH₃, —C(O)OCH₂CH₃, and —C(O)OH. In an exemplary embodiment, R^{1′}, R^{2′} and R^{3′} are as described herein, and A is

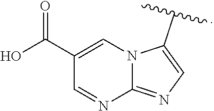 [SMILES: *c1cnc2ncc(C(=O)O)cn12]

In an exemplary embodiment of the formula (II), R^{1′}, R^{2′} and R^{3′} are as described herein, and A is

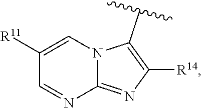

wherein R¹¹ comprises —C(O)OH, and R¹⁴ is substituted or unsubstituted alkyl. In an exemplary embodiment, R^{1′}, R^{2′} and R^{3′} are as described herein, and A is

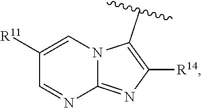

wherein R¹¹ comprises —C(O)OH, and R¹⁴ is unsubstituted alkyl. In an exemplary embodiment, R^{1′}, R^{2′} and R^{3′} are as described herein, and A is

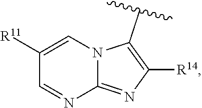

wherein R¹¹ is —C(O)OH, and R¹⁴ is methyl.

In an exemplary embodiment of the formula (II), R^{1′}, R^{2′} and R^{3′} are as described herein, and A is

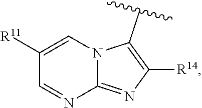

wherein R¹¹ is substituted or unsubstituted alkyl, and R¹⁴ comprises —C(O)OH. In an exemplary embodiment, R^{1′}, R^{2′} and R^{3′} are as described herein, and A is

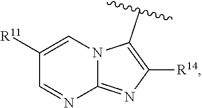

wherein R¹¹ is unsubstituted alkyl, and R¹⁴ comprises —C(O)OH. In an exemplary embodiment, R^{1′}, R^{2′} and R^{3′} are as described herein, and A is

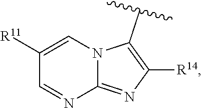

wherein R¹¹ is methyl, and R¹⁴ is —C(O)OH.

In an exemplary embodiment of the formula (II), R^{1′}, R^{2′} and R^{3′} are as described herein, and A is

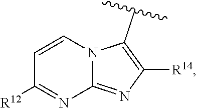

wherein R¹² comprises —C(O)OH, and R¹⁴ is substituted or unsubstituted alkyl. In an exemplary embodiment, R^{1′}, R^{2′} and R^{3′} are as described herein, and A is

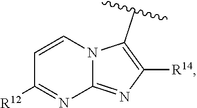

wherein R¹² comprises —C(O)OH, and R¹⁴ is unsubstituted alkyl. In an exemplary embodiment, R^{1′}, R^{2′} and R^{3′} are as described herein, and A is

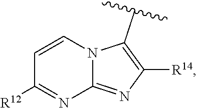

wherein R¹² is —C(O)OH, and R¹⁴ is methyl.

In an exemplary embodiment of the formula (II), R^{1′}, R^{2′} and R^{3′} are as described herein, and A is

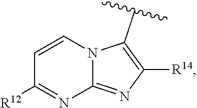

wherein R¹² is substituted or unsubstituted alkyl, and R¹⁴ comprises —C(O)OH. In an exemplary embodiment, R^{1′}, R^{2′} and R^{3′} are as described herein, and A is

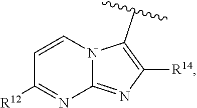

wherein R¹² is unsubstituted alkyl, and R¹⁴ comprises —C(O)OH. In an exemplary embodiment, R^{1′}, R^{2′} and R^{3′} are as described herein, and A is

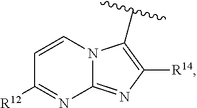

wherein R¹² is methyl, and R¹⁴ is —C(O)OH.

In an exemplary embodiment of the formula (II), R^{1′}, R^{2′} and R^{3′} are as described herein, and A is

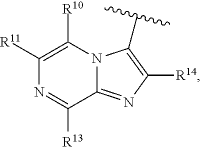

wherein R¹⁰, R¹¹, R¹³ and R¹⁴ are each independently selected from H, halogen, cyano, nitro, OR^{10a}, NR^{10a}R^{11a}, SR^{10a}, —C(O)OR^{10a}, —C(O)R^{10a}, —C(O)NR^{10a}R^{11a}, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl, wherein R^{10a} and R^{11a} are independently selected from the group consisting of H, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. In an exemplary embodiment, R^{1′}, R^{2′} and R^{3′} are as described herein, and A is

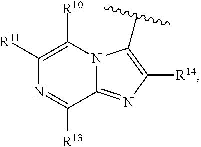

wherein R¹⁰, R¹¹, R¹³ and R¹⁴ are each independently as described herein, with the proviso that at least one member selected from R¹⁰, R¹¹, R¹³ and R¹⁴ comprises —C(O)OH.

In an exemplary embodiment of the formula (II), R^{1′}, R^{2′} and R^{3′} are as described herein, and A is

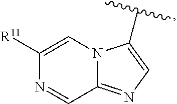

wherein R¹¹ is selected from the group consisting of H, halogen, OR^{10a}, —C(O)OR^{10a}, and substituted or unsubstituted alkyl, wherein Rica is H or substituted or unsubstituted alkyl. In an exemplary embodiment, R^{1′}, R^{2′} and R^{3′} are as described herein, and A is

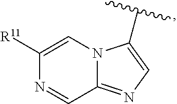

wherein R¹¹ is selected from the group consisting of F, Cl, halogen-substituted alkyl, unsubstituted alkyl, OR^{10a}, and —C(O)OR^{10a}, wherein Rica is unsubstituted alkyl. In an exemplary embodiment, R^{1′}, R^{2′} and R^{3′} are as described herein, and A is

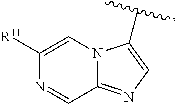

wherein R¹¹ is selected from the group consisting of F, Cl, —CF₃, —CH₃, —OCH₃, —C(O)OCH₂CH₃, —C(O)OH. In an exemplary embodiment, R^{1′}, R^{2′} and R^{3′} are as described herein, and A is

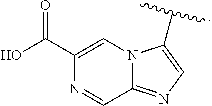 [SMILES: *c1cnc2cnc(C(=O)O)cn12]

In an exemplary embodiment of the formula (II), R^{1′}, R^{2′} and R^{3′} are as described herein, and A is

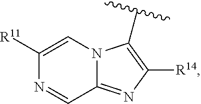

wherein R¹¹ comprises —C(O)OH, and R¹⁴ is substituted or unsubstituted alkyl. In an exemplary embodiment, R^{1′}, R^{2′} and R^{3′} are as described herein, and A is

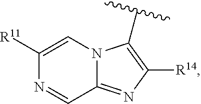

wherein R¹¹ comprises —C(O)OH, and R¹⁴ is unsubstituted alkyl. In an exemplary embodiment, R^{1′}, R^{2′} and R^{3′} are as described herein, and A is

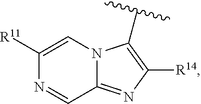

wherein R¹¹ is —C(O)OH, and R¹⁴ is methyl.

In an exemplary embodiment of the formula (II), R^{1′}, R^{2′} and R^{3′} are as described herein, and A is

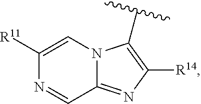

wherein R¹¹ is substituted or unsubstituted alkyl, and R¹⁴ comprises —C(O)OH. In an exemplary embodiment, R^{1′}, R^{2′} and R^{3′} are as described herein, and A is

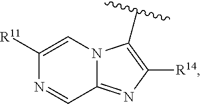

wherein R¹¹ is unsubstituted alkyl, and R¹⁴ comprises —C(O)OH. In an exemplary embodiment, R^{1′}, R^{2′} and R^{3′} are as described herein, and A is

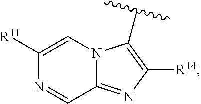

wherein R¹¹ is methyl, and R¹⁴ is —C(O)OH.

In an exemplary embodiment of the formula (II), R^{1′}, R^{2′} and R^{3′} are as described herein, and A is

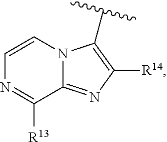

wherein R¹³ comprises —C(O)OH, and R¹⁴ is substituted or unsubstituted alkyl. In an exemplary embodiment, R^{1′}, R^{2′} and R³ are as described herein, and A is

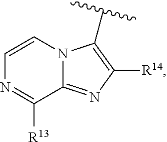

wherein R¹³ comprises —C(O)OH, and R¹⁴ is unsubstituted alkyl. In an exemplary embodiment, R^{1′}, R^{2′} and R^{3′} are as described herein, and A is

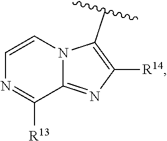

wherein R¹³ is —C(O)OH, and R¹⁴ is methyl.

In an exemplary embodiment of the formula (II), R^{1′}, R^{2′} and R^{3′} are as described herein, and A is

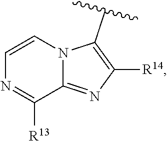

wherein R¹³ is substituted or unsubstituted alkyl, and R¹⁴ comprises —C(O)OH. In an exemplary embodiment, R^{1′}, R^{2′} and R^{3′} are as described herein, and A is

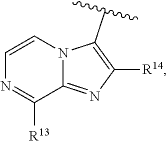

wherein R¹³ is unsubstituted alkyl, and R¹⁴ comprises —C(O)OH. In an exemplary embodiment, R^{1′}, R^{2′} and R^{3′} are as described herein, and A is

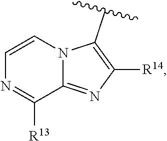

wherein R¹³ is methyl, and R¹⁴ is —C(O)OH.

In an exemplary embodiment of the formula (II), R^{1′}, R^{2′} and R^{3′} are as described herein, and A is

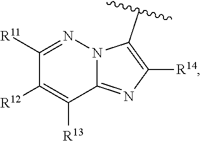

wherein R¹¹, R¹², R¹³ and R¹⁴ are each independently selected from H, halogen, cyano, nitro, OR^{10a}, NR^{10a}R^{11a}, SR^{10a}, —C(O)OR^{10a}, —C(O)R^{10a}, —C(O)NR^{10a}R^{11a}, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl, wherein R^{10a} and R^{11a} are independently selected from the group consisting of H, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. In an exemplary embodiment, R^{1′}, R^{2′} and R^{3′} are as described herein, and A is

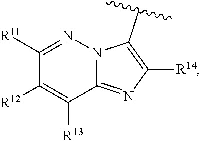

wherein R¹¹, R¹², R¹³ and R¹⁴ are each independently as described herein, with the proviso that at least one member selected from R¹¹, R¹², R¹³ and R¹⁴ comprises —C(O)OH.

In an exemplary embodiment of the formula (II), R^{1′}, R^{2′} and R^{3′} are as described herein, and A is

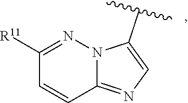

wherein R¹¹ is selected from the group consisting of H, halogen, OR^{10a}, —C(O)OR^{10a}, and substituted or unsubstituted alkyl, wherein Rica is H or substituted or unsubstituted alkyl. In an exemplary embodiment, R^{1′}, R^{2′} and R^{3′} are as described herein, and A is

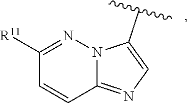

wherein R¹¹ is selected from the group consisting of F, Cl, halogen-substituted alkyl, unsubstituted alkyl, OR^{10a}, and —C(O)OR^{10a}, wherein Rica is unsubstituted alkyl. In an exemplary embodiment, R^{1′}, R^{2′} and R^{3′} are as described herein, and A is

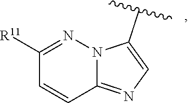

wherein R¹¹ is selected from the group consisting of F, Cl, —CF₃, —CH₃, —OCH₃, —C(O) OCH₂CH₃, and —C(O)OH. In an exemplary embodiment, R^{1′}, R^{2′} and R^{3′} are as described herein, and A is

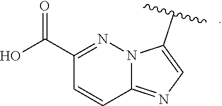 [SMILES: *c1cnc2ccc(C(=O)O)nn12]

In an exemplary embodiment of the formula (II), R^{1′}, R^{2′} and R^{3′} are as described herein, and A is

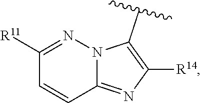

wherein R¹¹ comprises —C(O)OH, and R¹⁴ is substituted or unsubstituted alkyl. In an exemplary embodiment, R^{1′}, R^{2′} and R^{3′} are as described herein, and A is

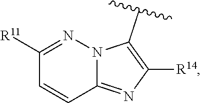

wherein R¹¹ comprises —C(O)OH, and R¹⁴ is unsubstituted alkyl. In an exemplary embodiment, R^{1′}, R^{2′} and R^{3′} are as described herein, and A is

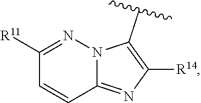

wherein R¹¹ is —C(O)OH, and R¹⁴ is methyl.

In an exemplary embodiment of the formula (II), R^{1′}, R^{2′} and R^{3′} are as described herein, and A is

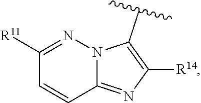

wherein R¹¹ is substituted or unsubstituted alkyl, and R¹⁴ comprises —C(O)OH. In an exemplary embodiment, R^{1′}, R^{2′} and R^{3′} are as described herein, and A is

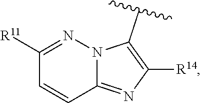

wherein R¹¹ is unsubstituted alkyl, and R¹⁴ comprises —C(O)OH. In an exemplary embodiment, R^{1′}, R^{2′} and R^{3′} are as described herein, and A is

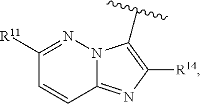

wherein R¹¹ is methyl, and R¹⁴ is —C(O)OH.

In an exemplary embodiment of the formula (II), R^{1′}, R^{2′} and R^{3′} are as described herein, and A is

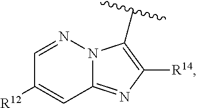

wherein R¹² comprises —C(O)OH, and R¹⁴ is substituted or unsubstituted alkyl. In an exemplary embodiment, R^{1′}, R^{2′} and R^{3′} are as described herein, and A is

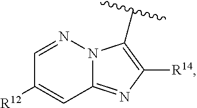

wherein R¹² comprises —C(O)OH, and R¹⁴ is unsubstituted alkyl. In an exemplary embodiment, R^{1′}, R^{2′} and R^{3′} are as described herein, and A is

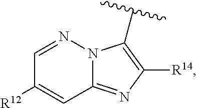

wherein R¹² is —C(O)OH, and R¹⁴ is methyl.

In an exemplary embodiment of the formula (II), R^{1′}, R^{2′} and R^{3′} are as described herein, and A is

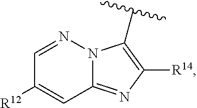

wherein R¹² is substituted or unsubstituted alkyl, and R¹⁴ comprises —C(O)OH. In an exemplary embodiment, R^{1′}, R^{2′} and R^{3′} are as described herein, and A is

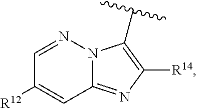

wherein R¹² is unsubstituted alkyl, and R¹⁴ comprises —C(O)OH. In an exemplary embodiment, R^{1′}, R^{2′} and R^{3′} are as described herein, and A is

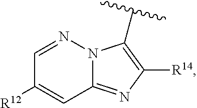

wherein R¹² is methyl, and R¹⁴ is —C(O)OH.

In an exemplary embodiment of the formula (II), the compound has the formula which is:

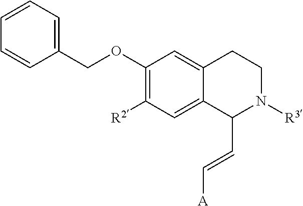

wherein R^{2′}, R^{3′} and A are as described herein, or a hydrate, solvate, or salt thereof.

In an exemplary embodiment of the formula (II), the compound has the formula which is:

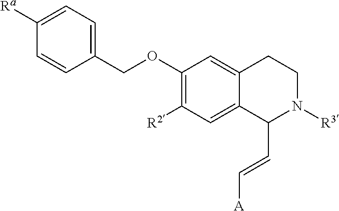

wherein R^{a}, R^{2′}, R^{3′} and A are as described herein, or a hydrate, solvate, or salt thereof.

In an exemplary embodiment of the formula (II), the compound has the formula which is:

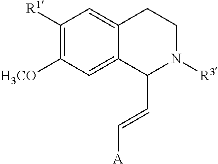

wherein R^{1′}, R^{3′} and A are as described herein, or a hydrate, solvate, or salt thereof.

In an exemplary embodiment of the formula (II), the compound has the formula which is:

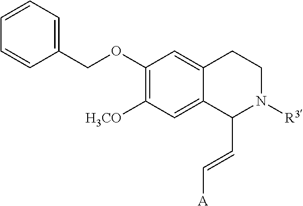

wherein R^{3′} and A are as described herein, or a hydrate, solvate, or salt thereof.

In an exemplary embodiment of the formula (II), the compound has the formula which is:

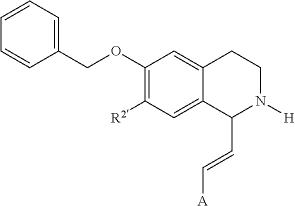

wherein R^{2′} and A are as described herein, or a hydrate, solvate, or salt thereof.

In an exemplary embodiment of the formula (II), the compound has the formula which is:

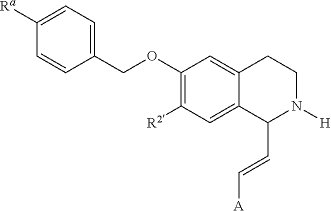

wherein R^{a}, R^{2′} and A are as described herein, or a hydrate, solvate, or salt thereof.

In an exemplary embodiment of the formula (II), the compound has the formula which is:

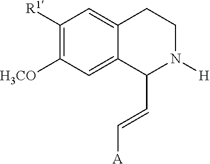

wherein R^{1′} and A are as described herein, or a hydrate, solvate, or salt thereof.

In an exemplary embodiment of the formula (II), the compound has the formula which is:

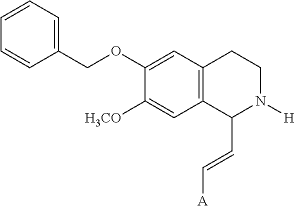

wherein A is as described herein, or a hydrate, solvate, or salt thereof.

In an exemplary embodiment of the formula (II), the compound has the formula which is:

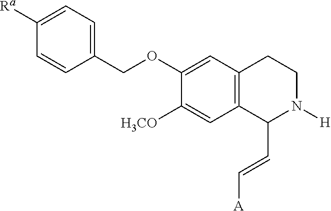

wherein R^{a} and A are as described herein, or a hydrate, solvate, or salt thereof.

In an exemplary embodiment of the formula (II), the compound has the formula which is:

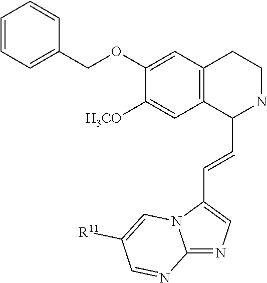

wherein R¹¹ is as described herein, or a hydrate, solvate, or salt thereof.

In an exemplary embodiment of the formula (II), the is compound has the formula which is:

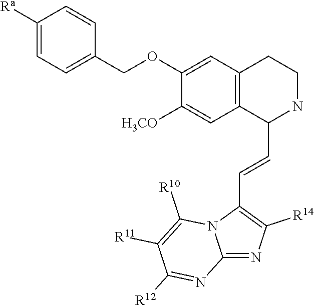

wherein R¹⁰, R¹¹, R¹² and R¹⁴ are each independently as described herein, with the proviso that at least one member selected from R¹⁰, R¹¹, R¹², and R¹⁴ comprises —C(O)OH, and R^{a} is as described herein, or a hydrate, solvate, or salt thereof.

Preferable embodiment of compound (I) includes the following compounds (I-A) to (I-C). Compound (I) is preferably compound (I-B) which is also referred as compound (IA), more preferably compound (I-C) which is also referred as the following compound (IA-A).

### [Compound (I-A)]

Compound (I) wherein

### R¹ is

(1) a C₁₋₆ alkyl group (e.g., methyl, ethyl) substituted by phenyl group(s) optionally substituted by 1 to 3 substituents selected from
(a) a halogen atom (e.g., a chlorine atom), and
(b) an optionally halogenated C₁₋₆ alkyl group (e.g., methyl, trifluoromethyl),
   (2) a C₁₋₆ alkyl group (e.g., methyl) substituted by C₃₋₁₀ cycloalkyl group(s) (e.g., cyclopropyl), or
   (3) a C₁₋₆ alkyl group (e.g., methyl) substituted by pyridyl group(s);
   R² is a C₁₋₆ alkoxy group (e.g., methoxy);

### R³ is H; and

### Ar is

(1) a phenyl group optionally substituted by 1 to 3 substituents selected from
(a) a halogen atom (e.g., a chlorine atom, a bromine atom),
(b) a C₁₋₆ alkyl group (e.g., methyl),
(c) a C₁₋₆ alkoxy group (e.g., methoxy) optionally substituted by 1 to 3 substituents selected from
   (i) a C₃₋₁₀ cycloalkyl group (e.g., cyclopropyl, cyclobutyl),
   (ii) a 5- or 6-membered monocyclic aromatic heterocyclic group (e.g., pyridyl, pyridazinyl) optionally substituted by 1 to 3 substituents selected from a halogen atom (e.g., a chlorine atom), an amino group, a C₁₋₆ alkyl group (e.g., methyl) and a C₁₋₆ alkoxy group (e.g., methoxy),
   (iii) a 3- to 8-membered monocyclic non-aromatic heterocyclic group (e.g., tetrahydropyranyl, tetrahydrofuryl, piperidyl) optionally substituted by 1 to 3 substituents selected from an oxo group and a C₁₋₆ alkyl group (e.g., methyl), and
   (iv) a 8- to 14-membered bicyclic aromatic heterocyclic group (e.g., imidazopyridyl, benzotriazolyl, indazolyl, quinolyl) optionally substituted by 1 to 3 C₁₋₆ alkyl groups (e.g., methyl),
(d) a C₂₋₆ alkynyloxy group (e.g., 2-propynyloxy),
(e) a C₁₋₃ alkylenedioxy group (e.g., methylenedioxy, ethylenedioxy),
(f) a C₆₋₁₄ aryl group (e.g., phenyl) optionally substituted by 1 to 3 substituents selected from
   (i) a C₁₋₆ alkoxy group (e.g., methoxy), and
   (ii) a 5- or 6-membered monocyclic aromatic heterocyclic group (e.g., pyrazolyl),
(g) a C₇₋₁₆ aralkyloxy group (e.g., benzyloxy) optionally substituted by 1 to 3 substituents selected from
   (i) a cyano group,
   (ii) a C₁₋₆ alkyl group (e.g., methyl) optionally substituted by 1 to 3 amino groups,
   (iii) an optionally halogenated C₁₋₆ alkoxy group (e.g., methoxy, difluoromethoxy),
   (iv) a C₁₋₆ alkyl-carbonyl group (e.g., acetyl),
   (v) a C₁₋₆ alkoxy-carbonyl group (e.g., methoxycarbonyl), and
   (vi) a C₁₋₆ alkylsulfonyl group (e.g., methylsulfonyl),
(h) a 5- or 6-membered monocyclic aromatic heterocyclic group (e.g., imidazolyl, pyrazolyl, isoxazolyl, pyridyl, pyrimidinyl, pyridazinyl, pyrazinyl) optionally substituted by 1 to 3 substituents selected from
   (i) a cyano group,
   (ii) an optionally halogenated C₁₋₆ alkyl group (e.g., methyl, trifluoromethyl),
   (iii) an optionally halogenated C₁₋₆ alkoxy group (e.g., methoxy, 2,2,2-trifluoroethoxy),
   (iv) a mono-C₁₋₆ alkylamino group (e.g., methylamino),
   (v) a C₁₋₆ alkoxy-carbonyl group (e.g., ethoxycarbonyl), and
   (vi) a C₇₋₁₆ aralkyl group (e.g., benzyl),
(i) a 8- to 14-membered bicyclic aromatic heterocyclic group (e.g., quinoxalinyl, pyrazolopyridyl, imidazopyridyl), and
(j) a 3- to 8-membered monocyclic non-aromatic heterocyclic group (e.g., dihydropyranyl),
   (2) a benzofuryl group optionally substituted by 1 to 3 C₁₋₆ alkoxy groups (e.g., methoxy),
   (3) a pyrrolopyridyl group (e.g., pyrrolo[2.3-b]pyridyl) optionally substituted by 1 to 3 substituents selected from
(a) a hydroxy group,
(b) a C₁₋₆ alkyl group (e.g., methyl),
(c) a C₁₋₆ alkoxy group (e.g., methoxy, ethoxy, propoxy) optionally substituted by 1 to 3 substituents selected from
   (i) a halogen atom (e.g., a fluorine atom),
   (ii) a hydroxy group,
   (iii) a 3- to 8-membered monocyclic non-aromatic heterocyclic group (e.g., diazirinyl) optionally substituted by 1 to 3 substituents selected from a C₁₋₆ alkyl group (e.g., methyl) and a C₂₋₆ alkynyl group (e.g., 3-butynyl), and
(d) a C₂₋₆ alkynyl group (e.g., 2-propynyl),
   (4) an imidazopyrimidinyl group (e.g., imidazo[1.2-a]pyrimidinyl) optionally substituted by 1 to 3 substituents selected from
(a) a halogen atom (e.g., a fluorine atom, a chlorine atom),
(b) a carboxy group,
(c) an optionally halogenated C₁₋₆ alkyl group (e.g., methyl, trifluoromethyl),
(d) a C₁₋₆ alkoxy group (e.g., methoxy), and
(e) a C₁₋₆ alkoxy-carbonyl group (e.g., ethoxycarbonyl),
   (5) an imidazopyrazinyl group (e.g., imidazo[1.2-a]pyrazinyl) optionally substituted by 1 to 3 carboxy groups,
   (6) an imidazopyridazinyl group (e.g., imidazo[1.2-b]pyridazinyl) optionally substituted by 1 to 3 substituents selected from
(a) a carboxy group, and
(b) a C₁₋₆ alkoxy-carbonyl group (e.g., methoxycarbonyl), or
   (7) an imidazopyridyl group (e.g., imidazo[1.2-a]pyridyl) optionally substituted by 1 to 3 substituents selected from
(a) a carboxy group, and
(b) a C-6 alkoxy-carbonyl group (e.g., methoxycarbonyl).

### [Compound (I-B)]

Compound (I) wherein

R¹ is a C₁₋₆ alkyl group (e.g., methyl) substituted by phenyl group(s) optionally substituted by 1 to 3 halogen atoms (e.g., a chlorine atom);
R² is a methoxy group;

### R³ is H; and

### Ar is

(1) a phenyl group substituted by 2 or 3 substituents selected from
(a) a C₁₋₆ alkyl group (e.g., methyl),
(b) a C₁₋₆ alkoxy group (e.g., methoxy) optionally substituted by 1 to 3 of 5- or 6-membered monocyclic aromatic heterocyclic groups (e.g., pyridyl) optionally substituted by 1 to 3 halogen atoms (e.g., a chlorine atom), and
(c) a 5- or 6-membered monocyclic aromatic heterocyclic group (e.g., pyrazolyl, pyrimidinyl) optionally substituted by 1 to 3 C₁₋₆ alkyl groups (e.g., methyl), or
   (2) a pyrrolopyridyl group (e.g., pyrrolo[2.3-b]pyridyl) substituted by 2 substituents selected from
(a) a C₁₋₆ alkyl group (e.g., methyl), and
(b) a C₁₋₆ alkoxy group (e.g., methoxy).

### [Compound (I-C)]

Compound (I) wherein

R¹ is a benzyl group optionally substituted by 1 to 3 halogen atoms (e.g., a chlorine atom);
R² is a methoxy group;

### R³ is H; and

### Ar is

(1) a phenyl group substituted by 2 or 3 substituents selected from
(a) a C₁₋₆ alkyl group (e.g., methyl),
(b) a C₁₋₆ alkoxy group (e.g., methoxy) optionally substituted by one pyridyl group optionally substituted by 1 to 3 halogen atoms (e.g., a chlorine atom), and
(c) a pyrazolyl or pyrimidinyl group optionally substituted by 1 to 3 C₁₋₆ alkyl groups (e.g., methyl), or
   (2) a pyrrolopyridyl group (e.g., pyrrolo[2.3-b]pyridyl) substituted by 2 substituents selected from
(a) a C₁₋₆ alkyl group (e.g., methyl), and
(b) a C₁₋₆ alkoxy group (e.g., methoxy).

Preferable embodiment of compound (IA) includes the following compound (IA-A).

### [Compound (IA-A)]

Compound (IA) wherein

R^{1A} is a benzyl group optionally substituted by 1 to 3 halogen atoms (e.g., a chlorine atom); and

### Ar^{A} is

(1) a phenyl group substituted by 2 or 3 substituents selected from
(a) a C₁₋₆ alkyl group (e.g., methyl),
(b) a C₁₋₆ alkoxy group (e.g., methoxy) optionally substituted by one pyridyl group optionally substituted by 1 to 3 halogen atoms (e.g., a chlorine atom), and
(c) a pyrazolyl or pyrimidinyl group optionally substituted by 1 to 3 C₁₋₆ alkyl groups (e.g., methyl), or
   (2) a pyrrolopyridyl group (e.g., pyrrolo[2.3-b]pyridyl) substituted by 2 substituents selected from
(a) a C₁₋₆ alkyl group (e.g., methyl), and
(b) a C₁₋₆ alkoxy group (e.g., methoxy).

Preferable embodiment of compound (IB) includes the following compound (IB-A) and compound (IB-B).

### [Compound (IB-A)]

Compound (IB) wherein

R^{1B} is a C₁₋₆ alkyl group (e.g., methyl, ethyl) substituted by phenyl group(s) optionally substituted by 1 to 3 of optionally halogenated C₁₋₆ alkyl group(s) (e.g., methyl, trifluoromethyl);
R² is a C₁₋₆ alkoxy group (e.g., methoxy);

### R³ is H; and

Ar^{B} is (1) imidazo[1.2-a]pyrimidinyl optionally substituted by 1 to 3 substituents selected from
(a) a halogen atom (e.g., a fluorine atom, a chlorine atom),
(b) a carboxy group,
(c) an optionally halogenated C₁₋₆ alkyl group (e.g., methyl, trifluoromethyl),
(d) a C₁₋₆ alkoxy group (e.g., methoxy), and
(e) a C₁₋₆ alkoxy-carbonyl group (e.g., ethoxycarbonyl),
   (2) imidazo[1.2-a]pyrazinyl optionally substituted by 1 to 3 carboxy groups,
   (3) imidazo[1.2-b]pyridazinyl optionally substituted by 1 to 3 substituents selected from
(a) a carboxy group, and
(b) a C₁₋₆ alkoxy-carbonyl group (e.g., methoxycarbonyl), or
   (4) imidazo[1.2-a]pyridyl optionally substituted by 1 to 3 substituents selected from
(a) a carboxy group, and
(b) a C₁₋₆ alkoxy-carbonyl group (e.g., methoxycarbonyl).

### [Compound (IB-B)]

Compound (IB) wherein

R^{1B} is a benzyl group;
R² is a methoxy group;

### R³ is H; and

Ar^{B} is imidazo[1.2-b]pyridazinyl substituted by one substituent selected from
(a) a carboxy group, and
(b) a C₁₋₆ alkoxy-carbonyl group (e.g., methoxycarbonyl).

Specific examples of compound (I) include the compounds of Examples AA to AL, BA, CA, CB, DA to DD, and 1 to 108.

Among them, preferable examples of compound (I) include the following compounds.
6-(benzyloxy)-7-methoxy-1-[(E)-2-(5-methoxy-1-methyl-1H-pyrrolo[2,3-b]pyridin-3-yl)ethenyl]-1,2,3,4-tetrahydroisoquinoline, or a hydrate, solvate, or salt thereof (Example 7);
6-(benzyloxy)-7-methoxy-1-{(E)-2-[2-methyl-5-(pyrimidin-5-yl)phenyl]ethenyl}-1,2,3,4-tetrahydroisoquinoline, or a hydrate, solvate, or salt thereof (Example 17);
6-[(3-chlorophenyl)methoxy]-7-methoxy-1-[(E)-2-(5-methoxy-1-methyl-1H-pyrrolo[2,3-b]pyridin-3-yl)ethenyl]-1,2,3,4-tetrahydroisoquinoline, or a hydrate, solvate, or salt thereof (Example 24);
6-(benzyloxy)-7-methoxy-1-[(E)-2-{4-methoxy-2-methyl-5-[(pyridin-4-yl)methoxy]phenyl}ethenyl]-1,2,3,4-tetrahydroisoquinoline, or a hydrate, solvate, or salt thereof (Example 28);
6-(benzyloxy)-7-methoxy-1-{(E)-2-[2-methyl-4-(1-methyl-1H-pyrazol-4-yl)phenyl]ethenyl}-1,2,3,4-tetrahydroisoquinoline, or a hydrate, solvate, or salt thereof (Example 59); and
6-(benzyloxy)-7-methoxy-1-[(E)-2-{4-methoxy-5-[(6-methoxypyridin-2-yl)methoxy]-2-methylphenyl}ethenyl]-1,2,3,4-tetrahydroisoquinoline, or a hydrate, solvate, or salt thereof (Example 98).

In an exemplary embodiment, the invention provides a compound described herein, or a salt, hydrate or solvate thereof, or a combination thereof. In an exemplary embodiment, the invention provides a compound represented by a formula provided herein, or a salt, hydrate or solvate thereof, or a combination thereof. In an exemplary embodiment, the invention provides a compound described herein, or a salt, hydrate or solvate thereof. In an exemplary embodiment, the invention provides a compound described herein, or a salt thereof. In an exemplary embodiment, the salt is a pharmaceutically acceptable salt. In an exemplary embodiment, the invention provides a compound described herein, or a hydrate thereof. In an exemplary embodiment, the invention provides a compound described herein, or a solvate thereof. In an exemplary embodiment, the invention provides a compound described herein, or a prodrug thereof. In an exemplary embodiment, the invention provides a salt of a compound described herein. In an exemplary embodiment, the invention provides a pharmaceutically acceptable salt of a compound described herein. In an exemplary embodiment, the invention provides a hydrate of a compound described herein. In an exemplary embodiment, the invention provides a solvate of a compound described herein. In an exemplary embodiment, the invention provides a prodrug of a compound described herein.

In an exemplary embodiment, alkyl is linear alkyl. In another exemplary embodiment, alkyl is branched alkyl.

In an exemplary embodiment, heteroalkyl is linear heteroalkyl. In another exemplary embodiment, heteroalkyl is branched heteroalkyl.

### III.b) Preparation of Compound (I)

Compound (I) of use in the invention can be prepared using commercially available starting materials, known intermediates, or by using the synthetic methods described herein, or published in references described and incorporated by reference herein.

### A General Procedure to Make Compounds of the Invention

The raw material compound and reagent used and the compound obtained in each step in the following production method may be each in a form of a salt, and examples of such salt include those similar to the salts of compound (I) and the like.

When the compound obtained in each step is a free form, it can be converted to the objective salt according to a method known per se. When the compound obtained in each step is a salt, it can be converted to the objective free form or the other salt according to a method known per se.

The compound obtained in each step can be used directly as the reaction mixture or as a crude product for the next reaction. Alternatively, the compound obtained in each step can be isolated and purified from a reaction mixture according to a method known per se, for example, a separation means such as concentration, crystallization, recrystallization, distillation, solvent extraction, fractional distillation, chromatography and the like.

When the raw material compound and reagent used in each step are commercially available, the commercially available product can also be used directly.

In the reaction in each step, while the reaction time varies depending on the kind of the reagent and solvent to be used, it is generally 1 min-48 hr, preferably 10 min-8 hr, unless otherwise specified.

In the reaction in each step, while the reaction temperature varies depending on the kind of the reagent and solvent to be used, it is generally −78° C.-300° C., preferably −78° C.-150° C., unless otherwise specified.

In the reaction in each step, while the pressure varies depending on the kind of the reagent and solvent to be used, it is generally 1 atm-20 atm, preferably 1 atm-3 atm, unless otherwise specified.

Microwave synthesizer such as Initiator manufactured by Biotage and the like may be used for the reaction in each step. While the reaction temperature varies depending on the kind of the reagent and solvent to be used, it is generally room temperature—300° C., preferably 50° C.-250° C., unless otherwise specified. While the reaction time varies depending on the kind of the reagent and solvent to be used, it is generally 1 min-48 hr, preferably 1 min-8 hr, unless otherwise specified.

In the reaction in each step, the reagent is used in an amount of 0.5 equivalents-20 equivalents, preferably 0.8 equivalents-5 equivalents, relative to the substrate, unless otherwise specified. When the reagent is used as a catalyst, the reagent is used in an amount of 0.001 equivalent-1 equivalent, preferably 0.01 equivalent-0.2 equivalent, relative to the substrate. When the reagent is used as a reaction solvent, the reagent is used in a solvent amount.

Unless otherwise specified, the reaction in each step is carried out without solvent, or by dissolving or suspending the raw material compound in a suitable solvent. Examples of the solvent include those described in Examples and the following solvents.

alcohols: methanol, ethanol, tert-butyl alcohol, 2-methoxyethanol and the like;
ethers: diethyl ether, diphenyl ether, tetrahydrofuran, 1,2-dimethoxyethane and the like;
aromatic hydrocarbons: chlorobenzene, toluene, xylene and the like;
saturated hydrocarbons: cyclohexane, hexane and the like; amides: N,N-dimethylformamide, N-methylpyrrolidone and the like;
halogenated hydrocarbons: dichloromethane, carbon tetrachloride and the like;
nitriles: acetonitrile and the like;
sulfoxides: dimethyl sulfoxide and the like;
aromatic organic bases: pyridine and the like;
anhydrides: acetic anhydride and the like;
organic acids: formic acid, acetic acid, trifluoroacetic acid and the like;
inorganic acids: hydrochloric acid, sulfuric acid and the like;
esters: ethyl acetate and the like;
ketones: acetone, methyl ethyl ketone and the like; water.

The above-mentioned solvent can be used in a mixture of two or more kinds thereof in an appropriate ratio.

When a base is used for the reaction in each step, examples thereof include those described in Examples and the following bases.

inorganic bases: sodium hydroxide, magnesium hydroxide, sodium carbonate, calcium carbonate, sodium hydrogencarbonate, cesium carbonate, potassium acetate and the like;
organic bases: triethylamine, diethylamine, N,N-diisopropylethylamine (DIPEA), pyridine, 4-dimethylaminopyridine, N,N-dimethylaniline, 1,4-diazabicyclo[2.2.2]octane, 1,8-diazabicyclo[5.4.0]-7-undecene, imidazole, piperidine and the like;
metal alkoxides: sodium ethoxide, potassium tert-butoxide and the like;
alkali metal hydrides: sodium hydride and the like;
metal amides: sodium amide, lithium diisopropylamide, lithium hexamethyldisilazide and the like;
organic lithiums: n-butyllithium and the like.

When an acid or an acid catalyst is used for the reaction in each step, examples thereof include those described in Examples and the following acids and acid catalysts.

inorganic acids: hydrochloric acid, sulfuric acid, nitric acid, hydrobromic acid, phosphoric acid and the like;
organic acids: acetic acid, trifluoroacetic acid, citric acid, p-toluenesulfonic acid, 10-camphorsulfonic acid and the like;
Lewis acid: boron trifluoride diethyl ether complex, zinc iodide, anhydrous aluminium chloride, anhydrous zinc chloride, anhydrous iron chloride and the like.

Unless otherwise specified, the reaction in each step is carried out according to a method known per se, for example, the method described in Jikken Kagaku Kouza, 5th Edition, vol. 13-19 (the Chemical Society of Japan ed.); Shin Jikken Kagaku Kouza, vol. 14-15 (the Chemical Society of Japan ed.); Fine Organic Chemistry, Revised 2nd Edition (L. F. Tietze, Th. Eicher, Nankodo); Organic Name Reactions, the Reaction Mechanism and Essence, Revised Edition (Hideo Togo, Kodansha); ORGANIC SYNTHESES Collective Volume I-VII (John Wiley & Sons Inc.); Modern Organic Synthesis in the Laboratory A Collection of Standard Experimental Procedures (Jie Jack Li, OXFORD UNIVERSITY); Comprehensive Heterocyclic Chemistry III, Vol. 1-Vol. 14 (Elsevier Japan); Strategic Applications of Named Reactions in Organic Synthesis (translated by Kiyoshi Tomioka, Kagakudojin); Comprehensive Organic Transformations (VCH Publishers Inc.), 1989, or the like, or the method described in Examples.

In each step, the protection or deprotection reaction of a functional group is carried out according to a method known per se, for example, the method described in “Protective Groups in Organic Synthesis, 4th Ed”, Wiley-Interscience, Inc., 2007 (Theodora W. Greene, Peter G. M. Wuts); “Protecting Groups 3rd Ed.” Thieme, 2004 (P. J. Kocienski), or the like, or the method described in Examples.

Examples of the protecting group for a hydroxy group of an alcohol and the like and a phenolic hydroxy group include ether-type protecting groups such as methoxymethyl ether, benzyl ether, tert-butyldimethylsilyl ether, tetrahydropyranyl ether and the like; carboxylate ester-type protecting groups such as acetate ester and the like; sulfonate ester-type protecting groups such as methanesulfonate ester and the like; carbonate ester-type protecting groups such as tert-butylcarbonate and the like, and the like.

Examples of the protecting group for a carbonyl group of an aldehyde include acetal-type protecting groups such as dimethylacetal and the like; cyclic acetal-type protecting groups such as 1,3-dioxane and the like, and the like.

Examples of the protecting group for a carbonyl group of a ketone include ketal-type protecting groups such as dimethylketal and the like; cyclic ketal-type protecting groups such as 1,3-dioxane and the like; oxime-type protecting groups such as O-methyloxime and the like; hydrazone-type protecting groups such as N,N-dimethylhydrazone and the like, and the like.

Examples of the protecting group for a carboxyl group include ester-type protecting groups such as methyl ester and the like; amide-type protecting groups such as N,N-dimethylamide and the like, and the like.

Examples of the protecting group for a thiol include ether-type protecting groups such as benzyl thioether and the like; ester-type protecting groups such as thioacetate ester, thiocarbonate, thiocarbamate and the like, and the like.

Examples of the protecting group for an amino group and an aromatic heterocycle such as imidazole, pyrrole, indole and the like include carbamate-type protecting groups such as benzyl carbamate and the like; amide-type protecting groups such as acetamide and the like; alkyl amine-type protecting groups such as N-triphenylmethylamine and the like; sulfonamide-type protecting groups such as methanesulfonamide and the like, and the like.

The protecting groups can be removed according to a method known per se, for example, by employing a method using acid, base, ultraviolet rays, hydrazine, phenylhydrazine, sodium N-methyldithiocarbamate, tetrabutylammonium fluoride, palladium acetate, trialkylsilyl halide (e.g., trimethylsilyl iodide, trimethylsilyl bromide) and the like, a reduction method, and the like.

When reduction reaction is carried out in each step, examples of the reducing agent to be used include metal hydrides such as lithium aluminium hydride, sodium triacetoxyborohydride, sodium cyanoborohydride, diisobutylaluminium hydride (DIBAL-H), sodium borohydride, tetramethylammonium triacetoxyborohydride and the like; boranes such as borane tetrahydrofuran complex and the like; Raney nickel; Raney cobalt; hydrogen; formic acid; triethylsilane and the like. When carbon-carbon double bond or triple bond is reduced, a method using a catalyst such as palladium-carbon, Lindlar's catalyst and the like may be employed.

When oxidation reaction is carried out in each step, examples of the oxidizing agent to be used include peroxides such as m-chloroperbenzoic acid (mCPBA), hydrogen peroxide, tert-butylhydroperoxide and the like; perchlorates such as tetrabutylammonium perchlorate and the like; chlorates such as sodium chlorate and the like; chlorites such as sodium chlorite and the like; periodates such as sodium periodate and the like; hypervalent iodine reagents such as iodosylbenzene and the like; reagents containing manganese such as manganese dioxide, potassium permanganate and the like; leads such as lead tetraacetate and the like; reagents containing chromium such as pyridinium chlorochromate (PCC), pyridinium dichromate (PDC), Jones reagent and the like; halogen compounds such as N-bromosuccinimide (NBS) and the like; oxygen; ozone; sulfur trioxide-pyridine complex; osmium tetroxide; selenium dioxide; 2,3-dichloro-5,6-dicyano-1,4-benzoquinone (DDQ) and the like.

When radical cyclization reaction is carried out in each step, examples of the radical initiator to be used include azo compounds such as azobisisobutyronitrile (AIBN) and the like; water-soluble radical initiators such as 4,4′-azobis-4-cyanopentanoic acid (ACPA) and the like; triethylboron in the presence of air or oxygen; benzoyl peroxide and the like. Examples of the radical reagent to be used include tributylstannane, tristrimethylsilylsilane, 1,1,2,2-tetraphenyldisilane, diphenylsilane, samarium iodide and the like.

When Wittig reaction is carried out in each step, examples of the Wittig reagent to be used include alkylidene phosphoranes and the like. The alkylidene phosphoranes can be prepared according to a method known per se, for example, by reacting a phosphonium salt with a strong base.

When Horner-Emmons reaction is carried out in each step, examples of the reagent to be used include phosphonoacetates such as methyl dimethylphosphonoacetate, ethyl diethylphosphonoacetate and the like; and bases such as alkali metal hydrides, organic lithiums and the like.

When Friedel-Crafts reaction is carried out in each step, examples of the reagent to be used include a combination of a Lewis acid and an acid chloride or a combination of a Lewis acid and an alkylating agent (e.g., an alkyl halide, an alcohol, an olefin etc.). Alternatively, an organic acid or an inorganic acid can also be used instead of a Lewis acid, and an anhydride such as acetic anhydride and the like can also be used instead of an acid chloride.

When aromatic nucleophilic substitution reaction is carried out in each step, a nucleophile (e.g., an amine, imidazole etc.) and a base (e.g., an organic base etc.) are used as reagents.

When nucleophilic addition reaction by a carbo anion, nucleophilic 1,4-addition reaction (Michael addition reaction) by a carbo anion or nucleophilic substitution reaction by a carbo anion is carried out in each step, examples of the base to be used for generation of the carbo anion include organic lithiums, metal alkoxides, inorganic bases, organic bases and the like.

When Grignard reaction is carried out in each step, examples of the Grignard reagent to be used include arylmagnesium halides such as phenylmagnesium bromide and the like; and alkylmagnesium halides such as methylmagnesium bromide and the like. The Grignard reagent can be prepared according to a method known per se, for example, by reacting an alkyl halide or an aryl halide with a metal magnesium or isopropylmagnesium chloride-lithium chloride complex in an ether, tetrahydrofuran and the like as a solvent.

When Knoevenagel condensation reaction is carried out in each step, a compound having an activated methylene group with two electron withdrawing groups (e.g., malonic acid, diethyl malonate, malononitrile etc.) and a base (e.g., an organic base, a metal alkoxide, an inorganic base) are used as a reagent.

When Vilsmeier-Haack reaction is carried out in each step, phosphoryl chloride and an amide derivative (e.g., N,N-dimethylformamide etc.) are used as a reagent.

When azidation reaction of an alcohol, an alkyl halide or a sulfonate is carried out in each step, examples of the azidating agent to be used include diphenylphosphorylazide (DPPA), trimethylsilylazide, sodium azide and the like. For example, for the azidation reaction of an alcohol, a method using diphenylphosphorylazide and 1,8-diazabicyclo[5.4.0]undec-7-ene (DBU), a method using trimethylsilylazide and a Lewis acid, a method using diphenylphosphorylazide, triphenylphosphine and azodicarboxylate, and the like are employed.

When reductive amination reaction is carried out in each step, examples of the reducing agent to be used include sodium triacetoxyborohydride, sodium cyanoborohydride, hydrogen, formic acid and the like. When the substrate is an amine compound, examples of the carbonyl compound to be used include paraformaldehyde, aldehydes such as acetaldehyde and the like, and ketones such as cyclohexanone and the like. When the substrate is a carbonyl compound, examples of the amine to be used include ammonia, primary amines such as methylamine and the like; secondary amines such as dimethylamine and the like, and the like.

When Mitsunobu reaction is carried out in each step, an azodicarboxylate (e.g., diethyl azodicarboxylate (DEAD), diisopropyl azodicarboxylate (DIAD) etc.) and triphenylphosphine are used as a reagent.

When esterification reaction, amidation reaction or ureation reaction is carried out in each step, examples of the reagent to be used include acyl halides such as acid chlorides, acid bromides and the like; activated carboxylic acids such as anhydrides, activated esters, sulfates and the like. Examples of the activating agent of the carboxylic acid include carbodiimide condensing agents such as 1-ethyl-3-(3-dimethylaminopropyl)carbodiimide hydrochloride (EDC HCl) and the like; triazine condensing agents such as 4-(4,6-dimethoxy-1,3,5-triazin-2-yl)-4-methylmorpholinium chloride n-hydrate (DMT-MM) and the like; carbonate condensing agents such as 1,1-carbonyldiimidazole (CDI) and the like; diphenylphosphorylazide (DPPA); benzotriazol-1-yloxy-trisdimethylaminophosphonium salt (BOP reagent); 2-chloro-1-methyl-pyridinium iodide (Mukaiyama reagent); thionyl chloride; lower alkyl haloformates such as ethyl chloroformate and the like; O-(7-azabenzotriazol-1-yl)-N,N,N′,N′-tetramethyluronium hexafluorophosphorate (HATU); sulfuric acid; combinations thereof and the like. When carbodiimide condensing agent is used, an additive such as 1-hydroxybenzotriazole (HOBt), N-hydroxysuccinimide (HOSu), dimethylaminopyridine (DMAP) and the like may be added to the reaction system.

When coupling reaction is carried out in each step, examples of the metal catalyst to be used include palladium compounds such as palladium(II) acetate, tetrakis(triphenylphosphine)palladium(0), dichlorobis(triphenylphosphine)palladium(II), dichlorobis(triethylphosphine)palladium(II), tris(dibenzylideneacetone)dipalladium(0), 1,1′-bis(diphenylphosphino)ferrocene palladium(II) chloride and the like; nickel compounds such as tetrakis(triphenylphosphine)nickel(0) and the like; rhodium compounds such as tris(triphenylphosphine)rhodium(III) chloride and the like; cobalt compounds; copper compounds such as copper oxide, copper(I) iodide and the like; platinum compounds and the like. In addition, a base can be added to the reaction system, and examples thereof include inorganic bases and the like.

When thiocarbonylation reaction is carried out in each step, phosphorus pentasulfide is typically used as the thiocarbonylating agent. Alternatively, a reagent having a 1,3,2,4-dithiadiphosphetane-2,4-disulfide structure (e.g., 2,4-bis(4-methoxyphenyl)-1,3,2,4-dithiadiphosphetane-2,4-disulfide (Lawesson reagent) etc.) can also be used instead of phosphorus pentasulfide.

When halogenation reaction is carried out in each step, examples of the halogenating agent to be used include N-iodosuccinimide, N-bromosuccinimide (NBS), N-chlorosuccinimide (NCS), bromine, sulfuryl chloride, trichloroisocyanuric acid and the like. In addition, the reaction can be accelerated by subjecting a radical initiator such as heat, light, benzoyl peroxide, azobisisobutyronitrile and the like to the reaction system reaction.

When halogenation reaction of a hydroxy group is carried out in each step, examples of the halogenating agent to be used include hydrohalic acids and acid halides of inorganic acids, specifically, hydrochloric acid, thionyl chloride, phosphorus oxychloride and the like for chlorination, 48% hydrobromic acid and the like for bromination. In addition, a method of producing an alkyl halide by reacting an alcohol with triphenylphosphine and carbon tetrachloride or carbon tetrabromide or the like can be employed. Alternatively, a method of producing an alkyl halide via two steps comprising converting an alcohol to the corresponding sulfonate, and then reacting the sulfonate with lithium bromide, lithium chloride or sodium iodide can also be employed.

When Arbuzov reaction is carried out in each step, examples of the reagent to be used include alkyl halides such as ethyl bromoacetate and the like; and phosphites such as triethyl phosphite, tri(isopropyl) phosphite and the like.

When sulfonate esterification reaction is carried out in each step, examples of the sulfonating agent to be used include methanesulfonyl chloride, p-toluenesulfonyl chloride, methanesulfonic anhydride, p-toluenesulfonic anhydride and the like.

When hydrolysis reaction is carried out in each step, an acid or a base is used as a reagent. For acid hydrolysis reaction of tert-butyl ester, formic acid, triethylsilane and the like may be added to reductively-trap tert-butyl cation which is by-produced.

When dehydration reaction is carried out in each step, examples of the dehydrating agent to be used include sulfuric acid, diphosphorus pentaoxide, phosphorus oxychloride, N,N′-dicyclohexylcarbodiimide, alumina, polyphosphoric acid and the like.

When alkylation reaction is carried out in each step, an electrophile (e.g., an alkyl halide etc.) and a base (e.g., an organic base, an inorganic base, a metal alkoxide, a metal amide etc.) are used as reagents.

Compound (I) can be produced according to the production methods shown in the following Scheme 1, Scheme 2, Scheme 3, Scheme 4, Scheme 5, or Scheme 6. Each symbol in the formulas of the schemes is as defined above, unless otherwise specified.

Moreover, compound (I) can be produced by carrying out protection reaction, deprotection reaction, amidation reaction, sulfonamidation reaction, ureation reaction, carbamoylation reaction, alkylation reaction, Mitsunobu reaction, hydrogenation reaction, oxidation reaction, reduction reaction, halogenation reaction, coupling reaction, nucleophilic addition reaction by a carbo anion, Grignard reaction, deoxofluorination reaction, dehydration reaction and the like singly or two or more thereof in combination.

The below-mentioned compound (Ia) (compound (I) wherein R³ is H) can be produced according to the method shown in the following Scheme 1. P¹ is a protecting group (e.g., a Boc group). R^{x} is an unsubstituted C₁₋₆ alkyl group. The other symbols are as defined above.

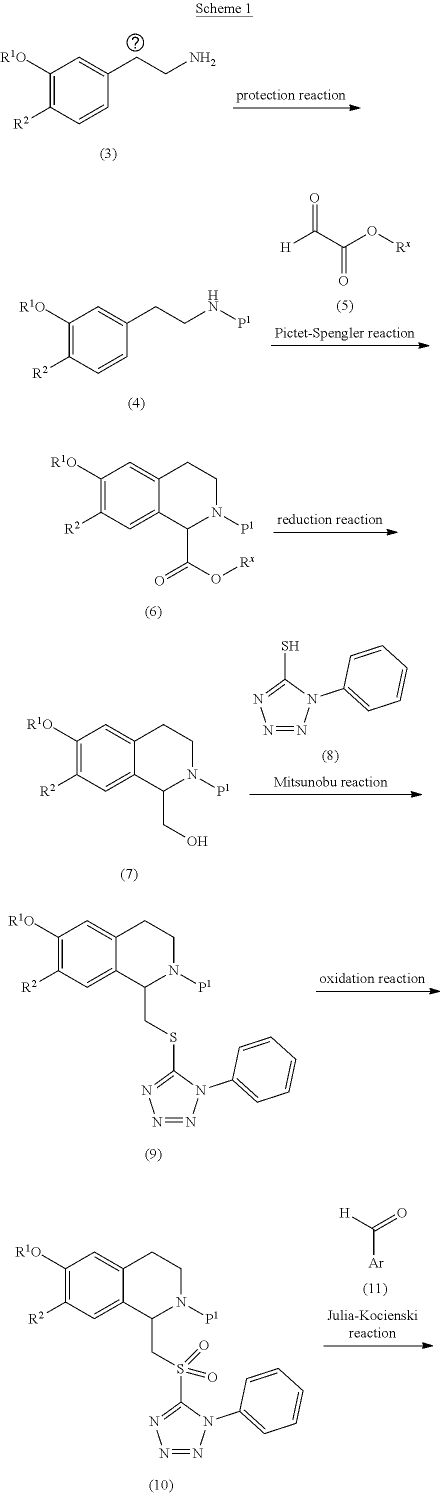

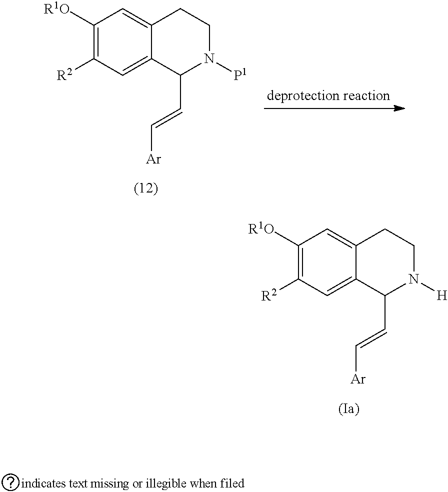

Compound (4) can be produced by subjecting compound (3) to a protection reaction. Examples of the reagent to be used for the protection reaction include a combination of di-tert-butyl decarbonate and triethylamine, and the like.

Compound (6) can be produced by subjecting compound (4) to Pictet-Spengler reaction with glyoxylic acid ester (5). Examples of reagent to be used include boron trifluoride diethyl ether complex and the like.

Compound (7) can be produced by subjecting compound (6) to a reduction reaction. Examples of the reagent to be used include borane tetrahydrofuran complex and the like.

Compound (9) can be produced by subjecting compound (7) to Mitsunobu reaction with 1-phenyl-1H-tetrazole-5-thiol (8). Examples of the reagent to be used include a combination of triphenylphosphine and diisopropyl azodicarboxylate (DIAD), and the like.

Compound (10) can be produced by subjecting compound (9) to an oxidation reaction. Examples of the reagent to be used include a combination of ammonium paramolybdate tetrahydrate and hydrogen peroxide, and the like.

Compound (12) can be produced by subjecting compound (10) to Julia-Kocienski reaction with aromatic aldehyde (11). Examples of the reagent to be used include sodium bis(trimethylsilyl)amide, lithium bis(trimethylsilyl)amide and the like.

Compound (Ia) can be produced by subjecting compound (12) to a deprotection reaction. Examples of the reagent to be used include hydrogen chloride in dioxane or in ethyl acetate and the like.

Compound (Ia) can also be produced according to the method shown in the following Scheme 2. The symbols are as defined above.

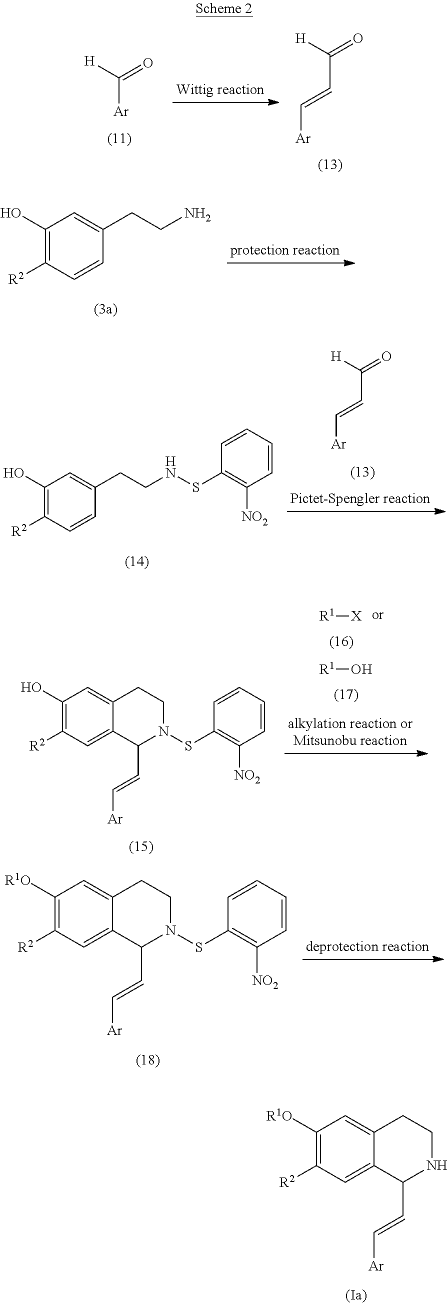

Compound (13) can be produced by subjecting compound (11) to Wittig reaction. Examples of reagent to be used include (formylmethylene)triphenylphosphorane and the like.

Compound (14) can be produced by subjecting compound (3a) (compound (3) wherein R¹ is a hydrogen atom) to a protection reaction. Examples of the reagent to be used include a combination of nitrophenylsulfenyl chloride and triethylamine, and the like.

Compound (15) can be produced by subjecting compound (14) to Pictet-Spengler reaction with aldehyde (13). Examples of the reagent to be used include a combination of 1,1′-binaphthyl-2,2′-diyl hydrogen phosphate (BNP acid) and 1,1′-bi-2-naphthol (BINOL), and the like.

Compound (18) can be produced by subjecting compound (15) to an alkylation reaction with alkyl halide (16) or Mitsunobu reaction with alcohol (17). Examples of the reagent to be used for the alkylation reaction include potassium carbonate, cesium carbonate, sodium hydride and the like. Examples of the reagent to be used for the Mitsunobu reaction include a combination of triphenylphosphine and an alkyl azodicarboxylate (e.g., diethyl azodicarboxylate (DEAD), diisopropyl azodicarboxylate (DIAD), and bis(2-methoxyethyl) azodicarboxylate (DMEAD)), and the like.

Compound (Ia) can be produced by subjecting compound (18) to a deprotection reaction. Examples of the reagent to be used include hydrochloric acid and the like.

Compound (Ia) can also be produced according to the method shown in the following Scheme 3. Each symbol is as defined above.

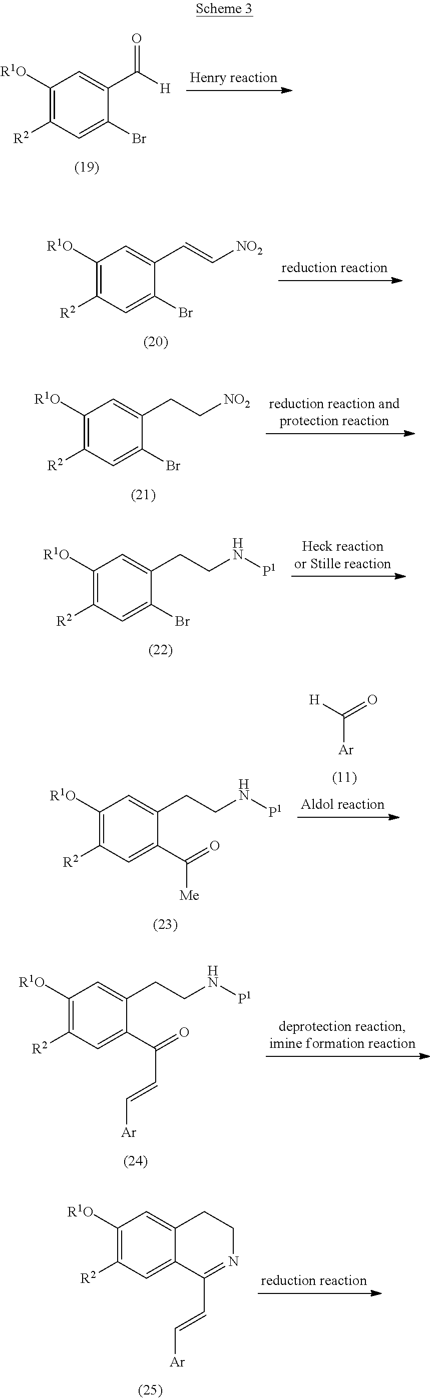

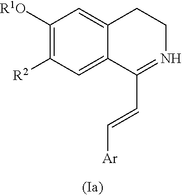

Compound (20) can be produced by subjecting compound (19) to Henry reaction with nitromethane. Example of reagent to be used include ammonium acetate and the like.

Compound (21) can be produced by subjecting compound (20) to a reduction reaction. Example of reagent to be used include sodium borohydride and the like.

Compound (22) can be produced by subjecting compound (21) to a reduction reaction, followed by a protection reaction. Examples of the reagent to be used for the reduction reaction include a combination of iron powder and ammonium chloride, and the like. Example of the reagent to be used for the protection reaction include di-tert-butyl dicarbonate and the like.

Compound (23) can be produced by subjecting compound (22) to Heck reaction with 1-(ethenyloxy)butane or Stille reaction with tributyl(1-ethoxyvinyl)stannan. Examples of the reagent to be used for the Heck reaction include a combination of palladium(II) acetate, 1,3-bis(diphenylphosphino)propane and potassium carbonate, and the like. Examples of the reagent to be used for the Stille reaction include a combination of tetrakis(triphenylphosphine)palladium(0) and lithium chloride, and the like.

Compound (24) can be produced by subjecting compound (23) to Aldol reaction with aromatic aldehyde (11). Examples of the reagent to be used include sodium hydroxide and the like.

Compound (25) can be produced by subjecting compound (24) to a deprotection reaction, followed by a spontaneous imine formation reaction. Examples of the reagent to be used include hydrogen chloride in ethyl acetate and the like.

Compound (Ia) can be produced by subjecting compound (25) to a reduction reaction. Examples of the reagent to be used include sodium borohydride, sodium triacetoxyborohydride and the like.

The below-mentioned compound (Ia-2) (compound (Ia) wherein Ar is a substituted or unsubstituted phenyl group having R^{b} group) can be produced according to the method shown in the following Scheme 4. R^{b} is a substituted or unsubstituted alkyl, alkenyl, alkynyl, cycloalkyl, cycloalkenyl, phenyl, aromatic heterocyclic or non-aromatic heterocyclic group. X is a halogen atom (e.g., Br or I). The other symbols are as defined above.

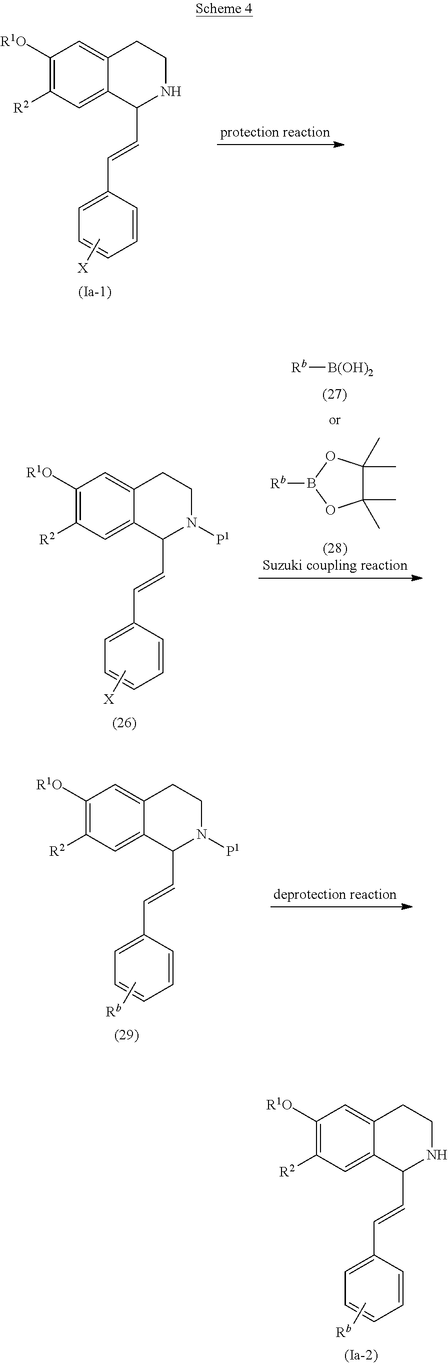

Compound (26) can be produced by subjecting compound (Ia-1) (compound (Ia) wherein Ar is a substituted or unsubstituted phenyl group having X group) to a protection reaction. Examples of reagent to be used include a combination of di-tert-butyl dicarbonate and triethylamine, and the like.

Compound (29) can be produced by subjecting compound (26) to Suzuki coupling reaction with boronic acid (27) or boronate (28). Examples of the reagent to be used include a combination of bis(di-tert-butyl(4-dimethylaminophenyl) phosphine) dichloropalladium(II) and cesium carbonate, and the like.

Compound (Ia-2) can be produced by subjecting compound (29) to a deprotection reaction. Example of the reagent to be used include hydrogen chloride in ethyl acetate and the like.

The below-mentioned compound (Ia-3) (compound (Ia) wherein Ar is a substituted or unsubstituted phenyl group having —O—R^{c} group) can be produced according to the method shown in the following Scheme 5. R^{c} is a substituted or unsubstituted alkyl, cycloalkyl, aromatic heterocyclic or non-aromatic heterocyclic group). P² is a protecting group (e.g., methoxymethyl group). The other symbols are as defined above.

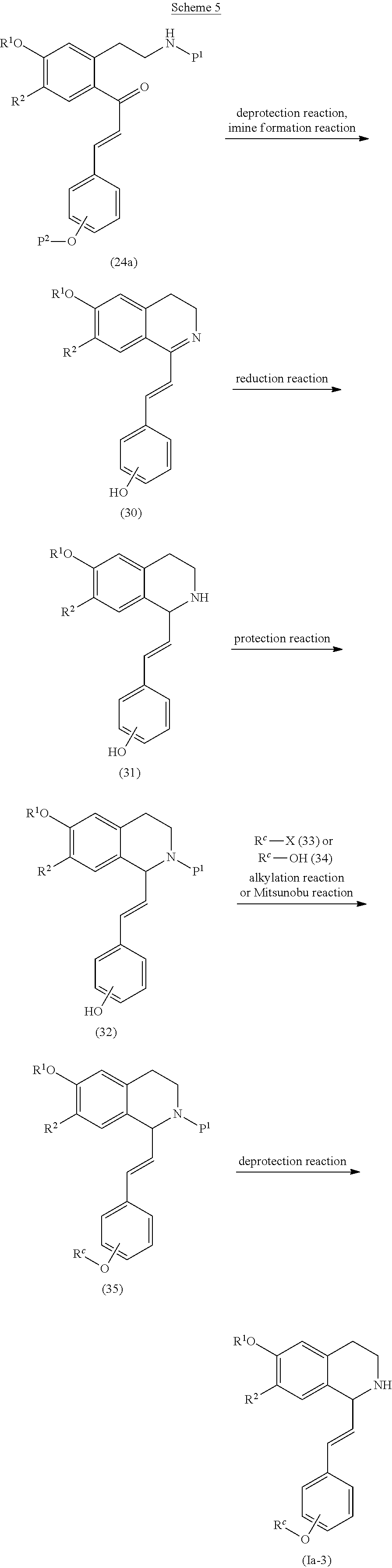

Compound (30) can be produced by subjecting compound (24a) (compound (24) wherein Ar is a substituted or unsubstituted phenyl group having —O—P² group) to a deprotection reaction, followed by a spontaneous imine formation reaction. Examples of the reagent to be used include hydrogen chloride in ethyl acetate and the like.

Compound (31) can be produced by subjecting compound (30) to a reduction reaction. Example of the reagent to be used include sodium borohydride and the like.

Compound (32) can be produced by subjecting compound (31) to a protection reaction. Examples of the reagent to be used include a combination of di-tert-butyl dicarbonate and triethylamine, and the like.

Compound (35) can be produced by subjecting compound (32) to an alkylation reaction with alkyl halide (33) or Mitsunobu reaction with alcohol (34). Examples of the reagent to be used for the alkylation reaction include potassium carbonate, cesium carbonate, sodium hydride and the like. Examples of the reagent to be used for the Mitsunobu reaction include a combination of triphenylphosphine and an alkyl azodicarboxylate (e.g., diethyl azodicarboxylate (DEAD), diisopropyl azodicarboxylate (DIAD), and bis(2-methoxyethyl) azodicarboxylate (DMEAD)), and the like.

Compound (Ia-3) can be produced by subjecting compound (34) to a deprotection reaction. Examples of the reagent to be used include hydrogen chloride in ethyl acetate and the like.

The below-mentioned compound (Ia-4) (compound (Ia) wherein R¹ is R^{1a}) can be also produced according to the method shown in the following Scheme 6. P³ is a protecting group (e.g., 2-(trimethylsilyl)ethoxymethyl). The other symbols are as defined above.

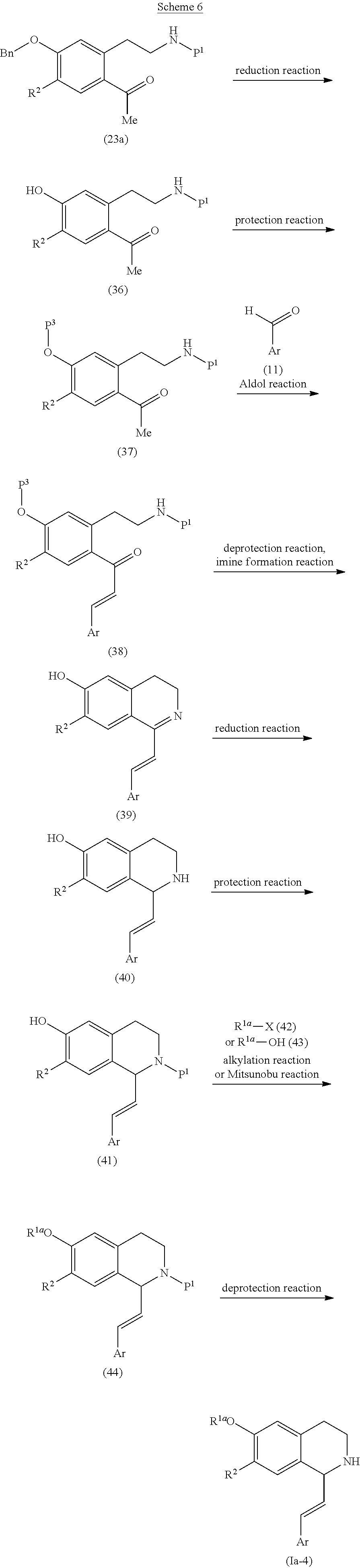

Compound (36) can be produced by subjecting compound (23a) (compound (23) wherein R¹ is a benzyl group) to a reduction reaction. Examples of the reagent to be used include a combination of hydrogen and palladium-activated carbon ethylenediamine complex, and the like.

Compound (37) can be produced by subjecting compound (36) to a protection reaction. Examples of the reagent to be used include a combination of sodium hydride and 2-(trimethylsilyl)ethoxymethyl chloride, and the like.

Compound (38) can be produced by subjecting compound (37) to Aldol reaction with aromatic aldehyde (11). Examples of the reagent to be used include sodium hydroxide and the like.

Compound (39) can be produced by subjecting compound (38) to a deprotection reaction, followed by a spontaneous imine formation reaction. Examples of the reagent to be used include hydrogen chloride in ethyl acetate and the like.

Compound (40) can be produced by subjecting compound (39) to a reduction reaction. Examples of the reagent to be used include sodium borohydride and the like.

Compound (41) can be produced by subjecting compound (40) to a protection reaction. Examples of the reagent to be used include a combination of di-tert-butyl dicarbonate and triethylamine, and the like.

Compound (44) can be produced by subjecting compound (41) to an alkylation reaction with alkyl halide (42) or Mitsunobu reaction with alcohol (43). Examples of the reagent to be used for the alkylation reaction include potassium carbonate, cesium carbonate, sodium hydride and the like. Examples of the reagent to be used for the Mitsunobu reaction include a combination of triphenylphosphine and alkyl azodicarboxylate (e.g., diethyl azodicarboxylate (DEAD), diisopropyl azodicarboxylate (DIAD), and bis(2-methoxyethyl) azodicarboxylate (DMEAD)), and the like.

Compound (Ia-4) can be produced by subjecting compound (44) to a deprotection reaction. Examples of the reagent to be used include hydrogen chloride in ethyl acetate and the like.

Compounds (3), (5), (8), (11), (16), (17), (19), (27), (28), (33), (34), (42) and (43) which are used as raw materials in each production method may be commercially easily available or can be produced according to a method known per se.

As for the configurational isomers (E, Z forms) of compound (I), they can be isolated and purified when isomerization occurs, for example, according to a conventional separation means such as extraction, recrystallization, distillation, chromatography and the like to obtain a pure compound. In addition, the corresponding pure isomer can also be obtained by isomerizing a double bond using heating, an acid catalyst, a transition metal complex, a metal catalyst, a radical catalyst, light irradiation, a strong base catalyst and the like, according to the method described in Shin Jikken Kagaku Kouza 14 (The Chemical Society of Japan ed.), pages 251 to 253, or 4th Edition Jikken Kagaku Kouza 19 (The Chemical Society of Japan ed.), pages 273 to 274, or a method analogous thereto.

Compound (I) contains a stereoisomer depending on the kind of a substituent, and each stereoisomer and a mixture thereof are encompassed in the present invention.

Compound (I) may be a hydrate or a non-hydrate.

When the objective product is obtained as a free form by the above-mentioned reaction, it can be converted to a salt according to a conventional method, or when the objective product is obtained as a salt, it can be converted to a free form or other salt according to a conventional method. The thus-obtained compound (I) can also be isolated and purified from a reaction mixture according to a known method such as phase transfer, concentration, solvent extraction, distillation, crystallization, recrystallization, chromatography and the like.

When compound (I) contains a configurational isomer, a diastereomer, a conformer and the like, each can be isolated according to the above-mentioned separation and purification methods, if desired. In addition, when compound (I) is racemic, d-form and l-form can be isolated according to a conventional optical resolution such as preparative high performance liquid chromatography (preparative HPLC), supercritical fluid chromatography (preparative SFC) and the like.

The thus-obtained compound (I), other reaction intermediate therefor and starting compounds thereof can be isolated and purified from a reaction mixture according to a method known per se, for example, extraction, concentration, neutralization, filtration, distillation, recrystallization, column chromatography, thin layer chromatography, preparative high performance liquid chromatography (preparative HPLC), moderate-pressure preparative liquid chromatography (moderate-pressure preparative LC) and the like.

A salt of compound (I) can be produced according to a method known per se. For example, when compound (I) is a basic compound, it can be produced by adding an inorganic acid or organic acid, or when compound (I) is an acidic compound, by adding an organic base or inorganic base.

When compound (I) contains an optical isomer, each optical isomer and a mixture thereof are encompassed in the scope of the present invention, and these isomers can be subjected to optical resolution or can be produced respectively, according to a method known per se, if desired.

Compound (I) may be a crystal.

The crystal of compound (I) can be produced according to a crystallization method known per se.

Examples of the crystallization method include crystallization method from a solution, crystallization method from vapor, crystallization method from a melt, and the like.

The “crystallization method from a solution” is typically a method of shifting a non-saturated state to supersaturated state by varying factors involved in solubility of compounds (solvent composition, pH, temperature, ionic strength, redox state, etc.) or the amount of solvent. Specific examples thereof include a concentration method, a slow cooling method, a reaction method (a diffusion method, an electrolysis method), a hydrothermal growth method, a flux method and the like. Examples of the solvent to be used include aromatic hydrocarbons (e.g., benzene, toluene, xylene, etc.), halogenated hydrocarbons (e.g., dichloromethane, chloroform, etc.), saturated hydrocarbons (e.g., hexane, heptane, cyclohexane, etc.), ethers (e.g., diethyl ether, diisopropyl ether, tetrahydrofuran, dioxane, etc.), nitriles (e.g., acetonitrile, etc.), ketones (e.g., acetone, etc.), sulfoxides (e.g., dimethyl sulfoxide, etc.), acid amides (e.g., N,N-dimethylformamide, etc.), esters (e.g., ethyl acetate, isopropyl acetate, etc.), alcohols (e.g., methanol, ethanol, 2-propanol, etc.), water and the like. These solvents are used alone or in a combination of two or more at a suitable ratio (e.g., 1:1 to 1:100 (a volume ratio)). Where necessary, a seed crystal can be used.

The “crystallization method from vapor” is, for example, a vaporization method (a sealed tube method, a gas stream method), a gas phase reaction method, a chemical transportation method and the like.

The “crystallization method from a melt” is, for example, a normal freezing method (a pulling method, a temperature gradient method, a Bridgman method), a zone melting method (a zone leveling method, a floating zone method), a special growth method (a VLS method, a liquid phase epitaxy method) and the like.

Preferable examples of the crystallization method include a method comprising dissolving compound (I) in a suitable solvent (e.g., alcohols such as methanol, ethanol etc.) at 20° C. to 120° C., and cooling the obtained solution to a temperature (e.g., 0 to 50° C., preferably 0 to 20° C.) not higher than the dissolution temperature, and the like.

The thus-obtained crystals of the present invention can be isolated, for example, by filtration and the like.

An analysis method of the obtained crystal is generally a method of crystal analysis by powder X-ray diffraction. As a method of determining crystal orientation, a mechanical method or an optical method and the like can also be used.

The crystal of compound (I) obtained by the above-mentioned production method may have high purity, high quality, and low hygroscopicity, may not be denatured even after a long-term preservation under general conditions, and may be expected to be superior in the stability. In addition, it may be also superior in the biological properties (e.g., pharmacokinetics (absorption, distribution, metabolism, excretion), efficacy expression etc.) and may be extremely useful as a medicament.

Compound (I) may be a prodrug. The prodrug of compound (I) means a compound which is converted to compound (I) with a reaction due to an enzyme, gastric acid and the like under the physiological condition in the living body, that is, a compound which is converted to compound (I) by enzymatic oxidation, reduction, hydrolysis and the like; a compound which is converted to compound (I) by hydrolysis and the like due to gastric acid, and the like. Examples of the prodrug for compound (I) include a compound obtained by subjecting an amino group in compound (I) to acylation, alkylation or phosphorylation (e.g., a compound obtained by subjecting an amino group in compound (I) to eicosanoylation, alanylation, pentylaminocarbonylation, (5-methyl-2-oxo-1,3-dioxolen-4-yl)methoxycarbonylation, tetrahydrofurylation, pyrrolidylmethylation, pivaloyloxymethylation or tert-butylation); a compound obtained by subjecting a hydroxy group in compound (I) to acylation, alkylation, phosphorylation or boration (e.g., a compound obtained by subjecting a hydroxy group in compound (I) to acetylation, palmitoylation, propanoylation, pivaloylation, succinylation, fumarylation, alanylation or dimethylaminomethylcarbonylation); a compound obtained by subjecting a carboxyl group in compound (I) to esterification or amidation (e.g., a compound obtained by subjecting a carboxyl group in compound (I) to ethyl esterification, phenyl esterification, carboxymethyl esterification, dimethylaminomethyl esterification, pivaloyloxymethyl esterification, ethoxycarbonyloxyethyl esterification, phthalidyl esterification, (5-methyl-2-oxo-1,3-dioxolen-4-yl)methyl esterification, cyclohexyloxycarbonylethyl esterification or methylamidation) and the like. These compounds can be produced from compound (I) according to a method known per se.

The prodrug of compound (I) may also be one which is converted to compound (I) under physiological conditions as described in “IYAKUHIN no KAIHATSU (Development of Pharmaceuticals)”, Vol. 7, Design of Molecules, p. 163-198, Published by HIROKAWA SHOTEN (1990).

In the present specification, compound (I) and the prodrug of compound (I) are sometimes collectively abbreviated as “the compound of the present invention”.

Compound (I) may be a hydrate, a non-hydrate, a solvate or a non-solvate.

In addition, compound (I) may be a compound labeled or substituted with an isotope (e.g., ²H, ³H, ¹¹C, ¹⁴C, ¹⁸F, ³⁵S, ¹²⁵I) and the like. The compound labeled or substituted with an isotope may be used, for example, as a tracer (PET tracer) used in positron emission tomography (PET), and useful in the field of medical diagnosis and the like.

Compound (I) also encompasses a deuterium conversion form wherein 1H is converted to ²H(D).

Compound (I) also encompasses a tautomer thereof.

Compound (I) may be a pharmaceutically acceptable cocrystal or a salt thereof. The cocrystal or a salt thereof means a crystalline substance constituted with two or more special solids at room temperature, each having different physical properties (e.g., structure, melting point, melting heat, hygroscopicity, solubility and stability). The cocrystal or a salt thereof can be produced according to a cocrystallization a method known per se.

### IV. Methods for Reducing Stress Granules and/or Preventing the Formulation of Stress Granules in a Neuron

The compounds of the invention exhibit the ability to reduce or prevent stress granules in a neuron. In a further aspect, the invention provides a method for reducing the presence of stress granules and/or preventing stress granules in a neuron, comprising contacting the neuron with a compound of the invention, thereby reducing the presence of stress granules and/or preventing stress granules in the neuron.

In an exemplary embodiment, the compound is described herein, or a salt, prodrug, hydrate or solvate thereof, or a combination thereof. In an exemplary embodiment, the invention provides a compound described herein, or a salt, hydrate or solvate thereof. In an exemplary embodiment, the invention provides a compound described herein, or a prodrug thereof. In an exemplary embodiment, the invention provides a compound described herein, or a salt thereof. In another exemplary embodiment, the compound of the invention is a compound described herein, or a pharmaceutically acceptable salt thereof. In another exemplary embodiment, the compound is described by a formula listed herein, or a pharmaceutically acceptable salt thereof. In an exemplary embodiment, the compound is part of a pharmaceutical composition described herein.

### V. Methods of Preventing and/or Treating Disease

The compounds and pharmaceutical composition of the invention prevent and/or treat diseases such as ALS in a mammal (e.g., mouse, rat, hamster, rabbit, cat, dog, bovine, sheep, monkey, human etc.) described herein.

In another aspect, the invention provides a method for treating a disease in a mammal (e.g., mouse, rat, hamster, rabbit, cat, dog, bovine, sheep, monkey, human etc.). The method includes administering to the mammal a therapeutically effective amount of the compound of the invention, sufficient to treat the disease. In an exemplary embodiment, the disease is amyotrophic lateral sclerosis, frontotemporal dementia, chronic traumatic encephalopathy, Alzheimer's disease, frontotemporal lobar degeneration, or multisystem proteinopathy. In an exemplary embodiment, the disease is amyotrophic lateral sclerosis, or ALS. In an exemplary embodiment, the disease is frontotemporal dementia. In an exemplary embodiment, the disease is chronic traumatic encephalopathy. In an exemplary embodiment, the disease is Alzheimer's disease. In an exemplary embodiment, the disease is frontotemporal lobar degeneration. In an exemplary embodiment, the disease is multisystem proteinopathy.

In another aspect, the invention provides a method for preventing a disease in a mammal (e.g., mouse, rat, hamster, rabbit, cat, dog, bovine, sheep, monkey, human etc.). The method includes administering to the mammal a prophylactically effective amount of the compound of the invention, sufficient to prevent the disease. In an exemplary embodiment, the disease is amyotrophic lateral sclerosis, frontotemporal dementia, chronic traumatic encephalopathy, Alzheimer's disease, frontotemporal lobar degeneration, or multisystem proteinopathy. In an exemplary embodiment, the disease is amyotrophic lateral sclerosis, or ALS. In an exemplary embodiment, the disease is frontotemporal dementia. In an exemplary embodiment, the disease is chronic traumatic encephalopathy. In an exemplary embodiment, the disease is Alzheimer's disease. In an exemplary embodiment, the disease is frontotemporal lobar degeneration. In an exemplary embodiment, the disease is multisystem proteinopathy.

In an exemplary embodiment, the invention provides a method for preventing and/or treating a disease, comprising administering an effective amount of a compound to a mammal (e.g., mouse, rat, hamster, rabbit, cat, dog, bovine, sheep, monkey, human etc.), wherein the mammal is not otherwise in need of treatment with the compound, wherein the disease is amyotrophic lateral sclerosis, frontotemporal dementia, chronic traumatic encephalopathy, Alzheimer's disease, frontotemporal lobar degeneration, or multisystem proteinopathy, wherein the compound is a compound represented by the formula (I):

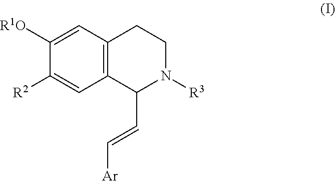

wherein
R¹ is unsubstituted C₁₋₆ alkyl, substituted or unsubstituted phenyl substituted C₁₋₆ alkyl, substituted or unsubstituted C₃₋₁₀ cycloalkyl substituted C₁₋₆ alkyl, or substituted or unsubstituted pyridyl substituted C₁₋₆ alkyl;
R² is H, CF₃, unsubstituted C₁₋₆ alkoxy, or phenyl substituted C₁₋₆ alkoxy;
R³ is H, —C(O)OR⁴, or —C(O)R⁴ or —C(O)NR⁴R⁵ wherein R⁴ and R⁵ are independently selected from unsubstituted C₁₋₆ alkyl, unsubstituted phenyl, and unsubstituted pyridyl; and
Ar is substituted or unsubstituted phenyl, substituted or unsubstituted benzofuryl, substituted or unsubstituted pyrrolopyridyl, substituted or unsubstituted imidazopyrimidinyl, substituted or unsubstituted imidazopyrazinyl, substituted or unsubstituted imidazopyridazinyl, or substituted or unsubstituted imidazopyridyl;
or a hydrate, solvate, or salt thereof.

In an exemplary embodiment, the compound is described herein, or a salt, prodrug, hydrate or solvate thereof, or a combination thereof. In an exemplary embodiment, the invention provides a compound described herein, or a salt, hydrate or solvate thereof. In an exemplary embodiment, the invention provides a compound described herein, or a prodrug thereof. In an exemplary embodiment, the invention provides a compound described herein, or a salt thereof. In another exemplary embodiment, the compound of the invention is a compound described herein, or a pharmaceutically acceptable salt thereof. In another exemplary embodiment, the compound is described by a formula listed herein, or a pharmaceutically acceptable salt thereof. In an exemplary embodiment, the compound is part of a pharmaceutical composition described herein. In another exemplary embodiment, the administration occurs under conditions which permit entry of the compound into the organism. Such conditions are known to one skilled in the art and specific conditions are set forth in the Examples appended hereto.

In another exemplary embodiment, the mammal is a human.

In an exemplary embodiment, the disease is prevented and/or treated through oral administration of the compound of the invention. In an exemplary embodiment, the disease is prevented and/or treated through intravenous administration of the compound of the invention. In an exemplary embodiment, the disease is prevented and/or treated through topical administration of the compound of the invention. In an exemplary embodiment, the disease is prevented and/or treated through intraperitoneal administration of the compound of the invention. In an exemplary embodiment, the compound is administered in a prophylactically effective amount. In an exemplary embodiment, the compound is administered in a therapeutically effective amount. In an exemplary embodiment, the compound is administered in a topically effective amount. In an exemplary embodiment, the compound is administered in an orally effective amount. In an exemplary embodiment, the pharmaceutical composition is administered in a prophylactically effective amount. In an exemplary embodiment, the pharmaceutical composition is administered in a therapeutically effective amount. In an exemplary embodiment, the pharmaceutical composition is administered in a topically effective amount. In an exemplary embodiment, the pharmaceutical composition is administered in an orally effective amount.

In an exemplary embodiment, the invention provides a method for preventing and/or treating a disease in a mammal (e.g., mouse, rat, hamster, rabbit, cat, dog, bovine, sheep, monkey, human etc.). The method includes administering to the mammal in need of treatment thereof a therapeutically and/or prophylactically effective amount of the compound of the invention, sufficient to treat the disease. In an exemplary embodiment, the mammal being administered the compound is in need of treatment with the compound of the invention. In an exemplary embodiment, the mammal being administered the compound is not otherwise in need of treatment with the compound of the invention.

In an exemplary embodiment, the invention provides a method for preventing and/or treating a disease in a mammal (e.g., mouse, rat, hamster, rabbit, cat, dog, bovine, sheep, monkey, human etc.). The method includes administering to the mammal in need of treatment thereof a therapeutically and/or prophylactically effective amount of the compound of the invention in combination with other active ingredients.

While the dose of the compound of the present invention varies depending on the administration route, symptom and the like, when, for example, the compound is orally administered to a patient with ALS disease (adult, body weight 40-80 kg, for example, 60 kg), it is, for example, 0.001-1000 mg/kg body weight/day, preferably 0.01-100 mg/kg body weight/day, more preferably 0.1-10 mg/kg body weight/day. This amount can be administered in 1 to 3 portions per day.

The compound of the present invention is superior in vivo kinetics (e.g., plasma drug half-life, intracerebral transferability, metabolic stability), shows low toxicity (e.g., more superior as a medicament in terms of acute toxicity, chronic toxicity, genetic toxicity, reproductive toxicity, cardiotoxicity, drug interaction, carcinogenicity etc.). The compound of the present invention is directly used as a medicament or a pharmaceutical composition mixed with a pharmaceutically acceptable carrier or the like to be orally or parenterally administered to mammals (e.g., humans, monkeys, cows, horses, pigs, mice, rats, hamsters, rabbits, cats, dogs, sheep and goats) in safety. Examples of the “parenteral” include intravenous, intramuscular, subcutaneous, intra-organ, intranasal, intradermal, instillation, intracerebral, intrarectal, intravaginal, intraperitoneal and intratumor administrations, administration to the vicinity of tumor etc. and direct administration to the lesion.

### VI. Formulations

In another aspect, the invention is a pharmaceutical composition which includes: (a) a pharmaceutically acceptable carrier; and (b) a compound of the invention. In another aspect, the pharmaceutical composition includes: (a) a pharmaceutically acceptable carrier; and (b) a compound according to a formula described herein. In another aspect, the pharmaceutical composition includes: (a) a pharmaceutically acceptable carrier; and (b) a compound described herein, or a salt, prodrug, hydrate or solvate thereof. In another aspect, the pharmaceutical composition includes: (a) a pharmaceutically acceptable carrier; and (b) a compound described herein, or a salt, hydrate or solvate thereof. In another aspect, the pharmaceutical composition includes: (a) a pharmaceutically acceptable carrier; and (b) a salt of a compound described herein. In an exemplary embodiment, the salt is a pharmaceutically acceptable salt. In another aspect, the pharmaceutical composition includes: (a) a pharmaceutically acceptable carrier; and (b) a prodrug of a compound described herein. In another aspect, the pharmaceutical composition includes: (a) a pharmaceutically acceptable carrier; and (b) a compound described herein. In an exemplary embodiment, the pharmaceutical composition is a unit dosage form. In an exemplary embodiment, the pharmaceutical composition is a single unit dosage form. In an exemplary embodiment, the pharmaceutical composition includes: (a) a compound described herein, or a salt, hydrate or solvate thereof; and (b) an other active ingredient.

In an exemplary embodiment, the pharmaceutical composition is useful in the prevention and/or treatment of a disease provided herein. In an exemplary embodiment, the pharmaceutical composition is useful in the prevention and/or treatment of amyotrophic lateral sclerosis, frontotemporal dementia, chronic traumatic encephalopathy, Alzheimer's disease, frontotemporal lobar degeneration, or multisystem proteinopathy. In an exemplary embodiment, the pharmaceutical composition is useful in the prevention and/or treatment of amyotrophic lateral sclerosis.

In an exemplary embodiment, the pharmaceutical composition is administered to a mammal in need of treatment with such a pharmaceutical composition. In an exemplary embodiment, the pharmaceutical composition is administered to a mammal not otherwise in need of treatment with the compound of the invention.

Information regarding excipients of use in the compositions of the invention can be found in Remington: The Science and Practice of Pharmacy, 21st Ed., Pharmaceutical Press (2011) which is incorporated herein by reference.

### Examples

The invention is further illustrated by the Examples and Experimental Examples that follow. The Examples and Experimental Examples are not intended to define or limit the scope of the invention.

The following Examples illustrate the synthesis of representative compounds used in the invention and the following Reference Examples illustrate the synthesis of intermediates in their preparation. These examples are not intended, nor are they to be construed, as limiting the scope of the invention. It will be clear that the invention may be practiced otherwise than as particularly described herein. Numerous modifications and variations of the invention are possible in view of the teachings herein and, therefore, are within the scope of the invention.

All temperatures are given in degrees Centigrade. Room temperature means about 10 to about 35° C. Reagents were purchased from commercial sources or prepared following standard literature procedures. Unless otherwise noted, reactions were carried out under a positive pressure of nitrogen. Reaction vessels were sealed with either rubber septa or Teflon screw caps. Nitrogen was introduced through Tygon tubing, fitted with a large bore syringe needle. Concentration under vacuum refers to the removal of solvent on a Büchi Rotary Evaporator. The ratios indicated for mixed solvents are volume mixing ratios, unless otherwise specified. % means wt %, unless otherwise specified.

The elution by column chromatography was performed under the observation by TLC (Thin Layer Chromatography) unless otherwise specified. In the observation by TLC, 60 F₂₅₄ manufactured by Merck was used as a TLC plate, the solvent used as an elution solvent in column chromatography was used as a developing solvent, and UV detector was used for the detection. In silica gel column chromatography, the indication of NH means so use of aminopropylsilane-bonded silica gel, and the indication of Diol means use of 3-(2,3-dihydroxypropoxy)propylsilane-bonded silica gel. In preparative HPLC (high performance liquid chromatography), the indication of C18 means use of octadecyl-bonded silica gel. The ratio for elution solvent is, unless otherwise specified, a volume mixing ratio.

Analytical HPLCs for Examples AA through DD were performed using a Supelco discovery C₁₈ 15 cm×4.6 mm/5 μm column coupled with an Agilent 1050 series VWD UV detector at 210 nm. Conditions: Solvent A: H₂O/1% acetonitrile/0.1% HCO₂H; Solvent B: methanol.

For the analysis of 1H NMR, ACD/SpecManager (trade name) software and the like were used. Peaks of a hydroxyl group, an amino group and the like, having very mild proton peak, are not sometimes described. 1H NMR spectra in Examples AA through DD were recorded on a Varian INOVA NMR spectrometer [400 MHz (¹H) or 500 MHz (¹H)] or Varian 400-MR [400 MHz (1H)]. All spectra were determined in the solvents indicated. Although chemical shifts are reported in ppm downfield of tetramethylsilane, they are referenced to the residual proton peak of the respective solvent peak for 1H NMR. Interproton coupling constants are reported in Hertz (Hz).

MS (mass spectrum) was measured by LC/MS (liquid chromatograph mass spectrometer). As the ionization method, ESI (Electron Spray Ionization) method, or APCI (Atmospheric Pressure Chemical Ionization) method was used. The data indicates actual measured value (found). While molecular ion peak is generally observed, a fragment ion is sometimes observed. In the case of a salt, a molecular ion peak or fragment ion peak of free form is generally observed. Elemental analysis value (Anal.) is described as calculated value (Calcd) and actual measured value (Found).

LCMS spectra in Examples AA through DD were obtained using a ThermoFinnigan AQA MS ESI instrument utilizing a Phenomenex Aqua 5 micron C₁₈ 125 Å 50×4.60 mm column. The spray setting for the MS probe was at 350 μL/min with a cone voltage at 25 mV and a probe temperature at 450° C. The spectra were recorded using ELS and UV (254 nm) detection. Alternatively, LCMS spectra were obtained using an Agilent 1200SL HPLC equipped with a 6130 mass spectrometer or Agilent 1200 series with a 6140 mass spectrometer operating with electrospray ionization.

Silica gel chromatography in Examples AA through DD was carried out on either a Teledyne ISCO CombiFlash Companion or Companion Rf Flash Chromatography System with a variable flow rate from 5-100 mL/min. The columns used were Teledyne ISCO RediSep Disposable Flash Columns (4, 12, 40, 80, or 120 g prepacked silica gel), which were run with a maximum capacity of 1 g crude sample per 10 g silica gel. Samples were preloaded on Celite in Analogix Sample Loading Cartridges with frits (1/in, 1/out). The eluent was 0-100% EtOAc in heptane or 0-10% MeOH in CH₂Cl₂ as a linear gradient over the length of the run (14-20 minutes). Peaks were detected by variable wavelength UV absorption (200-360 nm). The resulting fractions were analyzed, combined as appropriate, and evaporated under reduced pressure to provide purified material.

Powder X-RAY diffraction pattern was measured using Cu-Kα characteristic radiation from Rigaku Ultima IV, and characteristic peaks were described.

In the following Examples, the following abbreviations are used.
MS: mass spectrum
M: mol concentration
CDCl₃: deuterochloroform
DMSO-d₆: deuterodimethyl sulfoxide
¹H NMR: proton nuclear magnetic resonance
LC/MS: liquid chromatograph mass spectrometer
ESI: electron spray ionization
APCI: atmospheric pressure chemical ionization
AcOH: acetic acid
Boc₂O: di-tert-butyl dicarbonate
DEA: diethylamine
DME: 1,2-dimethoxyethane
DMF: N,N-dimethylformamide
DMSO: dimethyl sulfoxide
EtOAc: ethyl acetate
Et₃N: triethylamine
EtOH: ethanol
n-BuOH: 1-butanol
IPA: 2-propanol
IPE: diisopropyl ether
MeOH: methanol
Pd(OAc)₂: palladium(II) acetate
THF: tetrahydrofuran

### AA. (E)-6-(benzyloxy)-1-(2-(imidazo[1,2-a]pyrimidin-3-yl)vinyl)-7-methoxy-1,2,3,4-tetrahydroisoquinoline

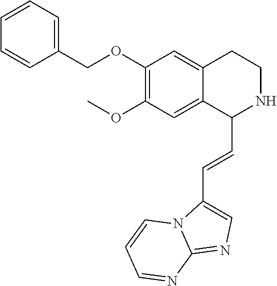 [SMILES: COc1cc2c(cc1OCc1ccccc1)CCNC2/C=C/c1cnc2ncccn12]

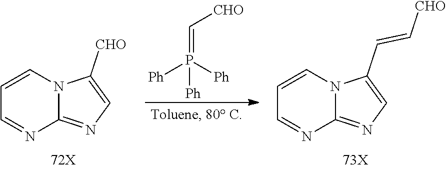 [SMILES: O=Cc1cnc2ncccn12 ; O=CC=P(c1ccccc1)(c1ccccc1)c1ccccc1 ; O=C/C=C/c1cnc2ncccn12]

Compound 73X. To the stirred solution of aldehyde 72X (0.357 g, 2.43 mmol) in toluene (5 mL) was added (formylmethylene)triphenylphosphorane (0.77 g, 2.43 mmol) and the reaction mass was stirred at 80° C. for 4 h. Solvent was removed and the crude product was purified by column chromatography (EtOAc/Hexane) to compound 73X (200 mg). MS (m/z); 174 [M+H]

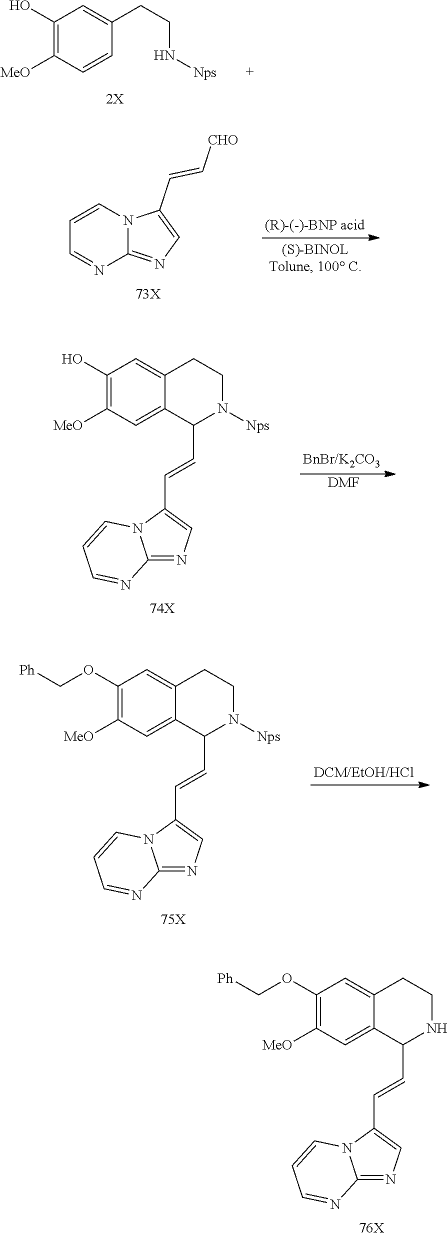

Compound 74X. To a solution of compound 2X (0.260 g, 0.813 mmol, 1.0 eq) in toluene (7 mL) were added BINOL (0.070 g, 0.244 mmol, 0.3 eq) and 1,1′-Binaphthyl-2,2′-diyl hydrogen phosphate (0.043 g, 0.122 mmol, 0.15 eq) and the reaction mixture was stirred under argon for 10 min. The reaction mixture was then heated to 90 to 100° C. and then the aldehyde 73X (141 mg, 0.813 mmol, 1.0 eq) was added. The reaction mixture was stirred at this temperature for 6 h. Solvent was removed and the residue was purified by column chromatography (EtOAc/hexane) to give the compound 74X. MS (m/z); 476 [M+H]

Compound 75X. To a solution of 6-hydroxy compound 74X (173 mg, 0.365 mmol, 1.0 eq) and Cs₂CO₃ (237 mg, 0.73 mmol, 2.0 eq) in DMF (8 mL) was added benzyl bromide (94 mg, 0.547 mmol, 1.5 eq) and the reaction mixture was stirred at room temperature for 10 h. Diluted with ethyl acetate and the organic layer was washed with water (3×50 mL), dried and evaporated to give a residue, which was purified by column chromatography to give compound 75X. MS (m/z); 564 [M+H]

Compound 76X. To a solution of 50 mg of the compound 75X in DCM (3 mL) and ethanol (3 mL) at 0° C. was added con. HCl (1 mL) and the reaction mixture was stirred at room for 1 hour. Solvent was removed under vacuum and product was purified with reverse phase chromatography to give compound 76X. MS (m/z); 413 [M+H]

### AB. (E)-6-(benzyloxy)-1-(2-(6-chloroimidazo[1,2-a]pyrimidin-3-yl)vinyl)-7-methoxy-1,2,3,4-tetrahydroisoquinoline

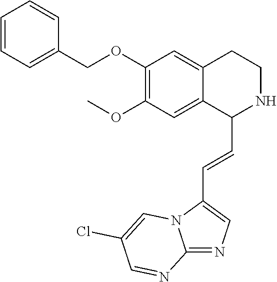 [SMILES: COc1cc2c(cc1OCc1ccccc1)CCNC2/C=C/c1cnc2ncc(Cl)cn12]

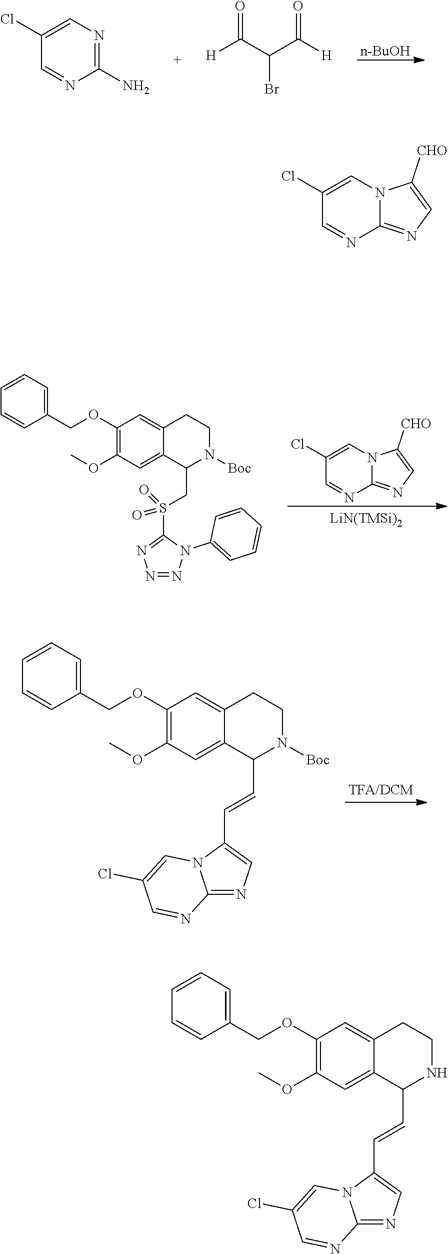

5-Chloro-2-aminopyrimidine (1.3 g, 10 mmol) and 2-bromomalonic dialdehyde (1.51 g, 10 mmol) were dissolved in n-BuOH (10 ml) and the mixture was refluxed with stirring at 120° C. for 8 h. The reaction progress was monitored by TLC. After the end of the reaction, the solvent was removed in vacuum, water (20 ml) was added, and the mixture neutralized with NaHCO₃ solution. The product was extracted with CHCl₃ (3×20 ml), the combined fractions were dried over Na₂SO₄, and evaporated to volume of 10 ml. The obtained mixture was purified by flash chromatography.

To a solution of tert-butyl 6-benzyloxy-7-methoxy-1-[(1-phenyltetrazol-5-yl)sulfonylmethyl]-3,4-dihydro-1H-isoquinoline-2-carboxylate (118 mg, 0.2 mmol) in THF (5 ml) with 6-chloroimidazo[1,2-a]pyrimidine (64 mg, 0.4 mmol) was added dropwise at −38° C. 1M LiN(TMSi)₂ (0.4 ml, 0.4 mmol). The reaction mixture was stirred at −38° C. for 60 min and then allowed to come to room temperature. The reaction was quenched with sat NH₄Cl (10 ml), extracted with EtOAc (20 ml), dried (MgSO₄) and the solvent removed. The residue was the treated chromatographed via reverse phase chromatography.

Product of reaction was dissolved in DCM (5 ml) and TFA (2 ml) was added. Reaction mixture was stirred at room temperature for 5 min. Solvent was removed under vacuum and product was purified with reverse phase chromatography. MS (m/z); 448 [M+H]

### AC. (E)-6-(benzyloxy)-1-(2-(6-fluoroimidazo[1,2-a]pyrimidin-3-yl)vinyl)-7-methoxy-1,2,3,4-tetrahydroisoquinoline

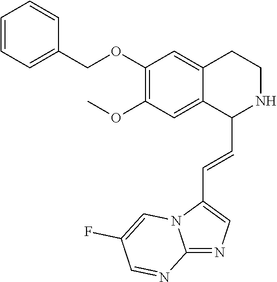 [SMILES: COc1cc2c(cc1OCc1ccccc1)CCNC2/C=C/c1cnc2ncc(F)cn12]

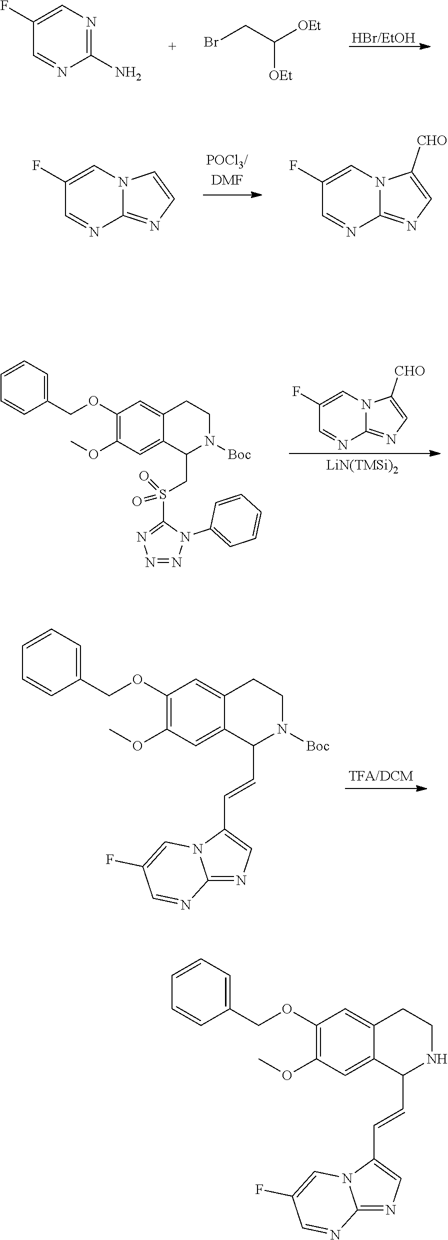

A solution of 5-fluoro-2-aminopyrimidine (0.5 g, 4.42 mmol), bromoacetaldehyde diethyl acetal (1.33 mL, 1.74 g, 8.84 mmol) and 48% aqueous hydrobromic acid (0.5 ml) in ethanol (5 ml) was heated at reflux for 18 h. The reaction mixture was cooled and purified by flash chromatography.

POCl₃ (0.48 mL, 5.21 mmol, 1.1 equiv) was added dropwise over 10 mL of dry DMF under N₂ for 10 minutes at RT and the mixture was stirred for 5 minutes. Then 6-fluoroimidazo[1,2-a]pyrimidine (650 mg, 4.74 mmol, 1.0 equiv) in dry DMF was added dropwise at 0° C. for 5 min. The mixture was stirred at 40° C. for 2 h. After this time, an aliquot was checked for reaction completeness. Next 10 g of ice was added and the reaction mixture (pH=3) was basified with sat. NaHCO₃ to pH ˜7-9 with rapid stirring. DMF was evaporated in vacuo, and the brown solid obtained was suspended in water (20 mL). This aqueous layer was extracted with EtOAc (3×20 mL); the organic layers were combined, washed with brine (10 mL), dried over Na₂SO₄, and concentrated.

To a solution of tert-butyl 6-benzyloxy-7-methoxy-1-[(1-phenyltetrazol-5-yl)sulfonylmethyl]-3,4-dihydro-1H-isoquinoline-2-carboxylate (118 mg, 0.2 mmol) in THF (5 ml) with 6-fluoroimidazo[1,2-a]pyrimidine (66.0 mg, 0.4 mmol) was added dropwise at −38° C. 1M LiN(TMSi)₂ (0.4 mL, 0.4 mmol). The reaction mixture was stirred at −38° C. for 60 min and then allowed to come to room temperature. The reaction was quenched with sat. NH₄Cl (10 ml), extracted with EtOAc (20 ml), dried (Na₂SO₄) and the solvent removed. The residue was the treated chromatographed via reverse phase chromatography.

Product of reaction was dissolved in DCM (5 ml) and TFA (2 ml) was added. Reaction mixture was stirred at room temperature for 5 min. Solvent was removed under vacuum and product was purified with reverse phase chromatography. MS (m/z); 432 [M+H]

### AD. (E)-6-(benzyloxy)-7-methoxy-1-(2-(6-(trifluoromethyl)imidazo[1,2-a]pyrimidin-3-yl)vinyl)-1,2,3,4-tetrahydroisoquinoline

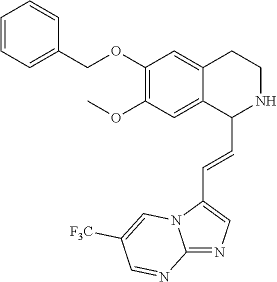 [SMILES: COc1cc2c(cc1OCc1ccccc1)CCNC2/C=C/c1cnc2ncc(C(F)(F)F)cn12]

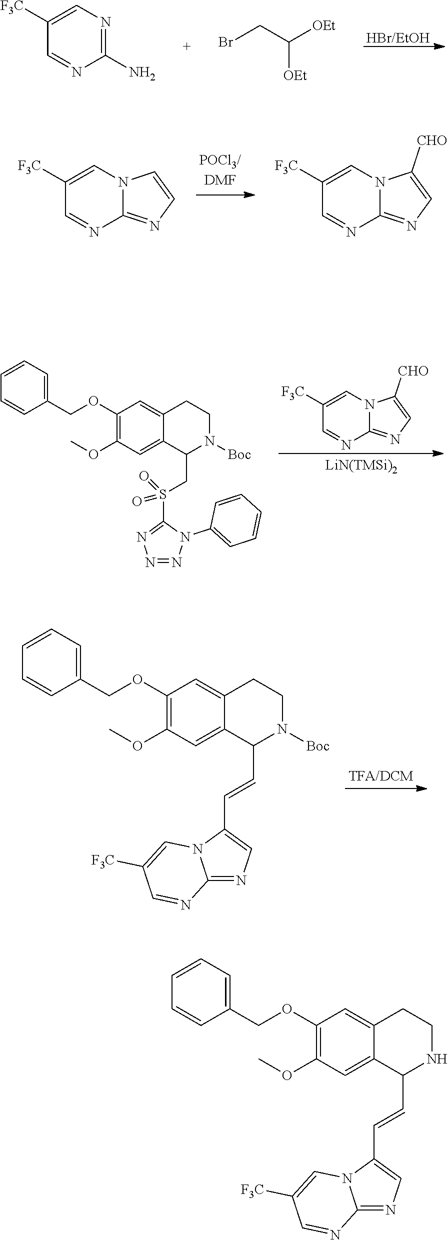

A solution of 5-trifluoromethyl-2-aminopyrimidine (0.5 g, 3.07 mmol), bromoacetaldehyde diethyl acetal (1.21 g, 6.14 mmol) and 48% aqueous hydrobromic acid (0.5 ml) in ethanol (5 ml) was heated at reflux for 18 h. The reaction mixture was cooled and purified by flash chromatography.

POCl₃ (0.28 mL, 2.94 mmol, 1.1 equiv) was added dropwise over 10 mL of dry DMF under N₂ for 10 minutes at RT and the mixture was stirred for 5 minutes. Then 6-trifluoromethylimidazo[1,2-a]pyrimidine (500 mg, 2.67 mmol, 1.0 equiv) in dry DMF was added dropwise at 0° C. for 5 min. The mixture was stirred at 40° C. for 2 h. After this time, an aliquot was checked for reaction completeness. Next 10 g of ice was added and the reaction mixture (pH=3) was basified with sat. NaHCO₃ to pH ˜7-9 with rapid stirring. DMF was evaporated in vacuo, and the brown solid obtained was suspended in water (20 mL). This aqueous layer was extracted with EtOAc (3×20 mL); the organic layers were combined, washed with brine (10 mL), dried over Na₂SO₄, and concentrated.

To a solution of tert-butyl 6-benzyloxy-7-methoxy-1-[(1-phenyltetrazol-5-yl)sulfonylmethyl]-3,4-dihydro-1H-isoquinoline-2-carboxylate (118 mg, 0.2 mmol) in THF (5 ml) with 6-trifluoromethylimidazo[1,2-a]pyrimidine (92.5 mg, 0.4 mmol) was added dropwise at −38° C. 1M LiN(TMSi)₂ (0.4 ml, 0.4 mmol). The reaction mixture was stirred at −38° C. for 60 min and then allowed to come to room temperature. The reaction was quenched with sat NH₄Cl (10 ml), extracted with EtOAc (20 ml), dried (MgSO₄) and the solvent removed. The residue was the treated chromatographed via reverse phase chromatography.

1a Product of reaction was dissolved in DCM (5 ml) and TFA (2 ml) was added. Reaction mixture was stirred at room temperature for 5 min. Solvent was removed under vacuum and product was purified with reverse phase chromatography. MS (m/z); 482 [M+H]

### AE. (E)-6-(benzyloxy)-7-methoxy-1-(2-(6-methoxyimidazo[1,2-a]pyrimidin-3-yl)vinyl)-1,2,3,4-tetrahydroisoquinoline

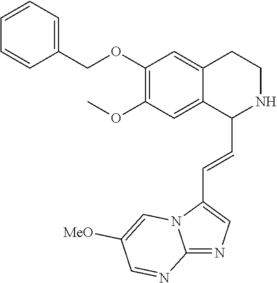 [SMILES: COc1cnc2ncc(/C=C/C3NCCc4cc(OCc5ccccc5)c(OC)cc43)n2c1]

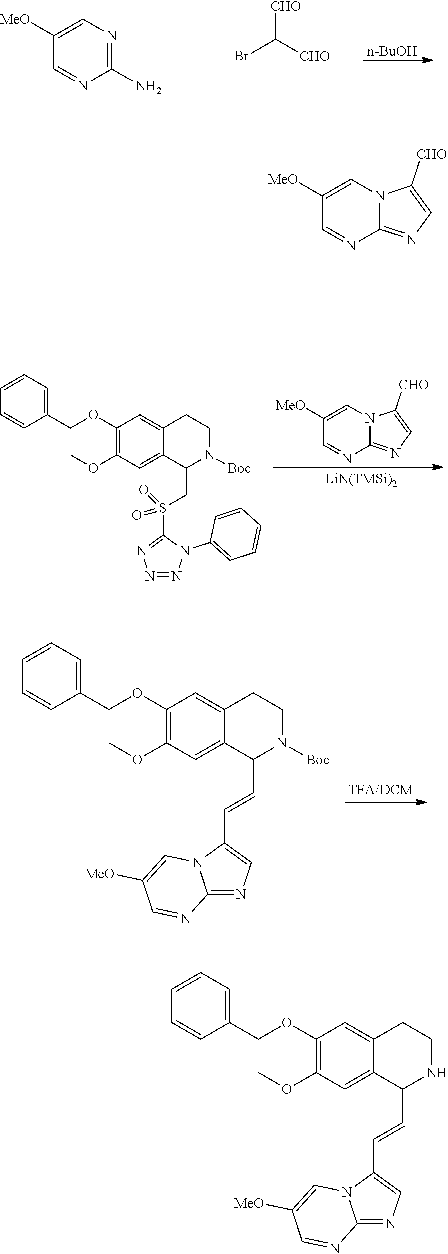

5-Methoxy-2-aminopyrimidine (500 mg, 4.0 mmol) and 2-bromomalonic dialdehyde (603 mg, 4.0 mmol) were dissolved in n-BuOH (10 ml) and the mixture was refluxed with stirring at 120° C. for 8 h. The reaction progress was monitored by TLC. After the end of the reaction, the solvent was removed in vacuum, water (20 ml) was added, and the mixture neutralized with NaHCO₃ solution. The product was extracted with CHCl₃ (3×20 ml), the combined fractions were dried over Na₂SO₄, and evaporated to volume of 10 ml. The obtained mixture was purified by flash chromatography.

To a solution of tert-butyl 6-benzyloxy-7-methoxy-1-[(1-phenyltetrazol-5-yl)sulfonylmethyl]-3,4-dihydro-1H-isoquinoline-2-carboxylate (118 mg, 0.2 mmol) in THF (5 ml) with 6-methoxyimidazo[1,2-a]pyrimidine (61.3 mg, 0.4 mmol) was added dropwise at −38° C. 1M LiN(TMSi)₂ (0.4 ml, 0.4 mmol). The reaction mixture was stirred at −38° C. for 60 min and then allowed to come to room temperature. The reaction was quenched with sat NH₄Cl (10 ml), extracted with EtOAc (20 ml), dried (MgSO₄) and the solvent removed. The residue was the treated chromatographed via reverse phase chromatography.

Product of reaction was dissolved in DCM (5 ml) and TFA (2 ml) was added. Reaction mixture was stirred at room temperature for 5 min. Solvent was removed under vacuum and product was purified with reverse phase chromatography. MS (m/z); 444 [M+H]

### AF. (E)-6-(benzyloxy)-7-methoxy-1-(2-(6-methylimidazo[1,2-a]pyrimidin-3-yl)vinyl)-1,2,3,4-tetrahydroisoquinoline

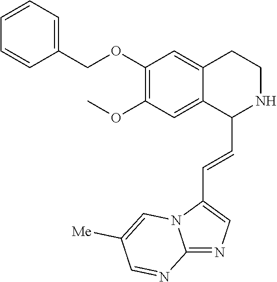 [SMILES: COc1cc2c(cc1OCc1ccccc1)CCNC2/C=C/c1cnc2ncc(C)cn12]

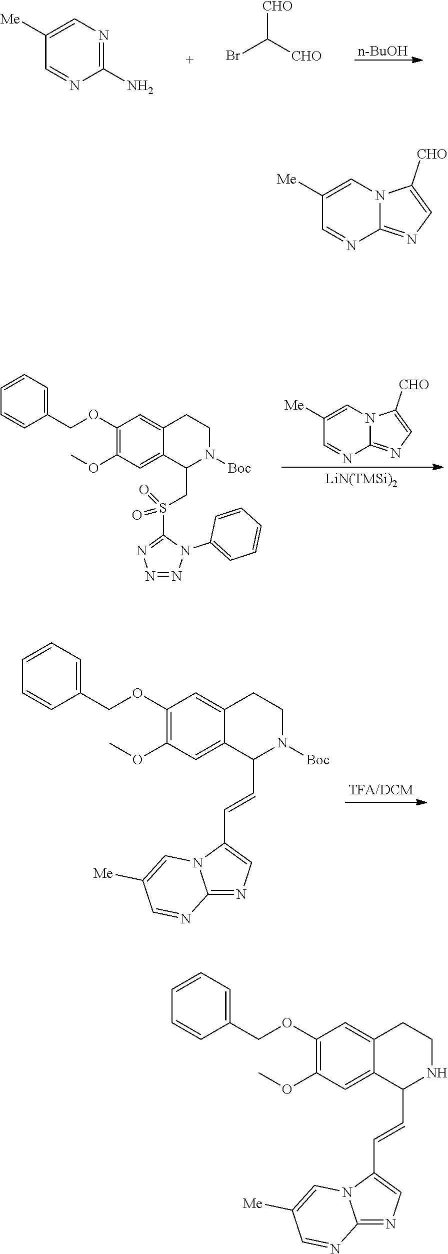

5-Methyl-2-aminopyrimidine (500 mg, 4.58 mmol) and 2-bromomalonic dialdehyde (692 mg, 4.58 mmol) were dissolved in n-BuOH (10 ml) and the mixture was refluxed with stirring at 120° C. for 8 h. The reaction progress was monitored by TLC. After the end of the reaction, the solvent was removed in vacuum, water (20 ml) was added, and the mixture neutralized with NaHCO₃ solution. The product was extracted with CHCl₃ (3×20 ml), the combined fractions were dried over Na₂SO₄, and evaporated to volume of 10 ml. The obtained mixture was purified by flash chromatography.

To a solution of tert-butyl 6-benzyloxy-7-methoxy-1-[(1-phenyltetrazol-5-yl)sulfonylmethyl]-3,4-dihydro-1H-isoquinoline-2-carboxylate (118 mg, 0.2 mmol) in THF (5 ml) with 6-methylimidazo[1,2-a]pyrimidine (66.9 mg, 0.4 mmol) was added dropwise at −38° C. 1M LiN(TMSi)₂ (0.4 ml, 0.4 mmol). The reaction mixture was stirred at −38° C. for 60 min and then allowed to come to room temperature. The reaction was quenched with sat NH₄Cl (10 ml), extracted with EtOAc (20 ml), dried (MgSO₄) and the solvent removed. The residue was the treated chromatographed via reverse phase chromatography.

Product of reaction was dissolved in DCM (5 ml) and TFA (2 ml) was added. Reaction mixture was stirred at room temperature for 5 min. Solvent was removed under vacuum and product was purified with reverse phase chromatography. MS (m/z); 428 [M+H]

### AG. Ethyl (E)-3-(2-(6-(benzyloxy)-7-methoxy-1,2,3,4-tetrahydroisoquinolin-1-yl)vinyl)imidazo[1,2-a]pyrimidine-6-carboxylate

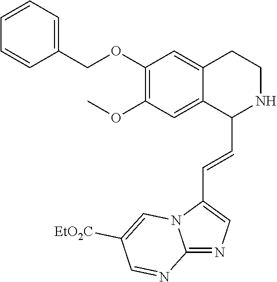 [SMILES: CCOC(=O)c1cnc2ncc(/C=C/C3NCCc4cc(OCc5ccccc5)c(OC)cc43)n2c1]

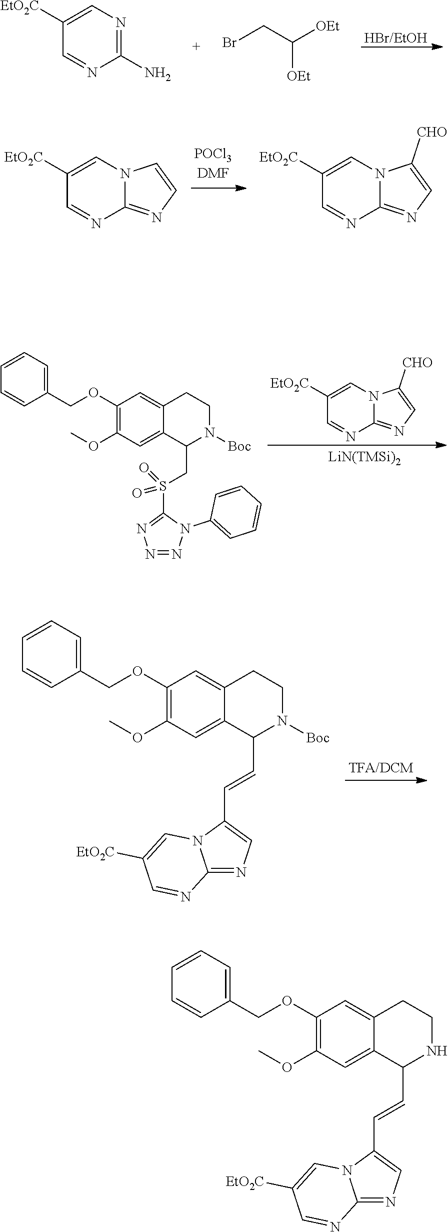

A solution of ethyl-2-aminopyrimidine-5-carboxylate (0.5 g, 2.29 mmol), bromoacetaldehyde diethyl acetal (0.9 mL, 1.18 g, 5.98 mmol) and 48% aqueous hydrobromic acid (0.5 ml) in ethanol (5 ml) was heated at reflux for 18 h. The reaction mixture was cooled and purified by flash chromatography.

POCl₃ (0.29 mL, 3.11 mmol, 1.1 equiv) was added dropwise over 10 mL of dry DMF under N₂ for 10 minutes at RT and the mixture was stirred for 5 minutes. Then ethyl imidazo[1,2-a]pyrimidine-6-carboxylate (540 mg, 2.82 mmol, 1.0 equiv) in dry DMF was added dropwise at 0° C. for 5 min. The mixture was stirred at 40° C. for 2 h. After this time, an aliquot was checked for reaction completeness. Next 10 g of ice was added and the reaction mixture (pH=3) was basified with sat. NaHCO₃ to pH ˜7-9 with rapid stirring. DMF was evaporated in vacuo, and the brown solid obtained was suspended in water (20 mL). This aqueous layer was extracted with EtOAc (3×20 mL); the organic layers were combined, washed with brine (10 mL), dried over Na₂SO₄, and concentrated.

To a solution of tert-butyl 6-benzyloxy-7-methoxy-1-[(1-phenyltetrazol-5-yl)sulfonylmethyl]-3,4-dihydro-1H-isoquinoline-2-carboxylate (237 mg, 0.4 mmol) in THF (5 ml) with ethyl imidazo[1,2-a]pyrimidine-6-carboxylate (175 mg, 0.8 mmol) was added dropwise at −38° C. 1M LiN(TMSi)₂ (0.8 mL, 0.8 mmol). The reaction mixture was stirred at −38° C. for 60 min and then allowed to come to room temperature. The reaction was quenched with sat NH₄Cl (10 ml), extracted with EtOAc (20 ml), dried (MgSO₄) and the solvent removed. The residue was the treated chromatographed via reverse phase chromatography.

Product of reaction was dissolved in DCM (5 ml) and TFA (2 ml) was added. Reaction mixture was stirred at room temperature for 5 min. Solvent was removed under vacuum and product was purified with reverse phase chromatography. MS (m/z); 486 [M+H]

### AH. (E)-3-(2-(6-(benzyloxy)-7-methoxy-1,2,3,4-tetrahydroisoquinolin-1-yl)vinyl)imidazo[1,2-a]pyrimidine-6-carboxylic acid

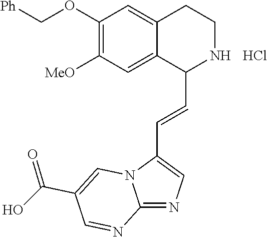 [SMILES: COc1cc2c(cc1OCc1ccccc1)CCNC2/C=C/c1cnc2ncc(C(=O)O)cn12 ; Cl]

### Preparation of Protected 3-hydroxy-4-methoxyphenylethylamine

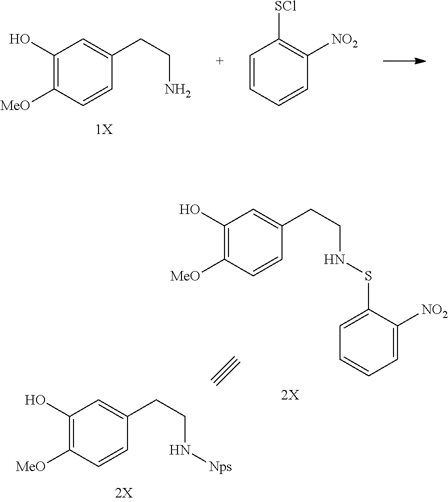

Compound 2X. To a solution of CHCl₃ (100 mL), MeOH (5 mL) and triethylamine (1.5 mL) was added 3-hydroxy-4-methoxyphenylethylamine (compound 1X) (2.03 g, 10.0 mmol, 1.0 equiv). The reaction mixture was heated at 60° C. to dissolve the amine. Addition of DIEA (1.5 mL) and MeOH (15 mL) increased the solubility and made it a clear homogeneous solution. The solution was then cooled to 0° C. To this cold solution was added saturated K₂CO₃ solution (100 mL) and the reaction mixture was stirred at this temperature for 5 min. Then 2-nitrophenylsulfenylchloride (2.28 g, 12.0 mmol, 1.2 eq) was added in three portions and the reaction mixture was stirred at room temperature for 5 h. The reaction mixture was extracted with DCM (100 mL, 2×). Combined organic layer was dried and evaporated to provide a residue, which was purified by column chromatography (EtOAc/Hexane) to provide compound 2X as a yellow solid. MS (m/z); 321 [M+H]

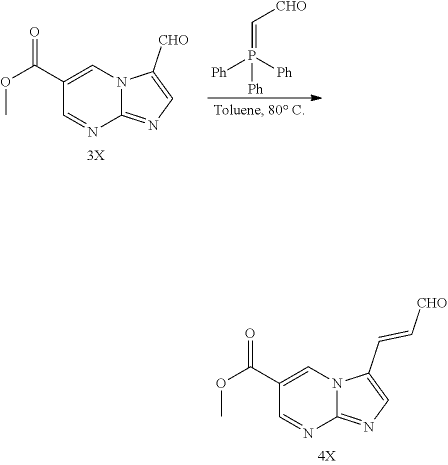 [SMILES: COC(=O)c1cnc2ncc(C=O)n2c1 ; O=CC=P(c1ccccc1)(c1ccccc1)c1ccccc1 ; COC(=O)c1cnc2ncc(/C=C/C=O)n2c1]

Compound 4X. To the stirred solution of aldehyde 3X (0.500 g, 2.43 mmol) in toluene (5 mL) was added (formylmethylene)triphenylphosphorane (0.77 g, 2.43 mmol) and the reaction mass was stirred at 80° C. for 4 h. Solvent was removed and the crude product was purified by column chromatography (EtOAc/Hexane) to compound 4X (250 mg). MS (m/z); 232 [M+H]

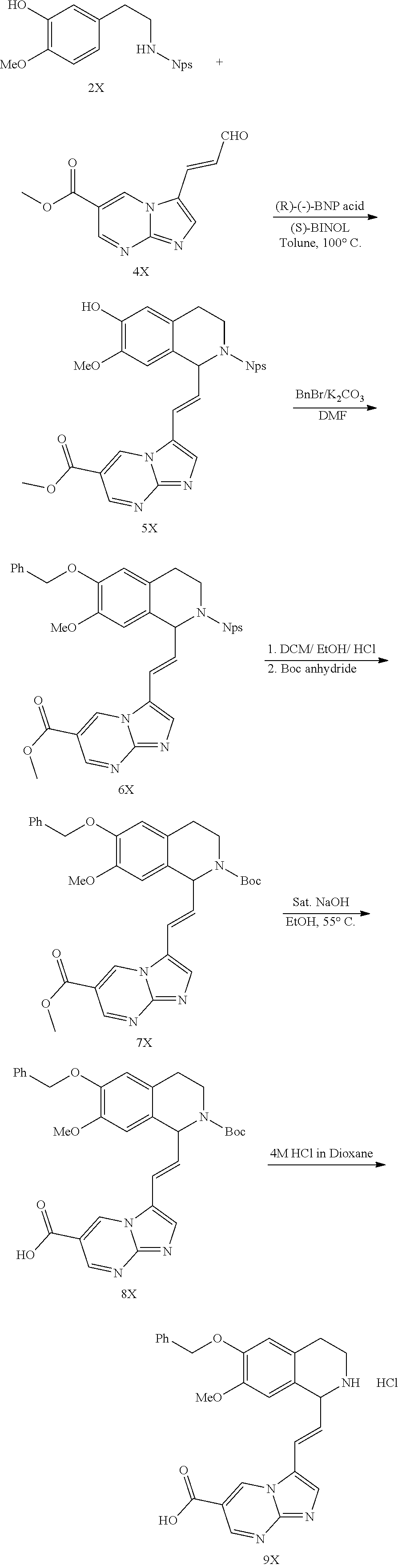

Compound 5X. To a stirred solution of compound 2X (4 g, 12.5 mmol, 1.0 eq) in toluene (20 mL) were added BINOL (1.48 g, 5 mmol, 0.4 eq), 1,1′-Binaphthyl-2,2′-diyl hydrogen phosphate (0.868 g, 2.5 mmol, 0.2 eq), and aldehyde 4X, and the reaction mixture was stirred at 90° C. for 24 h. Solvent was evaporated under vacuum to give a crude material, which was purified by column chromatography to give compound 5X. MS (m/z); 534 [M+H]

Compound 6X. To a solution of compound 5X (3.6 g, 6.74 mmol, 1.0 eq) in DMF (36 mL) were added K₂CO₃ (2.4 g, 16.85 mmol, 2.5 eq) and benzyl bromide (1.18 mL, 10.1 mmol, 1.5 eq) and the reaction mixture was stirred at room temperature for 5 h. The reaction mixture was diluted with ethyl acetate and the reaction mixture was washed with water (3×) and brine (1×). The organic layer was dried and evaporated under vacuum to give a crude compound, which was purified by column chromatography (EtOAc/Hexane) to 3.7 g compound 6X. MS (m/z); 624 [M+H]

Compound 7X. To a solution of compound 6X (3.7 g, 5.92 mmol) in DCM:Ethanol (1:1 25 mL) mixture at 0° C. was added concentrated HCl (5 mL) and the reaction mixture was stirred at RT for 1 h. Solvents were evaporated to give a crude solid, which was directly used in the next step without further purification. To a solution of the crude material (5.92 mmol) in DCM (35 mL) at 0° C. were added TEA (1.8 g, 17.76 mmol, 3.0 eq) and Boc₂O (1.94 g, 8.88 mmol, 1.5 eq) and the reaction mixture was stirred at room temperature for 3 h. The reaction mixture was concentrated under vacuum to give a residue, which was purified by column chromatography (EtOAc/Hexane) to afford 1.0 g of compound 7X. MS (m/z); 571 [M+H]

Compound 8X. To a solution of compound 7X (1.0 g, 1.75 mmol, 1.0 eq) in Methanol (10 mL) was added LiOH.H₂O (0.084 g, 3.50 mmol, 2.0 eq) at rt and the reaction mixture was stirred at room temperature for 3 h. The reaction mixture was concentrated and neutralized with aq. citric acid and then the reaction mixture was extracted with ethyl acetate (4×). The organic layer was washed with brine, dried and concentrated under vacuum to give a crude compound, which was purified by column chromatography to yield 0.35 g of compound 8X. MS (m/z); 557 [M+H].

Compound 9X. To a solution of compound 8X (0.50 g, 0.89 mmol) in Methanol (10 mL) was added 4M HCl in dioxane (10 mL) and the reaction mixture was stirred at room temperature for 12 h. Evaporation of the solvent under vacuum gave a residue, which was triturated with ether to give 0.34 g of compound 9X. MS (m/z); 457 [M+H].

### AI. (E)-3-(2-(7-methoxy-6-((4-methylbenzyl)oxy)-1,2,3,4-tetrahydroisoquinolin-1-yl)vinyl)imidazo[1,2-a]pyrimidine-6-carboxylic acid

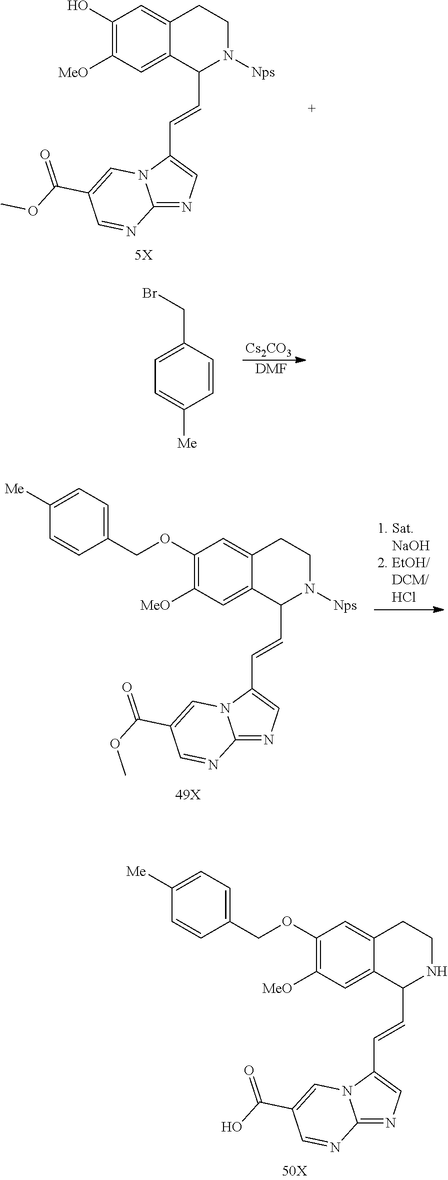

Compound 50X. To a solution of compound 5X (1.095 mmol, 1.0 eq) and Cs₂CO₃ (534 mg, 1.64 mmol, 1.5 eq) in DMF (10 mL) was added 4-methylbenzyl bromide (243 mg, 1.314 mmol, 1.2 eq) and the reaction mixture was stirred at room temperature for 10 h. Diluted with ethyl acetate and the organic layer was washed with water (3×50 mL), dried and evaporated to give a residue, which was purified by column chromatography to give compound 49X. To a solution of compound 49X (50 mg) in ethanol (3 mL) was added two drops of saturated aq. NaOH and the reaction mixture was heated 55° C. for 45 min. The reaction mixture was cooled to room temperature and then quenched with acetic acid (1 mL) and stirred for 10 min. Solvents were removed under vacuum to provide the crude acid, which was directly used in the next step. To a solution of the crude acid, DCM (10 mL) and Ethanol (10 mL) at 0° C. was added con. HCl (2 mL) and the reaction mixture was stirred at room for 2 hours. Solvent was removed under vacuum and product was purified with reverse phase chromatography to give compound 50X. MS (m/z); 471 [M+H]

### AJ. (E)-3-(2-(7-methoxy-6-((4-(trifluoromethyl)benzyl)oxy)-1,2,3,4-tetrahydroisoquinolin-1-yl)vinyl)imidazo[1,2-a]pyrimidine-6-carboxylic acid

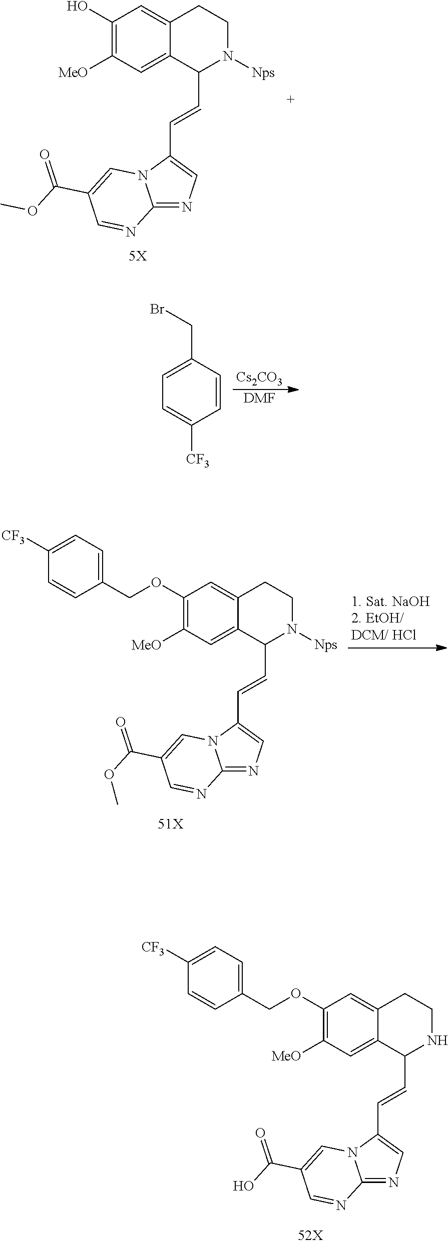

Compound 52X. To a solution of compound 5X (1.095 mmol, 1.0 eq) and Cs₂CO₃ (534 mg, 1.64 mmol, 1.5 eq) in DMF (10 mL) was added 4-trifluromethylbenzyl bromide (314 mg, 1.314 mmol, 1.2 eq) and the reaction mixture was stirred at room temperature for 10 h. Diluted with ethyl acetate and the organic layer was washed with water (3×50 mL), dried and evaporated to give a residue, which was purified by column chromatography to give compound 51X. To a solution of compound 51X (50 mg) in ethanol (3 mL) was added two drops of saturated aq. NaOH and the reaction mixture was heated 55° C. for 45 min. The reaction mixture was cooled to room temperature and then quenched with acetic acid (1 mL) and stirred for 10 min. Solvents were removed under vacuum to provide the crude acid, which was directly used in the next step. To a solution of the crude acid, DCM (10 mL) and Ethanol (10 mL) at 0° C. was added con. HCl (2 mL) and the reaction mixture was stirred at room for 2 hours. Solvent was removed under vacuum and product was purified with reverse phase chromatography to give compound 52X. MS (m/z); 525 [M+H].

### AK. (E)-3-(2-(7-methoxy-6-phenethoxy-1,2,3,4-tetrahydroisoquinolin-1-yl)vinyl)imidazo[1,2-a]pyrimidine-6-carboxylic acid

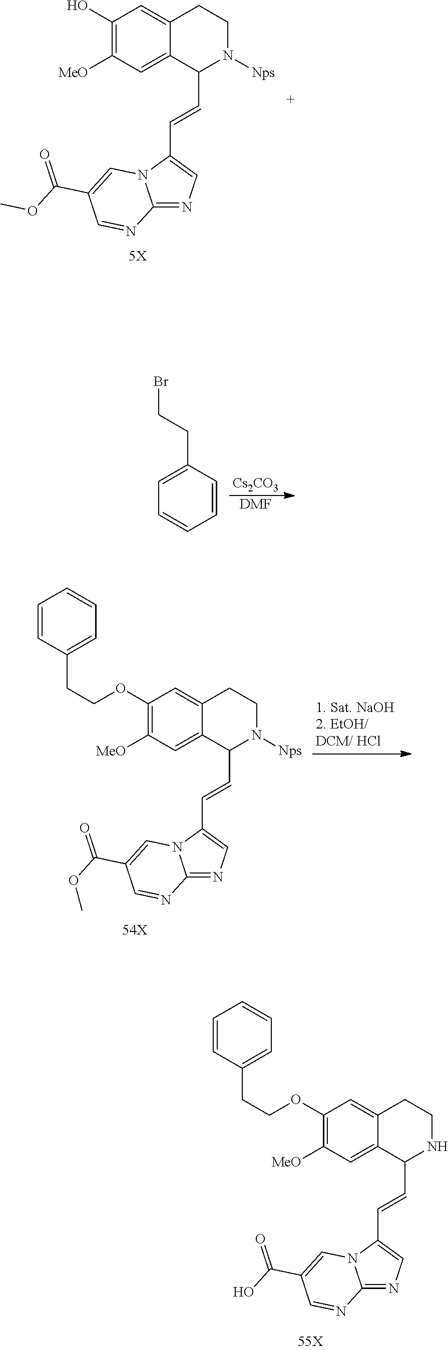

Compound 55X. To a solution of compound 5X (1.095 mmol, 1.0 eq) and Cs₂CO₃ (534 mg, 1.64 mmol, 1.5 eq) in DMF (10 mL) was added Phenethyl bromide (243 mg, 1.314 mmol, 1.2 eq) and the reaction mixture was stirred at room temperature for 10 h. Diluted with ethyl acetate and the organic layer was washed with water (3×50 mL), dried and evaporated to give a residue, which was purified by column chromatography to give compound 54X. To a solution of compound 54X (50 mg) in ethanol (3 mL) was added two drops of saturated aq. NaOH and the reaction mixture was heated 55° C. for 45 min. The reaction mixture was cooled to room temperature and then quenched with acetic acid (1 mL) and stirred for 10 min. Solvents were removed under vacuum to provide the crude acid, which was directly used in the next step. To a solution of the crude acid, DCM (10 mL) and Ethanol (10 mL) at 0° C. was added con. HCl (2 mL) and the reaction mixture was stirred at room for 2 hours solvent was removed under vacuum and product was purified with reverse phase chromatography to give compound 55X. MS (m/z); 471 [M+H]

### AL. (E)-3-(2-(6-(benzyloxy)-7-methoxy-1,2,3,4-tetrahydroisoquinolin-1-yl)vinyl)-2-methylimidazo[1,2-a]pyrimidine-6-carboxylic acid

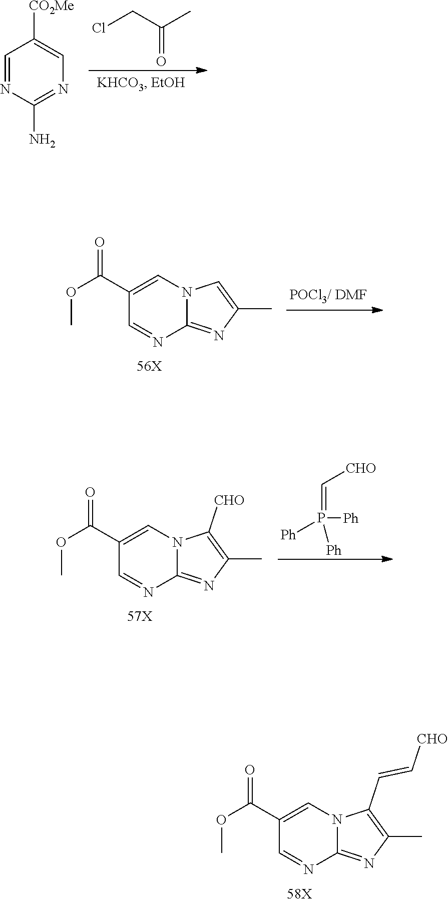

Compound 56X. To a solution of methyl 2-aminopyrimidine-5-carboxylate (2.0 g, 12.98 mmol, 1.0 eq) and potassium bicarbonate (3.89 g, 38.94 mmol, 3.0 eq) in Ethanol (40 mL) was added chloroacetone (2.4 g, 25.98 mmol, 2 eq) and the reaction mixture was heated at reflux for 12 h. An additional 2 equivalent of chloroacetone was added and the reaction mixture was heated at reflux for an additional 12 h. Cooled and the reaction mixture was filtered via celite and solvent was evaporated to give a residue, which was purified by column chromatography to give the compound 56X. MS (m/z); 192 [M+H]

Compound 58X. To a solution of dry DMF (8 mL) at room temperature was added POCl₃ (2.08 g, 13.61 mmol, 1.3 eq) drop wise and the reaction mixture was stirred at room temperature for 10 min. The reaction mixture was then cooled to 0° C. using an ice bath and compound 56X in DMF was added slowly over 10 min. After the addition, the reaction mixture was heat at 80-85° C. for 12 h. Excess POCl₃ was removed and then the flask was placed in an ice bath and ice was added into the reaction flask. Then saturated sodium bicarbonate solution was carefully added and the reaction mixture was stirred for 30 min. The reaction mixture was then extracted with DCM (4×50 mL). The combined layer was dried, solvent was removed to give a residue, which was purified by column chromatography (EtOAc/Hexane) to give the aldehyde 57X. To a solution of aldehyde 57X (186 mg, 0.85 mmol, 1.0 eq) in toluene (5 mL) was added (formylmethylene)triphenylphosphorane slowly. The reaction mixture was then heated at 80° C. for 6 h. Solvent was removed the residue was purified by column chromatography (EtOAc/Hexane) to give α,β-unsaturated aldehyde 58X. MS (m/z); 246 [M+H]

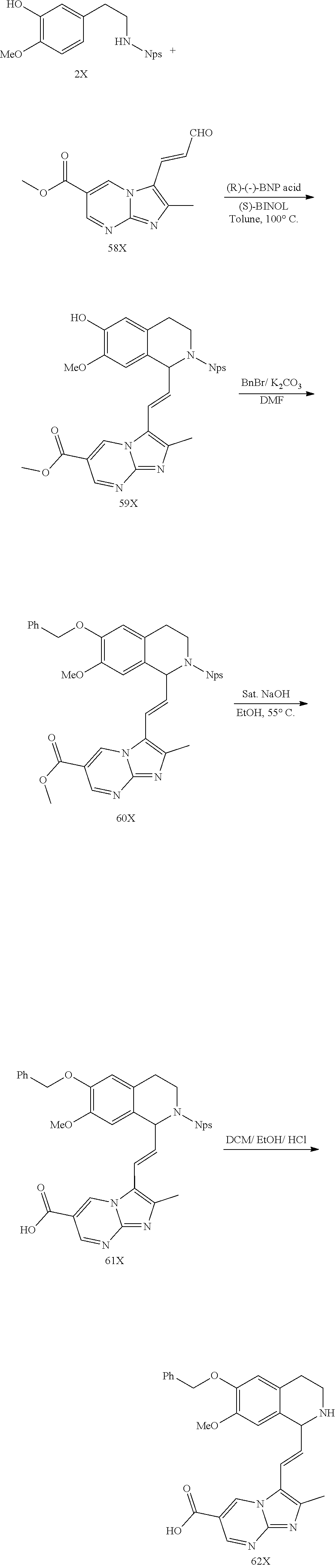

Compound 59X. To a solution of compound 2X (0.260 g, 0.813 mmol) in toluene (7 mL) were added BINOL (0.070 g, 0.244 mmol, 0.3 eq) and 1,1′-Binaphthyl-2,2′-diyl hydrogen phosphate, (0.043 g, 0.122 mmol, 0.15 eq) and the reaction mixture was stirred under argon for 10 min. The reaction mixture was then heated to 90 to 100° C. and then the aldehyde 58X (200 mg, 0.13 mmol, 1 eq) was added. The reaction mixture was stirred at this temperature for 6 h. Solvent was removed and the residue was purified by column chromatography (EtOAc/hexane) to give the compound 59X. MS (m/z); 548 [M+H]

Compound 60X. To a solution of 6-hydroxy compound 59X (200 mg, 0.365 mmol, 1.0 eq) and Cs₂CO₃ (237 mg, 0.73 mmol, 2.0 eq) in DMF (8 mL) was added benzyl bromide (94 mg, 0.547 mmol, 1.5 eq) and the reaction mixture was stirred at room temperature for 10 h. Diluted with ethyl acetate and the organic layer was washed with water (3×50 mL), dried and evaporated to give a residue, which was purified by column chromatography to give compound 60X.

Compound 62X. To a solution of 50 mg of the compound in ethanol (3 mL) was added two drops of saturated aq. NaOH and the reaction mixture was heated 55° C. for 45 min. The reaction mixture was cooled to room temperature and then quenched with acetic acid (1 mL) and stirred for 10 min. Solvents were removed under vacuum to provide the crude acid 61X, which was directly used in the next step. To a solution of the crude acid, DCM (10 mL) and Ethanol (10 mL) at 0° C. was added con. HCl (2 mL) and the reaction mixture was stirred at room for 2 hours. Solvent was removed under vacuum and product was purified with reverse phase chromatography to give compound 62X. MS (m/z); 471 [M+H]

### BA. (E)-3-(2-(6-(benzyloxy)-7-methoxy-1,2,3,4-tetrahydroisoquinolin-1-yl)vinyl)imidazo[1,2-a]pyrazine-6-carboxylic acid

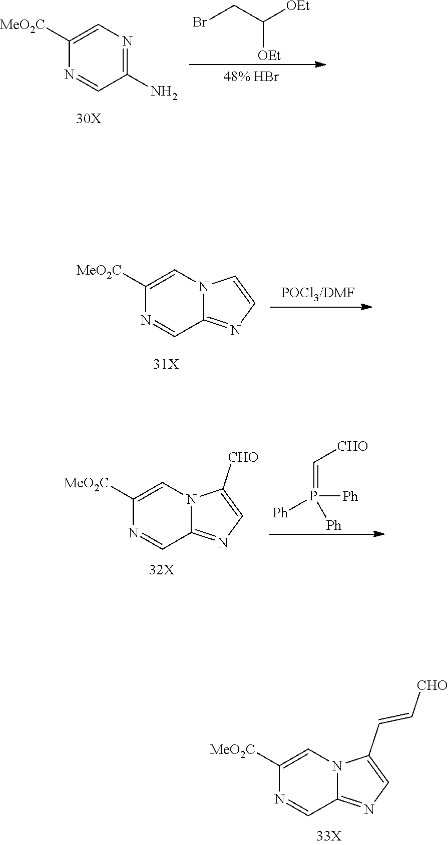

Compound 31X. To a solution of methyl compound 30X (1.0 g, 6.53 mmol, 1.0 eq) in Ethanol (10 mL) at rt were added 2-bromo-1,1-diethoxyethane (1.5 g, 7.83 mmol, 1.2 eq) and 48% HBr (2 mL) and the reaction mixture was heated at 80° C. for 16 hrs. Reaction mixture was concentrated and residue was neutralized by slow addition of aq. NaHCO₃. The reaction mixture was extracted with ethyl acetate (3×) and the combined organic layer was dried and evaporated under vacuum to provide crude compound, which was purified by column chromatography to get 1.0 g of compound 31X. MS (m/z); 178 [M+H].

Compound 32X. To a solution of compound 31X (1.0 g, 5.64 mmol, 1.0 eq) in DMF (5 mL), was added POCl₃ (4.30 g, 28.24 mmol, 5.0 eq) slowly and the reaction mixture was heated at 80° C. for 4 h. Excess POCl₃ was removed under vacuum and the residue was cooled to 0° C. and then quenched by slow addition of aq. NaHCO₃ solution. The reaction mixture was extracted with ethyl acetate (3×) and the combined organic layer was washed with water (2×), dried and evaporated to give a crude compound, which was purified by column chromatography to give 0.80 g of aldehyde 32X. MS (m/z); 206 [M+H].

Compound 33X. To the stirred solution of aldehyde 32X (0.35 g, 1.70 mmol, 1.0 equiv) in toluene (5 mL) was added (formylmethylene)triphenylphosphorane (0.502 g, 1.71 mmol, 1.0 eq) and the reaction mixture was stirred at 80° C. for 4 h. Solvent was evaporated to give a residue, which was purified by column chromatography (EtOAc/Hexane) to provide 0.20 g of α,β-unsaturated aldehyde 33X. MS (m/z); 232 [M+H].

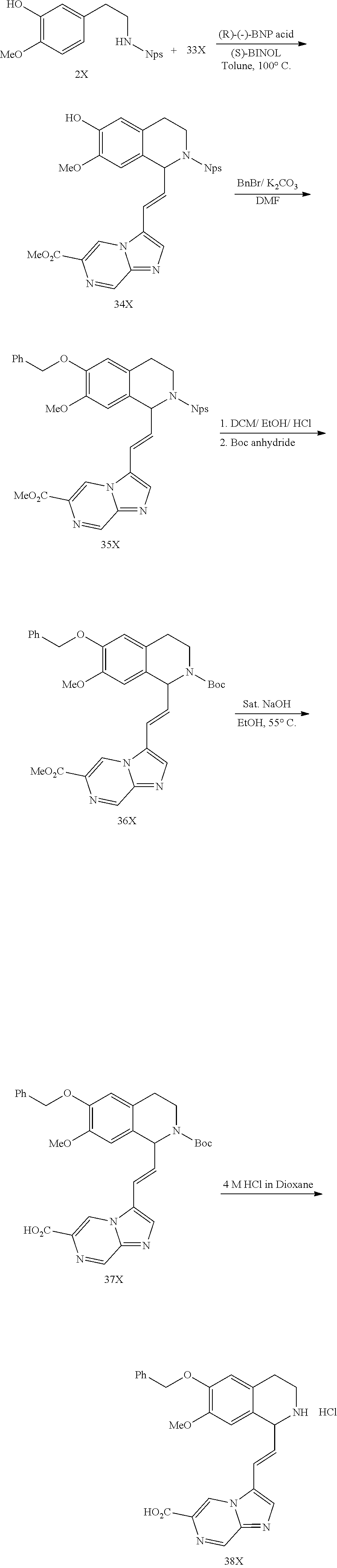

Compound 34X. To a solution of compound 2X (0.20 g, 0.63 mmol, 1.0 eq) in toluene (5 mL) were added BINOL (0.031 g, 0.12 mmol, 0.2 eq), 1,1′-Binaphthyl-2,2′-diyl hydrogen phosphate, (0.002 g, 0.06 mmol, 0.1 eq) and aldehyde 33X (0.19 g, 0.74 mmol, 1.2 eq) and the reaction mixture was heated at 90° C. for 4 h. Solvent was evaporated under vacuum and the residue was purified by column chromatography (ethyl acetate in hexane) to yield 0.12 g of compound 34X. MS (m/z); 534 [M+H].

Compound 35. To a solution of compound 34X (0.3 g, 0.67 mmol, 1.0 eq) and K₂CO₃ (0.24 g, 1.68 mmol, 2.5 eq) in DMF (10 mL) was added benzyl bromide (0.12 mL, 1.01 mmol, 1.5 eq) and the reaction mixture was stirred at room temperature for 10 h. The reaction mixture was diluted with ethyl acetate. The combined organic layer was washed with water (2×), dried and evaporated under vacuum to give the crude compound, which was purified by column chromatography to provide 0.37 g of the compound 35X. MS (m/z); 624 [M+H].

Compound 36X. To a stirred solution of compound 35X (0.1 g, 0.18 mmol) in DCM:Ethanol (3 mL:3 mL) mixture at 0° C. was added concentrated HCl (1 mL) and the reaction mixture was stirred at 0° C. for 1 h. Solvents were evaporated to provide a crude material, which was used in the next step without further purification. To a solution of the crude material (0.18 mmol, 1.0 eq) in DCM (5 mL) at 0° C. were added TEA (0.19 g, 0.54 mmol, 3.0 eq) and Boc₂O (0.059 g, 0.27 mmol, 1.5 eq). The reaction mixture was then stirred at room temperature for 3 h. Solvent was removed to provide a residue which was purified by column chromatography to yield 20 mg of compound 36X. MS (m/z); 571 [M+H]

Compound 38X. To a solution of compound 36X (75 mg, 0.132 mmol) in ethanol (5 mL) was added 3 drops of saturated aq. NaOH and the reaction mixture was heated 55° C. for 45 min. The reaction mixture was cooled to room temperature and then quenched with acetic acid (2 mL) and stirred for 10 min. Solvents were removed under vacuum to provide the crude acid 37X, which was directly used in the next step. To a solution of the crude acid, in MeOH (5 mL) was added 4M HCl in dioxane (5 mL) and the reaction mixture was stirred at room for 4 hours. Solvent was removed under vacuum and product was purified with reverse phase chromatography to give compound 38X. MS (m/z); 457 [M+H]

### CA. (E)-3-(2-(6-(benzyloxy)-7-methoxy-1,2,3,4-tetrahydroisoquinolin-1-yl)vinyl)imidazo[1,2-b]pyridazine-6-carboxylic acid

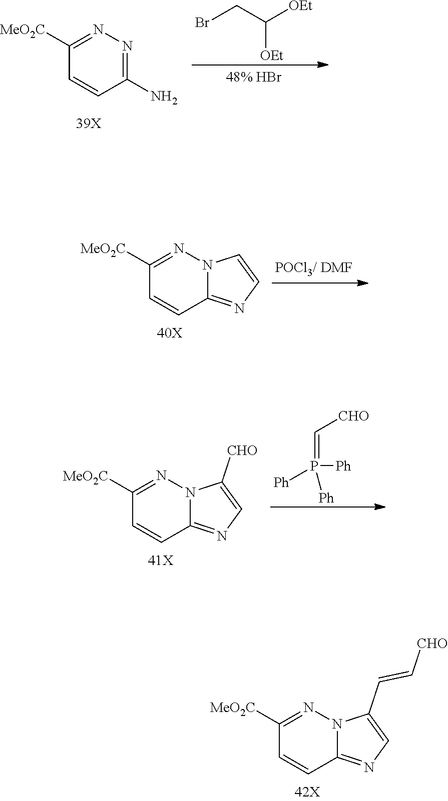 [SMILES: COC(=O)c1ccc(N)nn1 ; CCOC(CBr)OCC ; COC(=O)c1ccc2nccn2n1 ; COC(=O)c1ccc2ncc(C=O)n2n1 ; O=CC=P(c1ccccc1)(c1ccccc1)c1ccccc1 ; COC(=O)c1ccc2ncc(/C=C/C=O)n2n1]

Procedure: To a solution of compound 39X (1.0 g, 6.53 mmol, 1.0 eq) in ethanol (10 mL) were added 2-bromo-1,1-diethoxyethane (1.30 g, 6.53 mmol, 1.0 eq) and 48% HBr (2 mL) and the reaction mixture was heated at 80° C. for 16 hrs. Reaction mixture was concentrated and residue was neutralized by slow addition of aq. NaHCO₃. The reaction mixture was extracted with ethyl acetate (3×) and the combined organic layer was dried and evaporated under vacuum to provide crude compound, which was purified by column chromatography to get 1.0 g of compound 40X. MS (m/z); 178 [M+H].

Compound 41X. To a solution of compound 40X (1.0 g, 5.64 mmol, 1.0 eq) in DMF (5 mL), was added POCl₃ (4.30 g, 28.24 mmol, 5.0 eq) slowly and the reaction mixture was heated at 80° C. for 4 h. Excess POCl₃ was removed under vacuum and the residue was cooled to 0° C. and then quenched by slow addition of aq. NaHCO₃ solution. The reaction mixture was extracted with ethyl acetate (3×) and the combined organic layer was washed with water (2×), dried and evaporated to give a crude compound, which was purified by column chromatography to give 0.80 g of aldehyde 41X. MS (m/z); 206 [M+H].

Compound 42X. To a solution of aldehyde 41X (0.35 g, 1.70 mmol) in toluene (2 mL) was added (formylmethylene)triphenylphosphorane (0.502 g, 1.71 mmol, 1.0 eq) and the reaction mixture was stirred at 80° C. for 4 h. Solvent was evaporated to give a residue, which was purified by column chromatography (EtOAc/Hexane) to provide 0.19 g of α,β-unsaturated aldehyde 42X. MS (m/z); 232 [M+H].

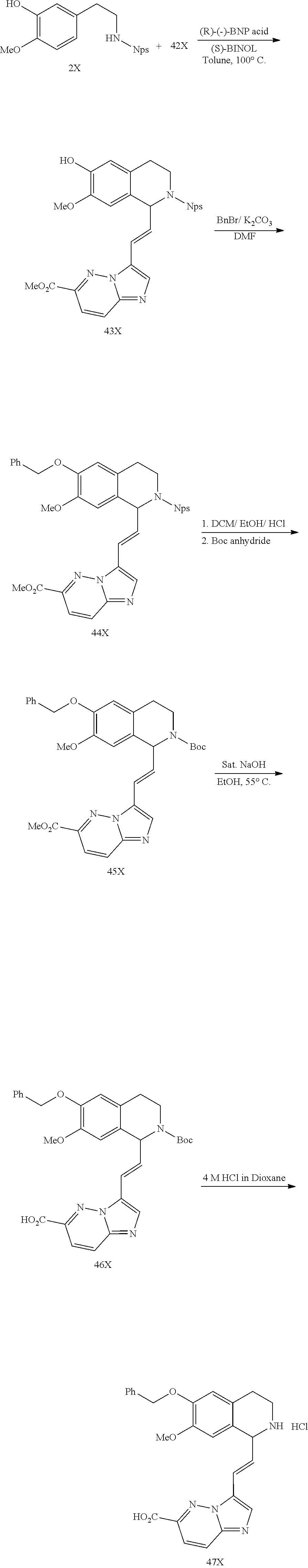

Compound 43X. To a solution of compound 2X (0.20 g, 0.63 mmol, 1.0 eq) in toluene (5 mL) were added BINOL (0.031 g, 0.12 mmol, 0.2 eq), 1,1′-Binaphthyl-2,2′-diyl hydrogen phosphate, (0.002 g, 0.06 mmol, 0.1 eq) and aldehyde 42X (0.19 g, 0.74 mmol, 1.2 eq) and the reaction mixture was heated at 90° C. for 4 h. Solvent was evaporated under vacuum and the residue was purified by column chromatography (ethyl acetate in hexane) to yield 0.12 g of compound 43X. MS (m/z); 534 [M+H].

Compound 44X. To a solution of compound 43X (0.3 g, 0.67 mmol, 1.0 eq) and K₂CO₃ (0.24 g, 1.68 mmol, 2.5 eq) in DMF (10 mL) was added benzyl bromide (0.12 mL, 1.01 mmol, 1.5 eq) and the reaction mixture was stirred at room temperature for 10 h. The reaction mixture was diluted with ethyl acetate. The combined organic layer was washed with water (2×), dried and evaporated under vacuum to give the crude compound, which was purified by column chromatography to provide 0.25 g of the compound 44X. MS (m/z); 624 [M+H].

Compound 45X. To a stirred solution of compound 44X (0.1 g, 0.18 mmol) in DCM:Ethanol (3 mL:3 mL) mixture at 0° C. was added concentrated HCl (1 mL) and the reaction mixture was stirred at 0° C. for 1 h. Solvents were evaporated to provide a crude material, which was used in the next step without further purification. To a solution of the crude material (0.18 mmol, 1.0 eq) in DCM (5 mL) at 0° C. were added TEA (0.19 g, 0.54 mmol, 3.0 eq) and Boc₂O (0.059 g, 0.27 mmol, 1.5 eq). The reaction mixture was then stirred at room temperature for 3 h. Solvent was removed to provide a residue which was purified by column chromatography to yield 20 mg of compound 45X. MS (m/z); 571 [M+H]

Compound 47X. To a solution of compound 45X (75 mg, 0.132 mmol) in ethanol (5 mL) was added 3 drops of saturated aq. NaOH and the reaction mixture was heated 55° C. for 45 min. The reaction mixture was cooled to room temperature and then quenched with acetic acid (2 mL) and stirred for 10 min. Solvents were removed under vacuum to provide the crude acid 46X, which was directly used in the next step. To a solution of the crude acid, in MeOH (5 mL) was added 4M HCl in dioxane (5 mL) and the reaction mixture was stirred at room for 4 hours. Solvent was removed under vacuum and product was purified with reverse phase chromatography to give compound 47X. MS (m/z); 457 [M+H]

### CB. Methyl (E)-3-(2-(6-(benzyloxy)-7-methoxy-1,2,3,4-tetrahydroisoquinolin-1-yl)vinyl)imidazo[1,2-b]pyridazine-6-carboxylate

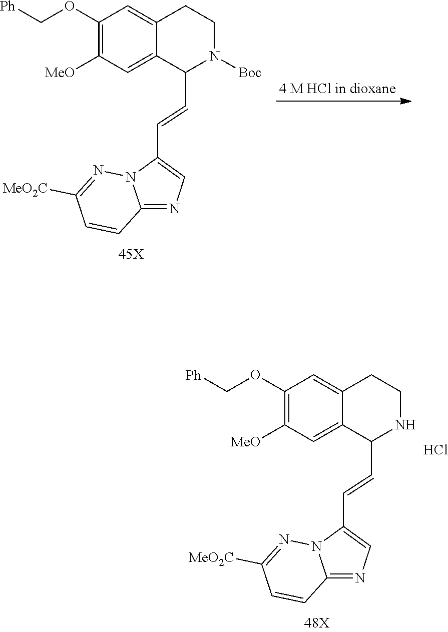 [SMILES: COC(=O)c1ccc2ncc(/C=C/C3c4cc(OC)c(OCc5ccccc5)cc4CCN3C(=O)OC(C)(C)C)n2n1 ; COC(=O)c1ccc2ncc(/C=C/C3NCCc4cc(OCc5ccccc5)c(OC)cc43)n2n1 ; Cl]

Compound 48X. To a stirred solution of compound 45X (0.01 g, 0.012 mmol) in MeOH (3 mL) was added 4 M HCl in dioxane (3 mL) and the reaction mixture was stirred at room temperature for 6 h. The reaction mixture was concentrated under vacuum to give a crude amine, which was triturated with ether to afford 6.0 mg of compound 48X. MS (m/z); 471 [M+H].

### DA. (E)-3-(2-(6-(benzyloxy)-7-methoxy-1,2,3,4-tetrahydroisoquinolin-1-yl)vinyl)imidazo[1,2-a]pyridine-6-carboxylic acid

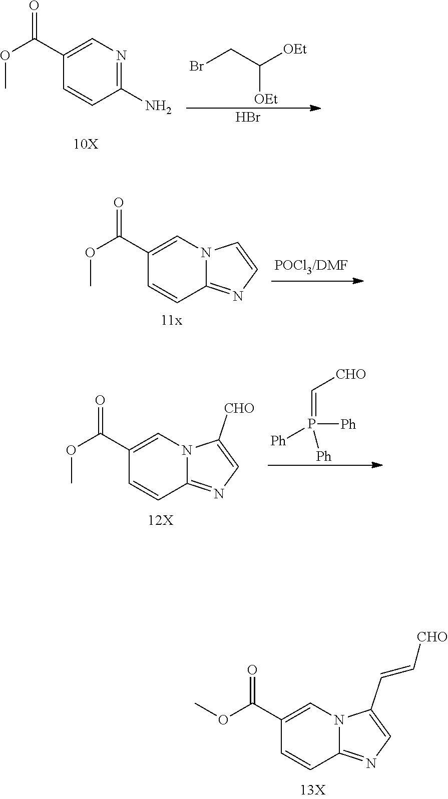

Compound 11X. To the stirred solution of methyl 6-aminonicotinate (compound 10X) (2.4 g, 15.78 mmol) in ethanol:water ((10:5 mL) were added 2-bromo-1,1-diethoxyethane (2.96 g, 18.95 mmol) and 48% HBr (2.5 mL) at rt and the reaction mixture was heated at 100° C. for 6 h. The reaction mixture was carefully neutralized with sat. NaHCO₃ solution. The reaction mixture was extracted with ethyl acetate (3×50 mL). The combined organic layer was dried and evaporated under vacuum to give 1.8 g of compound 11X. MS (m/z); 177 [M+H]

Compound 12X. To a stirred solution of compound 11X (2.0 g, 11.36 mmol) in DMF (25 mL) was added POCl₃ (10.45 g, 68.18 mmol) slowly. The reaction mass was heated at 80° C. for 4 h. Excess POCl₃ was removed under vacuum. The reaction mixture was then placed in an ice bath and saturated NaHCO₃ solution was carefully added and the reaction mixture was stirred for 30 min. The reaction mixture was then extracted with DCM (5×50 mL). The combined organic layer was washed with water (2×), dried and evaporated under vacuum to give a residue, which was purified by Purification by column chromatography (ethyl acetate/hexane) to afford 1.5 g of aldehyde 12X. MS (m/z); 205 [M+H]

Compound 13X. To a stirred solution of methyl 3-formylimidazo[1,2-a]pyridine-6-carboxylate (0.200 g, 0.980 mmol, 1.0 eq) in toluene (4 mL) was added (formylmethylene)triphenylphosphorane (0.447 g, 1.47 mmol, 1.5 eq) and the reaction mass was stirred at 80° C. for 4 h. Solvent was removed and the residue was purified by column chromatography (EtOAc/Hexane) to give α,β-unsaturated aldehyde 13X. MS (m/z); 231 [M+H]

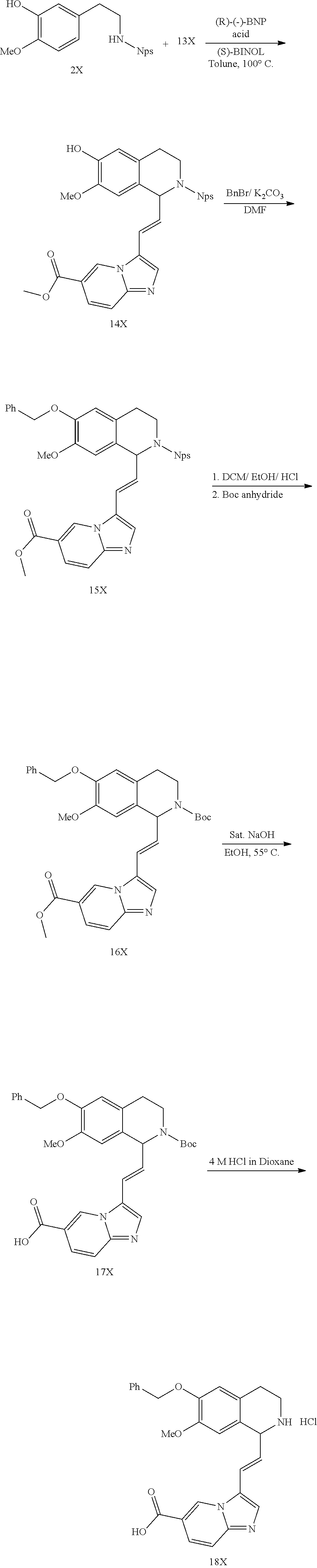

Compound 14X. To a solution of compound 2X (0.40 g, 3.12 mmol, 1.0 eq) in toluene (15 mL) were added BINOL (0.178 g, 0.62 mmol, 0.2 eq), 1,1′-Binaphthyl-2,2′-diyl hydrogen phosphate, (0.02 g, 0.31 mmol, 0.1 eq) and aldehyde 13X (0.30 g, 4.68 mmol, 1.5 eq) and the reaction mixture was heated at 90° C. for 4 h. Solvent was evaporated under vacuum and the residue was purified by column chromatography (ethyl acetate in hexane) to yield 0.3 g of compound 14X. MS (m/z); 533 [M+H]

Compound 15X. To a solution of compound 14X (0.3 g, 0.67 mmol, 1.0 eq) and K₂CO₃ (0.24 g, 1.68 mmol, 2.5 eq) in DMF (10 mL) was added benzyl bromide (0.12 mL, 1.01 mmol) and the reaction mixture was stirred at room temperature for 10 h. The reaction mixture was diluted with ethyl acetate. The combined organic layer was washed with water (2×), dried and evaporated under vacuum to give the crude compound, which was purified by column chromatography to provide compound 15X. MS (m/z); 623 [M+H]

Compound 16X. To a stirred solution of compound 15X (0.37 g, 0.59 mmol), in DCM:Ethanol (5 mL:5 mL) mixture at 0° C. was added concentrated HCl (2 mL) and the reaction mixture was stirred at this temperature for 1 h. Solvent was evaporated to give a crude material, which was used in the next step without further purification. To a stirred solution of the crude material (0.59 mmol, 1.0 eq) in DCM (10 mL) at 0° C. were added TEA (0.19 g, 1.8 mmol, 3.0 eq) and Boc₂O (0.193 g, 0.89 mmol, 1.5 eq). The reaction mixture was then stirred at room temperature for 3 h. Solvent was removed to provide a residue which was purified by column chromatography to yield 30 mg of compound 16X. MS (m/z); 570 [M+H]

Compound 17X. To a stirred solution of compound 16X (0.01 g, 0.012 mmol, 1.0 eq) in Ethanol:water (1:1 2 mL) was added LiOH.H₂O (0.005 g, 0.024 mmol, 2.0 eq) and the reaction mixture was stirred at room temperature for 3 h. The reaction mixture was concentrated and quenched with citric acid. The reaction mixture was extracted with ethyl acetate (3×) and the combined organic layer was dried and concentrated under vacuum to give the crude acid, which was purified by column chromatography (EtOAc/Hexane to EtOAc/MeOH) to afforded 0.008 g of compound 17X. MS (m/z); 556 [M+H].

Compound 18X. To a solution of compound 17X (25 mg, 0.045 mmol) in Methanol (5 mL) was added 4M HCl in dioxane (4 mL) and the reaction mixture was stirred at room temperature for 12 h. Evaporation of the solvent under vacuum gave a residue, which was triturated with ether to give 15 mg of compound 18X. MS (m/z); 456 [M+H].

### DB. Methyl (E)-3-(2-(6-(benzyloxy)-7-methoxy-1,2,3,4-tetrahydroisoquinolin-1-yl)vinyl)imidazo[1,2-a]pyridine-6-carboxylate

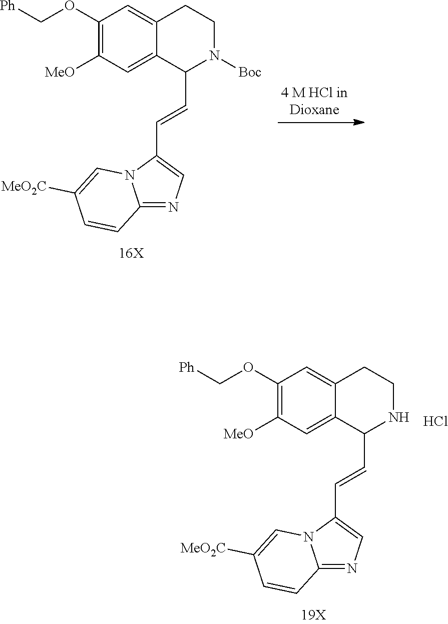 [SMILES: COC(=O)c1ccc2ncc(/C=C/C3c4cc(OC)c(OCc5ccccc5)cc4CCN3C(=O)OC(C)(C)C)n2c1 ; COC(=O)c1ccc2ncc(/C=C/C3NCCc4cc(OCc5ccccc5)c(OC)cc43)n2c1 ; Cl]

Compound 19X. To a stirred solution of compound 16X (0.01 g, 0.012 mmol) in MeOH (3 mL) was added 4 M HCl in dioxane (3 mL) and the reaction mixture was stirred at room temperature for 6 h. The reaction mixture was concentrated under vacuum to give a crude amine, which was triturated with ether to afford 8.0 mg of compound 19X. MS (m/z); 470 [M+H].

### DC. (E)-3-(2-(6-(benzyloxy)-7-methoxy-1,2,3,4-tetrahydroisoquinolin-1-yl)vinyl)imidazo[1,2-a]pyridine-7-carboxylic acid

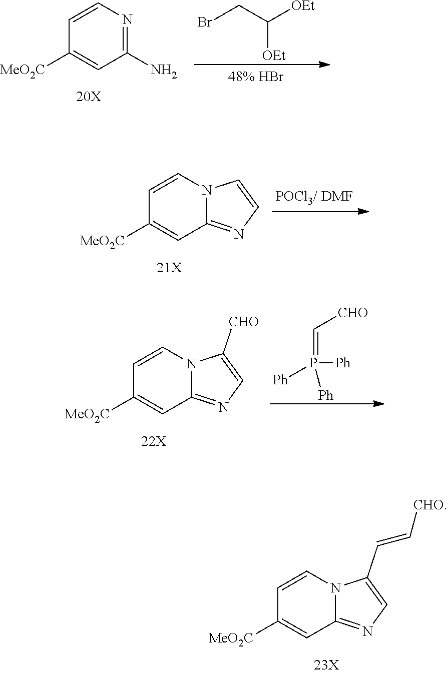

Compound 21X. To a solution of methyl 2-aminoisonicotinate 20X (5.0 g, 3.26 mmol) in Ethanol (25 mL) at room temperature were added 2-bromo-1,1-diethoxyethane (6.5 mL, 3.90 mmol) and 48% HBr solution (3 mL) and the reaction mixture was then heated at 80° C. for 16 hrs. Reaction mixture was concentrated and then quenched with aq. NaHCO₃ solution. The reaction mixture was then extracted with ethyl acetate (3×). Combined organic layer was washed with brine (1×), dried and evaporated to give the crude compound, which was purified by column chromatography to yield 2.50 g of compound 21X. MS (m/z); 177 [M+H]. Product.

Compound 22X. To a solution of compound 21X (2.50 g, 28.22 mmol, 1.0 eq) in DMF (25 mL) was added POCl₃ (12.4 g, 85.12 mmol, 3.0 eq) slowly and the reaction mixture was heated at 80° C. for 4 h. Excess POCl₃ was removed under vacuum and the residue was cooled to 0° C. and then quenched by slow addition of aq. NaHCO₃ solution. The reaction mixture was extracted with ethyl acetate (3×) and the combined organic layer was washed with water (2×), dried and evaporated to give a crude compound, which was purified by column chromatography (EtOAc/Hexane) to give 1.20 g of aldehyde 22X. MS (m/z); 205 [M+H].

Compound 23X. To a solution of compound 22X (1.20 g, 5.88 mmol, 1.0 eq) in toluene (10 mL) was added (formylmethylene)triphenylphosphorane (1.05 g, 5.88 mmol, 1.0 equiv) and the reaction mixture was stirred at 80° C. for 4 h. Solvent was evaporated to give a residue, which was purified by column chromatography (EtOAc/Hexane) to provide 0.50 g of α,β-unsaturated aldehyde 23X. MS (m/z); 231 [M+H].

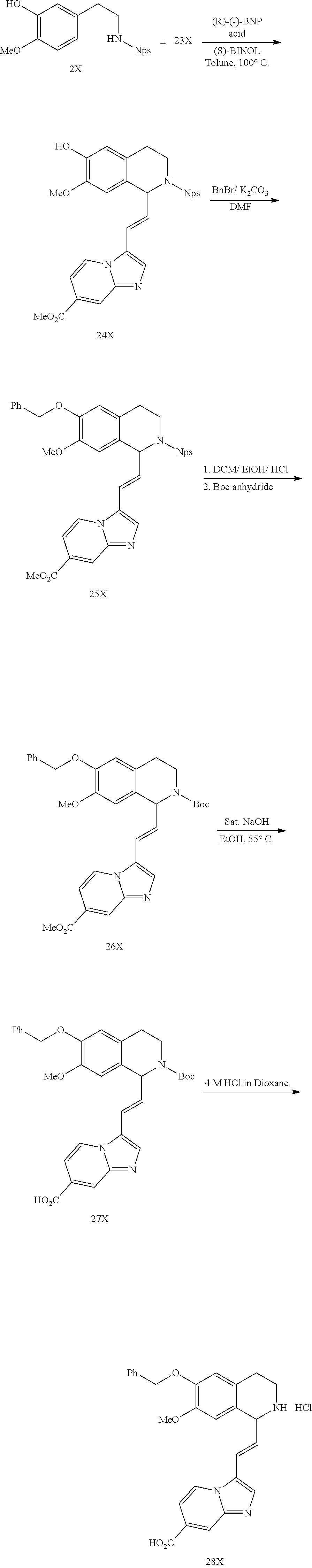

Compound 24X. To a solution of compound 2X (0.40 g, 3.12 mmol, 1.0 eq) in toluene (10 mL) were added BINOL (0.178 g, 0.62 mmol, 0.2 eq), 1,1′-Binaphthyl-2,2′-diyl hydrogen phosphate, (0.02 g, 0.31 mmol, 0.1 eq) and aldehyde 23X (0.30 g, 4.68 mmol, 1.5 eq) and the reaction mixture was heated at 90° C. for 4 h. Solvent was evaporated under vacuum and the residue was purified by column chromatography (ethyl acetate in hexane) to yield 0.3 g of compound 24X. MS (m/z); 533 [M+H].

Compound 25X. To a stirred mixture of compound 24X (0.3 g, 0.67 mmol, 1.0 eq) and K₂CO₃ (0.24 g, 1.68 mmol, 2.5 eq) in DMF (10 mL) was added benzyl bromide (0.12 mL, 1.01 mmol) and the reaction mixture was stirred at room temperature for 10 h. The reaction mixture was diluted with ethyl acetate. The combined organic layer was washed with water (2×), dried and evaporated under vacuum to give the crude compound, which was purified by column chromatography to provide 0.37 g of compound 25X. MS (m/z); 623 [M+H].

Compound 26X. To a stirred solution of compound 25X (0.37 g, 0.59 mmol) in DCM:Ethanol (5 mL:5 mL) mixture at 0° C. was added concentrated HCl (2 mL) and the reaction mixture was stirred at 0° C. for 1 h. Solvents were evaporated to provide a crude material, which was used in the next step without further purification. To a stirred solution of the crude material (0.59 mmol, 1.0 eq) in DCM (10 mL) at 0° C. were added TEA (0.19 g, 1.8 mmol, 3.0 eq) and Boc₂O (0.193 g, 0.89 mmol, 1.5 eq). The reaction mixture was then stirred at room temperature for 3 h. Solvent was removed to provide a residue which was purified by column chromatography to yield 25 mg of compound 26X. MS (m/z); 570 [M+H]

Compound 28X. To a solution of compound 26X (100 mg, 0.176 mmol) in ethanol (6 mL) was added 4 drops of saturated aq. NaOH and the reaction mixture was heated 55° C. for 45 min. The reaction mixture was cooled to room temperature and then quenched with acetic acid (2 mL) and stirred for 10 min. Solvents were removed under vacuum to provide the crude acid 27X, which was directly used in the next step. To a solution of the crude acid, in MeOH (5 mL) was added 4M HCl in dioxane (5 mL) and the reaction mixture was stirred at room for 4 hours. Solvent was removed under vacuum and product was purified with reverse phase chromatography. MS (m/z); 456 [M+H]

### DD. Methyl (E)-3-(2-(6-(benzyloxy)-7-methoxy-1,2,3,4-tetrahydroisoquinolin-1-yl)vinyl)imidazo[1,2-a]pyridine-7-carboxylate

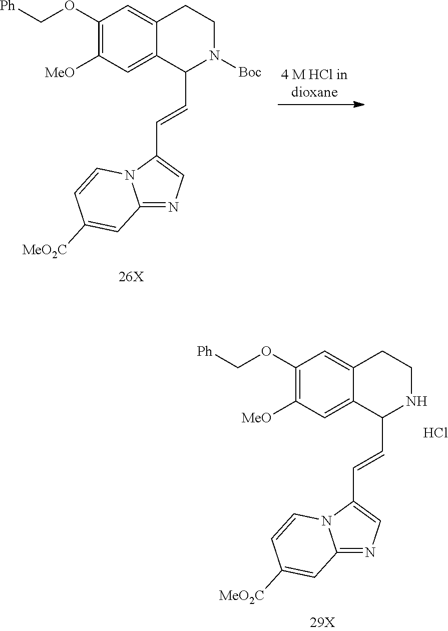 [SMILES: COC(=O)c1ccn2c(/C=C/C3c4cc(OC)c(OCc5ccccc5)cc4CCN3C(=O)OC(C)(C)C)cnc2c1 ; COC(=O)c1ccn2c(/C=C/C3NCCc4cc(OCc5ccccc5)c(OC)cc43)cnc2c1 ; Cl]

Compound 29X. To a stirred solution of compound 26X (0.01 g, 0.012 mmol) in MeOH (3 mL) was added 4 M HCl in dioxane (3 mL) and the reaction mixture was stirred at room temperature for 6 h. The reaction mixture was concentrated under vacuum to give a crude amine, which was triturated with ether to afford 6.0 mg of compound 29X. MS (m/z); 470 [M+H].

### Example 7: 6-(Benzyloxy)-7-methoxy-1-[(E)-2-(5-methoxy-1-methyl-1H-pyrrolo[2,3-b]pyridin-3-yl)ethenyl]-1,2,3,4-tetrahydroisoquinoline hydrochloride

### A) 5-(Benzyloxy)-2-bromo-4-methoxybenzaldehyde

To a solution of 2-bromo-5-hydroxy-4-methoxybenzaldehyde (50.0 g) in DMF (500 mL) was added Cs₂CO₃ (116 g) and benzyl bromide (32.1 mL) at 0° C. The mixture was stirred at 0° C. to room temperature overnight. To the mixture was added water (1000 mL) at room temperature, the precipitated solid was collected by filtration and air-dried overnight to give the title compound (69.5 g) as a white solid. MS: [M+H]+ 321.0, 323.0.

### B) 1-(Benzyloxy)-4-bromo-2-methoxy-5-[(E)-2-nitroethenyl]benzene

To a suspension of 5-(benzyloxy)-2-bromo-4-methoxybenzaldehyde (69.5 g) in AcOH (800 mL) was added ammonium acetate (66.7 g) and nitromethane (35.2 mL) at room temperature. The mixture was stirred at 100° C. for 5 h. To the mixture was added water (1000 mL) at room temperature. The mixture was stirred at room temperature over weekend. The precipitated solid was collected by filtration, washed with water and dried to give the title compound (83.0 g) as a yellow solid. MS: [M+H]⁺ 364.0, 366.0.

### C) 1-(Benzyloxy)-4-bromo-2-methoxy-5-(2-nitroethyl)benzene

To a suspension of 1-(benzyloxy)-4-bromo-2-methoxy-5-[(E)-2-nitroethenyl]benzene (79.0 g) in DMSO (1250 mL) and AcOH (250 mL) was added NaBH₄ (9.82 g) at room temperature. The mixture was stirred at room temperature overnight. The mixture was poured into water (2500 mL) at room temperature, and to the mixture was added solid NaHCO₃ at room temperature until gas evolution ceased. The precipitated solid was collected by filtration and washed with water. The solid was dissolved in EtOAc/THF (1/1), the mixture was filtrated, and insoluble material was washed with EtOAc/THF (1/1). The filtrate was dried over Na₂SO₄, filtrated again and concentrated. The residual solid was suspended in EtOAc/hexane (1/9, 500 mL) and the mixture was stirred overnight. The solid was collected by filtration and washed with EtOAc-hexane (1/9) to give the title compound (66.7 g) as a white solid. ¹H NMR (400 MHz, DMSO-d₆) δ 3.27 (2H, t, J=7.3 Hz), 3.77 (3H, s), 4.78 (2H, t, J=7.2 Hz), 5.05 (2H, s), 7.16 (2H, s), 7.29-7.50 (5H, m).

### D) tert-Butyl {2-[5-(benzyloxy)-2-bromo-4-methoxyphenyl]ethyl}carbamate

To a solution of 1-(benzyloxy)-4-bromo-2-methoxy-5-(2-nitroethyl)benzene (32.4 g) in EtOH (800 mL) and saturated aqueous NH₄Cl (160 mL) was added iron powder (74.1 g) at room temperature. The mixture was stirred at room temperature for 2 h. To the mixture was added Boc₂O (103 mL) at room temperature. The mixture was stirred at room temperature over weekend. The insoluble material was removed by celite filtration, and washed with EtOAc and water. The organic solvent was removed by evaporation. The mixture was extracted with EtOAc/THF (1/1). The organic layer was separated, washed with water and brine, dried over Na₂SO₄ and concentrated in vacuo. The residue was purified by column chromatography (silica gel, eluted with 2%-100% EtOAc in hexane) thrice to give the title compound (35.4 g) as a white solid. ¹H NMR (400 MHz, CDCl₃) δ 1.44 (9H, s), 2.82 (2H, br t, J=6.6 Hz), 3.31 (2H, br d, J=5.9 Hz), 3.85 (3H, s), 4.54 (1H, br s), 5.10 (2H, s), 6.78 (1H, br s), 7.03 (1H, s), 7.28-7.34 (1H, m), 7.34-7.40 (2H, m), 7.40-7.45 (2H, m).

### E) tert-Butyl {2-[2-acetyl-5-(benzyloxy)-4-methoxyphenyl]ethyl}carbamate

A mixture of tert-butyl {2-[5-(benzyloxy)-2-bromo-4-methoxyphenyl]ethyl}carbamate (20.0 g), K₂CO₃ (8.24 g), 1,3-bis(diphenylphosphino)propane (3.78 g) and butyl vinyl ether (29.7 mL) in n-BuOH (250 mL) was degassed and the reaction vessel was purged with Ar gas. To the mixture was added Pd(OAc)₂ (1.54 g) at room temperature and the mixture was stirred at 80° C. for 2 h under Ar. The mixture was cooled to 0° C. and diluted with THF (250 mL). To the mixture was added 1M hydrochloric acid (250 mL) dropwise at 0° C. and the mixture was stirred at room temperature for 30 min. The insoluble material was removed by filtration, and the filtrate was extracted with EtOAc thrice. The organic layers were combined, washed with saturated aqueous NaHCO₃, water and brine, dried over Na₂SO₄ and concentrated under reduced pressure. The residue was dissolved in EtOAc/THF (1/1) again and the insoluble material was removed by filtration. The filtrate was concentrated and passed through a pad of silica gel (eluted with hexane/EtOAc=1/1) and recrystallized from heptane/EtOAc to give the title compound (15.2 g) as a white solid. ¹H NMR (400 MHz, CDCl₃) δ 1.42 (9H, s), 2.57 (3H, s), 2.97 (2H, br t, J=6.5 Hz), 3.26-3.39 (2H, m), 3.91 (3H, s), 4.97 (1H, br s), 5.18 (2H, s), 6.81 (1H, br s), 7.25 (1H, s), 7.31-7.35 (1H, m), 7.36-7.42 (2H, m), 7.42-7.47 (2H, m).

### F) tert-Butyl (2-{5-(benzyloxy)-4-methoxy-2-[(2E)-3-(5-methoxy-1-methyl-1H-pyrrolo[2,3-b]pyridin-3-yl)prop-2-enoyl]phenyl}ethyl)carbamate

To a solution of tert-butyl {2-[2-acetyl-5-(benzyloxy)-4-methoxyphenyl]ethyl}carbamate (100 mg) and 5-methoxy-1-methyl-1H-pyrrolo[2,3-b]pyridine-3-carbaldehyde (48 mg) in MeOH (4 mL) was added sodium hydroxide (30 mg) at room temperature. The mixture was stirred at 70° C. for 1.5 h. The mixture was diluted with MeOH (20 mL), and the precipitated solid was collected by filtration to give the title compound (104 mg) as a yellow solid. ¹H NMR (400 MHz, DMSO-d₆) δ 1.32 (9H, s), 2.75-2.82 (2H, m), 3.14-3.16 (2H, m), 3.79-3.88 (6H, m), 3.91 (3H, s), 5.15 (2H, s), 6.86 (1H, s), 7.02 (1H, s), 7.15-7.22 (2H, m), 7.33-7.51 (5H, m) 7.72 (1H, m, J=16 Hz), 7.93 (1H, s), 8.08-8.21

### (2H, m).

### G) 6-(Benzyloxy)-7-methoxy-1-[(E)-2-(5-methoxy-1-methyl-1H-pyrrolo[2,3-b]pyridin-3-yl)ethenyl]-3,4-dihydroisoquinoline hydrochloride

To a solution of tert-butyl (2-{5-(benzyloxy)-4-methoxy-2-[(2E)-3-(5-methoxy-1-methyl-1H-pyrrolo[2,3-b]pyridin-3-yl)prop-2-enoyl]phenyl}ethyl)carbamate (104 mg) in EtOAc (6.0 mL) was added 4M hydrogen chloride in EtOAc (6.0 mL) at room temperature. The mixture was stirred at room temperature for 0.5 h. The mixture was concentrated under reduced pressure to give the title compound (112 mg, crude) as an off-yellow solid. The crude product was used in the next step without further purification. MS: [M+H]+ 454.3

### H) 6-(Benzyloxy)-7-methoxy-1-[(E)-2-(5-methoxy-1-methyl-1H-pyrrolo[2,3-b]pyridin-3-yl)ethenyl]-1,2,3,4-tetrahydroisoquinoline hydrochloride

To a solution of 6-(benzyloxy)-7-methoxy-1-[(E)-2-(5-methoxy-1-methyl-1H-pyrrolo[2,3-b]pyridin-3-yl)ethenyl]-3,4-dihydroisoquinoline hydrochloride (60 mg) in MeOH (6.0 mL) was added NaBH₄ (9.6 mg) at room temperature. The mixture was stirred at room temperature for 1 h. The reaction was quenched by water and concentrated under reduced pressure. The residue was purified by preparative HPLC (column: Phenomenex Gemini-NX C18 80×30 mm×5 μm; mobile phase A: water (0.05% hydrochloric acid), mobile phase B: CH₃CN; Gradient 10% B to 40% B in 10 min), most of CH₃CN was removed under reduced pressure, and lyophilization to give the title compound (3.5 mg) as a yellow solid. ¹H NMR (400 MHz, DMSO-d₆) δ 2.88-3.01 (2H, m), 3.43-3.54 (2H, m), 3.67 (3H, s), 3.78 (3H, s), 3.86 (3H, s), 5.09 (2H, s), 5.11-5.18 (1H, m), 6.29 (1H, dd, J=16.0, 8.0 Hz), 6.71 (1H, s), 6.89 (1H, d, J=16.0 Hz), 6.97 (1H, s), 7.31-7.36 (1H, m), 7.37-7.46 (4H, m), 7.76 (1H, s), 7.78-7.82 (1H, m), 8.09 (1H, d, J=2.8 Hz), 9.40-9.45 (2H, m).

### Example 17: 6-(Benzyloxy)-7-methoxy-1-{(E)-2-[2-methyl]-5-(pyrimidin-5-yl)phenyl]ethenyl}-1,2,3,4-tetrahydroisoquinoline hydrochloride

### A) tert-Butyl (2-{5-(benzyloxy)-2-[(2E)-3-(5-bromo-2-methylphenyl)prop-2-enoyl]-4-methoxyphenyl}ethyl)carbamate

To a solution of tert-butyl {2-[2-acetyl-5-(benzyloxy)-4-methoxyphenyl]ethyl}carbamate (190 mg) and 5-bromo-2-methylbenzaldehyde (284 mg) in EtOH (6.0 mL) was added sodium hydroxide (76 mg) at room temperature. The mixture was stirred at 50° C. for 3 h. The mixture was quenched with saturated aqueous NH₄Cl at room temperature and extracted with EtOAc. The organic layer was separated, washed with water and brine, dried over Na₂SO₄ and concentrated in vacuo. The residue was purified by column chromatography (silica gel, eluted with 2%-30% EtOAc in hexane) to give the title compound (257 mg) as a pale yellow solid. 1H NMR (400 MHz, CDCl₃) δ 1.42 (9H, s), 2.38 (3H, s), 2.90 (2H, br t, J=6.6 Hz), 3.29-3.48 (2H, m), 3.91 (3H, s), 5.13 (1H, br s), 5.20 (2H, s), 6.88 (1H, br s), 7.07-7.15 (3H, m), 7.30-7.49 (6H, m), 7.74 (1H, s), 7.81 (1H, d, J=15.8 Hz).

### B) 6-(Benzyloxy)-1-[(E)-2-(5-bromo-2-methylphenyl)ethenyl]-7-methoxy-1,2,3,4-tetrahydroisoquinoline

A mixture of tert-butyl (2-{5-(benzyloxy)-2-[(2E)-3-(5-bromo-2-methylphenyl)prop-2-enoyl]-4-methoxyphenyl}ethyl)carbamate (257 mg) and 4M hydrogen chloride in EtOAc (5.0 mL) was stirred at room temperature for 2 h. The mixture was concentrated and azeotroped with toluene. The residue was dissolved in MeOH (5.0 mL) at room temperature. To the solution was added NaBH₄ (17.8 mg) at 0° C. The mixture was stirred at 0° C. to room temperature for 2 h. The mixture was quenched with saturated aqueous NaHCO₃ at room temperature and extracted with EtOAc. The organic layer was separated, washed with water and brine, dried over Na₂SO₄ and concentrated in vacuo. The residue was purified by column chromatography (NH silica gel, eluted with 5%-100% EtOAc in hexane) and crystallized from EtOAc-hexane to give the title compound (108 mg) as a white solid. MS: [M+H]+ 464.2, 466.2.

### C) tert-Butyl 6-(benzyloxy)-1-[(E)-2-(5-bromo-2-methylphenyl)ethenyl]-7-methoxy-3,4-dihydroisoquinoline-2 (1H)-carboxylate

To a solution of 6-(benzyloxy)-1-[(E)-2-(5-bromo-2-methylphenyl)ethenyl]-7-methoxy-1,2,3,4-tetrahydroisoquinoline (131 mg) in THF (5.0 mL) were added Et₃N (0.041 mL) and Boc₂O (0.069 mL) at room temperature. The mixture was stirred at room temperature overnight. The mixture was concentrated, and the residue was purified by column chromatography (silica gel, eluted with 1%-30% EtOAc in hexane) to give the title compound (157 mg) as a white solid. ¹H NMR (400 MHz, CDCl₃) δ 1.50 (9H, s), 2.24 (3H, br s), 2.60 (1H, br d, J=15.9 Hz), 2.78-2.95 (1H, m), 3.13-3.25 (1H, m), 3.85 (3H, s), 4.01-4.30 (1H, m), 5.14 (2H, s), 5.51-5.79 (1H, m), 6.16 (1H, dd, J=15.7, 6.5 Hz), 6.56 (1H, br d, J=16.1 Hz), 6.67 (2H, s), 6.99 (1H, br d, J=8.3 Hz), 7.21-7.26 (1H, m), 7.28-7.33 (1H, m), 7.35-7.41 (2H, m), 7.42-7.46 (2H, m), 7.49 (1H, s).

### D) tert-Butyl 6-(benzyloxy)-7-methoxy-1-{(E)-2-[2-methyl-5-(pyrimidin-5-yl)phenyl]ethenyl}-3,4-dihydroisoquinoline-2 (1H)-carboxylate

A mixture of tert-butyl 6-(benzyloxy)-1-[(E)-2-(5-bromo-2-methylphenyl)ethenyl]-7-methoxy-3,4-dihydroisoquinoline-2(1H)-carboxylate (30 mg), pyrimidine-5-boronic acid (9.9 mg), Cs2CO3 (34.6 mg) and bis(di-tert-butyl(4-dimethylaminophenyl)phosphine)dichloropalladium(II) (7.2 mg) in DME (0.4 mL) and water (0.1 mL) was stirred at 100° C. for 2 h under microwave irradiation. The mixture was poured into water at room temperature and extracted with EtOAc. The organic layer was separated, washed with water and brine, dried over Na₂SO₄ and concentrated in vacuo. The residue was purified by column chromatography (silica gel, eluted with 1%-30% EtOAc in hexane) to give the title compound (28.8 mg) as a colorless oil. ¹H NMR (400 MHz, CDCl₃) δ 1.51 (9H, s), 2.37 (3H, br s), 2.57-2.67 (1H, m), 2.80-2.91 (1H, m), 3.16-3.32 (1H, m), 3.85 (3H, s), 4.09-4.30 (1H, m), 5.14 (2H, s), 5.57-5.87 (1H, m), 6.20-6.29 (1H, m), 6.66-6.76 (3H, m), 7.27-7.41 (5H, m), 7.42-7.48 (2H, m), 7.56 (1H, s), 8.92 (2H, s), 9.18 (1H, s).

### E) 6-(Benzyloxy)-7-methoxy-1-{(E)-2-[2-methyl-5-(pyrimidin-5-yl)phenyl]ethenyl}-1,2,3,4-tetrahydroisoquinoline hydrochloride

A mixture of tert-butyl 6-(benzyloxy)-7-methoxy-1-{(E)-2-[2-methyl-5-(pyrimidin-5-yl)phenyl]ethenyl}-3,4-dihydroisoquinoline-2(1H)-carboxylate (28.8 mg) and 4M hydrogen chloride in EtOAc (2.0 mL) was stirred at room temperature for 30 min. The mixture was concentrated, the residual solid was collected by filtration and washed with MeOH to give the title compound (11.0 mg) as a pale yellow solid. 1H NMR (400 MHz, DMSO-d₆) δ 2.44 (3H, s), 2.97 (2H, br t, J=5.5 Hz), 3.32-3.43 (1H, m), 3.48-3.59 (1H, m), 3.72 (3H, s), 5.10 (2H, s), 5.20-5.27 (1H, m), 6.60 (1H, dd, J=15.4, 8.2 Hz), 6.77 (1H, s), 6.97 (1H, s), 7.20 (1H, d, J=15.5 Hz), 7.31-7.47 (6H, m), 7.69 (1H, d, J=8.1 Hz), 7.96 (1H, s), 9.04-9.26 (3H, m), 9.40-9.49 (1H, m), 9.63-9.80 (1H, m).

### Example 18: 7-Methoxy-1-[(E)-2-(5-methoxy-1-methyl-1H-pyrrolo[2,3-b]pyridin-3-yl)ethenyl]-6-((pyridin-3-yl))methoxy]-1,2,3,4-tetrahydroisoquinoline hydrochloride

### A) tert-Butyl [2-(2-acetyl-5-hydroxy-4-methoxyphenyl)ethyl]carbamate

4% Palladium-fibroin (0.333 g) was added to a solution of tert-butyl {2-[2-acetyl-5-(benzyloxy)-4-methoxyphenyl]ethyl}carbamate (1.00 g) in MeOH (70 mL) at room temperature under Ar. The mixture was stirred at room temperature under H₂ (1 atm) for 2 h. Additional 80 mg of 4% palladium-fibroin was added to the mixture under Ar. The mixture was stirred at room temperature under H₂ (1 atm) for 1 h. The catalyst was removed by filtration and washed with MeOH, and the filtrate was concentrated under reduced pressure to yield the title compound (0.740 g) as a white powder. ¹H NMR (400 MHz, DMSO-d₆) δ ppm 1.35 (9H, s), 2.51 (3H, brs), 2.75-2.86 (2H, m), 3.00-3.10 (2H, m), 3.82 (3H, s), 6.65 (1H, s), 6.70-6.80 (1H, m), 7.35 (1H, s), 9.76 (1H, brs).

### B) tert-Butyl [2-(2-acetyl-4-methoxy-5-{[2-(trimethylsilyl)ethoxy]methoxy}phenyl)ethyl]carbamate

(2-(Chloromethoxy)ethyl)trimethylsilane (0.47 mL) was added to a mixture of tert-butyl [2-(2-acetyl-5-hydroxy-4-methoxyphenyl)ethyl]carbamate (740 mg) and K₂CO₃ (661 mg) in DMF (20 mL) at 0° C. The mixture was stirred at room temperature for 30 min. Additional (2-(chloromethoxy)ethyl)trimethylsilane (0.042 mL) was added to the mixture at room temperature. Additional (2-(chloromethoxy)ethyl)trimethylsilane (0.042 mL) was added to the mixture at room temperature. Water was poured into the mixture at room temperature and the mixture was extracted with EtOAc. The organic layer was separated, washed with water and brine, dried over MgSO₄, and concentrated in vacuo. The residue was purified by column chromatography (silica gel, eluted with 25% EtOAc in hexane) to give the title compound (1.04 g) as a colorless oil. ¹H NMR (400 MHz, DMSO-d₆) δ ppm 0.00 (9H, s), 0.92 (2H, t, J=8.07 Hz), 1.37 (9H, s), 2.57 (3H, s), 2.85 (2H, t, J=7.09 Hz), 3.05-3.16 (2H, m), 3.74 (2H, t, J=8.07 Hz), 3.84 (3H, s), 5.29 (2H, s), 6.79 (1H, br t, J=4.28 Hz), 6.96 (1H, s), 7.39 (1H, s).

### C) tert-Butyl [2-(4-methoxy-2-[(2E)-3-(5-methoxy-1-methyl-1H-pyrrolo[2,3-b]pyridin-3-yl)prop-2-enoyl]-5-{[2-(trimethylsilyl)ethoxy]methoxy}phenyl)ethyl]carbamate

Sodium hydroxide (0.378 g) was added to a solution of 5-methoxy-1-methyl-1H-pyrrolo[2,3-b]pyridine-3-carbaldehyde (0.540 g) and tert-butyl [2-(2-acetyl-4-methoxy-5-{[2-(trimethylsilyl)ethoxy]methoxy}phenyl)ethyl]carbamate (1.04 g) in EtOH (25 mL) at room temperature. The mixture was stirred at 70° C. for 3 h. The mixture was concentrated under reduced pressure, the residue was dissolved in EtOAc (100 mL) and filtrated. The filtrate was concentrated under reduced pressure, and the residue was purified by column chromatography (NH silica gel, eluted with 100% EtOAc) twice to give the title compound (1.29 g) as a yellow powder. MS: [M+H]+ 612.3.

### D) tert-Butyl 6-hydroxy-7-methoxy-1-[(E)-2-(5-methoxy-1-methyl-1H-pyrrolo[2,3-b]pyridin-3-yl)ethenyl]-3,4-dihydroisoquinoline-2(1H)-carboxylate

TFA (5.0 mL) was added to tert-butyl [2-(4-methoxy-2-[(2E)-3-(5-methoxy-1-methyl-1H-pyrrolo[2,3-b]pyridin-3-yl)prop-2-enoyl]-5-{[2-(trimethylsilyl)ethoxy]methoxy}phenyl)ethyl]carbamate (1.29 g) at room temperature, and the mixture was stirred at room temperature for 30 min. The mixture was concentrated, and azeotroped with toluene thrice. The residue was dissolved in MeOH (30 mL) at room temperature. To the mixture was added NaBH₄ (95 mg) at 0° C. The mixture was stirred at 0° C. for 10 min. To the mixture was added saturated aqueous NaHCO₃ (5 mL) at 0° C. Then Boc₂O (0.592 mL) was added to the mixture at 0° C. The mixture was stirred at 0° C. for 1 h. To the mixture was added saturated aqueous NaHCO₃ at 0° C. and extracted with EtOAc/THF (1/1). The organic layer was separated, washed with water and brine, dried over MgSO₄ and concentrated in vacuo. The residue was purified by column chromatography (silica gel, eluted with 30%-50% EtOAc in hexane) to give the title compound (201 mg) as a white solid. MS: [M+H]⁺ 466.1.

### E) tert-Butyl 7-methoxy-1-[(E)-2-(5-methoxy-1-methyl-1H-pyrrolo[2,3-b]pyridin-3-yl)ethenyl]-6-[(pyridin-3-yl)methoxy]-3,4-dihydroisoquinoline-2(1H)-carboxylate

3-(Bromomethyl)pyridine hydrobromide (16.3 mg) was added to a mixture of tert-butyl 6-hydroxy-7-methoxy-1-[(E)-2-(5-methoxy-1-methyl-1H-pyrrolo[2,3-b]pyridin-3-yl)ethenyl]-3,4-dihydroisoquinoline-2(1H)-carboxylate (25.0 mg) and K₂CO₃ (22.3 mg) in DMF (1 mL) at room temperature. The mixture was stirred at room temperature overnight. Additional 3-(bromomethyl)pyridine hydrobromide (8.0 mg) and K₂CO₃ (22.3 mg) were added to the mixture at room temperature. The mixture was stirred at 50° C. for 1 h and stirred at 80° C. for 1 h. Additional 3-(bromomethyl)pyridine hydrobromide (16.3 mg) and K₂CO₃ (22.3 mg) were added to the mixture at room temperature. The mixture was stirred at 80° C. for 1 h. Cs₂CO₃ (52.5 mg) was added to the mixture at room temperature. 3-(bromomethyl)pyridine hydrobromide (16.3 mg) and Cs₂CO₃ (52.5 mg) were added to the mixture at room temperature. The mixture was stirred at room temperature overnight. 3-(bromomethyl)pyridine hydrobromide (45.0 mg) and K₂CO₃ (66.0 mg) were added to the mixture at room temperature. The mixture was stirred at 80° C. for 1 h. To the mixture was added water at room temperature, and the mixture was extracted with EtOAc. The organic layer was separated, washed with water and brine, dried over MgSO₄ and concentrated in vacuo. The residue was purified by column chromatography (NH silica gel, eluted with 30%-50% EtOAc in hexane) to give the title compound (4.0 mg) as a white powder. MS: [M+H]⁺ 557.3

### F) 7-Methoxy-1-[(E)-2-(5-methoxy-1-methyl-1H-pyrrolo[2,3-b]pyridin-3-yl)ethenyl]-6-[(pyridin-3-yl)methoxy]-1,2,3,4-tetrahydroisoquinoline hydrochloride

4M Hydrogen chloride in EtOAc (0.3 mL) was added to a solution of tert-butyl 7-methoxy-1-[(E)-2-(5-methoxy-1-methyl-1H-pyrrolo[2,3-b]pyridin-3-yl)ethenyl]-6-[(pyridin-3-yl)methoxy]-3,4-dihydroisoquinoline-2(1H)-carboxylate (4.0 mg) in EtOAc (0.3 mL) at room temperature. The mixture was stirred at room temperature for 30 min. Solvent was removed under the reduced pressure to give the title compound (3.0 mg) as a yellow powder. 1H NMR (400 MHz, DMSO-d₆) δ ppm 2.91-3.05 (2H, m), 3.31-3.40 (1H, m), 3.47-3.54 (1H, m), 3.69 (3H, s), 3.78 (3H, s), 3.87 (3H, s), 5.12-5.18 (1H, m), 5.24 (2H, s), 6.32 (1H, dd, J=15.65, 7.83 Hz), 6.75 (1H, s), 6.90 (1H, d, J=15.89 Hz), 7.02 (1H, s), 7.72-7.79 (2H, m), 7.81 (1H, s), 8.09 (1H, s), 8.16-8.28 (1H, m), 8.73 (1H, br d, J=5.14 Hz), 8.82 (1H, s), 9.42-9.61 (2H, m).

### Example 22 and 23: (1R)-6-(benzyloxy)-7-methoxy-1-[(E)-2-(5-methoxy-1-methyl-1H-pyrrolo[2,3-b]pyridin-3-yl)ethenyl]-1,2,3,4-tetrahydroisoquinoline and (1S)-6-(benzyloxy)-7-methoxy-1-[(E)-2-(5-methoxy-1-methyl-1H-pyrrolo[2,3-b]pyridin-3-yl)ethenyl]-1,2,3,4-tetrahydroisoquinoline

Racemic 6-(benzyloxy)-7-methoxy-1-[(E)-2-(5-methoxy-1-methyl-1H-pyrrolo[2,3-b]pyridin-3-yl)ethenyl]-1,2,3,4-tetrahydroisoquinoline (Example 7) was separated into enantiomers by chiral HPLC (column: CHIRALPAK AS-H(VJ022) 4.6 mmID×250 mmL, 5 μm; mobile phase: hexane/2-propanol/DEA=400/600/1 (v/v/v)) to give (1R)-6-(benzyloxy)-7-methoxy-1-[(E)-2-(5-methoxy-1-methyl-1H-pyrrolo[2,3-b]pyridin-3-yl)ethenyl]-1,2,3,4-tetrahydroisoquinoline (retention time: 13.4 min) and (1S)-6-(benzyloxy)-7-methoxy-1-[(E)-2-(5-methoxy-1-methyl-1H-pyrrolo[2,3-b]pyridin-3-yl)ethenyl]-1,2,3,4-tetrahydroisoquinoline (retention time: 22.6 min). Absolute configuration of compound was determined by X-ray crystal structure analysis.

### Example 28: 6-(Benzyloxy)-7-methoxy-1-[(E)-2-{4-methoxy-2-methyl-5-[(pyridin-4-yl)methoxy]phenyl}ethenyl]-1,2,3,4-tetrahydroisoquinoline

### A) 4-Methoxy-5-(methoxymethoxy)-2-methylbenzaldehyde

To a solution of 2-bromo-4-methoxy-5-(methoxymethoxy)benzaldehyde (10.0 g) in DME (150 mL) and water (15 mL) were added 2,4,6-trimethyl-1,3,5,2,4,6-trioxatriborinane (10.2 mL), bis(di-tert-butyl(4-dimethylaminophenyl)phosphine)dichloropalladium(II) (1.22 g) and K₂CO₃ (15.1 g) at room temperature. The mixture was stirred at 100° C. under Ar for 2 h. The mixture was poured into water and extracted with EtOAc. The organic layer was separated, washed with brine, dried over Na₂SO₄ and concentrated in vacuo. The residue was purified by column chromatography (silica gel, eluted with 5%-20% EtOAc in hexane) to give the title compound (5.90 g) as a yellow oil. MS: [M+H]⁺ 211.0

### B) tert-Butyl {2-[5-(benzyloxy)-4-methoxy-2-{(2E)-3-[4-methoxy-5-(methoxymethoxy)-2-methylphenyl]prop-2-enoyl}phenyl]ethyl}carbamate

To a solution of tert-butyl {2-[2-acetyl-5-(benzyloxy)-4-methoxyphenyl]ethyl}carbamate (2.50 g) in MeOH (150 mL) were added 4-methoxy-5-(methoxymethoxy)-2-methylbenzaldehyde (1.58 g) and sodium hydroxide (751 mg) at room temperature. The mixture was stirred at 60° C. overnight. The mixture was concentrated to the half volume, and diluted with IPA. The resulting solid was collected by filtration washed with IPA to give the title compound (2.43 g) as a white solid. MS: [M+H]+ 592.3

### C) 5-{(E)-2-[6-(Benzyloxy)-7-methoxy-1,2,3,4-tetrahydroisoquinolin-1-yl]ethenyl}-2-methoxy-4-methylphenol

To a suspension of tert-butyl {2-[5-(benzyloxy)-4-methoxy-2-{(2E)-3-[4-methoxy-5-(methoxymethoxy)-2-methylphenyl]prop-2-enoyl}phenyl]ethyl}carbamate (2.85 g) in EtOAc (30 mL) was added 4M hydrogen chloride in EtOAc (30 mL) at room temperature. The mixture was stirred at room temperature overnight. The resulting solid was collected by filtration and washed with EtOAc to give a light brown powder. To a solution of the obtained powder in MeOH (250 mL) was added NaBH₄ (0.194 g) at 0° C. The mixture was stirred at 0° C. for 30 min. The mixture was quenched with saturated aqueous NaHCO₃ at 0° C. and concentrated in vacuo. To the residue was added water and the mixture was extracted with EtOAc. The organic layer was separated, washed with brine, dried over Na₂SO₄ and concentrated in vacuo. The residue was crystallized from EtOAc-IPE to give the title compound (0.97 g) as a white solid. MS: [M+H]⁺ 432.2

### D) tert-Butyl 6-(benzyloxy)-1-[(E)-2-(5-hydroxy-4-methoxy-2-methylphenyl)ethenyl]-7-methoxy-3,4-dihydroisoquinoline-2(1H)-carboxylate

To a solution of 5-{(E)-2-[6-(benzyloxy)-7-methoxy-1,2,3,4-tetrahydroisoquinolin-1-yl]ethenyl}-2-methoxy-4-methylphenol (0.97 g) in THF (10 mL) and water (2.5 mL) was added Boc₂O (0.522 mL) at room temperature. The mixture was stirred at room temperature for 5 h. The mixture was poured into water and extracted with EtOAc. The organic layer was separated, washed with brine, dried over Na₂SO₄ and concentrated in vacuo. The residue was purified by column chromatography (silica gel, eluted with 0%-30% EtOAc in hexane) to give the title compound (1.19 g) as a colorless oil. MS: [M+H]⁺ 532.2

### E) tert-Butyl 6-(benzyloxy)-7-methoxy-1-[(E)-2-{4-methoxy-2-methyl-5-[(pyridin-4-yl)methoxy]phenyl}ethenyl]-3,4-dihydroisoquinoline-2(1H)-carboxylate

Sodium hydride (60% in mineral oil, 2.6 mg) was added to a solution of tert-butyl 6-(benzyloxy)-1-[(E)-2-(5-hydroxy-4-methoxy-2-methylphenyl)ethenyl]-7-methoxy-3,4-dihydroisoquinoline-2(1H)-carboxylate (23.0 mg) in DMF (1.0 mL) at 0° C. The mixture was stirred at room temperature for 10 min. 4-(bromomethyl)pyridine hydrobromide (13.1 mg) was added to the mixture at room temperature. The mixture was stirred at room temperature for 2 h. Water was poured into the mixture at room temperature and the mixture was extracted with EtOAc. The organic layer was separated, washed with water and brine, dried over MgSO₄ and concentrated in vacuo. The residue was purified by column chromatography (NH silica gel, eluted with 30%-50% EtOAc in hexane) to give the title compound (21.0 mg, crude) as an off-white powder. The crude product was used in the next reaction without further purification. MS: [M+H]+ 623.4

### F) 6-(Benzyloxy)-7-methoxy-1-[(E)-2-{4-methoxy-2-methyl-5-[(pyridin-4-yl)methoxy]phenyl}ethenyl]-1,2,3,4-tetrahydroisoquinoline

4M Hydrogen chloride in EtOAc (0.5 mL) was added to tert-butyl 6-(benzyloxy)-7-methoxy-1-[(E)-2-{4-methoxy-2-methyl-5-[(pyridin-4-yl)methoxy]phenyl}ethenyl]-3,4-dihydroisoquinoline-2(1H)-carboxylate (21.0 mg) at room temperature. The mixture was stirred at room temperature overnight. Solvent was removed under reduced pressure, and the residue was purified by preparative HPLC (column: YMC-Actus Triart C18, 150×20 mmID, 5 μm; mobile phase A: 10 mM NH₄HCO₃ in H₂O, mobile phase B: CH₃CN, gradient: 50% B to 85% B in 7 min) to give the title compound (4.3 mg) as an off-white solid. ¹H NMR (300 MHz, DMSO-d₆) δ ppm 2.25 (3H, s), 2.55-2.67 (2H, m), 2.81-2.88 (1H, m), 3.07-3.11 (1H, m), 3.24-3.27 (1H, m), 3.64 (3H, s), 3.77 (3H, s), 4.46 (1H, br d, J=6.97 Hz), 5.03 (2H, s), 5.14 (2H, s), 6.09 (1H, dd, J=15.54, 7.82 Hz), 6.60-6.73 (2H, m), 6.76 (1H, s), 6.81 (1H, s), 7.10 (1H, s), 7.30-7.46 (7H, m), 8.51-8.57 (2H, m).

The compounds of Examples are shown in the following tables. MS in the tables means actual measured value. The compounds of Examples 1-6, 8-16, 19-21, 24-27, 29-108 in the following tables were produced according to the methods described in the above-mentioned Examples, or methods analogous thereto.

TABLE 1-1

| Example No. | Structure | compound name | MS |
|---|---|---|---|
| 1 | 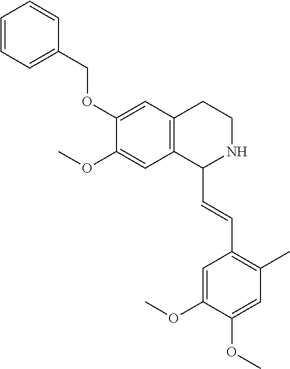 [SMILES: COc1cc(C)c(/C=C/C2NCCc3cc(OCc4ccccc4)c(OC)cc32)cc1OC] | 6-(benzyloxy)-1-[(E)-2-(4,5-dimethoxy-2- methylphenyl)ethenyl]-7-methoxy-1,2,3,4- tetrahydroisoquinoline | 446.2 |
| 2 | 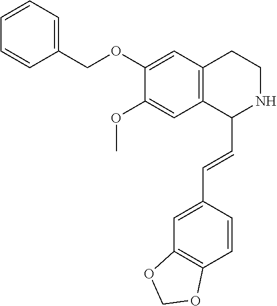 [SMILES: COc1cc2c(cc1OCc1ccccc1)CCNC2/C=C/c1ccc2c(c1)OCO2] | 1-[(E)-2-(2H-1,3-benzodioxol-5-yl)ethenyl]- 6-(benzyloxy)-7-methoxy-1,2,3,4- tetrahydroisoquinoline | 416.2 |
| 3 | 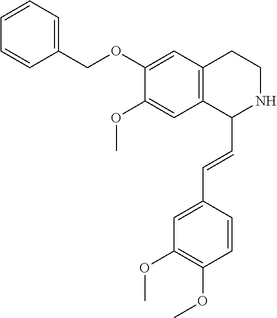 [SMILES: COc1ccc(/C=C/C2NCCc3cc(OCc4ccccc4)c(OC)cc32)cc1OC] | 6-(benzyloxy)-1-[(E)-2-(3,4- dimethoxyphenyl)ethenyl]-7-methoxy- 1,2,3,4-tetrahydroisoquinoline | 432.3 |
| 4 | 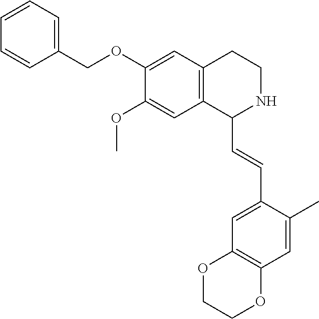 [SMILES: COc1cc2c(cc1OCc1ccccc1)CCNC2/C=C/c1cc2c(cc1C)OCCO2] | 6-(benzyloxy)-7-methoxy-1-[(E)-2-(7-methyl- 2,3-dihydro-1,4-benzodioxin-6-yl)ethenyl]- 1,2,3,4-tetrahydroisoquinoline | 444.3 |
| 5 | 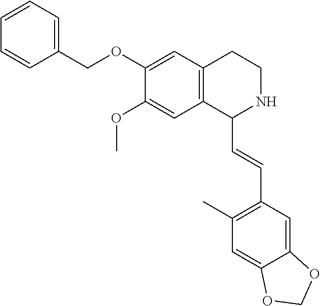 [SMILES: COc1cc2c(cc1OCc1ccccc1)CCNC2/C=C/c1cc2c(cc1C)OCO2] | 6-(benzyloxy)-7-methoxy-1-[(E)-2-(6-methyl- 2H-1,3-benzodioxol-5-yl)ethenyl]-1,2,3,4- tetrahydroisoquinoline | 430.3 |
| 6 | 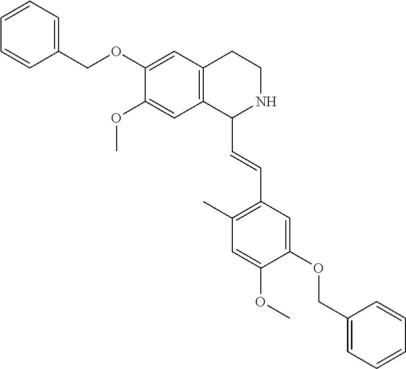 [SMILES: COc1cc(C)c(/C=C/C2NCCc3cc(OCc4ccccc4)c(OC)cc32)cc1OCc1ccccc1] | 6-(benzyloxy)-1-{(E)-2-[5-(benzyloxy)-4- methoxy-2-methylphenyl]ethenyl}-7- methoxy-1,2,3,4-tetrahydroisoquinoline | 522.3 |
| 7 | 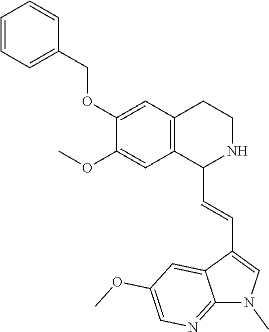 [SMILES: COc1cnc2c(c1)c(/C=C/C1NCCc3cc(OCc4ccccc4)c(OC)cc31)cn2C] | 6-(benzyloxy)-7-methoxy-1-[(E)-2-(5- methoxy-1-methyl-1H-pyrrolo[2,3-b]pyridin- 3-yl)ethenyl]-1,2,3,4-tetrahydroisoquinoline | 456.4 |

TABLE 1-2

| Example No. | Structure | compound name | MS |
|---|---|---|---|
| 8 | 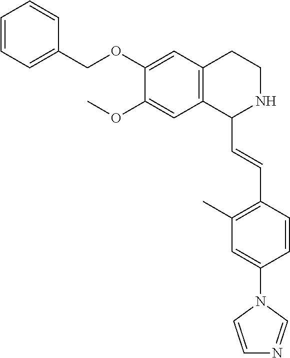 [SMILES: COc1cc2c(cc1OCc1ccccc1)CCNC2/C=C/c1ccc(-n2ccnc2)cc1C] | 6-(benzyloxy)-1-{(E)-2-[4-(1H-imidazol-1-yl)- 2-methylphenyl]ethenyl}-7-methoxy-1,2,3,4- tetrahydroisoquinoline | 452.4 |
| 9 | 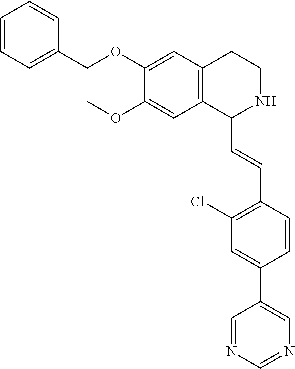 [SMILES: COc1cc2c(cc1OCc1ccccc1)CCNC2/C=C/c1ccc(-c2cncnc2)cc1Cl] | 6-(benzyloxy)-1-{(E)-2-[2-chloro-4- (pyrimidin-5-yl)phenyl]ethenyl}-7-methoxy- 1,2,3,4-tetrahydroisoquinoline | 484.4 |
| 10 | 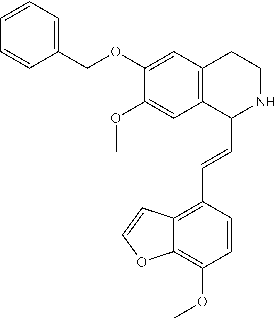 [SMILES: COc1cc2c(cc1OCc1ccccc1)CCNC2/C=C/c1ccc(OC)c2occc12] | 6-(benzyloxy)-7-methoxy-1-[(E)-2-(7- methoxy-1-benzofuran-4-yl)ethenyl]-1,2,3,4- tetrahydroisoquinoline | 442.4 |
| 11 | 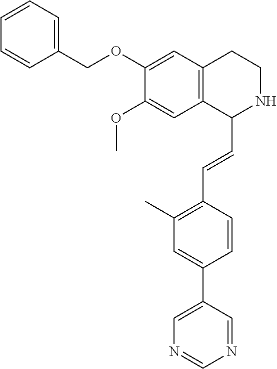 [SMILES: COc1cc2c(cc1OCc1ccccc1)CCNC2/C=C/c1ccc(-c2cncnc2)cc1C] | 6-(benzyloxy)-7-methoxy-1-{(E)-2-[2-methyl- 4-(pyrimidin-5-yl)phenyl]ethenyl}-1,2,3,4- tetrahydroisoquinoline | 464.4 |
| 12 | 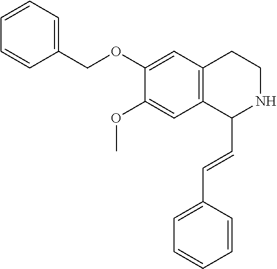 [SMILES: COc1cc2c(cc1OCc1ccccc1)CCNC2/C=C/c1ccccc1] | 6-(benzyloxy)-7-methoxy-1-[(E)-2- phenylethenyl]-1,2,3,4- tetrahydroisoquinoline | 372.1 |
| 13 | 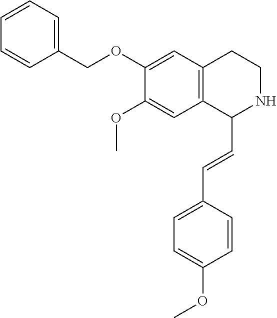 [SMILES: COc1ccc(/C=C/C2NCCc3cc(OCc4ccccc4)c(OC)cc32)cc1] | 6-(benzyloxy)-7-methoxy-1-[(E)-2-(4- methoxyphenyl)ethenyl]-1,2,3,4- tetrahydroisoquinoline | 402.2 |
| 14 | 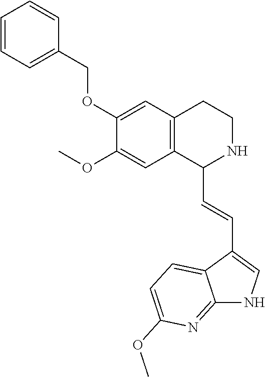 [SMILES: COc1ccc2c(/C=C/C3NCCc4cc(OCc5ccccc5)c(OC)cc43)c[nH]c2n1] | 6-(benzyloxy)-7-methoxy-1-[(E)-2-(6- methoxy-1H-pyrrolo[2,3-b]pyridin-3- yl)ethenyl]-1,2,3,4-tetrahydroisoquinoline | 442.1 |

TABLE 1-3

| Example No. | Structure | compound name | MS |
|---|---|---|---|
| 15 | 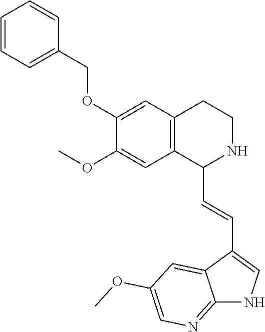 [SMILES: COc1cnc2[nH]cc(/C=C/C3NCCc4cc(OCc5ccccc5)c(OC)cc43)c2c1] | 6-(benzyloxy)-7-methoxy-1-[(E)-2-(5- methoxy-1H-pyrrolo[2,3-b]pyridin-3- yl)ethenyl]-1,2,3,4-tetrahydroisoquinoline | 442.2 |
| 16 | 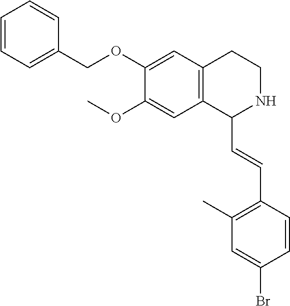 [SMILES: COc1cc2c(cc1OCc1ccccc1)CCNC2/C=C/c1ccc(Br)cc1C] | 6-(benzyloxy)-1-[(E)-2-(4-bromo-2- methylphenyl)ethenyl]-7-methoxy-1,2,3,4- tetrahydroisoquinoline | 463.9 |
| 17 | 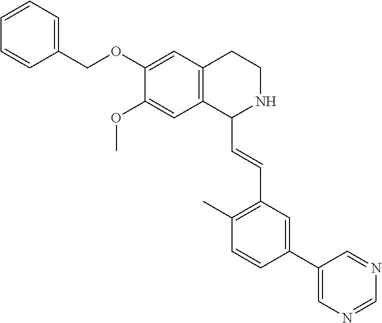 [SMILES: COc1cc2c(cc1OCc1ccccc1)CCNC2/C=C/c1cc(-c2cncnc2)ccc1C] | 6-(benzyloxy)-7-methoxy-1-{(E)-2-[2-methyl- 5-(pyrimidin-5-yl)phenyl]ethenyl}-1,2,3,4- tetrahydroisoquinoline | 464.1 |
| 18 | 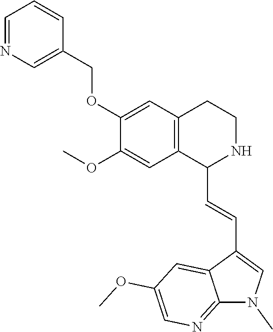 [SMILES: COc1cnc2c(c1)c(/C=C/C1NCCc3cc(OCc4cccnc4)c(OC)cc31)cn2C] | 7-methoxy-1-[(E)-2-(5-methoxy-1-methyl- 1H-pyrrolo[2,3-b]pyridin-3-yl)ethenyl]-6- [(pyridin-3-yl)methoxy]-1,2,3,4- tetrahydroisoquinoline | 457 |
| 19 | 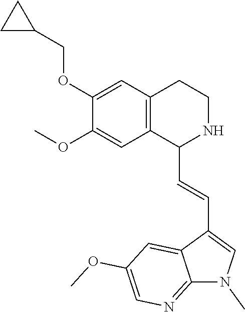 [SMILES: COc1cnc2c(c1)c(/C=C/C1NCCc3cc(OCC4CC4)c(OC)cc31)cn2C] | 6-(cyclopropylmethoxy)-7-methoxy-1-[(E)-2- (5-methoxy-1-methyl-1H-pyrrolo[2,3- b]pyridin-3-yl)ethenyl]-1,2,3,4- tetrahydroisoquinoline | 420.1 |
| 20 | 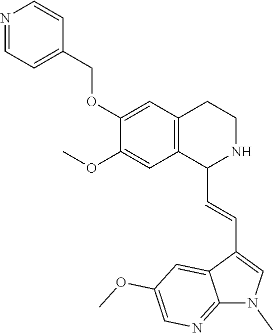 [SMILES: COc1cnc2c(c1)c(/C=C/C1NCCc3cc(OCc4ccncc4)c(OC)cc31)cn2C] | 7-methoxy-1-[(E)-2-(5-methoxy-1-methyl- 1H-pyrrolo[2,3-b]pyridin-3-yl)ethenyl]-6- [(pyridin-4-yl)methoxy]-1,2,3,4- tetrahydroisoquinoline | 457 |
| 21 | 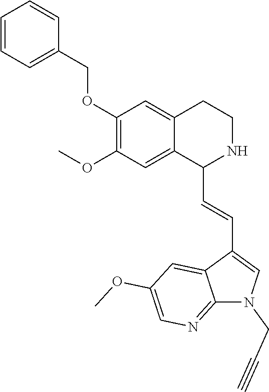 [SMILES: C#CCn1cc(/C=C/C2NCCc3cc(OCc4ccccc4)c(OC)cc32)c2cc(OC)cnc21] | 6-(benzyloxy)-7-methoxy-1-{(E)-2-[5- methoxy-1-(prop-2-yn-1-yl)-1H-pyrrolo[2,3- b]pyridin-3-yl]ethenyl}-1,2,3,4- tetrahydroisoquinoline | 480 |

TABLE 1-4

| Example No. | Structure | compound name | MS |
|---|---|---|---|
| 22 | 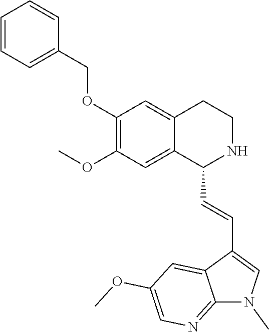 [SMILES: COc1cnc2c(c1)c(/C=C/[C@H]1NCCc3cc(OCc4ccccc4)c(OC)cc31)cn2C] | (1R)-6-(benzyloxy)-7-methoxy-1-[(E)-2-(5- methoxy-1-methyl-1H-pyrrolo[2,3-b]pyridin- 3-yl)ethenyl]-1,2,3,4-tetrahydroisoquinoline | 456.1 |
| 23 | 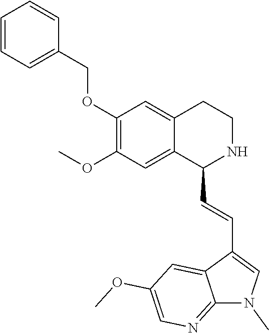 [SMILES: COc1cnc2c(c1)c(/C=C/[C@@H]1NCCc3cc(OCc4ccccc4)c(OC)cc31)cn2C] | (1S)-6-(benzyloxy)-7-methoxy-1-[(E)-2-(5- methoxy-1-methyl-1H-pyrrolo[2,3-b]pyridin- 3-yl)ethenyl]-1,2,3,4-tetrahydroisoquinoline | 456.1 |
| 24 | 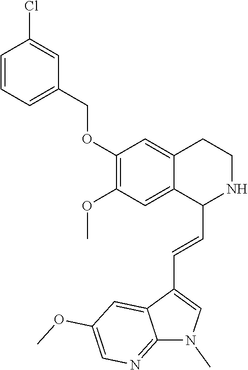 [SMILES: COc1cnc2c(c1)c(/C=C/C1NCCc3cc(OCc4cccc(Cl)c4)c(OC)cc31)cn2C] | 6-[(3-chlorophenyl)methoxy]-7-methoxy-1- [(E)-2-(5-methoxy-1-methyl-1H-pyrrolo[2,3- b]pyridin-3-yl)ethenyl]-1,2,3,4- tetrahydroisoquinoline | 489.9 |
| 25 | 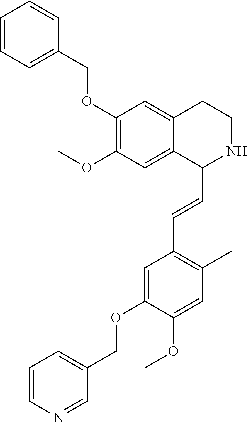 [SMILES: COc1cc(C)c(/C=C/C2NCCc3cc(OCc4ccccc4)c(OC)cc32)cc1OCc1cccnc1] | 6-(benzyloxy)-7-methoxy-1-[(E)-2-{4- methoxy-2-methyl-5-[(pyridin-3- yl)methoxy]phenyl}ethenyl]-1,2,3,4- tetrahydroisoquinoline | 523.1 |
| 26 | 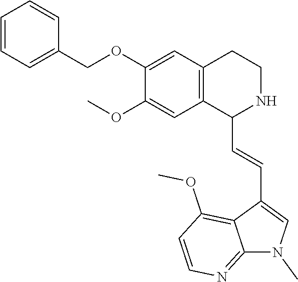 [SMILES: COc1cc2c(cc1OCc1ccccc1)CCNC2/C=C/c1cn(C)c2nccc(OC)c12] | 6-(benzyloxy)-7-methoxy-1-[(E)-2-(4- methoxy-1-methyl-1H-pyrrolo[2,3-b]pyridin- 3-yl)ethenyl]-1,2,3,4-tetrahydroisoquinoline | 456.4 |
| 27 | 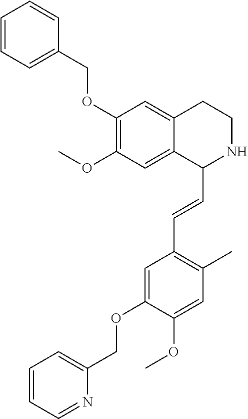 [SMILES: COc1cc2c(cc1OCc1ccccc1)CCNC2/C=C/c1cc(OCc2ccccn2)c(OC)cc1C] | 6-(benzyloxy)-7-methoxy-1-[(E)-2-{4- methoxy-2-methyl-5-[(pyridin-2- yl)methoxy]phenyl}ethenyl]-1,2,3,4- tetrahydroisoquinoline | 523.1 |
| 28 | 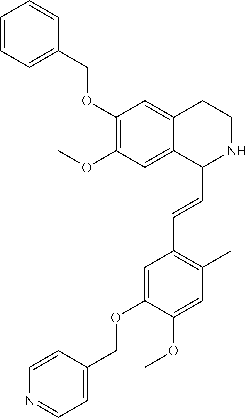 [SMILES: COc1cc(C)c(/C=C/C2NCCc3cc(OCc4ccccc4)c(OC)cc32)cc1OCc1ccncc1] | 6-(benzyloxy)-7-methoxy-1-[(E)-2-{4- methoxy-2-methyl-5-[(pyridin-4- yl)methoxy]phenyl}ethenyl]-1,2,3,4- tetrahydroisoquinoline | 523.1 |

TABLE 1-5

| Example No. | Structure | compound name | MS |
|---|---|---|---|
| 29 | 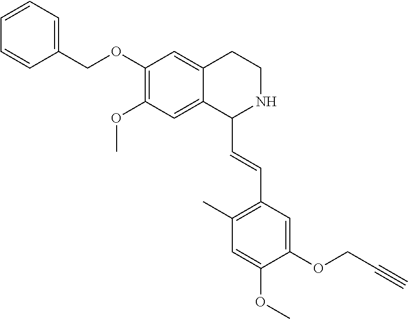 [SMILES: C#CCOc1cc(/C=C/C2NCCc3cc(OCc4ccccc4)c(OC)cc32)c(C)cc1OC] | 6-(benzyloxy)-7-methoxy-1-[(E)-2-{4- methoxy-2-methyl-5-[(prop-2-yn-1- yl)oxy]phenyl}ethenyl]-1,2,3,4- tetrahydroisoquinoline | 470.1 |
| 30 | 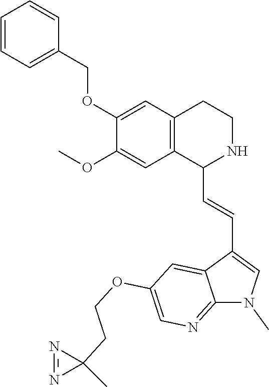 [SMILES: COc1cc2c(cc1OCc1ccccc1)CCNC2/C=C/c1cn(C)c2ncc(OCCC3(C)N=N3)cc12] | 6-(benzyloxy)-7-methoxy-1-[(E)-2-{1-methyl- 5-[2-(3-methyl-3H-diaziren-3-yl)ethoxy]-1H- pyrrolo[2,3-b]pyridin-3-yl}ethenyl]-1,2,3,4- tetrahydroisoquinoline | 524.1 |
| 31 | 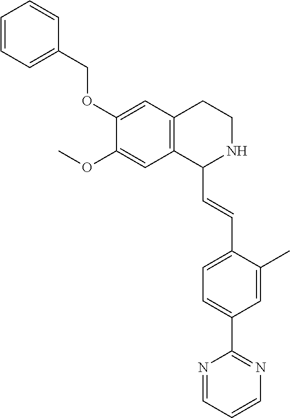 [SMILES: COc1cc2c(cc1OCc1ccccc1)CCNC2/C=C/c1ccc(-c2ncccn2)cc1C] | 6-(benzyloxy)-7-methoxy-1-{(E)-2-[2-methyl- 4-(pyrimidin-2-yl)phenyl]ethenyl}-1,2,3,4- tetrahydroisoquinoline | 404.2 |
| 32 | 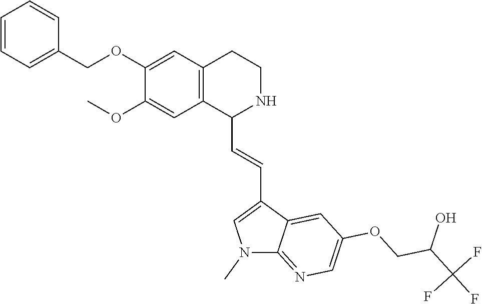 [SMILES: COc1cc2c(cc1OCc1ccccc1)CCNC2/C=C/c1cn(C)c2ncc(OCC(O)C(F)(F)F)cc12] | 3-[(3-{(E)-2-[6-(benzyloxy)-7-methoxy- 1,2,3,4-tetrahydroisoquinolin-1-yl]ethenyl}- 1-methyl-1H-pyrrolo[2,3-b]pyridin-5-yl)oxy]- 1,1,1-trifluoropropan-2-ol | 554.1 |
| 33 | 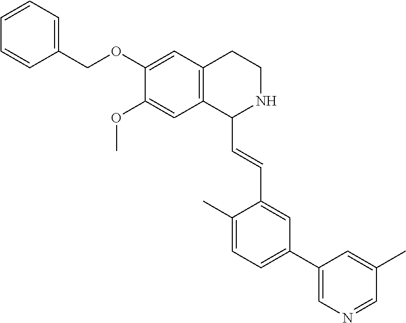 [SMILES: COc1cc2c(cc1OCc1ccccc1)CCNC2/C=C/c1cc(-c2cncc(C)c2)ccc1C] | 6-(benzyloxy)-7-methoxy-1-{(E)-2-[2-methyl- 5-(5-methylpyridin-3-yl)phenyl]ethenyl}- 1,2,3,4-tetrahydroisoquinoline | 477.1 |
| 34 | 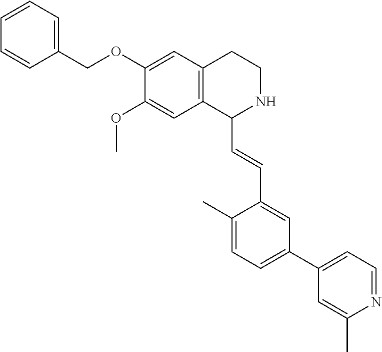 [SMILES: COc1cc2c(cc1OCc1ccccc1)CCNC2/C=C/c1cc(-c2ccnc(C)c2)ccc1C] | 6-(benzyloxy)-7-methoxy-1-{(E)-2-[2-methyl- 5-(2-methylpyridin-4-yl)phenyl]ethenyl}- 1,2,3,4-tetrahydroisoquinoline | 477.1 |
| 35 | 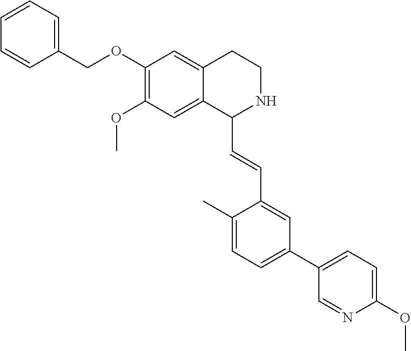 [SMILES: COc1ccc(-c2ccc(C)c(/C=C/C3NCCc4cc(OCc5ccccc5)c(OC)cc43)c2)cn1] | 6-(benzyloxy)-7-methoxy-1-{(E)-2-[5-(6- methoxypyridin-3-yl)-2- methylphenyl]ethenyl}-1,2,3,4- tetrahydroisoquinoline | 493.1 |

TABLE 1-6

| Example No. | Structure | compound name | MS |
|---|---|---|---|
| 36 | 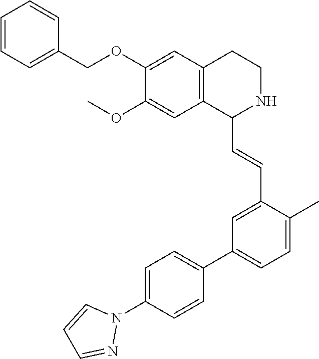 [SMILES: COc1cc2c(cc1OCc1ccccc1)CCNC2/C=C/c1cc(-c2ccc(-n3cccn3)cc2)ccc1C] | 6-(benzyloxy)-7-methoxy-1-{(E)-2-[4-methyl- 4′-(1H-pyrazol-1-yl)[1,1′-biphenyl]-3- yl]ethenyl}-1,2,3,4-tetrahydroisoquinoline | 528.1 |
| 37 | 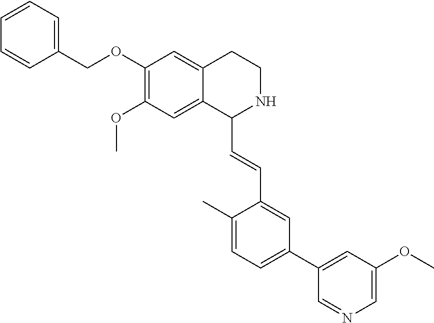 [SMILES: COc1cncc(-c2ccc(C)c(/C=C/C3NCCc4cc(OCc5ccccc5)c(OC)cc43)c2)c1] | 6-(benzyloxy)-7-methoxy-1-{(E)-2-[5-(5- methoxypyridin-3-yl)-2- methylphenyl]ethenyl}-1,2,3,4- tetrahydroisoquinoline | 493.1 |
| 38 | 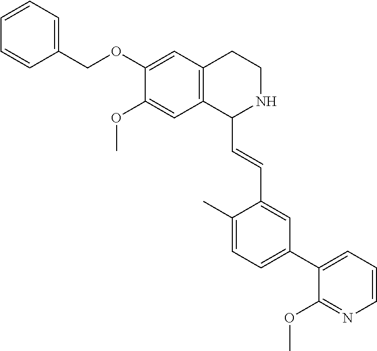 [SMILES: COc1cc2c(cc1OCc1ccccc1)CCNC2/C=C/c1cc(-c2cccnc2OC)ccc1C] | 6-(benzyloxy)-7-methoxy-1-{(E)-2-[5-(2- methoxypyridin-3-yl)-2- methylphenyl]ethenyl}-1,2,3,4- tetrahydroisoquinoline | 493.1 |
| 39 | 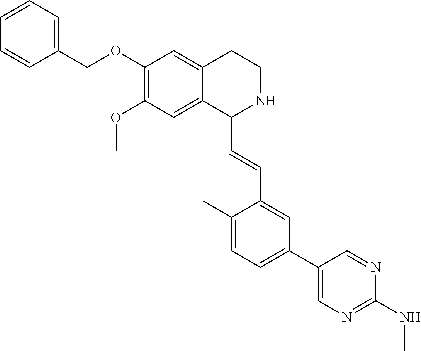 [SMILES: CNc1ncc(-c2ccc(C)c(/C=C/C3NCCc4cc(OCc5ccccc5)c(OC)cc43)c2)cn1] | 5-(3-{(E)-2-[6-(benzyloxy)-7-methoxy- 1,2,3,4-tetrahydroisoquinolin-1-yl]ethenyl}- 4-methylphenyl)-N-methylpyrimidin-2-amine | 493.1 |
| 40 | 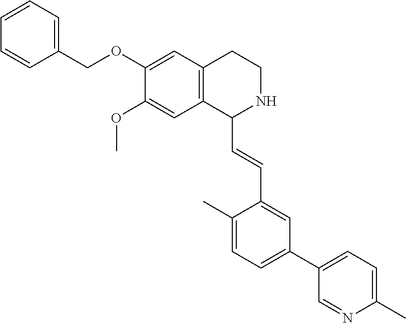 [SMILES: COc1cc2c(cc1OCc1ccccc1)CCNC2/C=C/c1cc(-c2ccc(C)nc2)ccc1C] | 6-(benzyloxy)-7-methoxy-1-{(E)-2-[2-methyl- 5-(6-methylpyridin-3-yl)phenyl]ethenyl}- 1,2,3,4-tetrahydroisoquinoline | 477.1 |
| 41 | 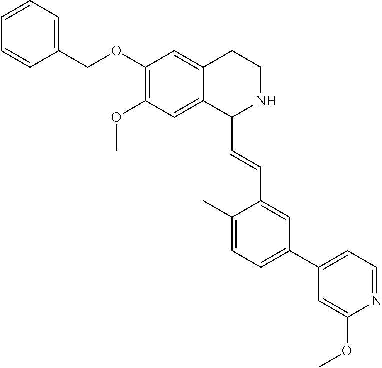 [SMILES: COc1cc(-c2ccc(C)c(/C=C/C3NCCc4cc(OCc5ccccc5)c(OC)cc43)c2)ccn1] | 6-(benzyloxy)-7-methoxy-1-{(E)-2-[5-(2- methoxypyridin-4-yl)-2- methylphenyl]ethenyl}-1,2,3,4- tetrahydroisoquinoline | 493.1 |
| 42 | 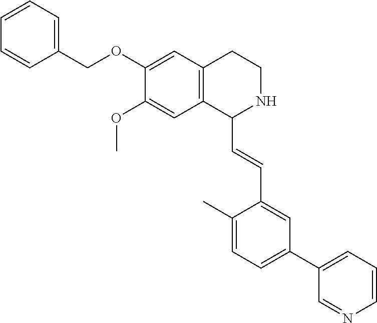 [SMILES: COc1cc2c(cc1OCc1ccccc1)CCNC2/C=C/c1cc(-c2cccnc2)ccc1C] | 6-(benzyloxy)-7-methoxy-1-{(E)-2-[2-methyl- 5-(pyridin-3-yl)phenyl]ethenyl}-1,2,3,4- tetrahydroisoquinoline | 463.1 |

TABLE 1-7

| Example No. | Structure | compound name | MS |
|---|---|---|---|
| 43 | 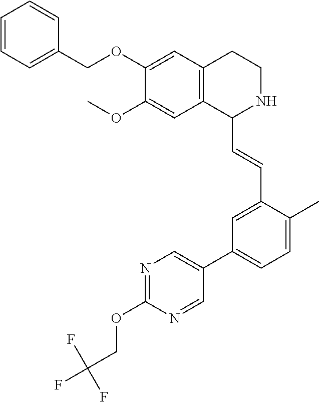 [SMILES: COc1cc2c(cc1OCc1ccccc1)CCNC2/C=C/c1cc(-c2cnc(OCC(F)(F)F)nc2)ccc1C] | 6-(benzyloxy)-7-methoxy-1-[(E)-2-{2-methyl- 5-[2-(2,2,2-trifluoroethoxy)pyrimidin-5- yl]phenyl}ethenyl]-1,2,3,4- tetrahydroisoquinoline | 562 |
| 44 | 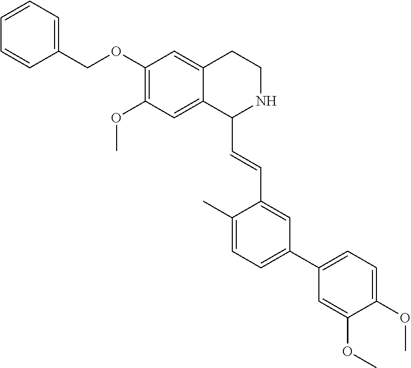 [SMILES: COc1ccc(-c2ccc(C)c(/C=C/C3NCCc4cc(OCc5ccccc5)c(OC)cc43)c2)cc1OC] | 6-(benzyloxy)-1-[(E)-2-(3′,4′-dimethoxy-4- methyl[1,1′-biphenyl]-3-yl)ethenyl]-7- methoxy-1,2,3,4-tetrahydroisoquinoline | 522.1 |
| 45 | 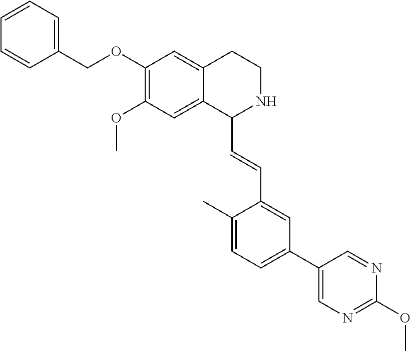 [SMILES: COc1ncc(-c2ccc(C)c(/C=C/C3NCCc4cc(OCc5ccccc5)c(OC)cc43)c2)cn1] | 6-(benzyloxy)-7-methoxy-1-{(E)-2-[5-(2- methoxypyrimidin-5-yl)-2- methylphenyl]ethenyl}-1,2,3,4- tetrahydroisoquinoline | 494.2 |
| 46 | 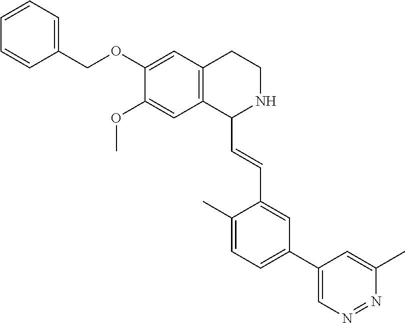 [SMILES: COc1cc2c(cc1OCc1ccccc1)CCNC2/C=C/c1cc(-c2cnnc(C)c2)ccc1C] | 6-(benzyloxy)-7-methoxy-1-{(E)-2-[2-methyl- 5-(6-methylpyridazin-4-yl)phenyl]ethenyl}- 1,2,3,4-tetrahydroisoquinoline | 478.1 |
| 47 | 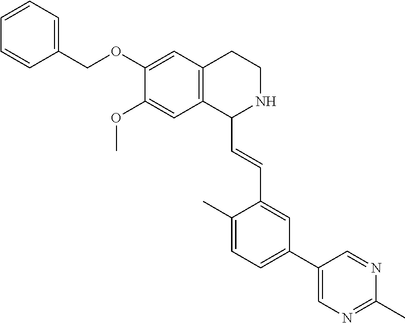 [SMILES: COc1cc2c(cc1OCc1ccccc1)CCNC2/C=C/c1cc(-c2cnc(C)nc2)ccc1C] | 6-(benzyloxy)-7-methoxy-1-{(E)-2-[2-methyl- 5-(2-methylpyrimidin-5-yl)phenyl]ethenyl}- 1,2,3,4-tetrahydroisoquinoline | 478.1 |
| 48 | 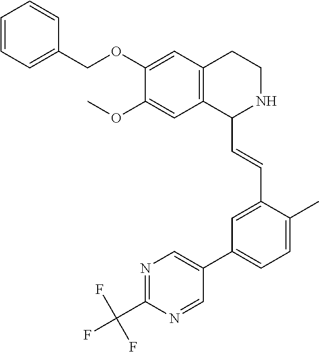 [SMILES: COc1cc2c(cc1OCc1ccccc1)CCNC2/C=C/c1cc(-c2cnc(C(F)(F)F)nc2)ccc1C] | 6-(benzyloxy)-7-methoxy-1-[(E)-2-{2-methyl- 5-[2-(trifluoromethyl)pyrimidin-5- yl]phenyl}ethenyl]-1,2,3,4- tetrahydroisoquinoline | 532.1 |
| 49 | 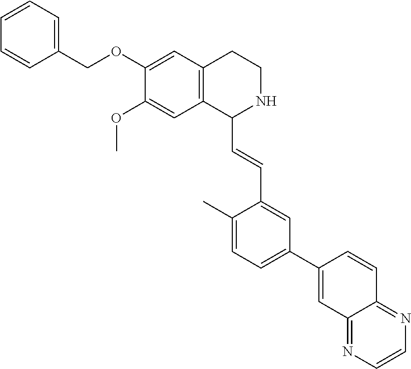 [SMILES: COc1cc2c(cc1OCc1ccccc1)CCNC2/C=C/c1cc(-c2ccc3nccnc3c2)ccc1C] | 6-(3-{(E)-2-[6-(benzyloxy)-7-methoxy- 1,2,3,4-tetrahydroisoquinolin-1-yl]ethenyl}- 4-methylphenyl)quinoxaline | 514.1 |

TABLE 1-8

| Example No. | Structure | compound name | MS |
|---|---|---|---|
| 50 | 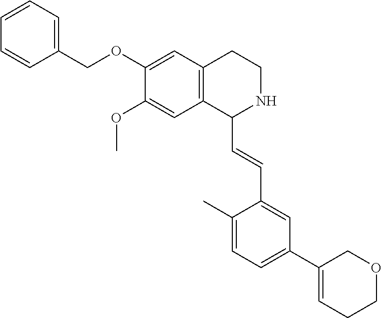 [SMILES: COc1cc2c(cc1OCc1ccccc1)CCNC2/C=C/c1cc(C2=CCCOC2)ccc1C] | 6-(benzyloxy)-1-{(E)-2-[5-(5,6-dihydro-2H- pyran-3-yl)-2-methylphenyl]ethenyl}-7- methoxy-1,2,3,4-tetrahydroisoquinoline | 468.2 |
| 51 | 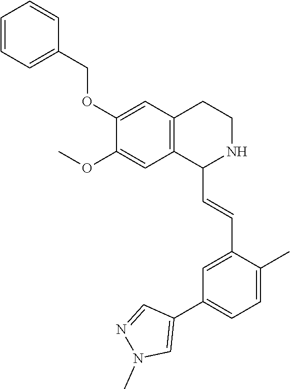 [SMILES: COc1cc2c(cc1OCc1ccccc1)CCNC2/C=C/c1cc(-c2cnn(C)c2)ccc1C] | 6-(benzyloxy)-7-methoxy-1-{(E)-2-[2-methyl- 5-(1-methyl-1H-pyrazol-4-yl)phenyl]ethenyl}- 1,2,3,4-tetrahydroisoquinoline | 466.1 |
| 52 | 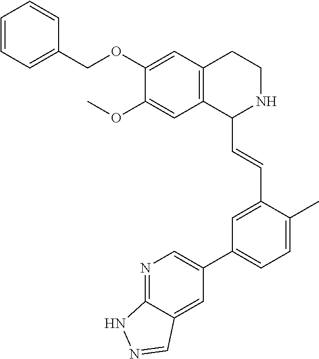 [SMILES: COc1cc2c(cc1OCc1ccccc1)CCNC2/C=C/c1cc(-c2cnc3[nH]ncc3c2)ccc1C] | 6-(benzyloxy)-7-methoxy-1-{(E)-2-[2-methyl- 5-(1H-pyrazolo[3,4-b]pyridin-5- yl)phenyl]ethenyl}-1,2,3,4- tetrahydroisoquinoline | 503.1 |
| 53 | 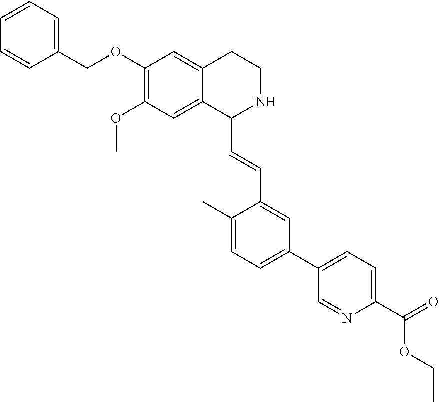 [SMILES: CCOC(=O)c1ccc(-c2ccc(C)c(/C=C/C3NCCc4cc(OCc5ccccc5)c(OC)cc43)c2)cn1] | ethyl 5-(3-{(E)-2-[6-(benzyloxy)-7-methoxy- 1,2,3,4-tetrahydroisoquinolin-1-yl]ethenyl}- 4-methylphenyl)pyridine-2-carboxylate | 535.1 |
| 54 | 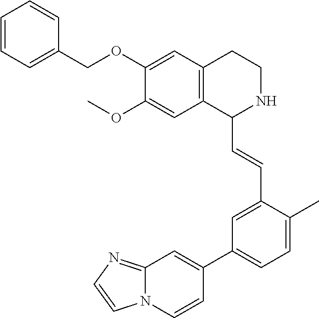 [SMILES: COc1cc2c(cc1OCc1ccccc1)CCNC2/C=C/c1cc(-c2ccn3ccnc3c2)ccc1C] | 6-(benzyloxy)-1-{(E)-2-[5-(imidazo[1,2- a]pyridln-7-yl)-2-methylphenyl]ethenyl}-7- methoxy-1,2,3,4-tetrahydroisoquinoline | 502.1 |
| 55 | 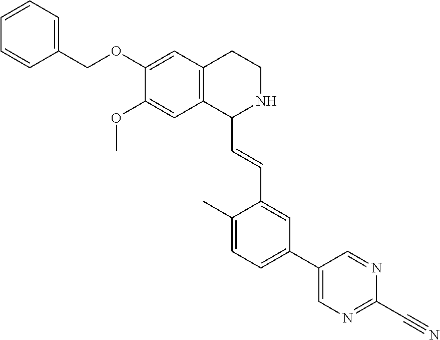 [SMILES: COc1cc2c(cc1OCc1ccccc1)CCNC2/C=C/c1cc(-c2cnc(C#N)nc2)ccc1C] | 5-(3-{(E)-2-[6-(benzyloxy)-7-methoxy- 1,2,3,4-tetrahydroisoquinolin-1-yl]ethenyl}- 4-methylphenyl)pyrimidine-2-carbonitrile | 489.1 |
| 56 | 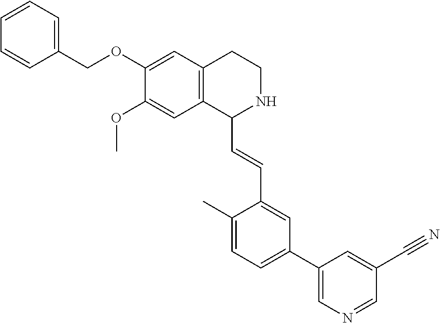 [SMILES: COc1cc2c(cc1OCc1ccccc1)CCNC2/C=C/c1cc(-c2cncc(C#N)c2)ccc1C] | 5-(3-{(E)-2-[6-(benzyloxy)-7-methoxy- 1,2,3,4-tetrahydroisoquinolin-1-yl]ethenyl}- 4-methylphenyl)pyridine-3-carbonitrile | 488.1 |

TABLE 1-9

| Example No. | Structure | compound name | MS |
|---|---|---|---|
| 57 | 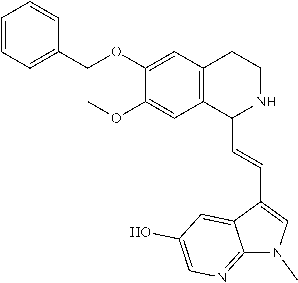 [SMILES: COc1cc2c(cc1OCc1ccccc1)CCNC2/C=C/c1cn(C)c2ncc(O)cc12] | 3-{(E)-2-[6-(benzyloxy)-7-methoxy-1,2,3,4- tetrahydroisoquinolin-1-yl]ethenyl}-1-methyl- 1H-pyrrolo[2,3-b]pyridin-5-ol | 442.1 |
| 58 | 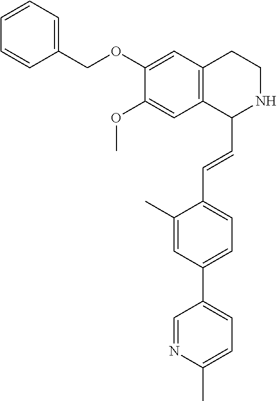 [SMILES: COc1cc2c(cc1OCc1ccccc1)CCNC2/C=C/c1ccc(-c2ccc(C)nc2)cc1C] | 6-(benzyloxy)-7-methoxy-1-{(E)-2-[2-methyl- 4-(6-methylpyridin-3-yl)phenyl]ethenyl}- 1,2,3,4-tetrahydroisoquinoline | 477.2 |
| 59 | 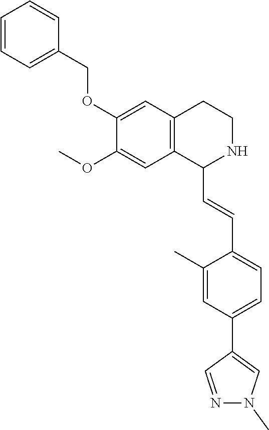 [SMILES: COc1cc2c(cc1OCc1ccccc1)CCNC2/C=C/c1ccc(-c2cnn(C)c2)cc1C] | 6-(benzyloxy)-7-methoxy-1-{(E)-2-[2-methyl- 4-(1-methyl-1H-pyrazol-4-yl)phenyl]ethenyl}- 1,2,3,4-tetrahydroisoquinoline | 466.1 |
| 60 | 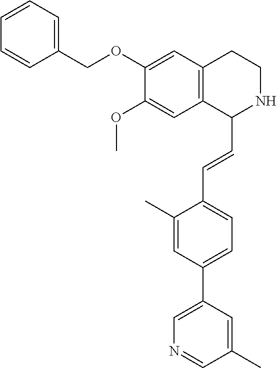 [SMILES: COc1cc2c(cc1OCc1ccccc1)CCNC2/C=C/c1ccc(-c2cncc(C)c2)cc1C] | 6-(benzyloxy)-7-methoxy-1-{(E)-2-[2-methyl- 4-(5-methylpyridin-3-yl)phenyl]ethenyl}- 1,2,3,4-tetrahydroisoquinoline | 477.1 |
| 61 | 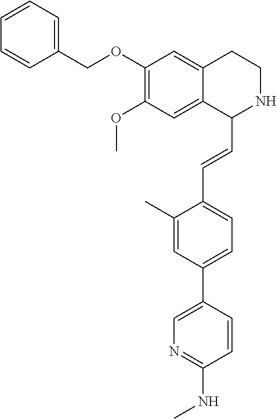 [SMILES: CNc1ccc(-c2ccc(/C=C/C3NCCc4cc(OCc5ccccc5)c(OC)cc43)c(C)c2)cn1] | 5-(4-{(E)-2-[6-(benzyloxy)-7-methoxy- 1,2,3,4-tetrahydroisoquinolin-1-yl]ethenyl}- 3-methylphenyl)-N-methylpyrimidin-2-amine | 493.1 |
| 62 | 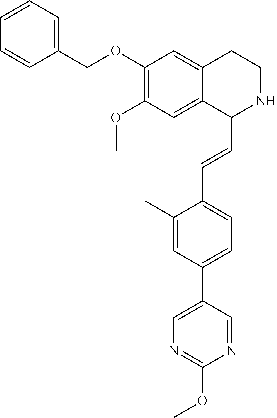 [SMILES: COc1ncc(-c2ccc(/C=C/C3NCCc4cc(OCc5ccccc5)c(OC)cc43)c(C)c2)cn1] | 6-(benzyloxy)-7-methoxy-1-{(E)-2-[4-(2- methoxypyrimidin-5-yl)-2- methylphenyl]ethenyl}-1,2,3,4- tetrahydroisoquinoline | 494.1 |
| 63 | 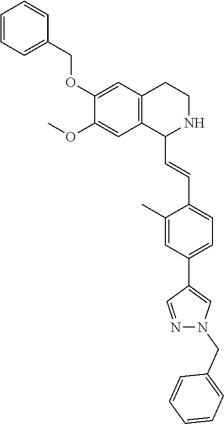 [SMILES: COc1cc2c(cc1OCc1ccccc1)CCNC2/C=C/c1ccc(-c2cnn(Cc3ccccc3)c2)cc1C] | 6-(benzyloxy)-1-{(E)-2-[4-(1-benzyl-1H- pyrazol-4-yl)-2-methylphenyl]ethenyl}-7- methoxy-1,2,3,4-tetrahydroisoquinoline | 542.1 |

TABLE 1-10

| Example No. | Structure | compound name | MS |
|---|---|---|---|
| 64 | 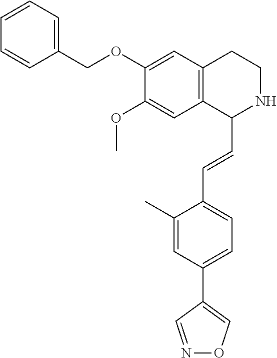 [SMILES: COc1cc2c(cc1OCc1ccccc1)CCNC2/C=C/c1ccc(-c2cnoc2)cc1C] | 6-(benzyloxy)-7-methoxy-1-{(E)-2-[2-methyl- 4-(1,2-oxazol-4-yl)phenyl]ethenyl}-1,2,3,4- tetrahydroisoquinoline | 453.1 |
| 65 | 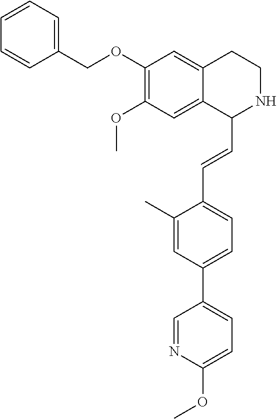 [SMILES: COc1ccc(-c2ccc(/C=C/C3NCCc4cc(OCc5ccccc5)c(OC)cc43)c(C)c2)cn1] | 6-(benzyloxy)-7-methoxy-1-{(E)-2-[4-(6- methoxypyridin-3-yl)-2- methylphenyl]ethenyl}-1,2,3,4- tetrahydroisoquinoline | 493.1 |
| 66 | 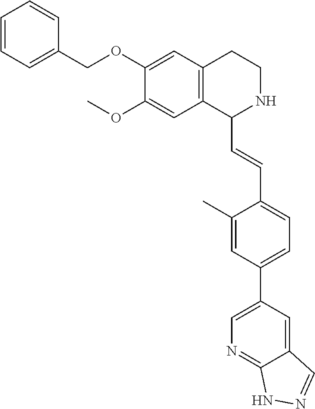 [SMILES: COc1cc2c(cc1OCc1ccccc1)CCNC2/C=C/c1ccc(-c2cnc3[nH]ncc3c2)cc1C] | 6-(benzyloxy)-7-methoxy-1-{(E)-2-[2-methyl- 4-(1H-pyrazolo[3,4-b]pyridin-5- yl)phenyl]ethenyl}-1,2,3,4- tetrahydroisoquinoline | 503.1 |
| 67 | 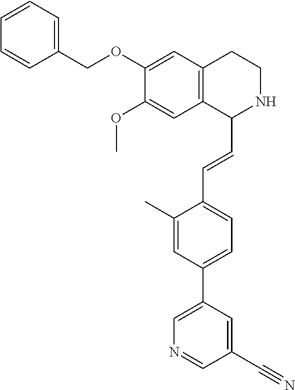 [SMILES: COc1cc2c(cc1OCc1ccccc1)CCNC2/C=C/c1ccc(-c2cncc(C#N)c2)cc1C] | 5-(4-{(E)-2-[6-(benzyloxy)-7-methoxy- 1,2,3,4-tetrahydroisoquinolin-1-yl]ethenyl}- 3-methylphenyl)pyridine-3-carbonitrile | 488.1 |
| 68 | 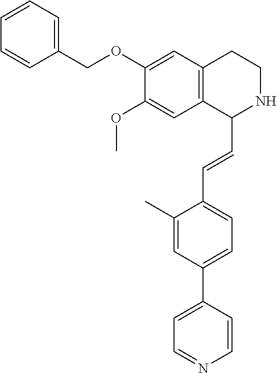 [SMILES: COc1cc2c(cc1OCc1ccccc1)CCNC2/C=C/c1ccc(-c2ccncc2)cc1C] | 6-(benzyloxy)-7-methoxy-1-{(E)-2-[2-methyl- 4-(pyridin-4-yl)phenyl]ethenyl}-1,2,3,4- tetrahydroisoquinoline | 463.1 |
| 69 | 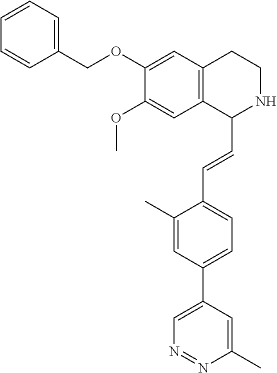 [SMILES: COc1cc2c(cc1OCc1ccccc1)CCNC2/C=C/c1ccc(-c2cnnc(C)c2)cc1C] | 6-(benzyloxy)-7-methoxy-1-{(E)-2-[2-methyl- 4-(6-methylpyridazin-4-yl)phenyl]ethenyl}- 1,2,3,4-tetrahydroisoquinoline | 478.1 |
| 70 | 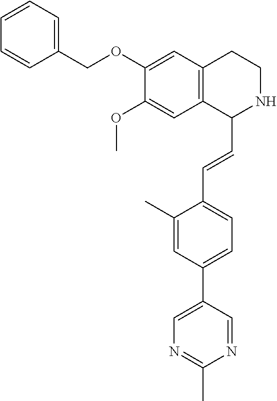 [SMILES: COc1cc2c(cc1OCc1ccccc1)CCNC2/C=C/c1ccc(-c2cnc(C)nc2)cc1C] | 6-(benzyloxy)-7-methoxy-1-{(E)-2-[2-methyl- 4-(2-methylpyrimidin-5-yl)phenyl]ethenyl}- 1,2,3,4-tetrahydroisoquinoline | 478.2 |

TABLE 1-11

| Example No. | Structure | compound name | MS |
|---|---|---|---|
| 71 | 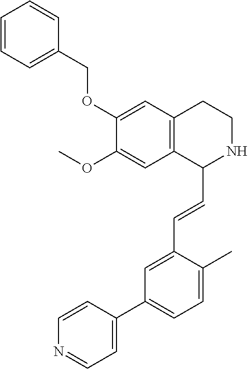 [SMILES: COc1cc2c(cc1OCc1ccccc1)CCNC2/C=C/c1cc(-c2ccncc2)ccc1C] | 6-(benzyloxy)-7-methoxy-1-{(E)-2-[2-methyl- 5-(pyridin-4-yl)phenyl]ethenyl}-1,2,3,4- tetrahydroisoquinoline | 463.1 |
| 72 | 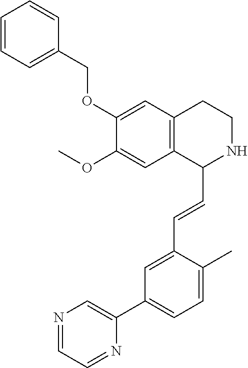 [SMILES: COc1cc2c(cc1OCc1ccccc1)CCNC2/C=C/c1cc(-c2cnccn2)ccc1C] | 6-(benzyloxy)-7-methoxy-1-{(E)-2-[2-methyl- 5-(pyrazin-2-yl)phenyl]ethenyl}-1,2,3,4- tetrahydroisoquinoline | 464.1 |
| 73 | 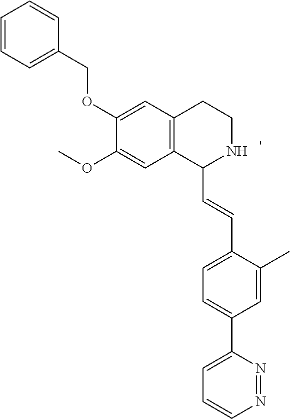 [SMILES: COc1cc2c(cc1OCc1ccccc1)CCNC2/C=C/c1ccc(-c2cccnn2)cc1C] | 6-(benzyloxy)-7-methoxy-1-{(E)-2-[2-methyl- 4-(pyridazin-3-yl)phenyl]ethenyl}-1,2,3,4- tetrahydroisoquinoline | 464.1 |
| 74 | 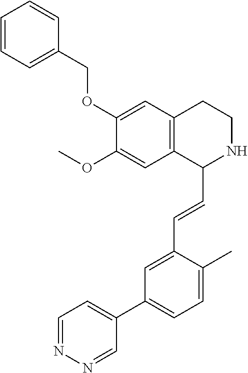 [SMILES: COc1cc2c(cc1OCc1ccccc1)CCNC2/C=C/c1cc(-c2ccnnc2)ccc1C] | 6-(benzyloxy)-7-methoxy-1-{(E)-2-[2-methyl- 5-(pyridazin-4-yl)phenyl]ethenyl}-1,2,3,4- tetrahydroisoquinoline | 464.2 |
| 75 | 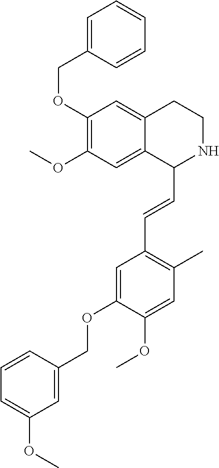 | 6-(benzyloxy)-7-methoxy-1-[(E)-2-{4- methoxy-5-[(3-methoxyphenyl)methoxy]-2- methylphenyl}ethenyl]-1,2,3,4- tetrahydroisoquinoline | 552.2 |
| 76 | 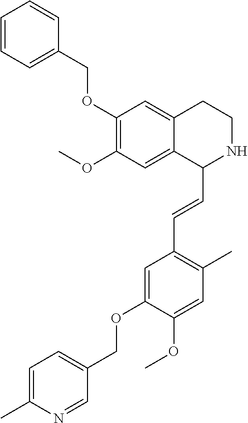 | 6-(benzyloxy)-7-methoxy-1-[(E)-2-{4- methoxy-2-methyl-5-[(6-methylpyridin-3- yl)methoxy]phenyl}ethenyl]-1,2,3,4- tetrahydroisoquinoline | 537.4 |
| 77 | 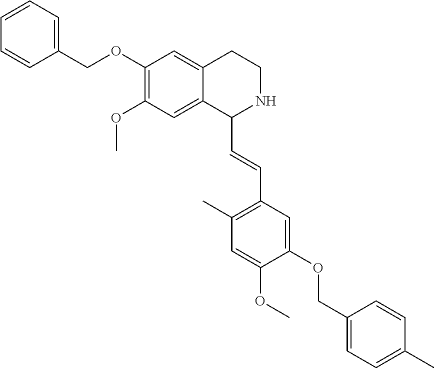 [SMILES: COc1cc(C)c(/C=C/C2NCCc3cc(OCc4ccccc4)c(OC)cc32)cc1OCc1ccc(C)cc1] | 6-(benzyloxy)-7-methoxy-1-[(E)-2-{4- methoxy-2-methyl-5-[(4- methylphenyl)methoxy]phenyl}ethenyl]- 1,2,3,4-tetrahydroisoquinoline | 536.4 |

TABLE 1-12

| Example No. | Structure | compound name | MS |
|---|---|---|---|
| 78 | 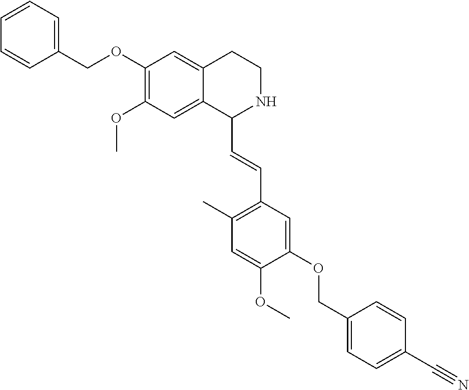 [SMILES: COc1cc(C)c(/C=C/C2NCCc3cc(OCc4ccccc4)c(OC)cc32)cc1OCc1ccc(C#N)cc1] | 4-[(5-{(E)-2-[6-(benzyloxy)-7-methoxy- 1,2,3,4-tetrahydroisoquinolin-1-yl]ethenyl}- 2-methoxy-4- methylphenoxy)methyl]benzonitrile | 547.4 |
| 79 | 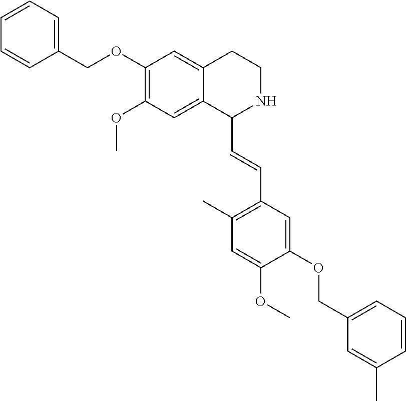 [SMILES: COc1cc(C)c(/C=C/C2NCCc3cc(OCc4ccccc4)c(OC)cc32)cc1OCc1cccc(C)c1] | 6-(benzyloxy)-7-methoxy-1-[(E)-2-{4- methoxy-2-methyl-5-[(3- methylphenyl)methoxy]phenyl}ethenyl]- 1,2,3,4-tetrahydroisoquinoline | 536.3 |
| 80 | 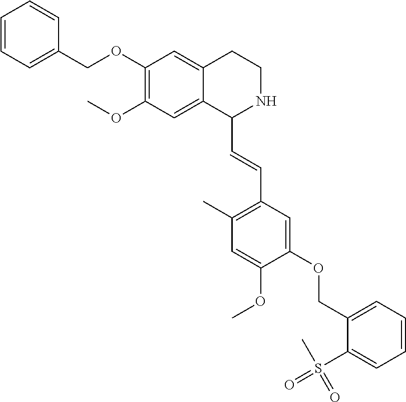 [SMILES: COc1cc2c(cc1OCc1ccccc1)CCNC2/C=C/c1cc(OCc2ccccc2S(C)(=O)=O)c(OC)cc1C] | 6-(benzyloxy)-1-[(E)-2-(5-{[2- (methanesulfonyl)phenyl]methoxy}-4- methoxy-2-methylphenyl)ethenyl]-7- methoxy-1,2,3,4-tetrahydroisoquinoline | 600.3 |
| 81 | 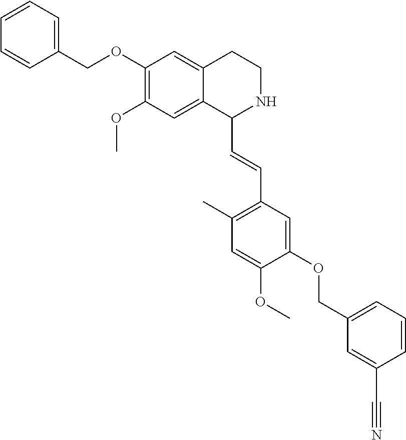 [SMILES: COc1cc(C)c(/C=C/C2NCCc3cc(OCc4ccccc4)c(OC)cc32)cc1OCc1cccc(C#N)c1] | 3-[(5-{(E)-2-[6-(benzyloxy)-7-methoxy- 1,2,3,4-tetrahydroisoquinolin-1-yl]ethenyl}- 2-methoxy-4- methylphenoxy)methyl]benzonitrile | 547.3 |
| 82 | 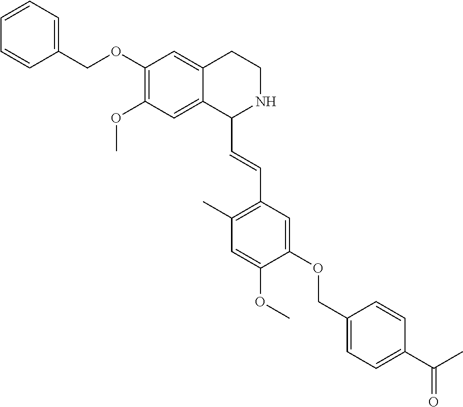 [SMILES: COc1cc(C)c(/C=C/C2NCCc3cc(OCc4ccccc4)c(OC)cc32)cc1OCc1ccc(C(C)=O)cc1] | 1-{4-[(5-{(E)-2-[6-(benzyloxy)-7-methoxy- 1,2,3,4-tetrahydroisoquinolin-1-yl]ethenyl}- 2-methoxy-4- methylphenoxy)methyl]phenyl}ethan-1-one | 564.3 |
| 83 | 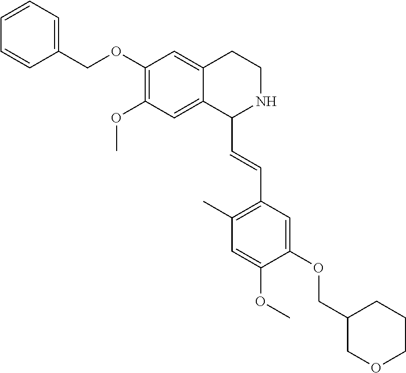 [SMILES: COc1cc2c(cc1OCc1ccccc1)CCNC2/C=C/c1cc(OCC2CCCOC2)c(OC)cc1C] | 6-(benzyloxy)-7-methoxy-1-[(E)-2-{4- methoxy-2-methyl-5-{(oxan-3- yl)methoxy]phenyl}ethenyl]-1,2,3,4- tetrahydroisoquinoline | 530.3 |
| 84 | 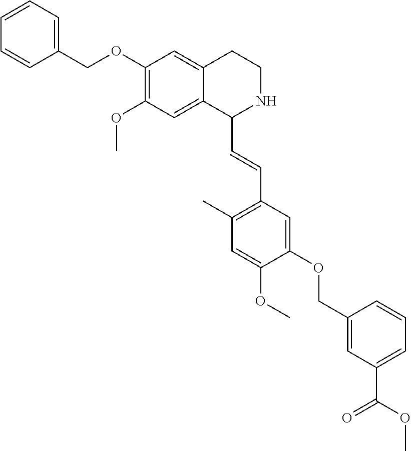 [SMILES: COC(=O)c1cccc(COc2cc(/C=C/C3NCCc4cc(OCc5ccccc5)c(OC)cc43)c(C)cc2OC)c1] | methyl 3-[(5-{(E)-2-[6-(benzyloxy)-7- methoxy-1,2,3,4-tetrahydroisoquinolin-1- yl]ethenyl}-2-methoxy-4- methylphenoxy)methyl]benzoate | 580.3 |

TABLE 1-13

| Example No. | Structure | compound name | MS |
|---|---|---|---|
| 85 | 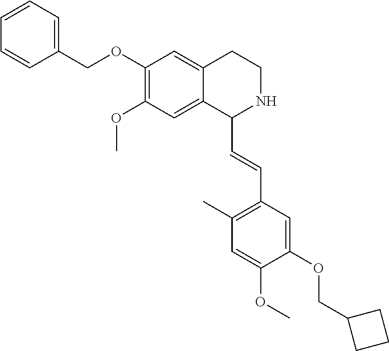 [SMILES: COc1cc2c(cc1OCc1ccccc1)CCNC2/C=C/c1cc(OCC2CCC2)c(OC)cc1C] | 6-(benzyloxy)-1-{(E)-2-[5- (cyclobutylmethoxy)-4-methoxy-2- methylphenyl]ethenyl}-7-methoxy-1,2,3,4- tetrahydroisoquinoline | 500.4 |
| 86 | 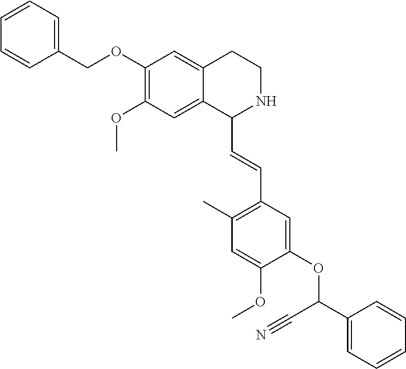 [SMILES: COc1cc2c(cc1OCc1ccccc1)CCNC2/C=C/c1cc(OC(C#N)c2ccccc2)c(OC)cc1C] | (5-{(E)-2-[6-(benzyloxy)-7-methoxy-1,2,3,4- tetrahydroisoquinolin-1-yl]ethenyl}-2- methoxy-4- methylphenoxy)(phenyl)acetonitrile | 547.3 |
| 87 | 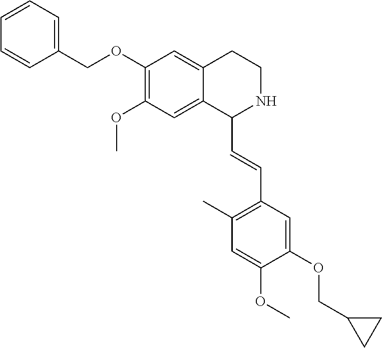 [SMILES: COc1cc2c(cc1OCc1ccccc1)CCNC2/C=C/c1cc(OCC2CC2)c(OC)cc1C] | 6-(benzyloxy)-1-{(E)-2-[5- (cyclopropylmethoxy)-4-methoxy-2- methylphenyl]ethenyl}-7-methoxy-1,2,3,4- tetrahydroisoquinoline | 486.3 |
| 88 | 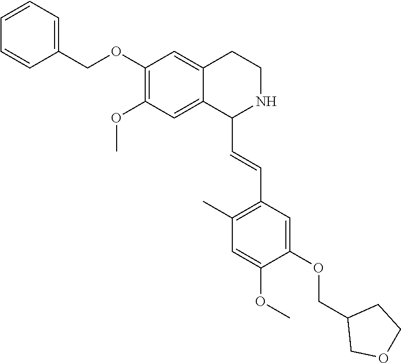 [SMILES: COc1cc2c(cc1OCc1ccccc1)CCNC2/C=C/c1cc(OCC2CCOC2)c(OC)cc1C] | 6-(benzyloxy)-7-methoxy-1-[(E)-2-{4- methoxy-2-methyl-5-[(oxolan-3- yl)methoxy]phenyl}ethenyl]-1,2,3,4- tetrahydroisoquinoline | 516.3 |
| 89 | 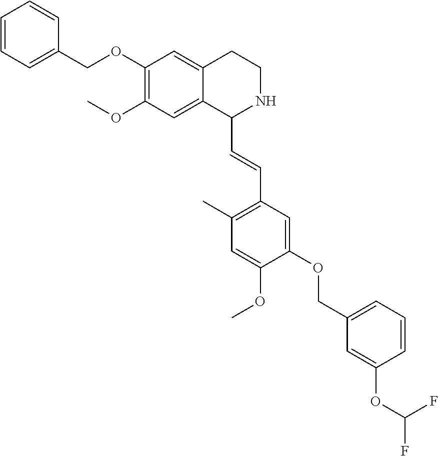 [SMILES: COc1cc(C)c(/C=C/C2NCCc3cc(OCc4ccccc4)c(OC)cc32)cc1OCc1cccc(OC(F)F)c1] | 6-(benzyloxy)-1-[(E)-2-(5-{[3- (difluoromethoxy)phenyl]methoxy}-4- methoxy-2-methylphenyl)ethenyl]-7- methoxy-1,2,3,4-tetrahydroisoquinoline | 588.3 |
| 90 | 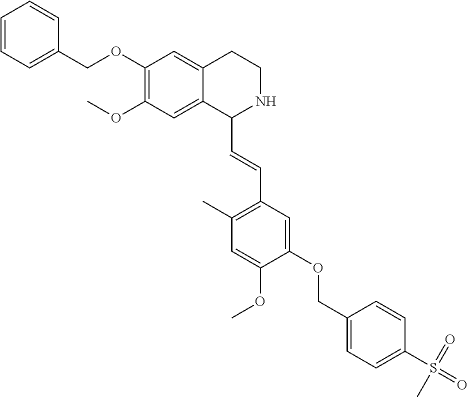 [SMILES: COc1cc(C)c(/C=C/C2NCCc3cc(OCc4ccccc4)c(OC)cc32)cc1OCc1ccc(S(C)(=O)=O)cc1] | 6-(benzyloxy)-1-[(E)-2-(5-{[4- (methanesulfonyl)phenyl]methoxy}-4- methoxy-2-methylphenyl)ethenyl]-7- methoxy-1,2,3,4-tetrahydroisoquinoline | 600.2 |
| 91 | 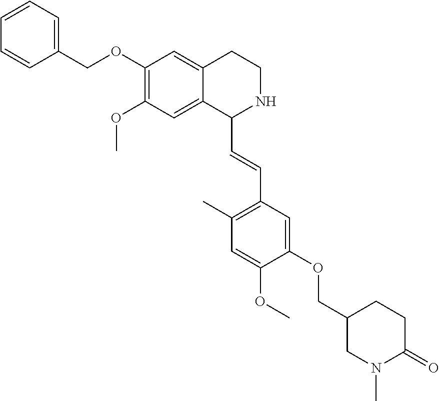 [SMILES: COc1cc2c(cc1OCc1ccccc1)CCNC2/C=C/c1cc(OCC2CCC(=O)N(C)C2)c(OC)cc1C] | 5-[(5-{(E)-2-[6-(benzyloxy)-7-methoxy- 1,2,3,4-tetrahydroisoquinolin-1-yl]ethenyl}- 2-methoxy-4-methylphenoxy)methyl]-1- methylpiperidin-2-one | 557.4 |

TABLE 1-14

| Example No. | Structure | compound name | MS |
|---|---|---|---|
| 92 | 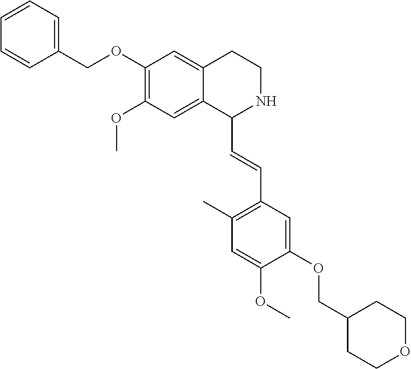 [SMILES: COc1cc2c(cc1OCc1ccccc1)CCNC2/C=C/c1cc(OCC2CCOCC2)c(OC)cc1C] | 6-(benzyloxy)-7-methoxy-1-[(E)-2-{4- methoxy-2-methyl-5-[(oxan-4- yl)methoxy]phenyl}ethenyl]-1,2,3,4- tetrahydroisoquinoline | 530.3 |
| 93 | 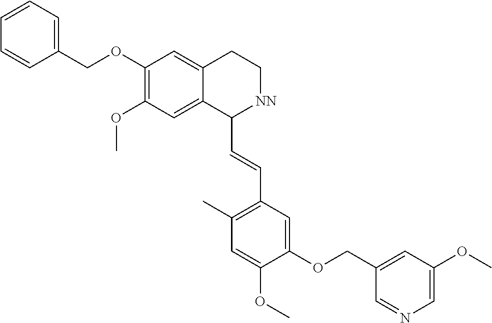 | 6-(benzyloxy)-7-methoxy-1-[(E)-2-{4- methoxy-5-[(5-methoxypyridin-3- yl)methoxy]-2-methylphenyl}ethenyl]- 1,2,3,4-tetrahydroisoquinoline | 553.3 |
| 94 | 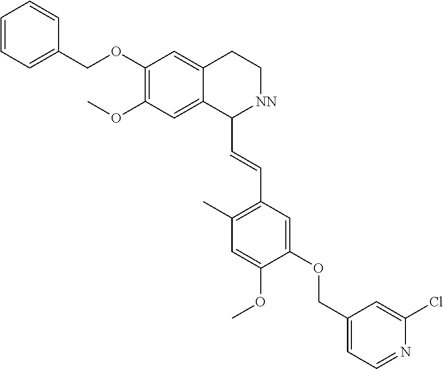 | 6-(benzyloxy)-1-[(E)-2-{5-[(2-chloropyridin-4- yl)methoxy]-4-methoxy-2- methylphenyl}ethenyl]-7-methoxy-1,2,3,4- tetrahydroisoquinoline | 557.2 |
| 95 | 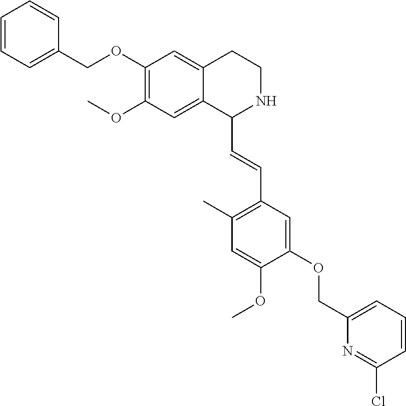 [SMILES: COc1cc2c(cc1OCc1ccccc1)CCNC2/C=C/c1cc(OCc2cccc(Cl)n2)c(OC)cc1C] | 6-(benzyloxy)-1-[(E)-2-{5-[(6-chloropyridin-2- yl)methoxy]-4-methoxy-2- methylphenyl}ethenyl]-7-methoxy-1,2,3,4- tetrahydroisoquinoline | 557.3 |
| 96 | 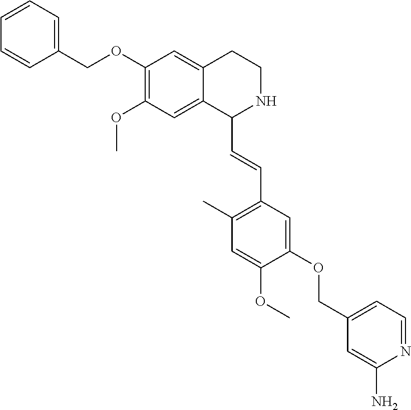 [SMILES: COc1cc(C)c(/C=C/C2NCCc3cc(OCc4ccccc4)c(OC)cc32)cc1OCc1ccnc(N)c1] | 4-[(5-{(E)-2-[6-(benzyloxy)-7-methoxy- 1,2,3,4-tetrahydroisoquinolin-1-yl]ethenyl}- 2-methoxy-4-methylphenoxy)methyl]pyridin- 2-amine | 538.3 |
| 97 | 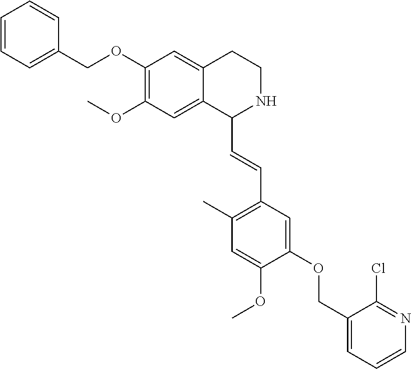 [SMILES: COc1cc2c(cc1OCc1ccccc1)CCNC2/C=C/c1cc(OCc2cccnc2Cl)c(OC)cc1C] | 6-(benzyloxy)-1-[(E)-2-{5-[(2-chloropyridin-3- yl)methoxy]-4-methoxy-2- methylphenyl}ethenyl]-7-methoxy-1,2,3,4- tetrahydroisoquinoline | 557.2 |
| 98 | 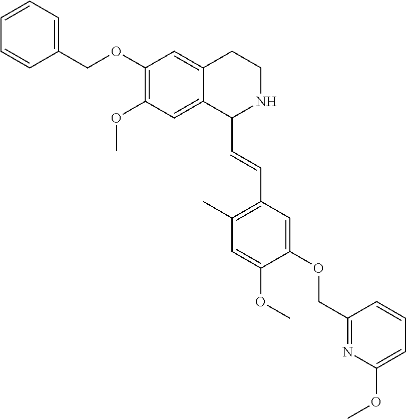 [SMILES: COc1cccc(COc2cc(/C=C/C3NCCc4cc(OCc5ccccc5)c(OC)cc43)c(C)cc2OC)n1] | 6-(benzyloxy)-7-methoxy-1-[(E)-2-{4- methoxy-5-[(6-methoxypyridin-2- yl)methoxy]-2-methylphenyl}ethenyl]- 1,2,3,4-tetrahydroisoquinoline | 553.3 |

TABLE 1-15

| Example No. | Structure | compound name | MS |
|---|---|---|---|
| 99 | 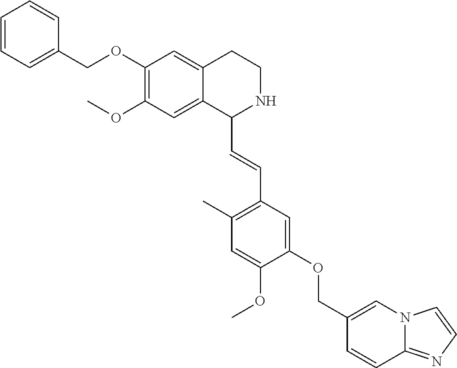 [SMILES: COc1cc(C)c(/C=C/C2NCCc3cc(OCc4ccccc4)c(OC)cc32)cc1OCc1ccc2nccn2c1] | 6-(benzyloxy)-1-[(E)-2-{5-[(imidazo[1,2- a]pyridin-6-yl)methoxy]-4-methoxy-2- methylphenyl}ethenyl]-7-methoxy-1,2,3,4- tetrahydroisoquinoline | 562.4 |
| 100 | 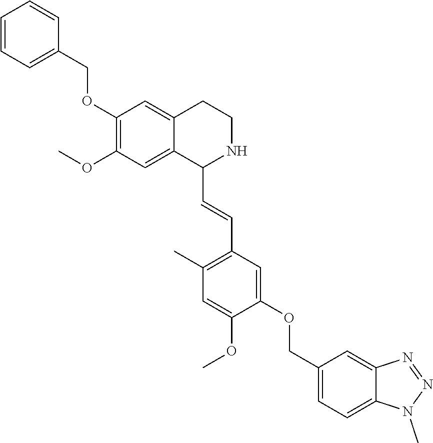 [SMILES: COc1cc(C)c(/C=C/C2NCCc3cc(OCc4ccccc4)c(OC)cc32)cc1OCc1ccc2c(c1)nnn2C] | 6-(benzyloxy)-7-methoxy-1-[(E)-2-{4- methoxy-2-methyl-5-[(1-methyl-1H- benzotriazol-5-yl)methoxy]phenyl}ethenyl]- 1,2,3,4-tetrahydroisoquinoline | 577.3 |
| 101 | 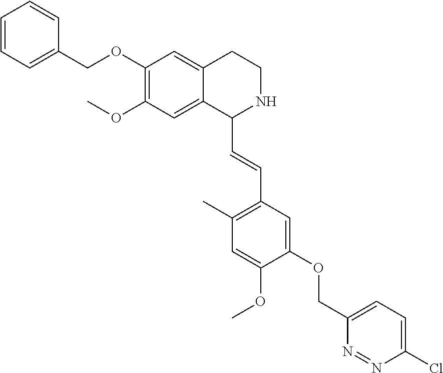 | 6-(benzyloxy)-1-[(E)-2-{5-[(6- chloropyridazin-3-yl)methoxy]-4-methoxy-2- methylphenyl}ethenyl]-7-methoxy-1,2,3,4- tetrahydroisoquinoline | 558.3 |
| 102 | 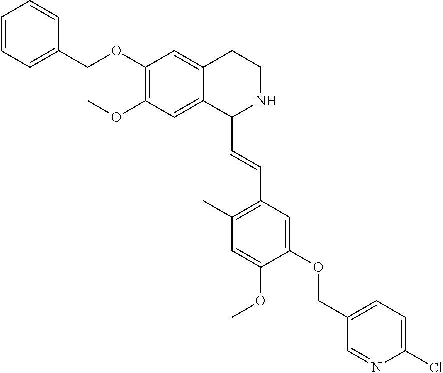 [SMILES: COc1cc(C)c(/C=C/C2NCCc3cc(OCc4ccccc4)c(OC)cc32)cc1OCc1ccc(Cl)nc1] | 6-(benzyloxy)-1-[(E)-2-{5-[(6-chloropyridin-3- yl)methoxy]-4-methoxy-2- methylphenyl}ethenyl]-7-methoxy-1,2,3,4- tetrahydroisoquinoline | 557.3 |
| 103 | 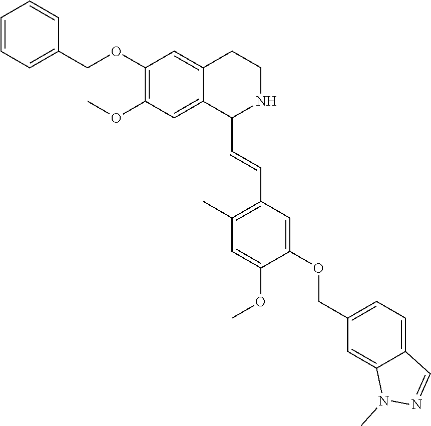 [SMILES: COc1cc(C)c(/C=C/C2NCCc3cc(OCc4ccccc4)c(OC)cc32)cc1OCc1ccc2cnn(C)c2c1] | 6-(benzyloxy)-7-methoxy-1-[(E)-2-{4- methoxy-2-methyl-5-[(1-methyl-1H-indazol- 6-yl)methoxy]phenyl}ethenyl]-1,2,3,4- tetrahydroisoquinoline | 576.2 |
| 104 | 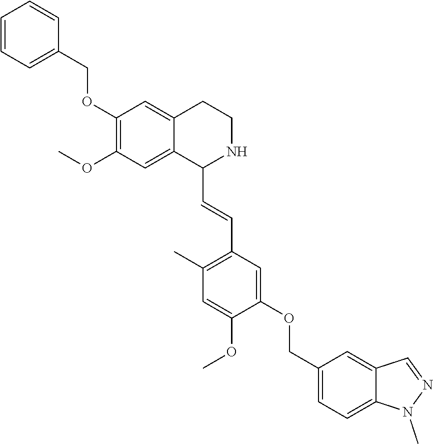 [SMILES: COc1cc(C)c(/C=C/C2NCCc3cc(OCc4ccccc4)c(OC)cc32)cc1OCc1ccc2c(cnn2C)c1] | 6-(benzyloxy)-7-methoxy-1-[(E)-2-{4- methoxy-2-methyl-5-[(1-methyl-1H-indazol- 5-yl)methoxy]phenyl}ethenyl]-1,2,3,4- tetrahydroisoquinoline | 576.3 |
| 105 | 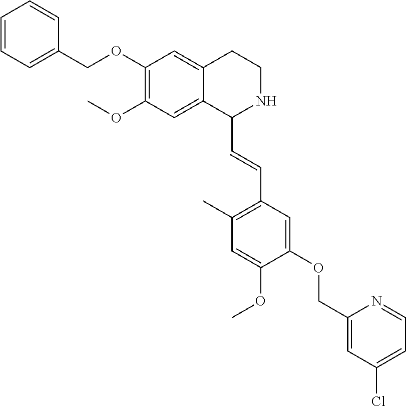 [SMILES: COc1cc2c(cc1OCc1ccccc1)CCNC2/C=C/c1cc(OCc2cc(Cl)ccn2)c(OC)cc1C] | 6-(benzyloxy)-1-[(E)-2-{5-[(4-chloropyridin-2- yl)methoxy]-4-methoxy-2- methylphenyl}ethenyl]-7-methoxy-1,2,3,4- tetrahydroisoquinoline | 557.4 |

TABLE 1-16

| Exam- | | | |
|---|---|---|---|
| ple No. | Structure | compound name | MS |
| 106 | 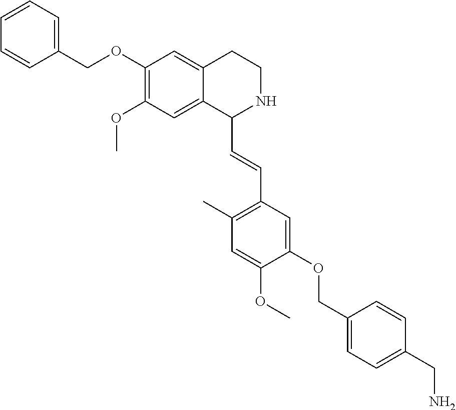 [SMILES: COc1cc(C)c(/C=C/C2NCCc3cc(OCc4ccccc4)c(OC)cc32)cc1OCc1ccc(CN)cc1] | 1-4-[(5-{(E)-2-[6-(benzyloxy)-7-methoxy- 1,2,3,4-tetrahydroisoquinolin-1-yl]ethenyl}- 2-methoxy-4- methylphenoxy)methyl]phenyl}methanamine | 551.3 |
| 107 | 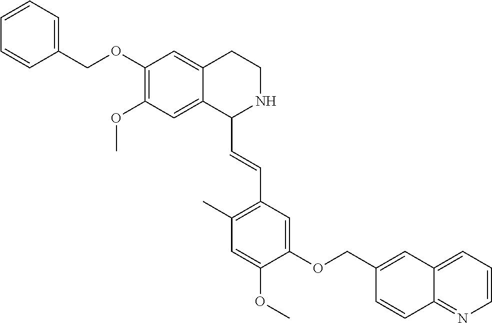 [SMILES: COc1cc(C)c(/C=C/C2NCCc3cc(OCc4ccccc4)c(OC)cc32)cc1OCc1ccc2ncccc2c1] | 6-[(5-{(E)-2-[6-(benzyloxy)-7-methoxy- 1,2,3,4-tetrahydroisoquinolin-1-yl]ethenyl}- 2-methoxy-4- methylphenoxy)methyl]quinoline | 573.5 |
| 108 | 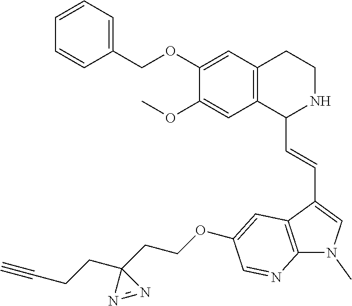 [SMILES: C#CCCC1(CCOc2cnc3c(c2)c(/C=C/C2NCCc4cc(OCc5ccccc5)c(OC)cc42)cn3C)N=N1] | 6-(benzyloxy)-l-[(E)-2-(5-{2-[3-(but-3-yn-l-yl)- 3H-diazirin-3-yl]ethoxy}-l-methyl-lH- pyrrolo[2,3-b] pyridin-3-yl)ethenyl]-7- methoxy-1,2,3,4-tetrahydroisoquinoline | 561.3 |

### Experimental Example 1

1. C. elegans Protocols

Transgenic C. elegans expressing the human TDP-43 wild-type or mutant TDP-43 M337V animal model that mimic aspects of TDP-43 specific ALS disease pathogenesis were generated. The transgenic C. elegans had a single copy of the human TDP-43 gene integrated into its genome. The expression was controlled by an unc-47 promoter and hence human TDP-43 protein was specifically expressed only in the C. elegans motor neurons. Age-synchronized transgenic C. elegans worms at larval stage 4 (L4) was used for screening small-molecules in a swimming induced paralysis (SWIP) assay (Protocol 3). We observed correlation in activity between small-molecules that are active in the TDP-43 cellular assay and those effective in rescuing transgenic C. elegans from swimming induced paralysis.

### 1.1 Transgenic Human TDP-43 (Wild-Type) or TDP-43 (M337V Mutant)

MosSCI homologous-recombination transgenesis was used to create an unc-47p::hTDP-43::unc-54utr or unc-47p::hTDP-43(mutant M337V)::unc-54utr transgenic. Transgenesis requires MOSSCI plasmid inserted with unc-47p::hTDP-43::unc-54utr or unc-47p::hTDP-43(mutant M337V)::unc-54utr. Injection mix uses Standard MosSCI mix. Injections are performed into mos1 ttTi5605 background strain. Extrachromosomal array lines are isolated. Crawling transgenics screened as non-red homozygotes are verified by PCR for insertion/replacement at target locus resulting verified single copy integrated strains.

1.2 Age-Synchronizing C. elegans

Use filtered deionized water to wash worms off of plates and into 15 ml tubes. Spin worms at 1200 rpm for 2 minutes×2. Aspirate supernatant and add 5 ml of NaOH+bleach solution. Vortex gently about every minute and monitor by microscope. The adult worms will split open and their eggs will be released. The adult worms will also dissolve into the solution. Once all adult worms have dissolved, neutralize the reaction by adding 5 ml of M9 buffer. Spin at 2500 rpm for 2 minutes, perform this 3 times. After one wash with 10 ml of water aspirate all but about 200-1000 μl from the 15 ml tube with your pellet. Re-suspend the pellet in your leftover water and drop it onto your plates evenly. This ensures that the larva that hatches will have enough food while they grow over the next few days, store plates at 20° C.

### 1.3 Swimming-Induced Paralysis (SWIP) Assay

The age-synchronized worms are washed off NGM plates in S-media that contains 0.02% Triton. This allows for a more consistent number of worms while pipetting, as less worms stick to the plastic pipette tips. Adjust your volume with S-media until you have 60-70 worms per 20 μl. Worms are scored as paralyzed if their body cannot make a bending “S” movement. Paralyzed worms can often still make small movements with their head or tail. The videos are analyzed using an imageJ C. elegans motility analysis software.

Level of Activity was denoted based on improvement in swimming induced paralysis (SWIP) in human TDP-43 transgenic C. elegans disease model. The automation data measuring paralysis measured average body bends per second of a population. Improvement in SWIP from control in the population of worms was observed.

### Experimental Example 2

### Protocol for Endogenous TDP-43 in SH-SY5Y Cellular Assay Using Immuno-Staining

### Immuno-Staining

### Day 1:

Seed 25,000-30,000 cells (SH-SY5Y cells) per well in 24 well glass bottom plate.

### Day 2:

Add compounds at the desired final concentration in fresh media to the respective wells.
Add equivalent amount of DMSO to control wells.

### Day 3:

Sodium arsenite treatment—Add sodium arsenite at a final concentration of 50 μM to each well. Incubate at 37° C. for 90 mins.
Wash 1× with PBS.
Fixation—Add 4% para formaldehyde (prepared freshly, methanol free) for 15 mins at room temperature.
Wash 3× with PBS.
Blocking and Permeabilization—Add 1% BSA+0.5% saponin for ˜1 hour.
Immunostaining-Add the following primary antibodies in 1% BSA (in PBS) and incubate it overnight at 4° C. rabbit polyclonal TDP-43 C-terminal antibody (Proteintech 12892-1-AP)-1:450
   mouse monoclonal HuR antibody (Santa Cruz sc-5261)-1:500

### Day 4:

Wash 3× with PBST (PBS+0.1% Tween).
Add the following secondary antibodies from Thermofisher Scientific (1:500) in 1% BSA (in PBS) and keep it in dark for 1-2 hours at room temperature.
Alexa 594 anti-rabbit (highly cross-adsorbed)
Alexa 488 anti-mouse (highly cross-adsorbed)
Wash 3× with PBST in dark.
Add DAPI for nuclear staining.

### Imaging

The immuno-stained images were imaged with Nikon Ti inverted fluorescence microscope having CSU-22 spinning disk confocal and EMCCD camera. Plan Apo VC 60×/1.4 Oil objective and NIS-Elements AR software were used for image acquisition. Each image acquired (in .nd format) is converted into three individual channel images (for DAPI, TDP-43, HuR) in .tiff format.

### Image Analysis

The images were analyzed by the open source image analysis software CellProfiler 2.1.1 (offered by Broad Institute of Harvard and MIT—www.cellprofiler.org). This software contains various modules which can be used to analyze images in different ways. A Cell Profiler pipeline from few of these modules was established to analyze our images in order to quantify the number of cytoplasmic TDP-43 positive HuR stress granules. The outline of the Cell Profiler pipeline is described below—

1) The images are loaded and named DAPI, TDP-43 or HuR.
2) Identify primary objects—The DAPI image is used to identify the nucleus as an object.
3) Identify secondary objects—The nucleus is used to identify the cell boundary in the TDP-43 image.
4) Identify tertiary objects—Based on the nucleus and the cell boundary, cytoplasm is identified.
5) Mask images—Using the cytoplasm object, the TDP-43 and HuR images are masked such that only cytoplasmic signal will remain.
6) Enhance features—Enhance the signal from TDP-43 and HuR aggregates in cytoplasm.
7) Identify primary objects—The TDP-43 aggregates in the cytoplasm were identified from the TDP-43 image and HuR aggregates were identified from the HuR image.
8) Relate objects—This module enables the calculation of the number of TDP-43 aggregates which has HuR and vice versa.
9) Export to spreadsheet—This module exports all data into Excel.

This pipeline has to be optimized for every experiment with respect to the intensity threshold and size criteria for primary and secondary objects. From the analyzed data, we calculate the number of TDP-43 positive HuR stress granules.

### Experimental Example 3

### Endogenous TDP-43 Stress Granule (SG) in SH-SY5Y Cells or Patient Fibroblast Cells Using Immunostaining:

In this assay, TDP-43 aggregates in stress granules were quantified after 24 hrs of drug treatment followed by 90 minutes of 100 μM arsenite stress.

On day 1 20,000 patient derived fibroblasts per well were seeded in a 24 well glass bottom plate or 6000 cells per well in a 96 well plate. On day 2 compounds were added at the desired final concentration in fresh media to the respective wells. On day 3 sodium arsenite was added at a final concentration of 500 μM. Incubated at 37° C. for 60 mins. On day 9 wells were washed with PBS and fixed cells using 4% para formaldehyde (in PBS, prepared freshly, methanol free) for 15 mins at room temperature. Washed with PBS, blocked and permeabilized cells with 1% BSA+1% saponin (prepared in PBS) for 1 hour. For Immunostaining primary antibodies were made in 1% BSA (in PBS) and incubated overnight at 4° C. Rabbit polyclonal TDP-43 C-terminal antibody (Proteintech 12892-1-AP)-1:350; mouse monoclonal HuR antibody (Santa Cruz sc-5261)-1:500. On the next day, added the following secondary antibodies Alexa 594 anti-rabbit (Thermofisher Scientific) and Alexa 488 anti-mouse (Thermofisher Scientific) (1:500) in 1% BSA (in PBS) and kept in dark for 1-2 hours at room temperature. Washed and added DAPI in PBS for nuclear staining. The images were analyzed by the open source image analysis software CellProfiler 2.1.1 (offered by Broad Institute of Harvard and MIT—www.cellprofiler.org). This software contains various modules which can be used to analyze images in different ways.

Table 2 provides data from this assay. The lower the number, the fewer the TDP-43 aggregates compared to vehicle (DMSO) alone, which is set at 100.

TABLE 2
Stress granule assay in FTD affected fibroblasts

| | Normalized average | Normalized average |
|---|---|---|
| | number of TDP-43 | number of TDP-43 |
| | positive HuR aggregates | positive HuR aggregates |
| Example | per cell at 1 μM | per cell at 1.5 μM |
| 1 | 67 | |
| 4 | 70.9 | |
| 5 | 83.3 | |
| 7 | 40 | |
| 26 | 59 | |
| AD | 83 | |
| AA | 85 | |
| AC | 85.75 | |
| AG | 87 | |
| AF | | 65.5 |
| AH | | 71.4 |
| AD | | 71.00 |
| AE | | 73.75 |

From the results in Table 2, it is clear that the compound of the present invention has an excellent effect to reduce the TDP-43 aggregation.

### Experimental Example 4

### Nucleocytoplasmic Assay (NCA) in FTD and ALS Patient Fibroblasts Cells:

Skin-derived fibroblasts cells from Amnyotrophic Lateral Sclerosis (ALS) and Frontotemporal Degeneration (FTD) patients or affected individual acquired from the National Institute of Neurological Disorders and Stroke (NINDS #ND32947, ND41006, ND42717) were grown in HyClone DMEM High Glucose (GE Healthcare Life Sciences) supplemented with 15% FBS and 1% NEAA (Non-Essential Amino Acids), at 37° C. in a humidified atmosphere of 5% CO₂. On day 1600 cells per well were seeded in a 96 well glass bottom plate or 1200 cells per well in a 24 well glass bottom plate. Incubated for 4 days, at 37° C. in a humidified atmosphere of 5% CO₂, allowed cells to grow for 4 days. On day added compounds including vehicle control at the desired final concentration in fresh media to the respective wells, allowed cells to grow for an additional 4 days. On day 9 washed wells with PBS and fixed cells using 4% para formaldehyde (in PBS, prepared freshly, methanol free) for 15 mins at room temperature. Washed with PBS, blocked and permeabilized cells with 1% BSA+1% saponin (prepared in PBS) for 1 hour. For immunostaining the following primary antibodies were added in 1% BSA (in PBS) and incubated overnight at 4° C. Rabbit polyclonal TDP-43 C-terminal antibody (Proteintech 12892-1-AP)-1:350; mouse monoclonal HuR antibody (Santa Cruz sc-5261)-1:500. On the next day, added the following secondary antibodies Alexa 594 anti-rabbit (Thermofisher Scientific) and Alexa 488 anti-mouse (Thermofisher Scientific) (1:500) in 1% BSA (in PBS) and kept it in dark for 1-2 hours at room temperature. Wash and add DAPI in PBS for nuclear staining.

The immuno-stained cells were imaged with Nikon Ti inverted fluorescence microscope having CSU-22 spinning disk confocal and EMCCD camera. Plan Apo 20×/0.75 objective and NIS-Elements AR software were used for image acquisition. At least 15 images per well were taken. The exposure times for TDP-43 and HuR remained constant across one experiment. Each image acquired (in .nd format) was exported into three individual channel images (for DAPI, TDP-43, HuR) in .tiff format. The images were analyzed by the open source image analysis software Cell Profiller to calculate the signal intensities of TDP-43 present in the nucleus and cytoplasm in each cell. The DAPI image was used to count the total number of cells. The TDP-43 and HuR images are used to count the number of cells containing TDP-43 and/or HuR nuclear staining.

Table 3 provides data from this assay. The nucleus/cytoplasm ratio of intensities of TDP-43 in each compound-treated (at 1 μM) cells comparing to DMSO-treated cell (which is set as 100) were shown.

TABLE 3
Nucleocytoplasmic assay in patient Fibroblast

| | Mutant TDP-43 | Mutant | Fibroblast from FTD |
|---|---|---|---|
| Ex. | fibroblast | VCP fibroblast | affected individual |
| 1 | 147 | 153 | 148 |
| 4 | 130 | — | — |
| 5 | 137 | 179 | 139 |
| 7 | 143 | 191 | 175 |
| 9 | — | 200 | — |
| 26 | — | 256 | 146 |

From the results in Table 3, it is clear that the compound of the present invention has an excellent effect to relocalize the TDP-43 in cytoplasm into nucleus.

### Formulation Examples

Medicaments containing the compound of the present invention as an active ingredient can be produced, for example, by the following formulations.

1. capsule

| | | |
|---|---|---|
| (1) compound obtained in Example 1 | 10 | mg |
| (2) lactose | 90 | mg |
| (3) microcrystalline cellulose | 70 | mg |
| (4) magnesium stearate | 10 | mg |
| 1 capsule | 180 | mg |

The total amount of the above-mentioned (1), (2) and (3) and 5 mg of (4) are blended and granulated, and 5 mg of the remaining (4) is added. The whole mixture is sealed in a gelatin capsule.

2. tablet

| | | |
|---|---|---|
| (1) compound obtained in Example 1 | 10 | mg |
| (2) lactose | 35 | mg |
| (3) cornstarch | 150 | mg |
| (4) microcrystalline cellulose | 30 | mg |
| (5) magnesium stearate | 5 | mg |
| 1 tablet | 230 | mg |

The total amount of the above-mentioned (1), (2) and (3), 20 mg of (4) and 2.5 mg of (5) are blended and granulated, and 10 mg of the remaining (4) and 2.5 mg of the remaining (5) are added and the mixture is compression formed to give a tablet.

It is understood that the examples and embodiments described herein are for illustrative purposes only and that various modifications or changes in light thereof will be suggested to persons skilled in the art and are to be included within the spirit and purview of this application and scope of the appended claims. It is to be understood that the present invention covers all combinations of aspects and/or embodiments, as well as suitable, convenient and preferred groups described herein. All publications, patents, and patent applications cited herein are hereby incorporated by reference in their entirety for all purposes.

This application is based on patent application No. 63/214,395 filed on Jun. 24, 2021 in USA, the contents of which are hereby incorporated by reference.

1. A method for preventing and/or treating amyotrophic lateral sclerosis, frontotemporal dementia, chronic traumatic encephalopathy, Alzheimer's disease, frontotemporal lobar degeneration, or multisystem proteinopathy in a mammal, which comprises administering an effective amount of a compound represented by the formula (I): 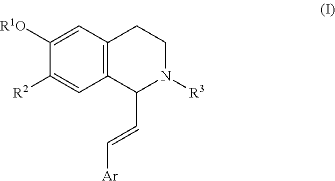
wherein
R¹ is unsubstituted C₁₋₆ alkyl, substituted or unsubstituted phenyl substituted C₁₋₆ alkyl, substituted or unsubstituted C₃₋₁₀ cycloalkyl substituted C₁₋₆ alkyl, or substituted or unsubstituted pyridyl substituted C₁₋₆ alkyl;
R² is H, CF₃, unsubstituted C₁₋₆ alkoxy, or phenyl substituted C₁₋₆ alkoxy;
R³ is H, —C(O)OR⁴, or —C(O)R⁴ or —C(O)NR⁴R⁵ wherein R⁴ and R⁵ are independently selected from unsubstituted C₁₋₆ alkyl, unsubstituted phenyl, and unsubstituted pyridyl; and
Ar is substituted or unsubstituted phenyl, substituted or unsubstituted benzofuryl, substituted or unsubstituted pyrrolopyridyl, substituted or unsubstituted imidazopyrimidinyl, substituted or unsubstituted imidazopyrazinyl, substituted or unsubstituted imidazopyridazinyl, or substituted or unsubstituted imidazopyridyl;
or a hydrate, solvate, or salt thereof, to the mammal.

2. A compound represented by the formula (IA): 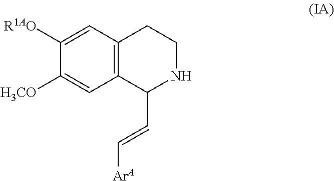
wherein
R^{1A} is a C₁₋₆ alkyl group substituted by phenyl group(s) optionally substituted by 1 to 3 halogen atoms; and
Ar^{A} is
(1) a phenyl group substituted by 2 or 3 substituents selected from
(a) a C₁₋₆ alkyl group,
(b) a C₁₋₆ alkoxy group optionally substituted by 1 to 3 of 5- or 6-membered monocyclic aromatic heterocyclic groups optionally substituted by 1 to 3 halogen atoms, and
(c) a 5- or 6-membered monocyclic aromatic heterocyclic group optionally substituted by 1 to 3 C₁₋₆ alkyl groups, or
(2) a pyrrolopyridyl group substituted by 2 substituents selected from
(a) a C₁₋₆ alkyl group, and
(b) a C₁₋₆ alkoxy group,
or a hydrate, solvate, or salt thereof.

3. A compound represented by the formula (IB): 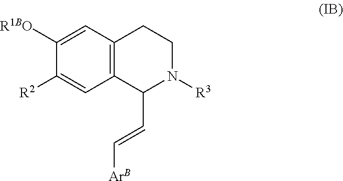
wherein
R^{1B} is unsubstituted C₁₋₆ alkyl, or substituted or unsubstituted phenyl substituted C₁₋₆ alkyl;
R² is H, CF₃, unsubstituted C₁₋₆ alkoxy, or phenyl substituted C₁₋₆ alkoxy;
R³ is H, —C(O)OR⁴, or —C(Q)R⁴ or —C(O)NR⁴R⁵ wherein R⁴ and R⁵ are independently selected from unsubstituted C₁₋₆ alkyl, unsubstituted phenyl, and unsubstituted pyridyl; and
Ar^{B} is substituted or unsubstituted imidazo[1,2-a]pyrimidinyl, substituted or unsubstituted imidazo[1,2-a]pyrazinyl, substituted or unsubstituted imidazo[1,2-b]pyridazinyl, or substituted or unsubstituted imidazo[1,2-a]pyridyl;
or a hydrate, solvate, or salt thereof.
